SEQUENCE LISTING (2 sequences)

SEQ ID NO: 1 - protein, 462 aa, Artificial Sequence
  Synthetic polypeptide
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 2 - protein, 463 aa, Artificial Sequence
  Synthetic polypeptide
MGKEKTHINIVVIGHVDSGKSTTTGHLIYKCGGIDKRTIEKFEKEAAEMGKGSFKYAWVLDKLKAERERGITIDISLWKFETTKYYITIIDAPGHRDFIKNMITGTSQADCAVLIVAAGVGEFEAGISKNGQTREHALLAYTLGVKQLIVGVNKMDSTEPAYSEKRYDEIVKEVSAYIKKIGYNPATVPFVPISGWHGDNMLEPSPNMPWFKGWKVERKEGNASGVSLLEALDTILPPTRPTDKPLRLPLQDVYKIGGIGTVPVGRVETGILRPGMVVTFAPVNITTEVKSVEMHHEALSEALPGDNVGFNVKNVSVKDIRRGNVCGDSKSDPPQEAAQFTSQVIILNHPGQISAGYSPVIDCHTAHIACKFAELKEKIDRRSGKKLEDNPKSLKSGDAAIVEMVPGKPMCVESFSQYPPLGRFAVRDMRQTVAVGVIKNVEKKSGGAGKVTKSAQKAQKAGK

(19) United States
(12) Patent Application Publication
(10) Pub. No.: US 2023/0135758 A1
(43) Pub. Date: May 4, 2023

(54) ELONGATION FACTOR 1-ALPHA INHIBITORS AND USES THEREOF

(71) Applicant: The Regents of the University of California, Oakland, CA (US)

(72) Inventors: John Taunton, San Francisco, CA (US); Davide Ruggero, San Francisco, CA (US); Haoyuan Wang, San Francisco, CA (US); Keely Oltion, San Francisco, CA (US); Haojun Yang, San Francisco, CA (US)

(21) Appl. No.: 17/760,090

(22) PCT Filed: Feb. 5, 2021
(86) PCT No.: PCT/US21/16790

§ 371 Date: Aug. 3, 2022

Related U.S. Application Data
(60) Provisional application No. 62/970,979, filed on Feb. 6, 2020.
(60) Provisional application No. 63/031,233, filed on May 28, 2020.

(51) Int. Cl. C07K 7/64; A61K 45/06; A61K 38/12

(52) U.S. Cl. CPC C07K 7/64; A61K 45/06; A61K 38/12

(57) ABSTRACT

Disclosed herein, inter alia, are compounds for inhibiting Elongation Factor 1-alpha and uses thereof.

### CROSS-REFERENCES TO RELATED APPLICATIONS

This application claims the benefit of U.S. Provisional Application No. 62/970,979, filed Feb. 6, 2020, and U.S. Provisional Application No. 63/031,233, filed May 28, 2020, which are incorporated herein by reference in their entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-679001WO_Sequence_Listing_ST25.txt, created Feb. 1, 2021, 8,529 bytes, machine format IBM-PC, MS Windows operating system, is hereby incorporated by reference.

### BACKGROUND

Natural products continue to stimulate the identification of new biological targets and therapeutic strategies. Among natural products, cyclic peptides are unique owing to their diverse but constrained structures as well as high target affinity and selectivity. The cyclic peptide A3, first isolated in 2010 from an Aspergillus fungus in Malaysia, displays extraordinary cytotoxicity to cancer cells. However, in the original report, 7 out of 11 stereocenters in A3 were ambiguous and therefore unassigned, making it difficult to pursue chemical synthesis and biological studies. Disclosed herein, inter alia, are solutions to these and other problems known in the art.

### BRIEF SUMMARY OF THE INVENTION

In an aspect is provided a compound having the formula:

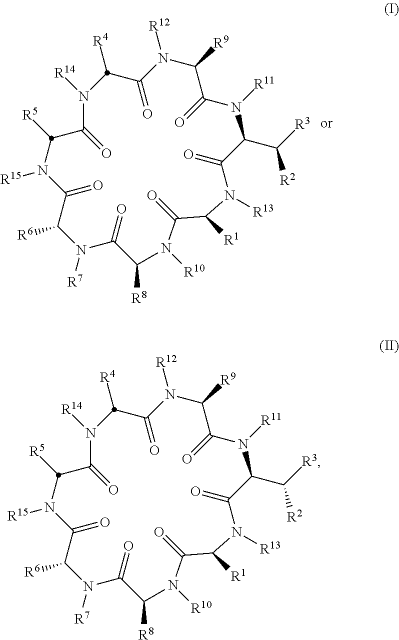

pharmaceutically acceptable salt thereof.

R¹ is substituted or unsubstituted alkyl or substituted or unsubstituted heteroalkyl.

R² is —OCX²₃, —OCH₂X², —OCHX²₂, —SR^{2B}, —NR^{2A}R^{2B}, or —OR^{2B}.

R^{2A} and R^{2B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{2A} and R^{2B} substituents bonded to the same nitrogen atom may optionally be joined to form a substituted or unsubstituted heterocycloalkyl or substituted or unsubstituted heteroaryl.

R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁴ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶ and R⁷ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶ and R⁷ substituents may optionally be joined to form, in combination with the —CHN— connecting the two substituents, a substituted or unsubstituted heterocycloalkyl.

R⁸ and R⁹ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroary.

R¹⁰, R¹¹, R¹², R¹³, R¹⁴, and R¹¹ are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X² is independently —F, —Cl, —Br, or —I.

In an aspect is provided a pharmaceutical composition including a compound as described herein, including embodiments, and a pharmaceutically acceptable excipient.

In an aspect is provided a method of decreasing the level of Elongation Factor 1-alpha protein activity in a subject, the method including administering a compound described herein to the subject.

In an aspect is provided a method of inhibiting cancer growth in a subject in need thereof, the method including administering to the subject in need thereof an effective amount of a compound described herein, or a pharmaceutically acceptable salt thereof.

In an aspect is provided a method of inhibiting cancer cell growth, the method including contacting the cancer cell with an effective amount of a compound described herein.

In an aspect is provided a method of treating cancer in a subject in need thereof, the method including administering to the subject in need thereof an effective amount of a compound described herein.

In an aspect is provided a method of treating a viral infection in a subject in need thereof, the method including administering to the subject in need thereof an effective amount of a compound described herein.

In an aspect is provided a method of treating acute respiratory distress syndrome (ARDS) in a subject in need thereof, the method including administering to the subject in need thereof an effective amount of a compound described herein.

In an aspect is provided a method of treating a coronavirus disease in a subject in need thereof, the method including administering to the subject in need thereof an effective amount of a compound described herein.

In an aspect is provided a method of treating arrhythmia in a subject in need thereof, the method including administering to the subject in need thereof an effective amount of a compound described herein.

In an aspect is provided a method of treating a SARS-CoV-2 infection in a subject in need thereof, the method including administering to the subject in need thereof an effective amount of a compound described herein.

In an aspect is provided a method of treating a SARS-CoV-2 associated disease in a subject in need thereof, the method including administering to the subject in need thereof an effective amount of a compound described herein.

### BRIEF DESCRIPTION OF THE DRAWINGS

FIG. 1. Chemical structures of “A3” (ambiguous stereochemistry indicated with squiggle bonds), (S,S)-A3, and (S,R)-A3.

FIGS. 2A-2C. Effects of dA3, (S,S)-A3, and (S,R)-A3 on cell proliferation under continuous treatment conditions. HCT116, H929, and MM1S cells were treated continuously with the indicated compounds for 72 h, and cell viability was assessed relative to DMSO controls using the Alamar Blue assay (triplicate values, mean±SD). Note “dA3” is equivalent to “ternatin-4” in the figures, descriptions of the figures, and examples.

FIGS. 3A-3C. Effects of dA3, (S,S)-A3, and (S,R)-A3 on cell proliferation under washout conditions. HCT116, H929, and MM1S cells were briefly treated with the indicated compounds (HCT116: 100 nM for 4 h; H929: 100 nM for 1 h; MM1S: 200 nM for 1 h), followed by stringent washout and incubation in compound-free media. After indicated time points post-washout, cell viability was assessed using the CellTiter Glo assay (triplicate values, mean±SD). Note “dA3” is equivalent to “ternatin-4” in the figures, descriptions of the figures, and examples.

FIGS. 4A-4C. Effects of dA3, (S,S)-A3, and (S,R)-A3 on protein synthesis. Cellular protein synthesis rates were measured by 0-propargyl puromycin (OPP) labeling after (FIG. 4A) 10 min or (FIG. 4B) 24 h of continuous treatment with the indicated compounds (normalized to 0.1% DMSO control), or (FIG. 4C) transient exposure (100 nM for 4 h), followed by washout into compound-free media and incubation for 24 h. Cycloheximide (CHX) was used at 50 μg/mL. In (FIG. 4C), protein synthesis rates were measured at the indicated time points after washout and normalized to cells treated with 0.1% DMSO for 4 h, followed by washout into compound-free media and incubation for 24 h (triplicate values, mean±SD). Note “dA3” is equivalent to “ternatin-4” in the figures, descriptions of the figures, and examples.

FIGS. 5A-5C. (S,R)-A3 is efficacious in the Eμ-Myc mouse lymphoma model. (FIG. 5A) Mouse survival vs. treatment day (treatment longer survival). (FIG. 5B) Photographs of mice on treatment day 26. (FIG. 5C) Body weight vs. treatment day. BW gain in the vehicle group (treatment days 14-28) reflects rapid tumor growth.

FIG. 6. SRA3 prolongs survival in a MYC-driven lymphoma model. Two weeks after IV injection of Ep-Myc transgenic tumor cells, mice were randomized into vehicle and SRA3 treatment groups (1.5 and 2 mg/kg IP injection, 3 doses/week). P<0.0002 Curves left to right Vehicle, 1.5 mpk (S,R)-A3, 2mpk (S,R)-A3.

FIGS. 7A-7B. (FIG. 7A) Screening conditions to synthesize Boc-dhML-OMe 3 via Cu(I)-promoted SN2′ reaction. (FIG. 7B) Synthesis of Fmoc-dhML 5.

FIGS. 8A-8B. Solid-phase synthesis and macrocyclization strategy. (FIG. 8A) Identification of alternative cyclization sites. (FIG. 8B) Scheme for solid-phase synthesis of linear heptapeptide precursors, followed by solution-phase cyclization to provide ternatin-4, SR-A3, and SS-A3.

FIGS. 9A-9C. SR-A3 inhibits protein synthesis via eEF1A and exhibits a time-dependent potency shift. FIG. 9A) (eEF1A A399V higher curve, WT lower curve at 1000 nM) Wild-type and eEF1A-mutant (A399V) HCT116 cells were treated with SR-A3 for 72 h. Cell proliferation (% DMSO control) was quantified using AlamarBlue. FIG. 9B) (ternatin-4 and SR-A3 left overlapping curves at transition and SS-A3 right curve at transition from about 100 to about 10% protein synthesis) and FIG. 9C) (SR-A3 right curve at transition, SS-A3 middle curve at transition, ternatin-4 left curve at transition) HCT116 cells were treated with the indicated compounds for 24 h or 10 min, respectively, and protein synthesis (% DMSO control) was quantified after pulse labeling with 0-propargyl puromycin for 1 h (see Supporting Information). Data points (% DMSO control) are mean values±SD (n=3). Note “dA3” is equivalent to “ternatin-4” in the figures, descriptions of the figures, and examples.

FIGS. 10A-10B. N-Me-β-OH-Leu stereospecifically endows SR-A3 with increased cellular residence time. FIG. 10A) (ternatin-4 top curve, SS-A3 middle curve, SR-A3 lowest curve at 24 hours) HCT116 cells were treated with the indicated compounds (100 nM) or DMSO for 4 h, followed by rigorous washout into compound-free media. At the indicated time points post-washout, cells were pulse-labeled with OPP (1 h), and OPP incorporation was quantified. Normalized data (% DMSO control) are mean values±SD (n=3). FIG. 10B) (DMSO highest, SS-A3 second highest, ternatin-4 third highest, SR-A3 lowest curve at 70 hours) HCT116 cells were treated with the indicated compounds (100 nM) or DMSO for 4 h, followed by rigorous washout into compound-free media. At the indicated time points post-washout, cell proliferation was quantified using the CellTiter-Glo assay. Normalized data (% DMSO control at t=0 h post-washout) are mean values+SD (n=3). ***, P<0.001; ****, P<0.0001. Note “dA3” is equivalent to “ternatin-4” in the figures, descriptions of the figures, and examples.

FIG. 11. SRA3 has higher liver microsome stability than SSA3 or ternatin-4. Liver microsome stability analysis. Percent remaining was determined after 30 min incubation.

FIG. 12. Low-dose SRA3 inhibits proliferation without significantly affecting global protein synthesis. HCT116 cells were treated for 24 hrs. Protein synthesis curve is the right curve and proliferation is the left curve.

FIG. 13. SRA3 analogs

FIG. 14. SRA3 pharmacokinetic data. PK studies were performed in C57BL/6 mice

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. In embodiments, an alkenyl includes one or more double bonds. An alkynyl may include more than one triple bond and/or one or more double bonds in addition to the one or more triple bonds. In embodiments, an alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. In embodiments, the alkylene is fully saturated. In embodiments, the alkylene is monounsaturated. In embodiments, the alkylene is polyunsaturated. In embodiments, an alkenylene includes one or more double bonds. In embodiments, an alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. In embodiments, a heteroalkenylene includes one or more double bonds. In embodiments, a heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. In embodiments, a bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, a bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl. In embodiments, the term “heterocycloalkyl” means a monocyclic, bicyclic, or a multicyclic heterocycloalkyl ring system. In embodiments, heterocycloalkyl groups are fully saturated. In embodiments, a bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. In embodiments, a fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). In embodiments, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings. In embodiments, a fused ring heteroaryl group is multiple rings fused together wherein at least one of the fused rings is a heteroaromatic ring and wherein the multiple rings are attached to the parent molecular moiety through any atom contained within a heteroaromatic ring of the multiple rings. A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

[symbol]

The symbol “C” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

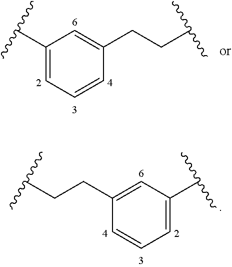 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₂₀ alkyl, C₁-C₁₂ alkyl, C₁-C₈ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₂₀ alkyl, C₁-C₁₂ alkyl, C₁-C₅ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (b) alkyl (e.g., C₁-C₂₀ alkyl, C₁-C₁₂ alkyl, C₁-C₅ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth herein, for example in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on a R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{2.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1} As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g. R¹¹, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g. R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g. R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B. etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g. when R^{WW.1} is R^{WW.2} substituted alkyl, examples of groups so formed include but are not limited to itself optionally substituted by 1 or more R^{WW.2}, which R^{WW.2} is optionally substituted by one or more R^{WW.3}. By way of example when R^{WW.1} is alkyl, groups that could be formed, include but are not limited to:

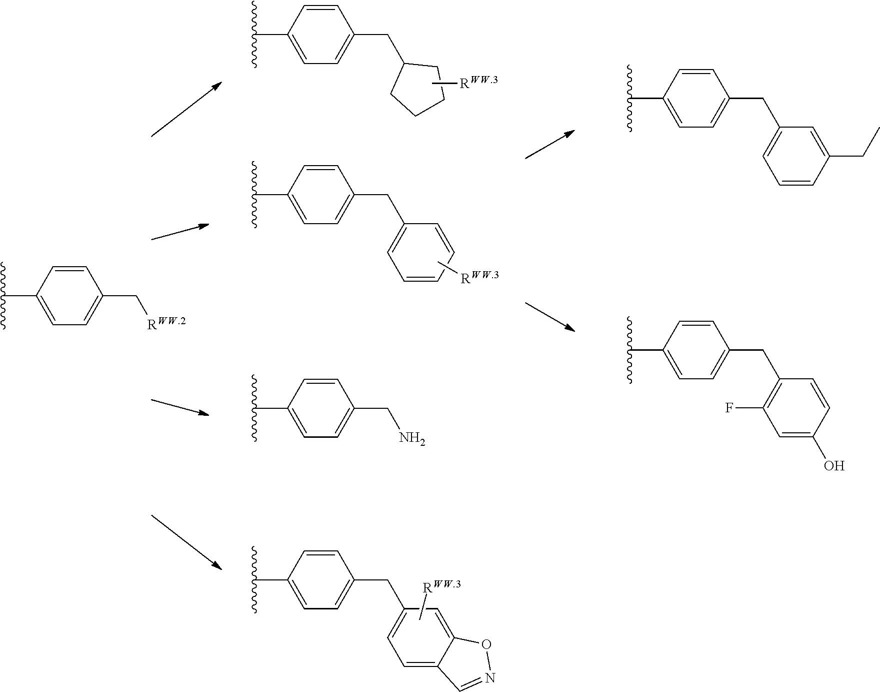

R^{WW.1} is independently oxo,

halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo,
halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo,

halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g. substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo,

halogen, —CX^{LWW.1}₃, —CHX^{LWW.1}₂, CH₂X^{LWW.1}, —OCX^{LWW.1}₃, —OCH₂X^{LWW.1}, —OCHX^{LWW1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo,
halogen, —CX^{LWW.1}₃, —CHX^{LWW.1}₂, CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo,

halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo,
halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo,

halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g. 1, 2, 3, 1A, 2A, 3A, 1B, 2B, 3B. etc.). R^{WW.1}, R^{WW.2}, and R^{WW.3}, are as defined above.

In the event that any L linker group recited in a claim or chemical formula description set forth herein (i.e. an L^{WW} substituent) is not explicitly defined, then that L group (L^{WW} group) is herein defined as independently —O—, —NH—, —C(O)—, —C(O)NH—, —NHC(O)—, —NHC(O)NH—, —C(O)O—, —OC(O)—, —S—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B. etc.). R^{LWW.1}, as well as R^{LWW.2} and R^{LWW.3}, are as defined above. Alternatively, the L group is a bond.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers. Compounds described herein may be drawn using any representation method known in the field of chemistry. For example, stereochemistry may be drawn using a solid black dot on an atom (e.g., carbon atom) showing the presence of a hydrogen atom attached to the atom (e.g., carbon atom) with the black dot, wherein the hydrogen atom is projecting forward and out of the plane toward the viewer.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein may be bound, for example, by covalent bond, linker (e.g. a first linker of second linker), or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions, and the like). In embodiments, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g. phosphines) to form, for example, phosphate diester bonds;
(n) azides coupled to alkynes using copper catalyzed cycloaddition click chemistry; and
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

A “Eukaryotic Translation Elongation Factor 1 alpha inhibitor”, “Elongation Factor 1-alpha inhibitor” or “EEF1A inhibitor” refers to a compound (e.g. a compound described herein) that decreases the activity of Elongation Factor 1-alpha 1 and/or Elongation Factor 1-alpha 2 or decreases the level of activity of Elongation Factor 1-alpha 1 and/or Elongation Factor 1-alpha 2 (e.g., in a cell or in a subject in need; by reducing the level of Elongation Factor 1-alpha 1 and/or Elongation Factor 1-alpha 2 protein in the cell or subject in need) when compared to a control, such as absence of the compound or a compound with known inactivity. In embodiments, a “Elongation Factor 1-alpha inhibitor” refers to a compound (e.g. a compound described herein) that decreases the activity of (Elongation Factor 1-alpha 1 or Elongation Factor 1-alpha 2) or decreases the level of activity of (Elongation Factor 1-alpha 1 or Elongation Factor 1-alpha 2). In embodiments, a “Elongation Factor 1-alpha inhibitor” refers to a compound (e.g. a compound described herein) that decreases the activity of (Elongation Factor 1-alpha 1 and Elongation Factor 1-alpha 2) or decreases the level of activity of (Elongation Factor 1-alpha 1 and Elongation Factor 1-alpha 2).

A “Eukaryotic Translation Elongation Factor 1 alpha 1 inhibitor”, “Elongation Factor 1-alpha 1 inhibitor”, or “EEF1A1 inhibitor” refers to a compound (e.g. a compound described herein) that decreases the activity of Elongation Factor 1-alpha 1 or decreases the level of activity of Elongation Factor 1-alpha 1 (e.g., in a cell or in a subject in need; by reducing the level of Elongation Factor 1-alpha 1 protein in the cell or subject in need) when compared to a control, such as absence of the compound or a compound with known inactivity.

A “Eukaryotic Translation Elongation Factor 1 alpha 2 inhibitor”, “Elongation Factor 1-alpha 2 inhibitor”, or “EEF1A2 inhibitor” refers to a compound (e.g. a compound described herein) that decreases the activity of Elongation Factor 1-alpha 2 or decreases the level of activity of Elongation Factor 1-alpha 2 (e.g., in a cell or in a subject in need; by reducing the level of Elongation Factor 1-alpha 2 protein in the cell or subject in need) when compared to a control, such as absence of the compound or a compound with known inactivity.

The terms “inhibitor,” “repressor” or “antagonist” or “downregulator” interchangeably refer to a substance capable of detectably decreasing the expression or activity of a given gene or protein. In embodiments, an “inhibitor” refers to a compound (e.g. compounds described herein) that reduces activity when compared to a control, such as absence of the compound or a compound with known inactivity. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “Eukaryotic Translation Elongation Factor 1 alpha”, “Elongation Factor 1-alpha”, or “EEF1A” refers to the alpha 1subunit isoform and/or alpha 2 subunit isoform of the elongation factor-1 complex responsible for delivery of aminoacyl tRNA to the ribosome. The term includes any recombinant or naturally-occurring form of Elongation Factor 1-alpha 1 and/or Elongation Factor 1-alpha 2, including variants thereof that maintain Elongation Factor 1-alpha 1 and/or Elongation Factor 1-alpha 2 function or activity (e.g., within at least 30%, 40%, 50%, 60%, 70%, 80%, 90%, 95%, or 100% function or activity compared to wildtype Elongation Factor 1-alpha 1 and/or Elongation Factor 1-alpha 2, respectively). In embodiments, Elongation Factor 1-alpha is encoded by the EEF1A1 and/or EEF1A2 gene. In embodiments, Elongation Factor 1-alpha has the amino acid sequence corresponding to Elongation Factor 1-alpha 1 and/or Elongation Factor 1-alpha 2, as described herein, including in embodiments (e.g., Entrez 1915, UniProt P68104, RefSeq (protein) NP_001393, RefSeq (protein) NP_001393.1, Entrez 1917, UniProt Q05639, RefSeq (protein) NP_001949, and/or RefSeq (protein) NP_001949.1). In embodiments, the term “Eukaryotic Translation Elongation Factor 1 alpha” or “EEF1A” refers to the Elongation Factor 1-alpha 1 or Elongation Factor 1-alpha 2. In embodiments, the term “Eukaryotic Translation Elongation Factor 1 alpha” or “EEF1A” refers to the Elongation Factor 1-alpha 1 and Elongation Factor 1-alpha 2. In embodiments, the Elongation Factor 1-alpha is EEF1A1. In embodiments, the Elongation Factor 1-alpha is EEF1A2. In embodiments, the Elongation Factor 1-alpha is EEF1A1 and EEF1A2. In embodiments, the Elongation Factor 1-alpha is EEF1A1 or EEF1A2.

The term “Eukaryotic Translation Elongation Factor 1 alpha 1”, “Elongation Factor 1-alpha 1”, or “EEF1A1” refers to the alpha 1 subunit isoform of the elongation factor-1 complex responsible for delivery of aminoacyl tRNA to the ribosome. The term includes any recombinant or naturally-occurring form of Elongation Factor 1-alpha 1, including variants thereof that maintain Elongation Factor 1-alpha 1 function or activity (e.g., within at least 30%, 40%, 50%, 60%, 70%, 80%, 90%, 95%, or 100% function or activity compared to wildtype Elongation Factor 1-alpha 1). In embodiments, Elongation Factor 1-alpha 1 is encoded by the EEF1A1 gene. In embodiments, Elongation Factor 1-alpha 1 has the amino acid sequence set forth in or corresponding to Entrez 1915, UniProt P68104, RefSeq (protein) NP_001393. In embodiments, Elongation Factor 1-alpha 1 has the amino acid sequence set forth in or corresponding to RefSeq (protein) NP_001393.1. In embodiments, Elongation Factor 1-alpha 1 has the amino acid sequence.

(SEQ ID NO: 1)
MGKEKTHINIVVIGHVDSGKSTTTGHLIYKCGGIDKRTIEKFEKEAAEM
GKGSFKYAWVLDKLKAERERGITIDISLWKFETSKYYVTIIDAPGHRDF
IKNMITGTSQADCAVLIVAAGVGEFEAGISKNGQTREHALLAYTLGVKQ
LIVGVNKMDSTEPPYSQKRYEEIVKEVSTYIKKIGYNPDTVAFVPISGW
NGDNMLEPSANMPWFKGWKVTRKDGNASGTTLLEALDCILPPTRPTDKP
LRLPLQDVYKIGGIGTVPVGRVETGVLKPGMVVTFAPVNVTTEVKSVEM
HHEALSEALPGDNVGFNVKNVSVKDVRRGNVAGDSKNDPPMEAAGFTAQ
VIILNHPGQISAGYAPVLDCHTAHIACKFAELKEKIDRRSGKKLEDGPK
FLKSGDAAIVDMVPGKPMCVESFSDYPPLGRFAVRDMRQTVAVGVIKAV
DKKAAGAGKVTKSAQKAQKAK

The term “Eukaryotic Translation Elongation Factor 1 alpha 2”, “Elongation Factor 1-alpha 2”, or “EEF1A2” refers to the alpha 2 subunit isoform of the elongation factor-1 complex responsible for delivery of aminoacyl tRNA to the ribosome. The term includes any recombinant or naturally-occurring form of Elongation Factor 1-alpha 2, including variants thereof that maintain Elongation Factor 1-alpha 2 function or activity (e.g., within at least 30%, 40%, 50%, 60%, 70%, 80%, 90%, 95%, or 100% function or activity compared to wildtype Elongation Factor 1-alpha 2). In embodiments, Elongation Factor 1-alpha 2 is encoded by the EEF1A2 gene. In embodiments, Elongation Factor 1-alpha 2 has the amino acid sequence set forth in or corresponding to Entrez 1917, UniProt Q05639, RefSeq (protein) NP_001949. In embodiments, Elongation Factor 1-alpha 2 has the amino acid sequence set forth in or corresponding to RefSeq (protein) NP_001949.1. In embodiments, Elongation Factor 1-alpha 2 has the amino acid sequence:

(SEQ ID NO: 2)
MGKEKTHINIVVIGHVDSGKSTTTGHLIYKCGGIDKRTIEKFEKEAAEM
GKGSFKYAWVLDKLKAERERGITIDISLWKFETTKYYITIIDAPGHRDF
IKNMITGTSQADCAVLIVAAGVGEFEAGISKNGQTREHALLAYTLGVKQ
LIVGVNKMDSTEPAYSEKRYDEIVKEVSAYIKKIGYNPATVPFVPISGW
HGDNMLEPSPNMPWFKGWKVERKEGNASGVSLLEALDTILPPTRPTDKP
LRLPLQDVYKIGGIGTVPVGRVETGILRPGMVVTFAPVNITTEVKSVEM
HHEALSEALPGDNVGFNVKNVSVKDIRRGNVCGDSKSDPPQEAAQFTSQ
VIILNHPGQISAGYSPVIDCHTAHIACKFAELKEKIDRRSGKKLEDNPK
SLKSGDAAIVEMVPGKPMCVESFSQYPPLGRFAVRDMRQTVAVGVIKNV
EKKSGGAGKVTKSAQKAQKAGK

The term “MYC”, “bHLH transcription factor”, or “c-MYC” refers to the transcription factor MYC. The term includes any recombinant or naturally-occurring form of MYC, including variants thereof that maintain MYC function or activity (e.g., within at least 30%, 40%, 50%, 60%, 70%, 80%, 90%, 95%, or 100% function or activity compared to wildtype MYC). In embodiments, MYC is encoded by the MYC gene. In embodiments, MYC has the amino acid sequence set forth in or corresponding to Entrez 4609, UniProt P01106, RefSeq (protein) NP_002458, or RefSeq (protein) NP_001341799. In embodiments, MYC has the amino acid sequence set forth in or corresponding to RefSeq (protein) NP_002458.2. In embodiments, MYC has the amino acid sequence set forth in or corresponding to RefSeq (protein) NP_001341799.1.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator. In some embodiments, an Elongation Factor 1-alpha associated disease modulator is a compound that reduces the severity of one or more symptoms of a disease associated with Elongation Factor 1-alpha (e.g., EEF1A1, EEF1A2, or (EEF1A1 and EEF1A2)) (e.g. cancer). An Elongation Factor 1-alpha modulator is a compound that increases or decreases the activity or function or level of activity or level of function of Elongation Factor 1-alpha (e.g., EEF1A1, EEF1A2, or (EEF1A1 and EEF1A2)). In some embodiments, an Elongation Factor 1-alpha associated disease modulator is a compound that reduces the severity of one or more symptoms of a disease associated with Elongation Factor 1-alpha (e.g., EEF1A1, EEF1A2, or (EEF1A1 and EEF1A2)) (e.g. cancer). An Elongation Factor 1-alpha modulator is a compound that increases or decreases the activity or function or level of activity or level of function of Elongation Factor 1-alpha (e.g., EEF1A1, EEF1A2, or (EEF1A1 and EEF1A2)). In some embodiments, an Elongation Factor 1-alpha 1 associated disease modulator is a compound that reduces the severity of one or more symptoms of a disease associated with Elongation Factor 1-alpha 1 (e.g. cancer). An Elongation Factor 1-alpha 1 modulator is a compound that increases or decreases the activity or function or level of activity or level of function of Elongation Factor 1-alpha 1. In some embodiments, an Elongation Factor 1-alpha 1 associated disease modulator is a compound that reduces the severity of one or more symptoms of a disease associated with Elongation Factor 1-alpha 1 (e.g. cancer). An Elongation Factor 1-alpha 1 modulator is a compound that increases or decreases the activity or function or level of activity or level of function of Elongation Factor 1-alpha 1. In some embodiments, an Elongation Factor 1-alpha 2 associated disease modulator is a compound that reduces the severity of one or more symptoms of a disease associated with Elongation Factor 1-alpha 2 (e.g. cancer). An Elongation Factor 1-alpha 2 modulator is a compound that increases or decreases the activity or function or level of activity or level of function of Elongation Factor 1-alpha 2. In some embodiments, an Elongation Factor 1-alpha 2 associated disease modulator is a compound that reduces the severity of one or more symptoms of a disease associated with Elongation Factor 1-alpha 2 (e.g. cancer). An Elongation Factor 1-alpha 2 modulator is a compound that increases or decreases the activity or function or level of activity or level of function of Elongation Factor 1-alpha 2.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer associated with Elongation Factor 1-alpha activity, Elongation Factor 1-alpha associated cancer, Elongation Factor 1-alpha associated disease (e.g., cancer)) means that the disease (e.g. cancer) is caused by (in whole or in part), or a symptom of the disease is caused by (in whole or in part) the substance or substance activity or function. For example, a cancer associated with Elongation Factor 1-alpha activity or function may be a cancer that results (entirely or partially) from aberrant Elongation Factor 1-alpha function (e.g., EEF1A1, EEF1A2, or (EEF1A1 and EEF1A2)) (e.g. enzyme activity, protein-protein interaction, signaling pathway) or a cancer wherein a particular symptom of the disease is caused (entirely or partially) by aberrant Elongation Factor 1-alpha (e.g., EEF1A1, EEF1A2, or (EEF1A1 and EEF1A2)) activity or function. As used herein, what is described as being associated with a disease, if a causative agent, could be a target for treatment of the disease. For example, a cancer associated with Elongation Factor 1-alpha activity or function or a Elongation Factor 1-alpha associated disease (e.g., cancer), may be treated with a Elongation Factor 1-alpha modulator or Elongation Factor 1-alpha inhibitor, in the instance where increased Elongation Factor 1-alpha activity or function (e.g. signaling pathway activity) causes the disease (e.g., cancer). A cancer associated with Elongation Factor 1-alpha activity or function or a Elongation Factor 1-alpha associated disease (e.g., cancer), may be treated with a Elongation Factor 1-alpha modulator or Elongation Factor 1-alpha activator, in the instance where decreased Elongation Factor 1-alpha activity or function (e.g. signaling pathway activity) causes the disease (e.g., cancer).

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components. For example, binding of a Elongation Factor 1-alpha with a compound as described herein may reduce the level of a product of the Elongation Factor 1-alpha catalyzed reaction or the level of a downstream derivative of the product or binding may reduce the interactions between the Elongation Factor 1-alpha protein or a Elongation Factor 1-alpha reaction product and downstream effectors or signaling pathway components, resulting in changes in cell growth, proliferation, or survival.

In this disclosure, “comprises,” “comprising,” “containing” and “having” and the like can have the meaning ascribed to them in U.S. Patent law and can mean “includes,” “including,” and the like. “Consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The terms “disease” or “condition” refer to a state of being or health status of a patient or subject capable of being treated with the compounds or methods provided herein. The disease may be a cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, acute lymphoblastic leukemia (ALL), chronic lymphocytic leukemia (CLL), acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, acute myeloid leukemia (AML), chronic myeloid leukemia (CML), leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myelodysplastic syndrome (MDS), myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma (MCL), follicular lymphoma, marginal zone B-cell lymphoma (MZL), mucosa-associated lymphatic tissue lymphoma (MALT), extranodal lymphoma, nodal (monocytoid B-cell) lymphoma, splenic lymphoma, diffuse large cell B-lymphoma (DLBCL), activated B-cell subtype diffuse large B-cell lymphoma (ABC-DBLCL), germinal center B-cell like diffuse large B-cell lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungocides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment (e.g., the patient has a disease, the patient suffers from a disease).

The term “prevent” refers to a decrease in the occurrence of Elongation Factor 1-alpha (e.g., EEF1A1, EEF1A2, or (EEF1A1 and EEF1A2)) associated disease symptoms or Elongation Factor 1-alpha (e.g., EEF1A1, EEF1A2, or (EEF1A1 and EEF1A2)) associated disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

In embodiments, an “anticancer agent” as used herein refers to a molecule (e.g. compound, peptide, protein, or nucleic acid) used to treat cancer through destruction or inhibition of cancer cells or tissues. Anticancer agents may be selective for certain cancers or certain tissues. In embodiments, anticancer agents herein may include epigenetic inhibitors and multi-kinase inhibitors.

In embodiments, “Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), immunotherapy (e.g., cellular immunotherapy, antibody therapy, cytokine therapy, combination immunotherapy, etc.), radioimmunotherapy (e.g., anti-CD20 monoclonal antibody conjugated to ¹¹¹In, ⁹⁰Y, or ¹³¹I, etc.), immune checkpoint inhibitors (e.g., CTLA4 blockade, PD-1 inhibitors, PD-L1 inhibitors, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

The term “protecting group” is used in accordance with its ordinary meaning in organic chemistry and refers to a moiety covalently bound to a heteroatom to prevent reactivity of the heteroatom during one or more chemical reactions performed prior to removal of the protecting group. In embodiments, the protecting group is covalently bound to a heteroatom that is part of a heteroalkyl, heterocycloalkyl or heteroaryl moiety. Typically, a protecting group is bound to a heteroatom (e.g., O or N) during a part of a multistep synthesis wherein it is not desired to have the heteroatom react (e.g., a chemical reduction) with a reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS), tert-butyl dimethylsilyl (TBS)). In embodiments the protecting group is an amine protecting group. Non-limiting examples of amine protecting groups include carboxybenzyl (Cbz), p-methoxybenzyl carbonyl (Moz or MeOZ), tert-butyloxycarbonyl (Boc), 9-fluorenylmethyloxycarbonyl (Fmoc), acetyl (Ac), benzoyl (Bz), benzyl (Bn), carbamate, p-methoxybenzyl ether (PMB), 3,4-dimethoxybenzyl (DMPM), p-methoxyphenyl (PMP), pivaloyl (Piv), tosyl (Ts), and phthalimide.

### II. Compounds

In an aspect is provided a compound having the formula:

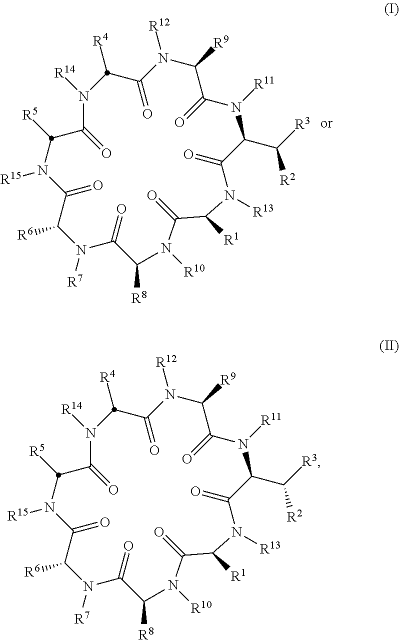

pharmaceutically acceptable salt thereof.

R¹ is substituted or unsubstituted alkyl or substituted or unsubstituted heteroalkyl.

R² is —OCX²₃, —OCH₂X², —OCHX²₂, —SR^{2B}, —NR^{2A}R^{2B}, or —OR^{2B}.

R^{2A} and R^{2B} are independently

hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{2A} and R^{2B} substituents bonded to the same nitrogen atom may optionally be joined to form a substituted or unsubstituted heterocycloalkyl or substituted or unsubstituted heteroaryl.

R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁴ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶ and R⁷ are independently hydrogen,

halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶ and R⁷ substituents may optionally be joined to form, in combination with the —CHN— connecting the two substituents, a substituted or unsubstituted heterocycloalkyl.

R⁸ and R⁹ are independently hydrogen,

halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCCl₃, —OCF₃, —OCBr₃, —OCl₃, —OC HCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroary.

R¹⁰, R¹¹, R¹², R¹³, R¹⁴, and R″ are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X² is independently —F, —Cl, —Br, or —I.

In embodiments, the compound has the formula:

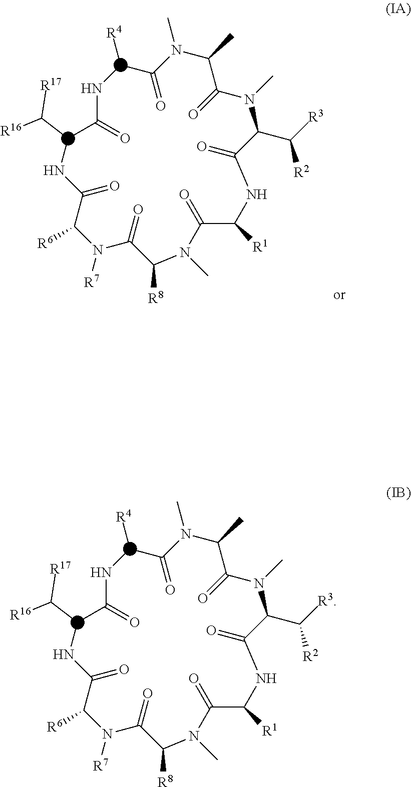

R¹, R², R³, R⁴, R⁶, R⁷, and R⁸ are as described herein, including in embodiments.

R¹⁶ is —OCX¹⁶₃, —OCH₂X¹⁶, —OCHX¹⁶₂, —SR^{16B}, —NR^{16A}R^{16B} or —OR^{16B};

R^{16A} and R^{16B} are independently

hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{16A} and R^{16B} substituents bonded to the same nitrogen atom may optionally be joined to form a substituted or unsubstituted heterocycloalkyl or substituted or unsubstituted heteroaryl;

R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X¹⁶ is independently —F, —Cl, —Br, or —I;

In embodiments, the compound has the formula:

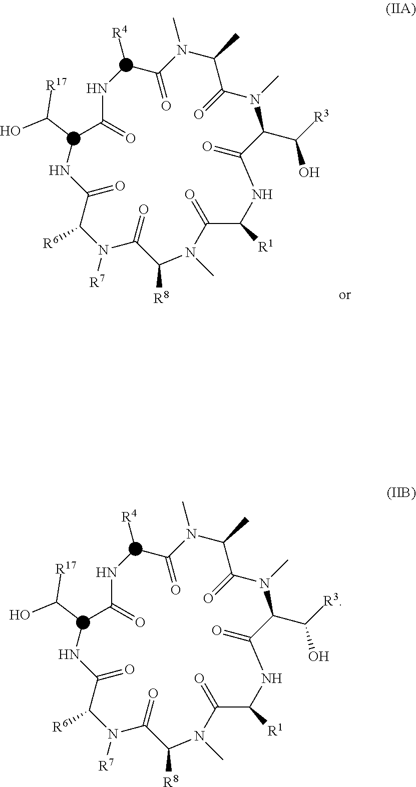

R¹, R³, R⁴, R⁶, R⁷, R⁸ and R¹⁷ are as described herein, including in embodiments.

In embodiments, the compound has the formula:

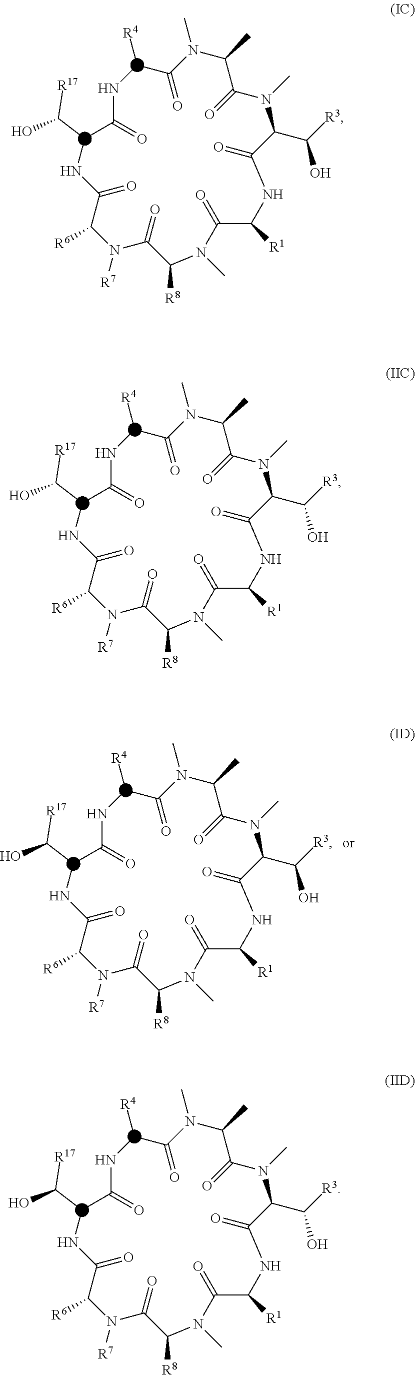

R¹, R³, R⁴, R⁶, R⁷, R⁸ and R¹⁷ are as described herein, including in embodiments.

In embodiments, the compound is

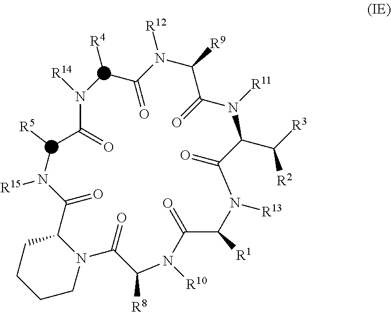

wherein R¹, R², R³, R⁴, R⁵, R⁸, R⁹, R¹⁰, R¹¹, R¹², R¹³, R¹⁴ and R¹⁵ are as described herein, including in embodiments. In embodiments, the compound is

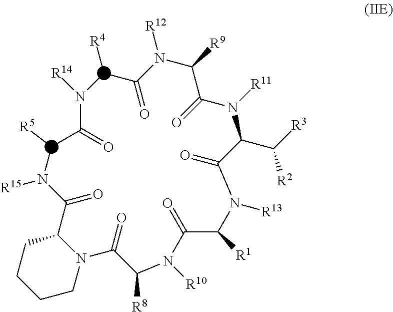

wherein R¹, R², R³, R⁴, R⁵, R⁸R⁹, R¹⁰, R¹¹, R¹², R¹³, R¹⁴ and R¹⁵ are as described herein, including in embodiments. In embodiments, the compound is

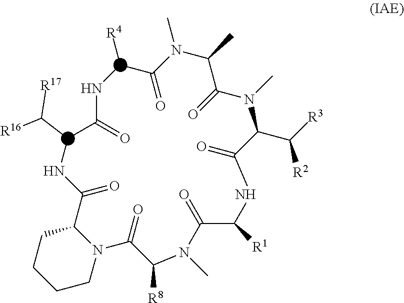

wherein R¹, R², R³, R⁴, R⁸, R¹⁶ and R¹⁷ are as described herein, including in embodiments. In embodiments, the compound is

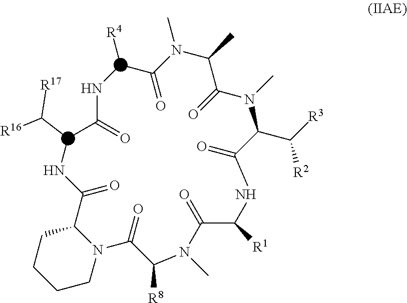

wherein R¹, R², R³, R⁴, R⁸, R¹⁶ and R¹⁷ are as described herein, including in embodiments. In embodiments, the compound is

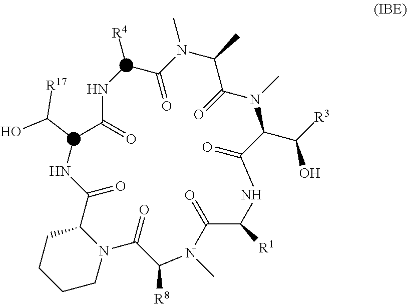

wherein R¹, R³, R⁴, R⁸, and R¹⁷ are as described herein, including in embodiments. In embodiments, the compound is

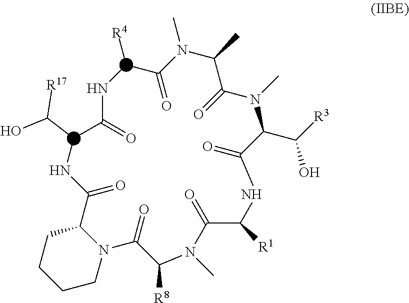

wherein R¹, R³, R⁴, R⁸, and R¹⁷ are as described herein, including in embodiments. In embodiments, the compound is

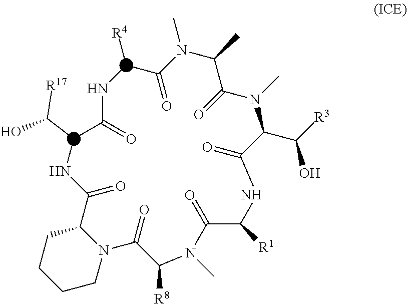

wherein R¹, R³, R⁴, R⁸, and R¹⁷ are as described herein,

including in embodiments. In embodiments, the compound is

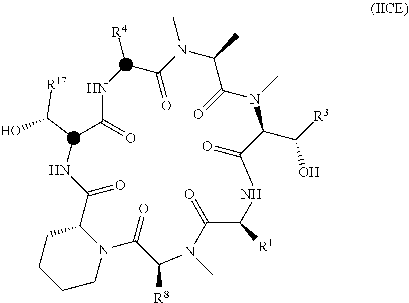

wherein R¹, R³, R⁴, R⁸, and R¹⁷ are as described herein, including in embodiments. In embodiments, the compound is

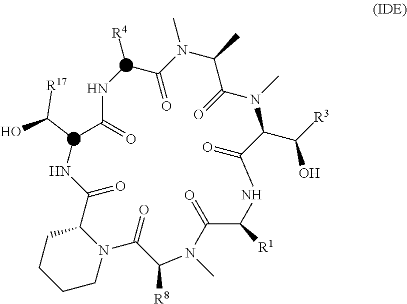

wherein R¹, R³, R⁴, R⁸, and R¹⁷ are as described herein, including in embodiments. In embodiments, the compound is

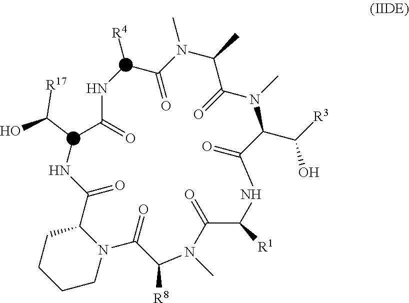

wherein R¹, R³, R⁴, R⁸, and R¹⁷ are as described herein, including in embodiments.

In an aspect is provided a compound having the formula:

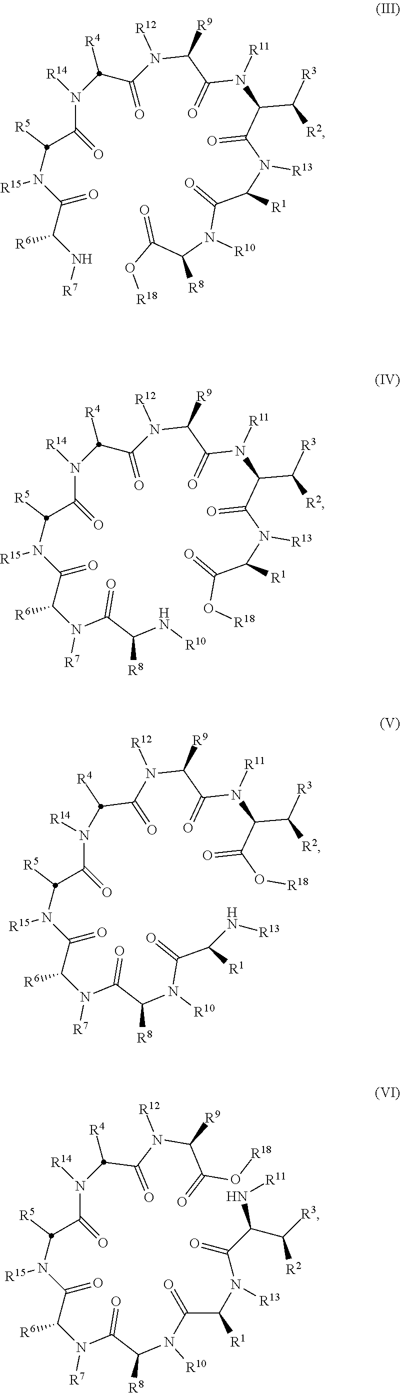

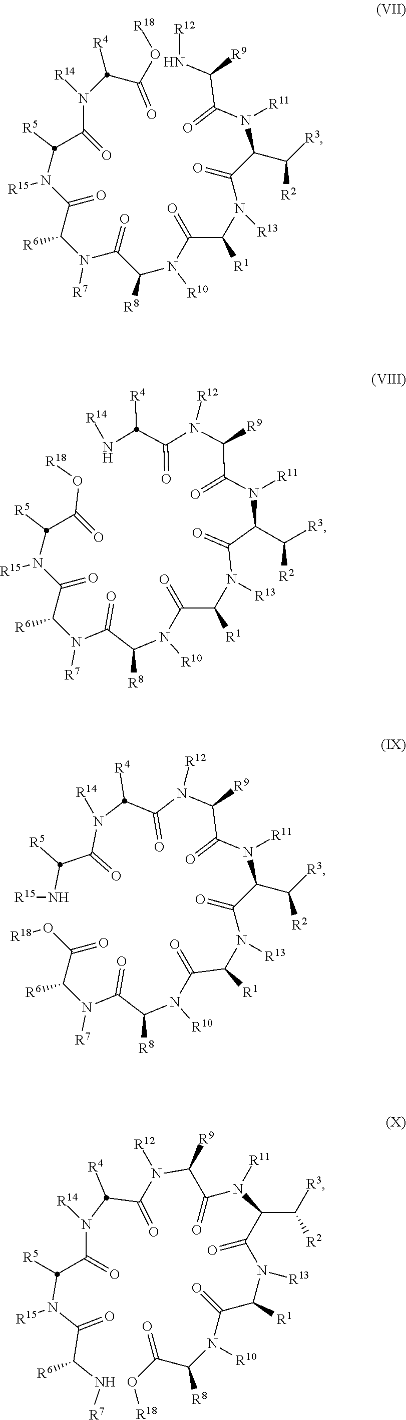

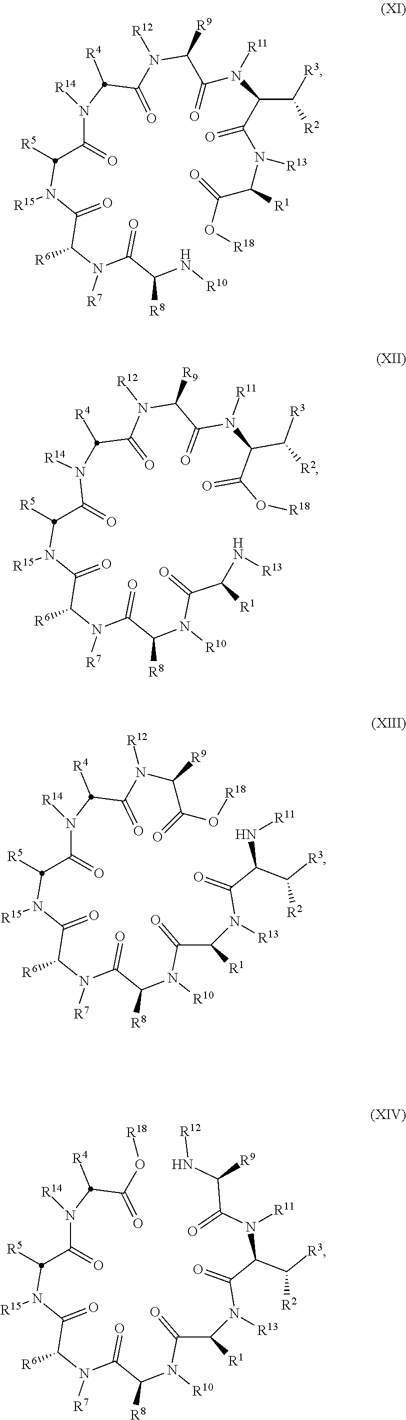

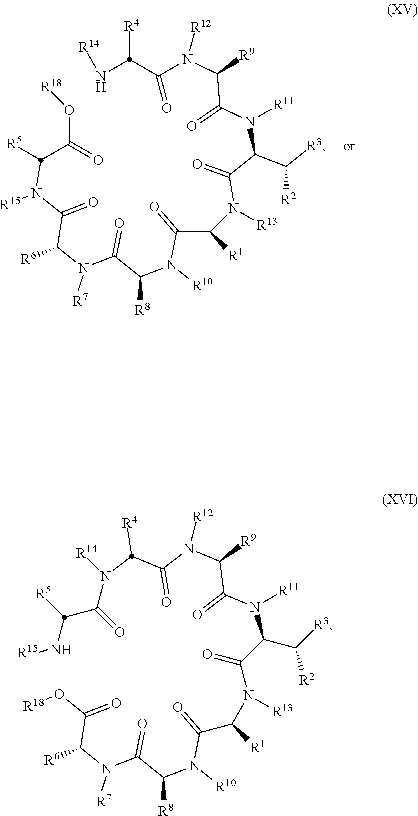

pharmaceutically acceptable salt thereof.

R¹, R², R³, R⁴, R⁵, R⁶, R⁷, R⁸, R⁹, R¹⁰, R¹¹, R¹², R¹³, R¹⁴, and R¹⁵ are as described herein, including in embodiments.

R¹⁸ is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a resin moiety.

In embodiments, the compound has the formula:

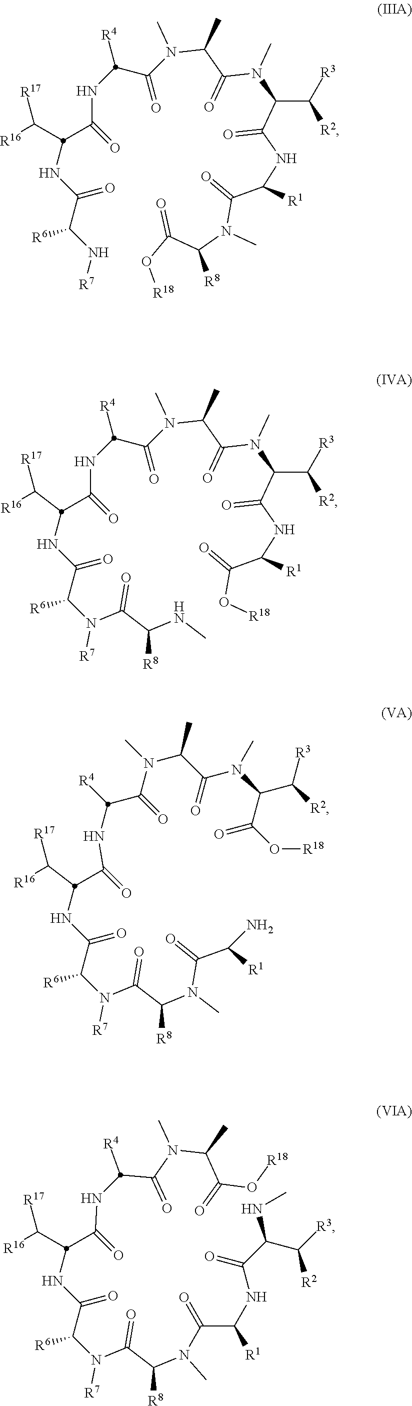

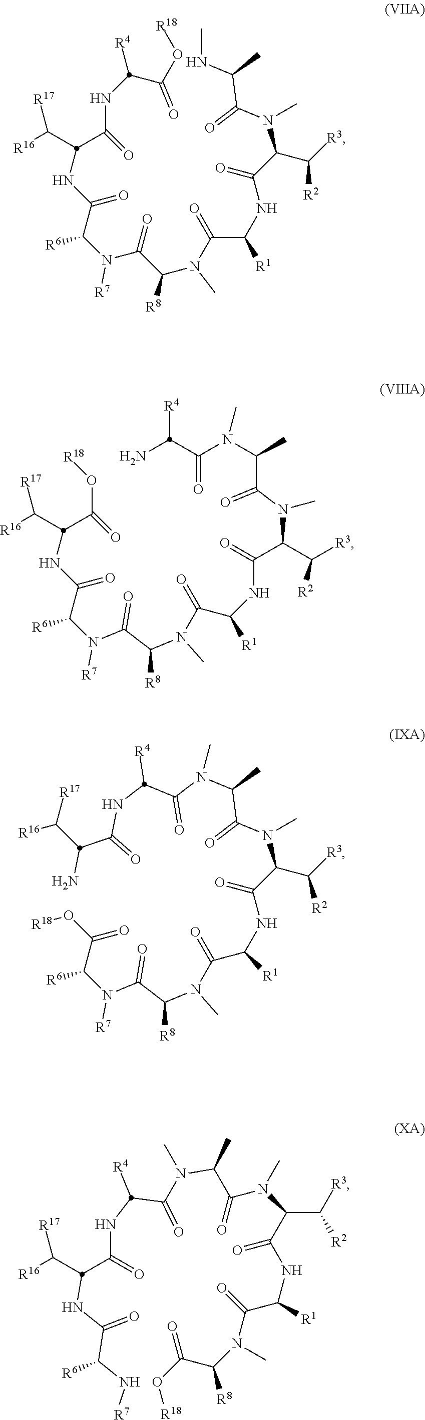

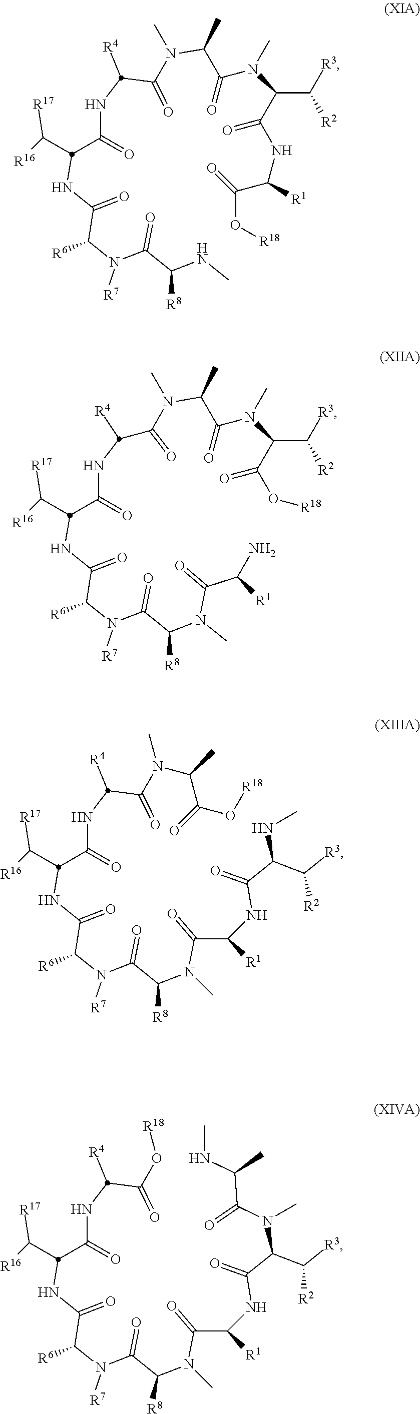

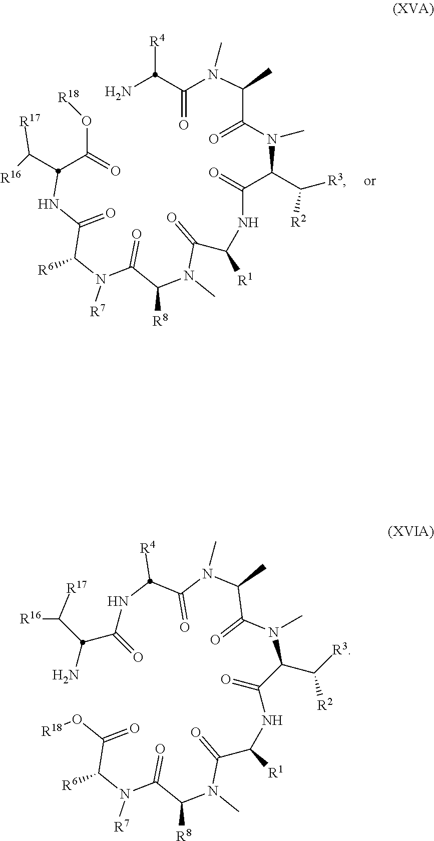

R¹, R², R³, R⁴, R⁶, R⁷, R⁸, R¹⁶, R¹⁷, and R¹⁸ are as described herein, including in embodiments.

In embodiments, the compound has the formula:

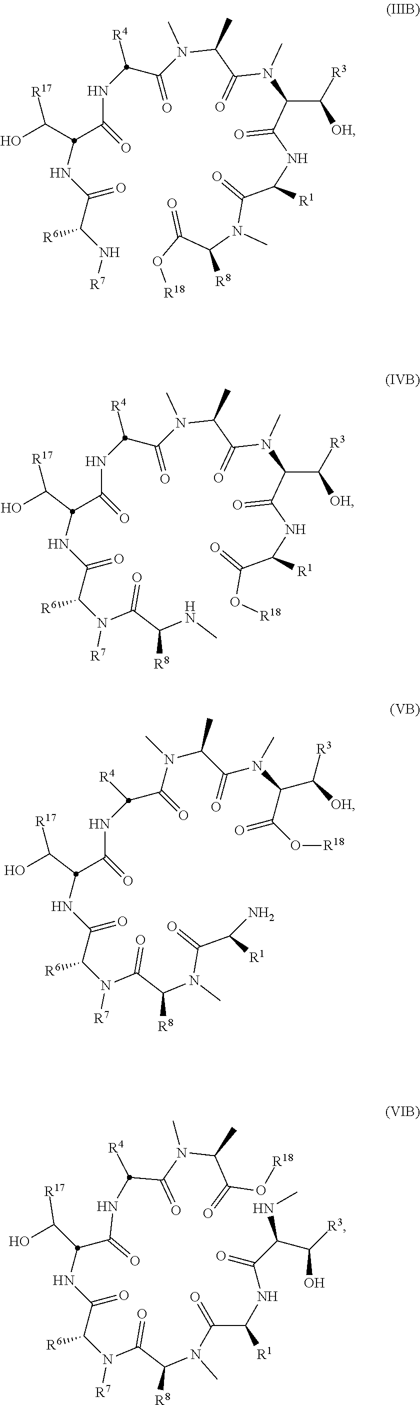

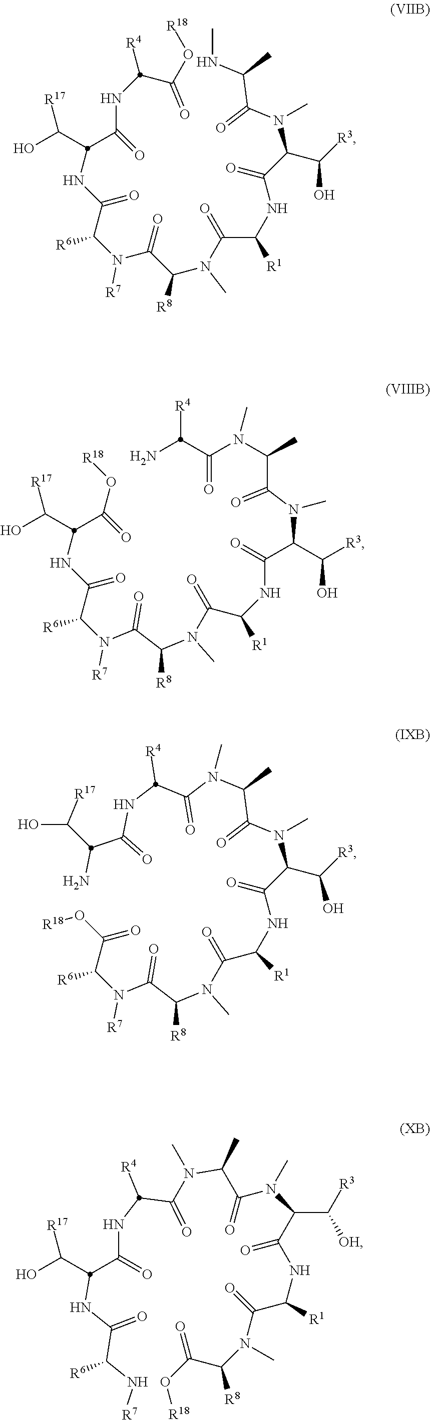

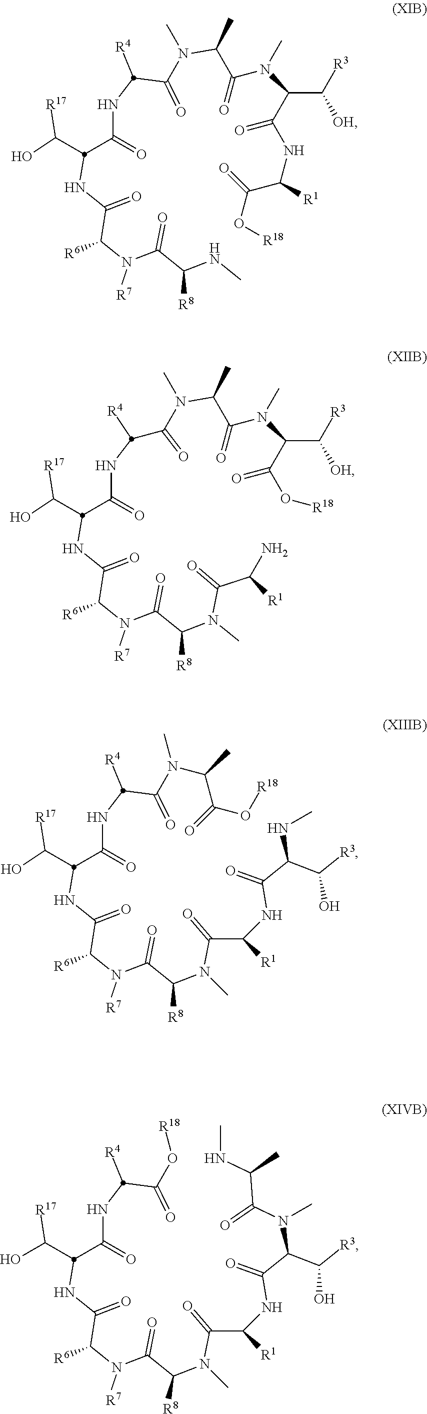

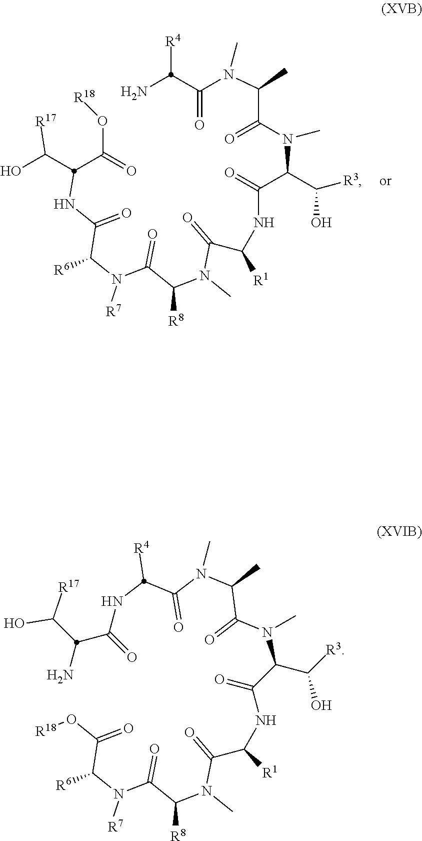

R¹, R³, R⁴, R⁶, R⁷, R⁸, R¹⁷, and R¹⁸ are as described herein, including in embodiments.

In embodiments, the compound has the formula:

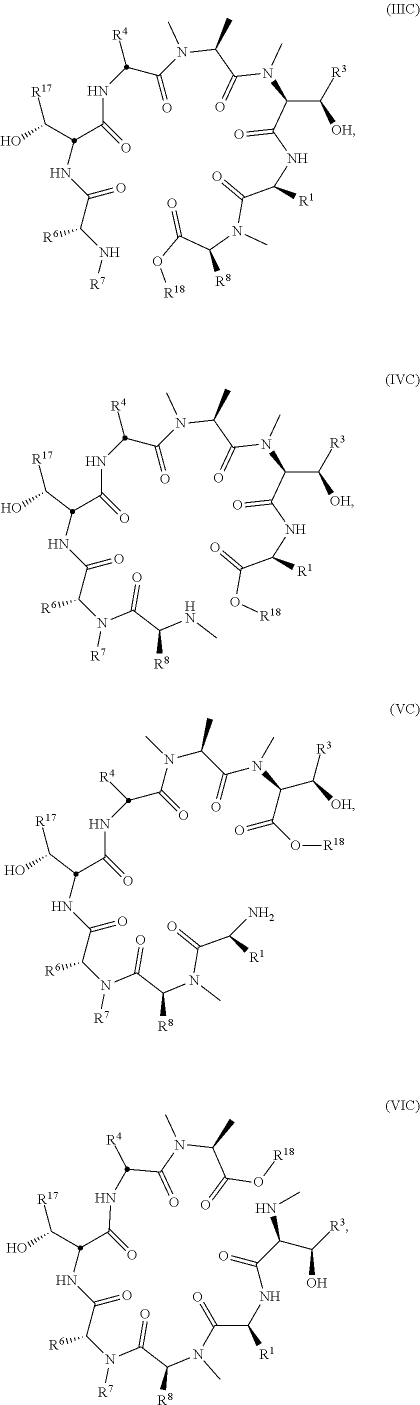

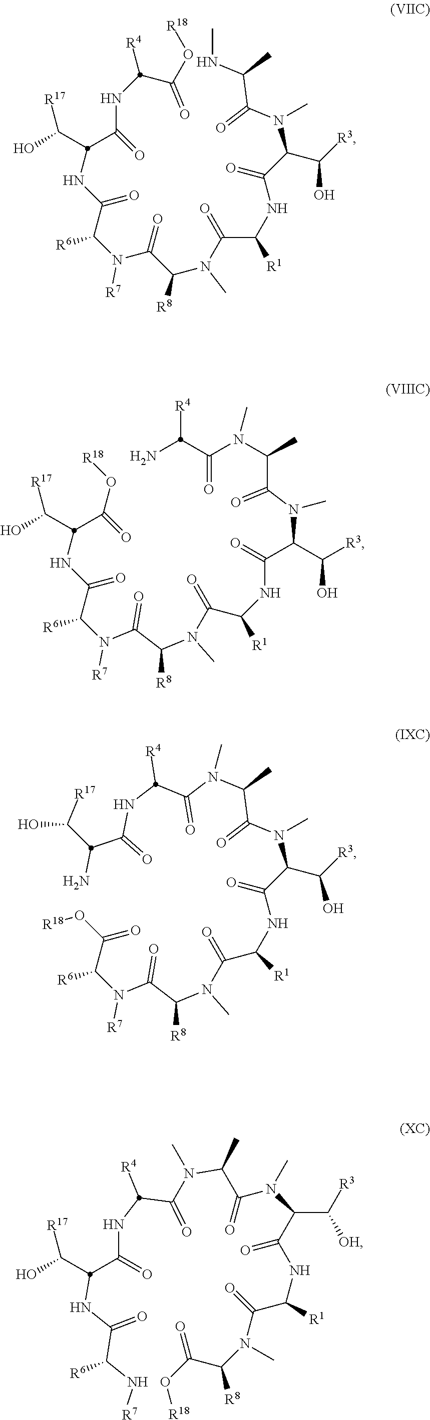

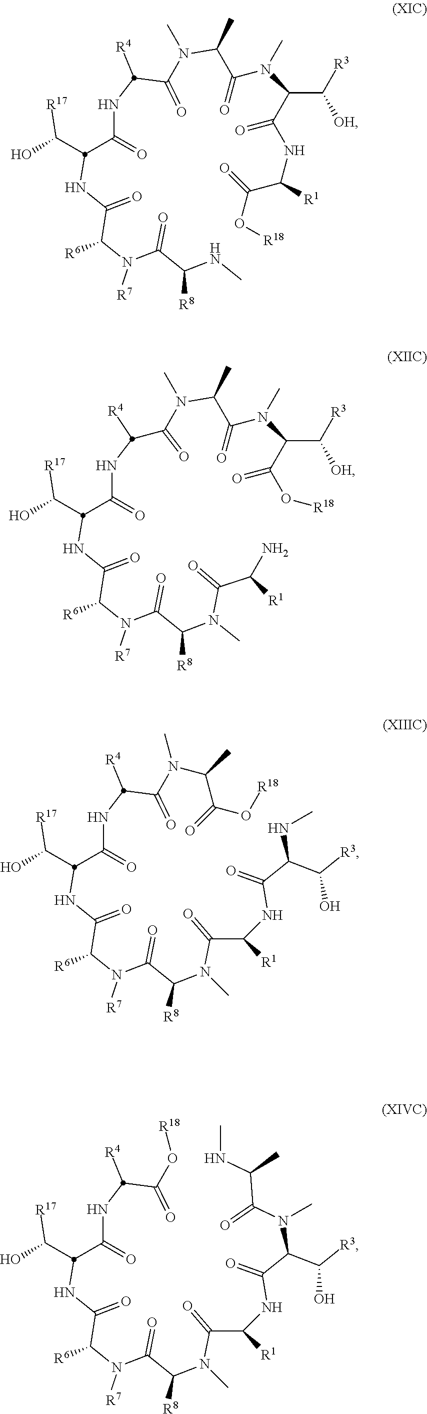

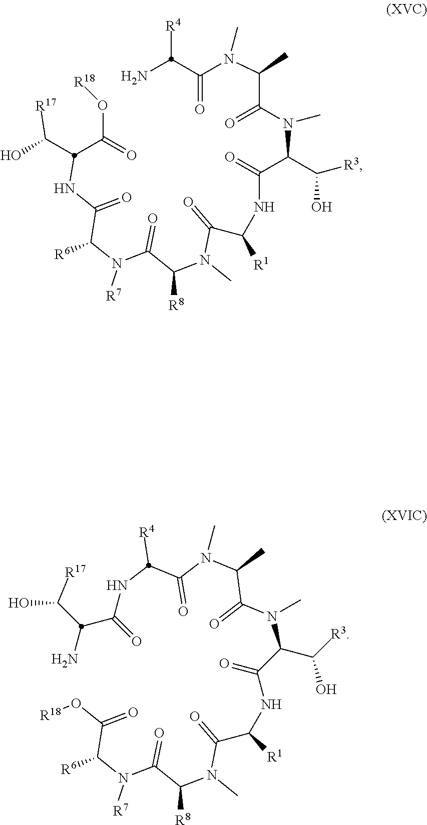

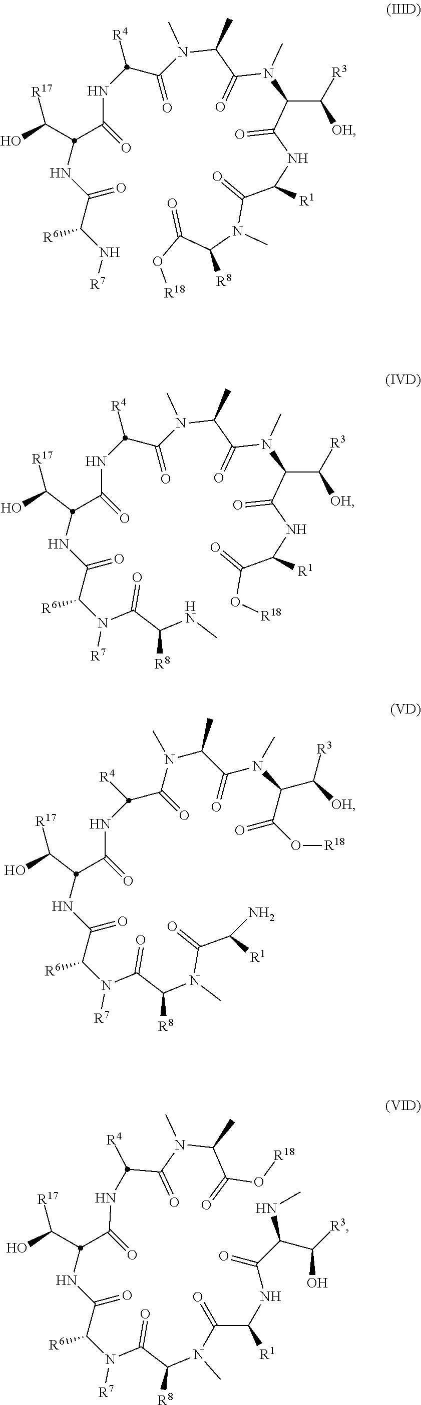

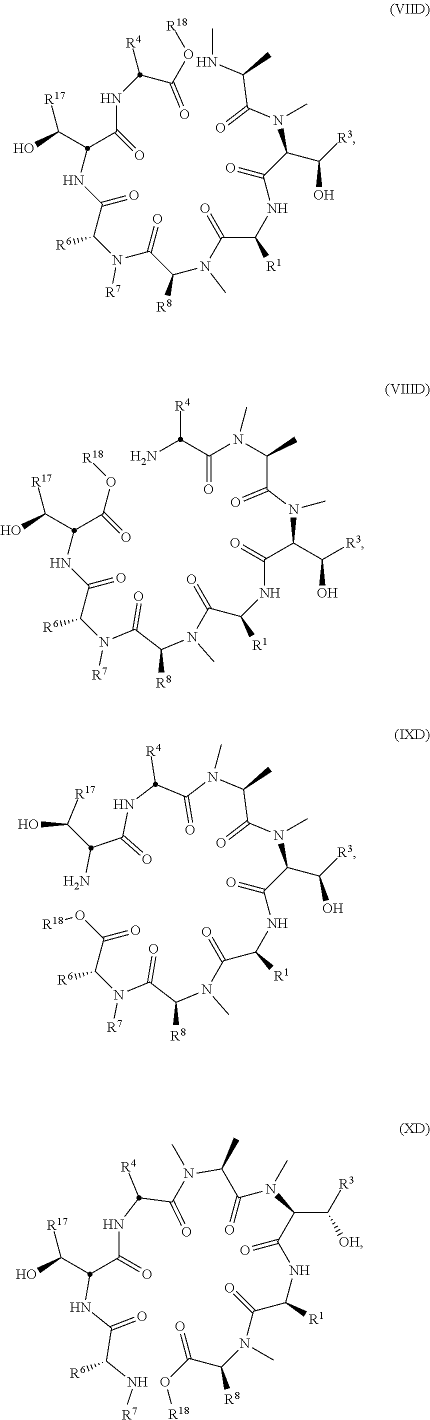

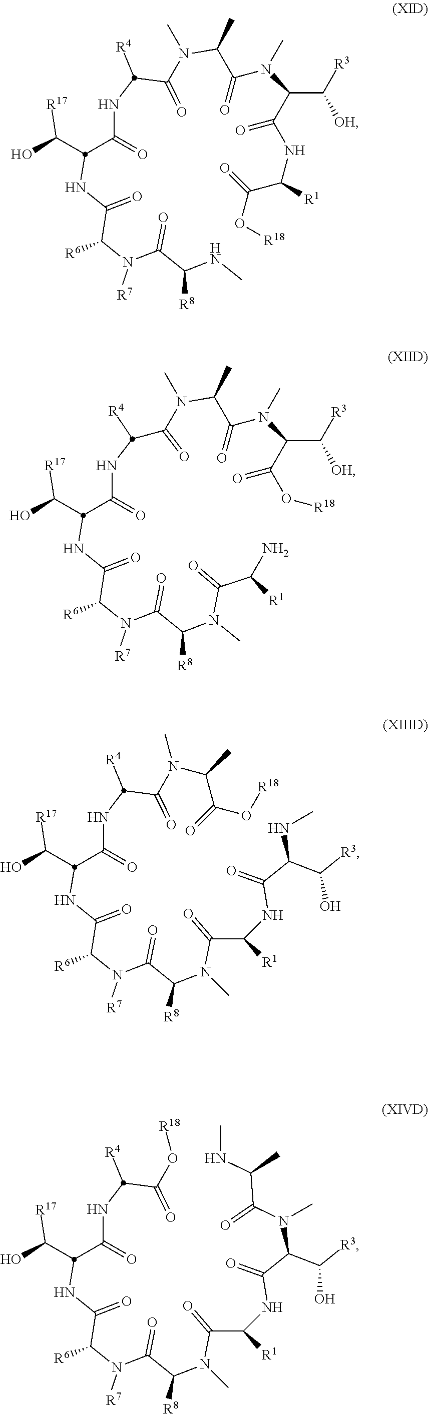

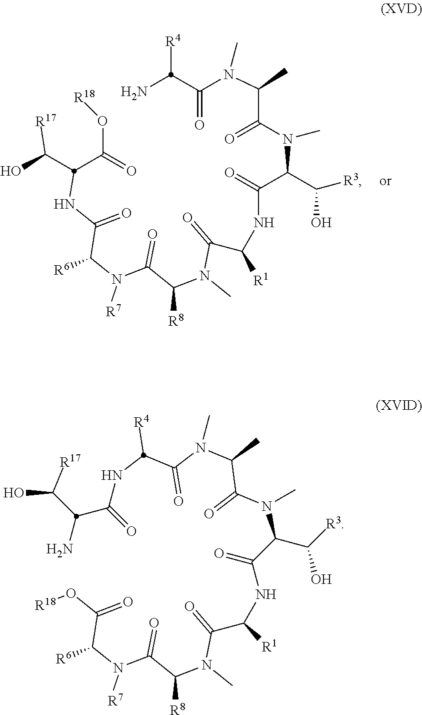

R¹, R³, R⁴, R⁶, R⁷, R⁸, R¹⁷, and R¹⁸ are as described herein, including in embodiments.

In embodiments, the compound has the formula:

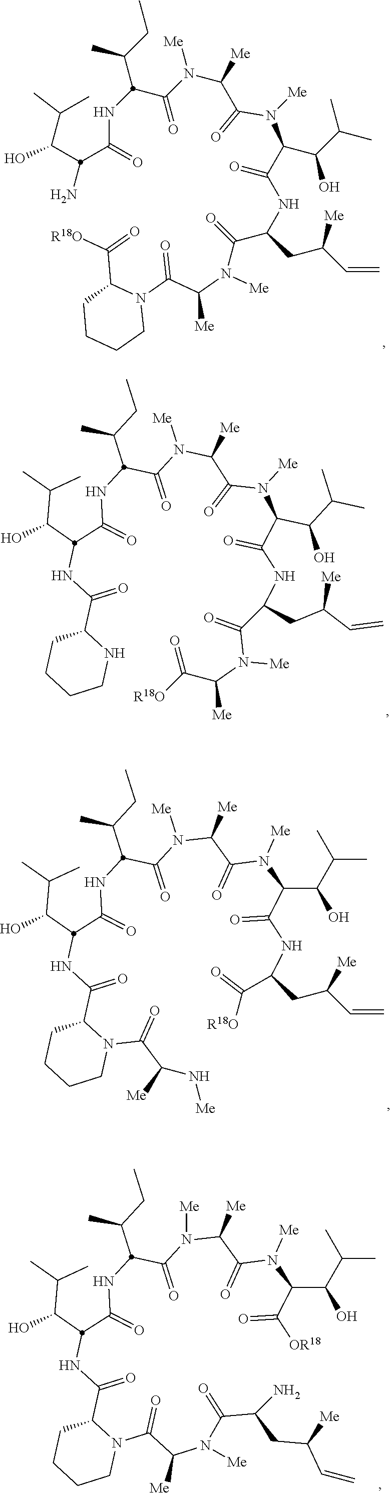

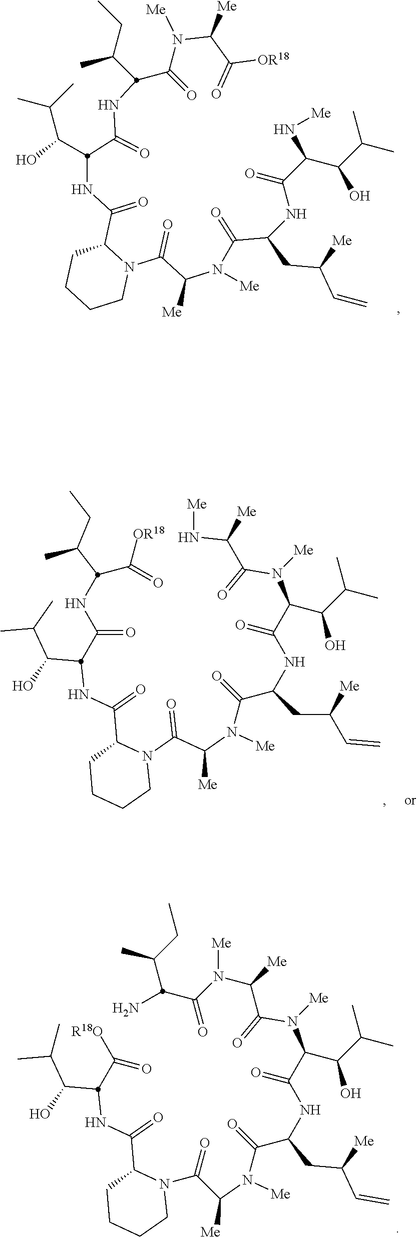

R¹⁸ is as described herein, including in embodiments.

In embodiments, the compound has the formula:

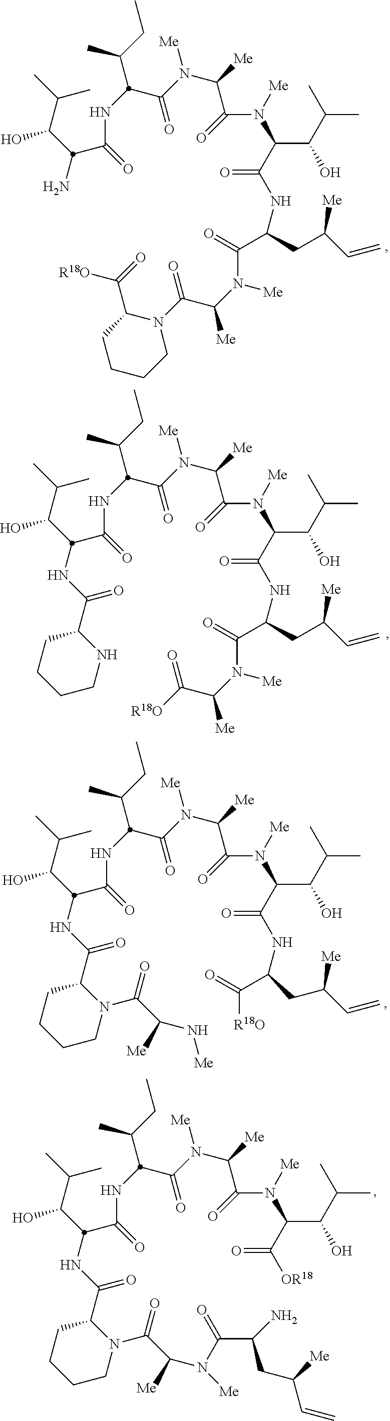

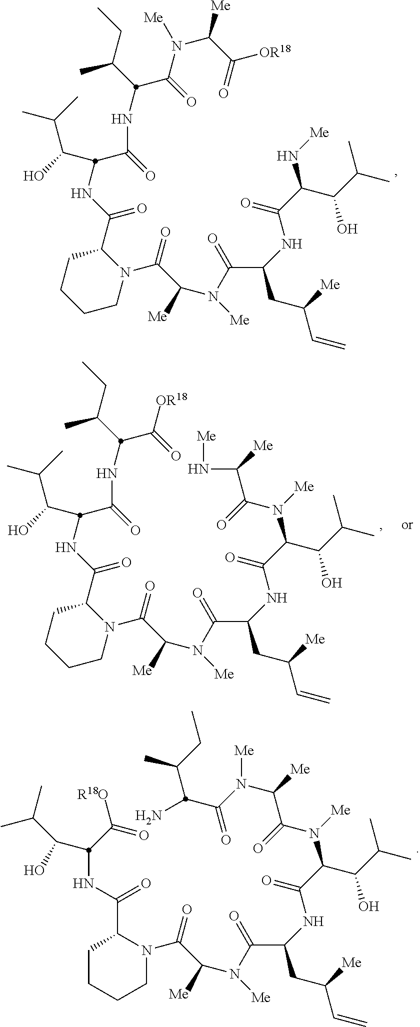

R¹⁸ is as described herein, including in embodiments.

In embodiments, the compound has the formula:

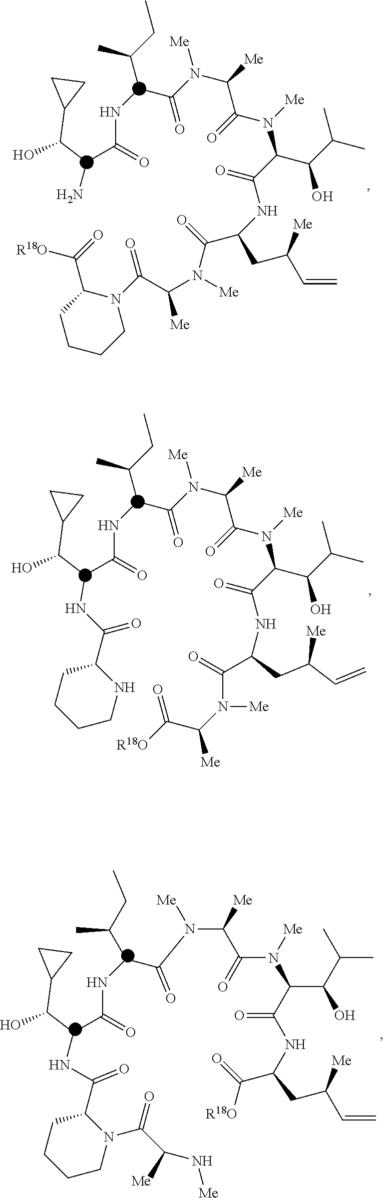

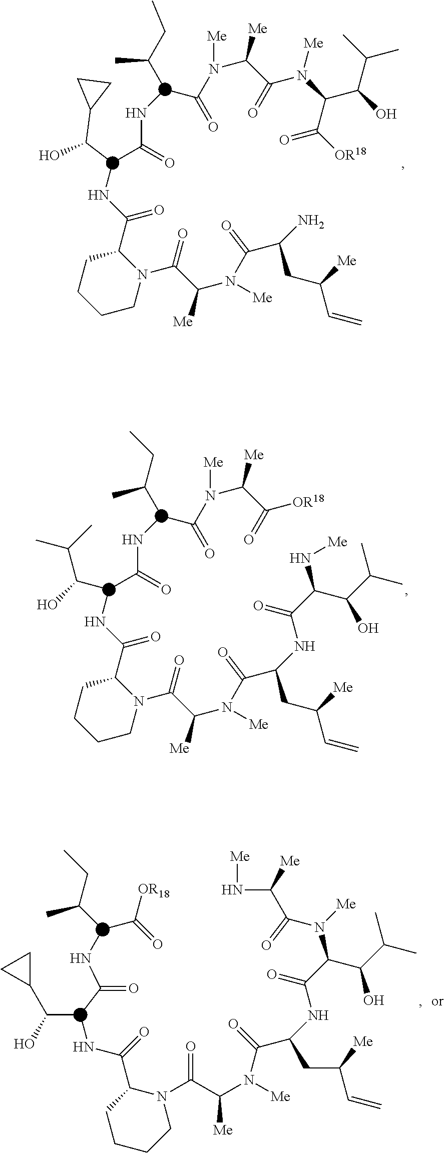

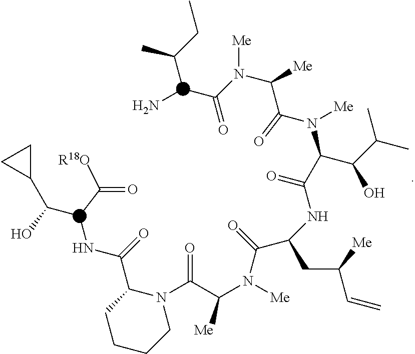

R¹⁸ is as described herein, including in embodiments.

In embodiments, the compound has the formula:

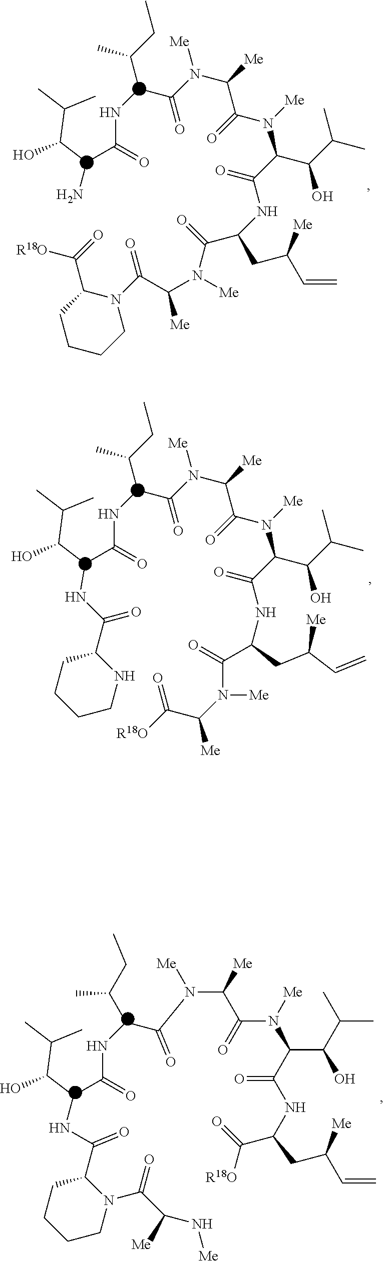

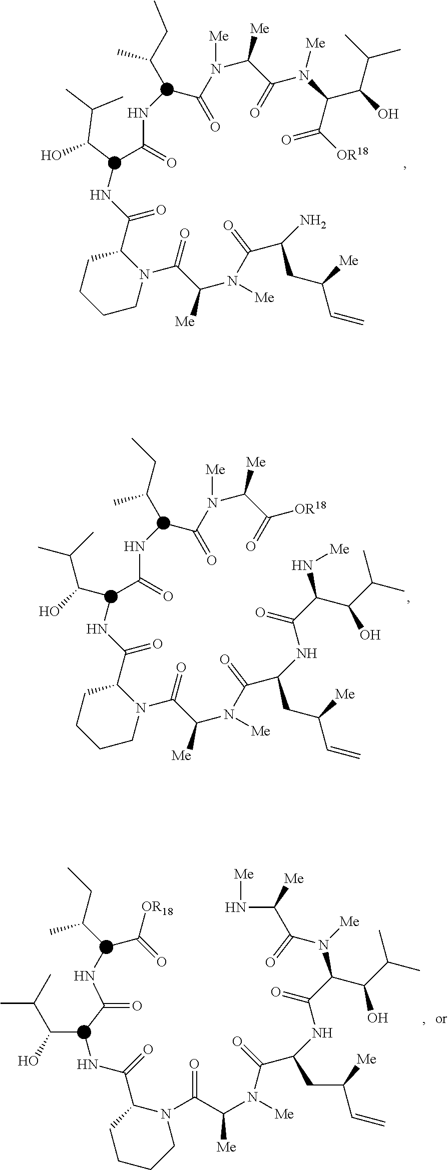

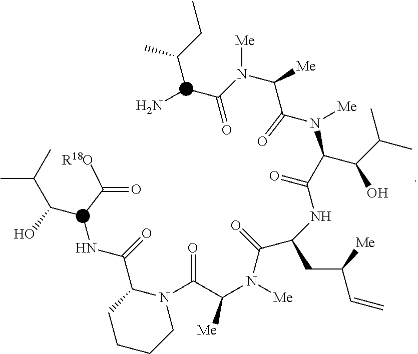

R¹⁸ is as described herein, including in embodiments.

In embodiments, R¹ is substituted or unsubstituted alkyl or substituted or unsubstituted heteroalkyl.

In embodiments, R¹ is independently substituted or unsubstituted C₁-C₆ alkyl. In embodiments, R¹ is substituted or unsubstituted C₃-C₅ alkyl. In embodiments, R¹ is independently substituted C₁-C₆ alkyl. In embodiments, R¹ is substituted C₃-C₅ alkyl. In embodiments, R¹ is independently unsubstituted C₁-C₆ alkyl. In embodiments, R¹ is unsubstituted C₃-C₅ alkyl. In embodiments, R¹ is independently fluoro-substituted C₁-C₆ alkyl. In embodiments, R¹ is fluoro-substituted C₃-C₅ alkyl. In embodiments, R¹ is fluoro-substituted or unsubstituted C₃-C₅ alkyl. In embodiments, R¹ is independently substituted C₂-C₆ alkenyl. In embodiments, R¹ is substituted C₃-C₅ alkenyl. In embodiments, R¹ is independently unsubstituted C₂-C₆ alkenyl. In embodiments, R¹ is unsubstituted C₃-C₅ alkyl. In embodiments, R¹ is independently fluoro-substituted C₂-C₆ alkenyl.

In embodiments, R¹ is fluoro-substituted C₃-C₅ alkenyl. In embodiments, R¹ is fluoro-substituted or unsubstituted C₃-C₅ alkenyl.

In embodiments, R¹ is independently substituted or unsubstituted 2 to 6 membered heteroalkyl. In embodiments, R¹ is independently substituted 2 to 6 membered heteroalkyl. In embodiments, R¹ is independently unsubstituted 2 to 6 membered heteroalkyl. In embodiments, R¹ is independently fluoro-substituted 2 to 6 membered heteroalkyl. In embodiments, R¹ is independently substituted 3 to 6 membered heteroalkyl. In embodiments, R¹ is independently unsubstituted 3 to 6 membered heteroalkyl. In embodiments, R¹ is independently fluoro-substituted 3 to 6 membered heteroalkyl. In embodiments, R¹ is independently substituted 2 to 6 membered heteroalkenyl. In embodiments, R¹ is independently unsubstituted 2 to 6 membered heteroalkenyl. In embodiments, R¹ is independently fluoro-substituted 2 to 6 membered heteroalkenyl. In embodiments, R¹ is independently substituted 3 to 6 membered heteroalkenyl. In embodiments, R¹ is independently unsubstituted 3 to 6 membered heteroalkenyl. In embodiments, R¹ is independently fluoro-substituted 3 to 6 membered heteroalkenyl.

In embodiments, R¹ is

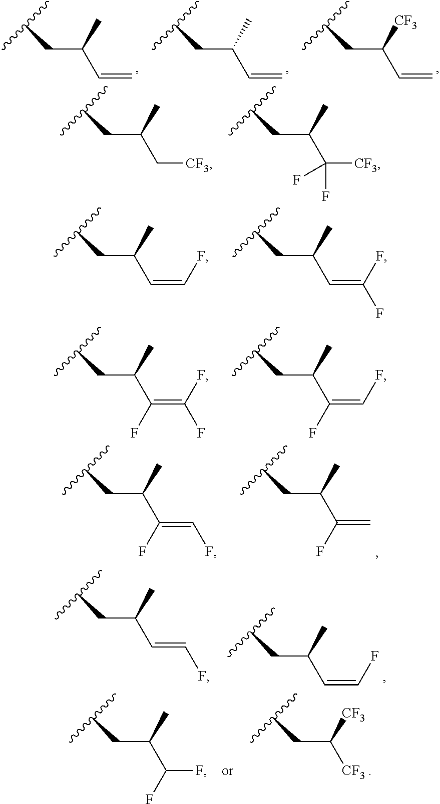

In embodiments, R¹ is

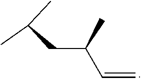

In embodiments, R¹ is

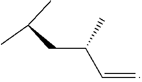

In embodiments, R¹ is

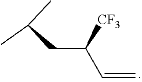

In embodiments, R¹ is

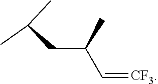

In embodiments, R¹ is

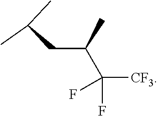

In embodiments, R¹ is

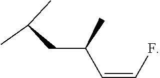

In embodiments, R¹ is

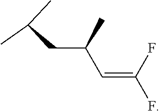

In embodiments, R¹ is

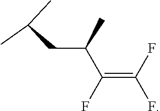

In embodiments, R¹ is

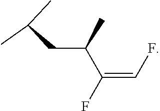

In embodiments, R¹ is

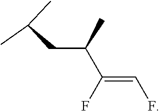

In embodiments, R¹ is

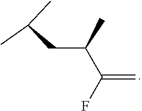

In embodiments, R¹ is

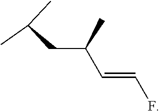

In embodiments, R¹ is

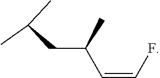

In embodiments, R¹ is

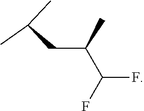

In embodiments, R¹ is

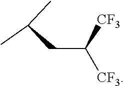

In embodiments R¹ is not —CH₂CH(CH₃)(CH₂CH₃). In embodiments R¹ is not —CH₂CH(CH₃)₂.

In embodiments, R² is —NR^{2A}R^{2B} or —OR^{2B}. In embodiments, R² is —OR^{2B}. In embodiments, R² is —OH. In embodiments, R² is —NH₂.

In embodiments, R² is —OCX²₃. In embodiments, R² is —OCH₂X². In embodiments, R² is —OCHX²₂. In embodiments, R² is —SR^{2B}. In embodiments, R² is —NR^{2A}R^{2B}.

In embodiments, X² is independently —F. In embodiments, X² is independently —Cl. In embodiments, X² is independently —Br. In embodiments, X² is independently —I.

In embodiments, R^{2A} and R^{2B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{2A} and R^{2B} substituents bonded to the same nitrogen atom are joined to form a substituted or unsubstituted heterocycloalkyl or substituted or unsubstituted heteroaryl. In embodiments, R^{2A} and R^{2B} are independently hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{2A} is independently hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{2A} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl. In embodiments, R^{2A} is independently hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl.

In embodiments, R^{2A} is independently hydrogen, substituted or unsubstituted C₁-C₄ alkyl, or substituted or unsubstituted 2 to 4 membered heteroalkyl. In embodiments, R^{2A} is independently hydrogen. In embodiments, R^{2A} is independently substituted or unsubstituted C₁-C₄ alkyl. In embodiments, R^{2A} is independently substituted or unsubstituted 2 to 4 membered heteroalkyl. In embodiments, R^{2A} is independently unsubstituted C₁-C₄ alkyl. In embodiments, R^{2A} is independently unsubstituted 2 to 4 membered heteroalkyl. In embodiments, R^{2A} is independently substituted or unsubstituted C₁-C₆ alkyl or substituted or unsubstituted 2 to 6 membered heteroalkyl. In embodiments, R^{2A} is independently —CH₃. In embodiments, R^{2A} is independently —CCl₃. In embodiments, R^{2A} is independently —CBr₃. In embodiments, R^{2A} is independently —CF₃. In embodiments, R^{2A} is independently —Cl₃. In embodiments, R^{2A} is independently —CHCl₂. In embodiments, R^{2A} is independently —CHBr₂. In embodiments, R^{2A} is independently —CHF₂. In embodiments, R^{2A} is independently —CHI₂. In embodiments, R^{2A} is independently —CH₂Cl. In embodiments, R^{2A} is independently —CH₂Br. In embodiments, R^{2A} is independently —CH₂F. In embodiments, R^{2A} is independently —CH₂I. In embodiments, R^{2A} is independently —CN. In embodiments, R^{2A} is independently —OCH₃. In embodiments, R^{2A} is independently —NH₂. In embodiments, R^{2A} is independently —COOH. In embodiments, R^{2A} is independently —COCH₃. In embodiments, R^{2A} is independently —CONH₂. In embodiments, R^{2A} is independently —OCCl₃. In embodiments, R^{2A} is independently —OCF₃. In embodiments, R^{2A} is independently —OCBr₃. In embodiments, R^{2A} is independently —OCI₃. In embodiments, R^{2A} is independently —OCHCl₂. In embodiments, R^{2A} is independently —OCHBr₂. In embodiments, R^{2A} is independently —OCHI₂. In embodiments, R^{2A} is independently —OCHF₂. In embodiments, R^{2A} is independently —OCH₂Cl. In embodiments, R^{2A} is independently —OCH₂Br. In embodiments, R^{2A} is independently —OCH₂I. In embodiments, R^{2A} is independently —OCH₂F. In embodiments, R^{2A} is independently unsubstituted methyl. In embodiments, R^{2A} is independently —OCH₃. In embodiments, R^{2A} is independently —OCH₂CH₃. In embodiments, R^{2A} is independently —OCH(CH₃)₂. In embodiments, R^{2A} is independently —OC(CH₃)₃. In embodiments, R^{2A} is independently —CH₃. In embodiments, R^{2A} is independently —CH₂CH₃. In embodiments, R^{2A} is independently —CH(CH₃)₂. In embodiments, R^{2A} is independently —C(CH₃)₃.

In embodiments, R^{2A} is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{2A} is independently substituted or unsubstituted alkyl. In embodiments, R^{2A} is independently unsubstituted alkyl. In embodiments, R^{2A} is independently unsubstituted methyl. In embodiments, R^{2A} is independently unsubstituted ethyl. In embodiments, R^{2A} is independently unsubstituted propyl. In embodiments, R^{2A} is independently substituted or unsubstituted heteroalkyl. In embodiments, R^{2A} is independently unsubstituted heteroalkyl. In embodiments, R^{2A} is independently substituted or unsubstituted cycloalkyl. In embodiments, R^{2A} is independently unsubstituted cycloalkyl. In embodiments, R^{2A} is independently substituted or unsubstituted heterocycloalkyl. In embodiments, R^{2A} is independently unsubstituted heterocycloalkyl. In embodiments, R^{2A} is independently substituted or unsubstituted aryl. In embodiments, R^{2A} is independently unsubstituted phenyl. In embodiments, R^{2A} is independently substituted or unsubstituted heteroaryl. In embodiments, R^{2A} is independently unsubstituted heteroaryl. In embodiments, R^{2A} is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R^{2A} is independently substituted or unsubstituted C₁-C₆ alkyl. In embodiments, R^{2A} is independently unsubstituted C₁-C₆ alkyl. In embodiments, R^{2A} is independently unsubstituted methyl. In embodiments, R^{2A} is independently unsubstituted ethyl. In embodiments, R^{2A} is independently unsubstituted propyl. In embodiments, R^{2A} is independently substituted or unsubstituted 2 to 6 membered heteroalkyl. In embodiments, R^{2A} is independently unsubstituted 2 to 6 membered heteroalkyl. In embodiments, R^{2A} is independently substituted or unsubstituted C₃-C₆ cycloalkyl. In embodiments, R^{2A} is independently unsubstituted C₃-C₆ cycloalkyl. In embodiments, R^{2A} is independently substituted or unsubstituted 3 to 6 membered heterocycloalkyl. In embodiments, R^{2A} is independently unsubstituted 3 to 6 membered heterocycloalkyl. In embodiments, R^{2A} is independently substituted or unsubstituted C₆-C₁₀ aryl. In embodiments, R^{2A} is independently unsubstituted C₆-C₁₀ aryl. In embodiments, R^{2A} is independently substituted phenyl. In embodiments, R^{2A} is independently unsubstituted phenyl. In embodiments, R^{2A} is independently substituted or unsubstituted 5 to 10 membered heteroaryl. In embodiments, R^{2A} is independently substituted or unsubstituted 5 to 6 membered heteroaryl. In embodiments, R^{2A} is independently unsubstituted 5 to 10 membered heteroaryl. In embodiments, R^{2A} is independently unsubstituted 5 to 6 membered heteroaryl.

In embodiments, R^{2B} is independently hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{2B} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCF₃, —OCBr₃, —OCI₃, —OCHCI₂, —OCHBr₂, —OCHI₂, —OCHF₂, —OCH₂Cl, —OCH₂Br, —OCH₂I, —OCH₂F, substituted or unsubstituted alkyl. In embodiments, R^{2B} is independently hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl.

In embodiments, R^{2B} is independently hydrogen, substituted or unsubstituted C₁-C₄ alkyl, or substituted or unsubstituted 2 to 4 membered heteroalkyl. In embodiments, R^{2B} is independently hydrogen. In embodiments, R^{2B} is independently substituted or unsubstituted C₁-C₄ alkyl. In embodiments, R^{2B} is independently substituted or unsubstituted 2 to 4 membered heteroalkyl. In embodiments, R^{2B} is independently unsubstituted C₁-C₄ alkyl. In embodiments, R^{2B} is independently unsubstituted 2 to 4 membered heteroalkyl. In embodiments, R^{2B} is independently substituted or unsubstituted C₁-C₆ alkyl or substituted or unsubstituted 2 to 6 membered heteroalkyl. In embodiments, R^{2B} is independently —CH₃. In embodiments, R^{2B} is independently —CCl₃. In embodiments, R^{2B} is independently —CBr₃. In embodiments, R^{2B} is independently —CF₃. In embodiments, R^{2B} is independently —CI₃. In embodiments, R^{2B} is independently —CHCl₂. In embodiments, R^{2B} is independently —CHBr₂. In embodiments, R^{2B} is independently —CHF₂. In embodiments, R^{2B} is independently —CHI₂. In embodiments, R^{2B} is independently —CH₂Cl. In embodiments, R^{2B} is independently —CH₂Br. In embodiments, R^{2B} is independently —CH₂F. In embodiments, R^{2B} is independently —CH₂I. In embodiments, R^{2B} is independently —CN. In embodiments, R^{2B} is independently —OCH₃. In embodiments, R^{2B} is independently —NH₂. In embodiments, R^{2B} is independently —COOH. In embodiments, R^{2B} is independently —COCH₃. In embodiments, R^{2B} is independently —CONH₂. In embodiments, R^{2B} is independently —OCCl₃. In embodiments, R^{2B} is independently —OCF₃. In embodiments, R^{2B} is independently —OCBr₃. In embodiments, R^{2B} is independently —OCI₃. In embodiments, R^{2B} is independently —OCHCl₂. In embodiments, R^{2B} is independently —OCHBr₂. In embodiments, R^{2B} is independently —OCHI₂. In embodiments, R^{2B} is independently —OCHF₂. In embodiments, R^{2B} is independently —OCH₂Cl. In embodiments, R^{2B} is independently —OCH₂Br. In embodiments, R^{2B} is independently —OCH₂I. In embodiments, R^{2B} is independently —OCH₂F. In embodiments, R^{2B} is independently unsubstituted methyl. In embodiments, R^{2B} is independently —OCH₃. In embodiments, R^{2B} is independently —OCH₂CH₃. In embodiments, R^{2B} is independently —OCH(CH₃)₂. In embodiments, R^{2B} is independently —OC(CH₃)₃. In embodiments, R^{2B} is independently —CH₃. In embodiments, R^{2B} is independently —CH₂CH₃. In embodiments, R^{2B} is independently —CH(CH₃)₂. In embodiments, R^{2B} is independently —C(CH₃)₃.

In embodiments, R^{2B} is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{2B} is independently substituted or unsubstituted alkyl. In embodiments, R^{2B} is independently unsubstituted alkyl. In embodiments, R^{2B} is independently unsubstituted methyl. In embodiments, R^{2B} is independently unsubstituted ethyl. In embodiments, R^{2B} is independently unsubstituted propyl. In embodiments, R^{2B} is independently substituted or unsubstituted heteroalkyl. In embodiments, R^{2B} is independently unsubstituted heteroalkyl. In embodiments, R^{2B} is independently substituted or unsubstituted cycloalkyl. In embodiments, R^{2B} is independently unsubstituted cycloalkyl. In embodiments, R^{2B} is independently substituted or unsubstituted heterocycloalkyl. In embodiments, R^{2B} is independently unsubstituted heterocycloalkyl. In embodiments, R^{2B} is independently substituted or unsubstituted aryl. In embodiments, R^{2B} is independently unsubstituted phenyl. In embodiments, R^{2B} is independently substituted or unsubstituted heteroaryl. In embodiments, R^{2B} is independently unsubstituted heteroaryl. In embodiments, R^{2B} is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R^{2B} is independently substituted or unsubstituted C₁-C₆ alkyl. In embodiments, R^{2B} is independently unsubstituted C₁-C₆ alkyl. In embodiments, R^{2B} is independently unsubstituted methyl. In embodiments, R^{2B} is independently unsubstituted ethyl. In embodiments, R^{2B} is independently unsubstituted propyl. In embodiments, R^{2B} is independently substituted or unsubstituted 2 to 6 membered heteroalkyl. In embodiments, R^{2B} is independently unsubstituted 2 to 6 membered heteroalkyl. In embodiments, R^{2B} is independently substituted or unsubstituted C₃-C₆ cycloalkyl. In embodiments, R^{2B} is independently unsubstituted C₃-C₆ cycloalkyl. In embodiments, R^{2B} is independently substituted or unsubstituted 3 to 6 membered heterocycloalkyl. In embodiments, R^{2B} is independently unsubstituted 3 to 6 membered heterocycloalkyl. In embodiments, R^{2B} is independently substituted or unsubstituted C₆-C₁₀ aryl. In embodiments, R^{2B} is independently unsubstituted C₆-C₁₀ aryl. In embodiments, R^{2B} is independently substituted phenyl. In embodiments, R^{2B} is independently unsubstituted phenyl. In embodiments, R^{2B} is independently substituted or unsubstituted 5 to 10 membered heteroaryl. In embodiments, R^{2B} is independently substituted or unsubstituted 5 to 6 membered heteroaryl. In embodiments, R^{2B} is independently unsubstituted 5 to 10 membered heteroaryl. In embodiments, R^{2B} is independently unsubstituted 5 to 6 membered heteroaryl.

In embodiments, R^{2A} and R^{2B} substituents bonded to the same nitrogen atom are joined to form a substituted or unsubstituted heterocycloalkyl or substituted or unsubstituted heteroaryl.

In embodiments, R^{2A} and R^{2B} substituents bonded to the same nitrogen atom are joined to form a substituted or unsubstituted heterocycloalkyl. In embodiments, R^{2A} and R^{2B} substituents bonded to the same nitrogen atom are joined to form an unsubstituted heterocycloalkyl. In embodiments, R^{2A} and R^{2B} substituents bonded to the same nitrogen atom are joined to form a substituted or unsubstituted heteroaryl. In embodiments, R^{2A} and R^{2B} substituents bonded to the same nitrogen atom are joined to form an unsubstituted heteroaryl. In embodiments, R^{2A} and R^{2B} substituents bonded to the same nitrogen atom are joined to form a substituted or unsubstituted 3 to 6 membered heterocycloalkyl or substituted or unsubstituted 5 to 10 membered heteroaryl. In embodiments, R^{2A} and R^{2B} substituents bonded to the same nitrogen atom are joined to form a substituted or unsubstituted 3 to 6 membered heterocycloalkyl. In embodiments, R^{2A} and R^{2B} substituents bonded to the same nitrogen atom are joined to form an unsubstituted 3 to 6 membered heterocycloalkyl. In embodiments, R^{2A} and R^{2B} substituents bonded to the same nitrogen atom are joined to form a substituted or unsubstituted 5 to 10 membered heteroaryl. In embodiments, R^{2A} and R^{2B} substituents bonded to the same nitrogen atom are joined to form a substituted or unsubstituted 5 to 6 membered heteroaryl. In embodiments, R^{2A} and R^{2B} substituents bonded to the same nitrogen atom are joined to form an unsubstituted 5 to 10 membered heteroaryl. In embodiments, R^{2A} and R^{2B} substituents bonded to the same nitrogen atom are joined to form an unsubstituted 5 to 6 membered heteroaryl.

In embodiments, R³ is substituted or unsubstituted alkyl or substituted or unsubstituted cycloalkyl. In embodiments, R³ is substituted or unsubstituted C₁-C₆ alkyl or substituted or unsubstituted C₃-C₆ cycloalkyl. In embodiments, R³ is fluoro-substituted or unsubstituted C₁-C₆ alkyl or fluoro-substituted or unsubstituted C₃-C₆ cycloalkyl.

In embodiments, R³ is independently hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCF₃, —OCBr₃, —OCI₃, —OCHCI₂, —OCHBr₂, —OCHI₂, —OCHF₂, —OCH₂Cl, —OCH₂Br, —OCH₂I, —OCH₂F, substituted or unsubstituted alkyl. In embodiments, R³ is independently hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl.

In embodiments, R³ is independently hydrogen, substituted or unsubstituted C₁-C₄ alkyl, or substituted or unsubstituted 2 to 4 membered heteroalkyl. In embodiments, R³ is independently hydrogen. In embodiments, R³ is independently substituted or unsubstituted C₁-C₄ alkyl. In embodiments, R³ is independently substituted or unsubstituted 2 to 4 membered heteroalkyl. In embodiments, R³ is independently unsubstituted C₁-C₄ alkyl. In embodiments, R³ is independently unsubstituted 2 to 4 membered heteroalkyl. In embodiments, R³ is independently substituted or unsubstituted C₁-C₆ alkyl or substituted or unsubstituted 2 to 6 membered heteroalkyl. In embodiments, R³ is independently —CH₃. In embodiments, R³ is independently —CCl₃. In embodiments, R³ is independently —CBr₃. In embodiments, R³ is independently —CF₃. In embodiments, R³ is independently —CI₃. In embodiments, R³ is independently —CHCl₂. In embodiments, R³ is independently —CHBr₂. In embodiments, R³ is independently —CHF₂. In embodiments, R³ is independently —CHI₂. In embodiments, R³ is independently —CH₂Cl. In embodiments, R³ is independently —CH₂Br. In embodiments, R³ is independently —CH₂F. In embodiments, R³ is independently —CH₂I. In embodiments, R³ is independently —CN. In embodiments, R³ is independently —OCH₃. In embodiments, R³ is independently —NH₂. In embodiments, R³ is independently —COOH. In embodiments, R³ is independently —COCH₃. In embodiments, R³ is independently —CONH₂. In embodiments, R³ is independently —OCCl₃. In embodiments, R³ is independently —OCF₃. In embodiments, R³ is independently —OCBr₃. In embodiments, R³ is independently —OCI₃. In embodiments, R³ is independently —OCHCl₂. In embodiments, R³ is independently —OCHBr₂. In embodiments, R³ is independently —OCHI₂. In embodiments, R³ is independently —OCHF₂. In embodiments, R³ is independently —OCH₂Cl. In embodiments, R³ is independently —OCH₂Br. In embodiments, R³ is independently —OCH₂I. In embodiments, R³ is independently —OCH₂F. In embodiments, R³ is independently unsubstituted methyl. In embodiments, R³ is independently —OCH₃. In embodiments, R³ is independently —OCH₂CH₃. In embodiments, R³ is independently —OCH(CH₃)₂. In embodiments, R³ is independently —OC(CH₃)₃. In embodiments, R³ is independently —CH₃. In embodiments, R³ is independently —CH₂CH₃. In embodiments, R³ is independently —CH(CH₃)₂. In embodiments, R³ is independently —C(CH₃)₃. In embodiments, R³ is independently —CH₂CH₂CH₂CH₃.

In embodiments, R³ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is independently substituted or unsubstituted alkyl. In embodiments, R³ is independently unsubstituted alkyl. In embodiments, R³ is independently unsubstituted methyl. In embodiments, R³ is independently unsubstituted ethyl. In embodiments, R³ is independently unsubstituted propyl. In embodiments, R³ is independently substituted or unsubstituted heteroalkyl. In embodiments, R³ is independently unsubstituted heteroalkyl. In embodiments, R³ is independently substituted or unsubstituted cycloalkyl. In embodiments, R³ is independently unsubstituted cycloalkyl. In embodiments, R³ is independently substituted or unsubstituted heterocycloalkyl. In embodiments, R³ is independently unsubstituted heterocycloalkyl. In embodiments, R³ is independently substituted or unsubstituted aryl. In embodiments, R³ is independently unsubstituted phenyl. In embodiments, R³ is independently substituted or unsubstituted heteroaryl. In embodiments, R³ is independently unsubstituted heteroaryl. In embodiments, R³ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R³ is independently substituted or unsubstituted C₁-C₆ alkyl. In embodiments, R³ is independently unsubstituted C₁-C₆ alkyl. In embodiments, R³ is independently unsubstituted methyl. In embodiments, R³ is independently unsubstituted ethyl. In embodiments, R³ is independently unsubstituted propyl. In embodiments, R³ is independently substituted or unsubstituted 2 to 6 membered heteroalkyl. In embodiments, R³ is independently unsubstituted 2 to 6 membered heteroalkyl. In embodiments, R³ is independently substituted or unsubstituted C₃-C₆ cycloalkyl. In embodiments, R³ is independently unsubstituted C₃-C₆ cycloalkyl. In embodiments, R³ is independently substituted or unsubstituted 3 to 6 membered heterocycloalkyl. In embodiments, R³ is independently unsubstituted 3 to 6 membered heterocycloalkyl. In embodiments, R³ is independently substituted or unsubstituted C₆-C₁₀ aryl. In embodiments, R³ is independently unsubstituted C₆-C₁₀ aryl. In embodiments, R³ is independently substituted phenyl. In embodiments, R³ is independently unsubstituted phenyl. In embodiments, R³ is independently substituted or unsubstituted 5 to 10 membered heteroaryl. In embodiments, R³ is independently substituted or unsubstituted 5 to 6 membered heteroaryl. In embodiments, R³ is independently unsubstituted 5 to 10 membered heteroaryl. In embodiments, R³ is independently unsubstituted 5 to 6 membered heteroaryl. In embodiments, R³ is independently substituted or unsubstituted C₃-C₅ cycloalkyl. In embodiments, R³ is independently unsubstituted C₃-C₈ cycloalkyl. In embodiments, R³ is independently substituted C₃-C₈ cycloalkyl. In embodiments, R³ is independently substituted or unsubstituted C₄-C₈ cycloalkyl. In embodiments, R³ is independently unsubstituted C₄-C₅ cycloalkyl. In embodiments, R³ is independently substituted C₄-C₈ cycloalkyl. In embodiments, R³ is independently substituted or unsubstituted C₅-C₈ cycloalkyl. In embodiments, R³ is independently unsubstituted C₅-C₈ cycloalkyl. In embodiments, R³ is independently substituted C₅-C₅ cycloalkyl. In embodiments, R³ is independently substituted or unsubstituted C₄-C₆ cycloalkyl. In embodiments, R³ is independently unsubstituted C₄-C₆ cycloalkyl. In embodiments, R³ is independently substituted C₄-C₆ cycloalkyl. In embodiments, R³ is independently substituted or unsubstituted C₅-C₆ cycloalkyl. In embodiments, R³ is independently unsubstituted C₅-C₆ cycloalkyl. In embodiments, R³ is independently substituted C₅-C₆ cycloalkyl.

In embodiments, R³ is independently substituted or unsubstituted C₁-C₆ alkyl. In embodiments, R³ is substituted or unsubstituted C₃-C₅ alkyl. In embodiments, R³ is independently substituted C₁-C₆ alkyl. In embodiments, R³ is substituted C₃-C₅ alkyl. In embodiments, R³ is independently unsubstituted C₁-C₆ alkyl. In embodiments, R³ is unsubstituted C₃-C₅ alkyl. In embodiments, R³ is independently fluoro-substituted C₁-C₆ alkyl. In embodiments, R³ is fluoro-substituted C₃-C₅ alkyl. In embodiments, R³ is fluoro-substituted or unsubstituted C₃-C₅ alkyl. In embodiments, R³ is independently substituted C₂-C₆ alkenyl. In embodiments, R³ is substituted C₃-C₅ alkenyl. In embodiments, R³ is independently unsubstituted C₂-C₆ alkenyl. In embodiments, R³ is unsubstituted C₃-C₅ alkyl. In embodiments, R³ is independently fluoro-substituted C₂-C₆ alkenyl. In embodiments, R³ is fluoro-substituted C₃-C₅ alkenyl. In embodiments, R³ is fluoro-substituted or unsubstituted C₃-C₅ alkenyl.

In embodiments, R³ is independently substituted or unsubstituted 2 to 6 membered heteroalkyl. In embodiments, R³ is independently substituted 2 to 6 membered heteroalkyl. In embodiments, R³ is independently unsubstituted 2 to 6 membered heteroalkyl. In embodiments, R³ is independently fluoro-substituted 2 to 6 membered heteroalkyl. In embodiments, R³ is independently substituted 3 to 6 membered heteroalkyl. In embodiments, R³ is independently unsubstituted 3 to 6 membered heteroalkyl. In embodiments, R³ is independently fluoro-substituted 3 to 6 membered heteroalkyl. In embodiments, R³ is independently substituted 2 to 6 membered heteroalkenyl. In embodiments, R³ is independently unsubstituted 2 to 6 membered heteroalkenyl. In embodiments, R³ is independently fluoro-substituted 2 to 6 membered heteroalkenyl. In embodiments, R³ is independently substituted 3 to 6 membered heteroalkenyl. In embodiments, R³ is independently unsubstituted 3 to 6 membered heteroalkenyl. In embodiments, R³ is independently fluoro-substituted 3 to 6 membered heteroalkenyl.

In embodiments, R³ is

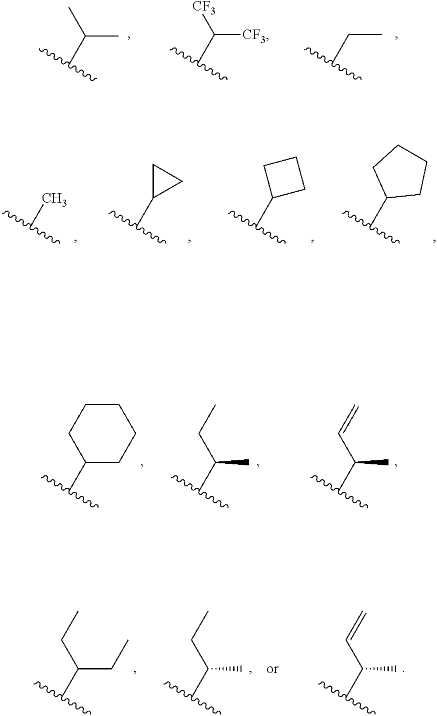

In embodiments, R³ is

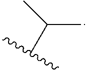 [SMILES: *C(C)C]

In embodiments, R³ is

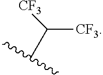 [SMILES: *C(C(F)(F)F)C(F)(F)F]

In embodiments, R³ is

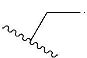 [SMILES: *CC]

In embodiments, R³ is

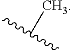

In embodiments, R³ is

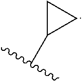 [SMILES: *C1CC1]

In embodiments, R³ is

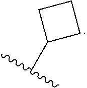 [SMILES: *C1CCC1]

In embodiments, R³ is

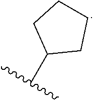 [SMILES: *C1CCCC1]

In embodiments, R³ is

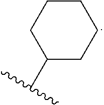 [SMILES: *C1CCCCC1]

In embodiments, R³ is

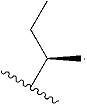 [SMILES: *[C@H](C)CC]

In embodiments, R³ is

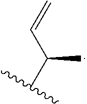 [SMILES: *[C@H](C)C=C]

In embodiments, R³ is

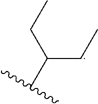 [SMILES: *C(CC)CC]

In embodiments, R³ is

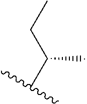 [SMILES: *[C@@H](C)CC]

In embodiments, R³ is

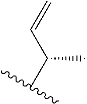 [SMILES: *[C@@H](C)C=C]

In embodiments, R³ is not

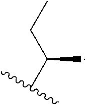 [SMILES: *[C@H](C)CC]

In embodiments, R³ is not

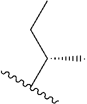 [SMILES: *[C@@H](C)CC]

In embodiments, R⁴ is —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁴ is substituted or unsubstituted alkyl or substituted or unsubstituted cycloalkyl. In embodiments, R⁴ is substituted or unsubstituted C₁-C₆ alkyl or substituted or unsubstituted C₃-C₆ cycloalkyl. In embodiments, R⁴ is fluoro-substituted or unsubstituted C₁-C₆ alkyl or fluoro-substituted or unsubstituted C₃-C₆ cycloalkyl.

In embodiments, R⁴ is substituted or unsubstituted alkyl or substituted or unsubstituted cycloalkyl. In embodiments, R⁴ is substituted or unsubstituted C₁-C₆ alkyl or substituted or unsubstituted C₃-C₆ cycloalkyl. In embodiments, R⁴ is fluoro-substituted or unsubstituted C₁-C₆ alkyl or fluoro-substituted or unsubstituted C₃-C₆ cycloalkyl.

In embodiments, R⁴ is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁴ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R⁴ is independently hydrogen, —CCl₃, —CF₃, —CHF₂, —CH₂F, or substituted or unsubstituted alkyl.

In embodiments, R⁴ is independently hydrogen, substituted or unsubstituted C₁-C₄ alkyl, or substituted or unsubstituted 2 to 4 membered heteroalkyl. In embodiments, R⁴ is independently hydrogen. In embodiments, R⁴ is independently substituted or unsubstituted C₁-C₄ alkyl. In embodiments, R⁴ is independently substituted or unsubstituted 2 to 4 membered heteroalkyl. In embodiments, R⁴ is independently unsubstituted C₁-C₄ alkyl. In embodiments, R⁴ is independently unsubstituted 2 to 4 membered heteroalkyl. In embodiments, R⁴ is independently substituted or unsubstituted C₁-C₆ alkyl or substituted or unsubstituted 2 to 6 membered heteroalkyl. In embodiments, R⁴ is independently —CH₃. In embodiments, R⁴ is independently —CCl₃. In embodiments, R⁴ is independently —CBr₃. In embodiments, R⁴ is independently —CF₃. In embodiments, R⁴ is independently —CI₃. In embodiments, R⁴ is independently —CHCl₂. In embodiments, R⁴ is independently —CHBr₂. In embodiments, R⁴ is independently —CHF₂. In embodiments, R⁴ is independently —CHI₂. In embodiments, R⁴ is independently —CH₂Cl. In embodiments, R⁴ is independently —CH₂Br. In embodiments, R⁴ is independently —CH₂F. In embodiments, R⁴ is independently —CH₂I. In embodiments, R⁴ is independently —COOH. In embodiments, R⁴ is independently —COCH₃. In embodiments, R⁴ is independently —CONH₂. In embodiments, R⁴ is independently unsubstituted methyl. In embodiments, R⁴ is independently —CH₃. In embodiments, R⁴ is independently —CH₂CH₃. In embodiments, R⁴ is independently —CH(CH₃)₂. In embodiments, R⁴ is independently —C(CH₃)₃.

In embodiments, R⁴ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R⁴ is independently substituted or unsubstituted C₁-C₆ alkyl. In embodiments, R⁴ is independently unsubstituted C₁-C₆ alkyl. In embodiments, R⁴ is independently unsubstituted methyl. In embodiments, R⁴ is independently unsubstituted ethyl. In embodiments, R⁴ is independently unsubstituted propyl. In embodiments, R⁴ is independently substituted or unsubstituted 2 to 6 membered heteroalkyl. In embodiments, R⁴ is independently unsubstituted 2 to 6 membered heteroalkyl. In embodiments, R⁴ is independently substituted or unsubstituted C₃-C₆ cycloalkyl. In embodiments, R⁴ is independently unsubstituted C₃-C₆ cycloalkyl. In embodiments, R⁴ is independently substituted or unsubstituted 3 to 6 membered heterocycloalkyl. In embodiments, R⁴ is independently unsubstituted 3 to 6 membered heterocycloalkyl. In embodiments, R⁴ is independently substituted or unsubstituted C₆-C₁₀ aryl. In embodiments, R⁴ is independently unsubstituted C₆-C₁₀ aryl. In embodiments, R⁴ is independently substituted phenyl. In embodiments, R⁴ is independently unsubstituted phenyl. In embodiments, R⁴ is independently substituted or unsubstituted 5 to 10 membered heteroaryl. In embodiments, R⁴ is independently substituted or unsubstituted 5 to 6 membered heteroaryl. In embodiments, R⁴ is independently unsubstituted 5 to 10 membered heteroaryl. In embodiments, R⁴ is independently unsubstituted 5 to 6 membered heteroaryl. In embodiments, R⁴ is independently substituted or unsubstituted C₃-C₅ cycloalkyl. In embodiments, R⁴ is independently unsubstituted C₃-C₈ cycloalkyl. In embodiments, R⁴ is independently substituted C₃-C₈ cycloalkyl. In embodiments, R⁴ is independently substituted or unsubstituted C₄-C₈ cycloalkyl. In embodiments, R⁴ is independently unsubstituted C₄-C₅ cycloalkyl. In embodiments, R⁴ is independently substituted C₄-C₈ cycloalkyl. In embodiments, R⁴ is independently substituted or unsubstituted C₅-C₅ cycloalkyl. In embodiments, R⁴ is independently unsubstituted C₅-C₅ cycloalkyl. In embodiments, R⁴ is independently substituted C₅-C₈ cycloalkyl. In embodiments, R⁴ is independently substituted or unsubstituted C₄-C₆ cycloalkyl. In embodiments, R⁴ is independently unsubstituted C₄-C₆ cycloalkyl. In embodiments, R⁴ is independently substituted C₄-C₆ cycloalkyl. In embodiments, R⁴ is independently substituted or unsubstituted C₅-C₆ cycloalkyl. In embodiments, R⁴ is independently unsubstituted C₅-C₆ cycloalkyl. In embodiments, R⁴ is independently substituted C₅-C₆ cycloalkyl.

In embodiments, R⁴ is independently substituted or unsubstituted C₁-C₆ alkyl. In embodiments, R⁴ is substituted or unsubstituted C₃-C₅ alkyl. In embodiments, R⁴ is independently substituted C₁-C₆ alkyl. In embodiments, R⁴ is substituted C₃-C₅ alkyl. In embodiments, R⁴ is independently unsubstituted C₁-C₆ alkyl. In embodiments, R⁴ is unsubstituted C₃-C₅ alkyl. In embodiments, R⁴ is independently substituted or unsubstituted C₄-C₆ alkyl. In embodiments, R⁴ is substituted or unsubstituted C₄-C₅ alkyl. In embodiments, R⁴ is independently substituted C₄-C₆ alkyl. In embodiments, R⁴ is substituted C₄-C₅ alkyl. In embodiments, R⁴ is independently unsubstituted C₄-C₆ alkyl. In embodiments, R⁴ is unsubstituted C₄-C₅ alkyl. In embodiments, R⁴ is independently substituted or unsubstituted C₁-C₈ alkyl. In embodiments, R⁴ is substituted or unsubstituted C₄-C₅ alkyl. In embodiments, R⁴ is independently substituted C₁-C₅ alkyl. In embodiments, R⁴ is substituted C₄-C₅ alkyl. In embodiments, R⁴ is independently unsubstituted C₁-C₈ alkyl. In embodiments, R⁴ is unsubstituted C₄-C₅ alkyl. In embodiments, R⁴ is independently fluoro-substituted C₁-C₆ alkyl. In embodiments, R⁴ is fluoro-substituted C₃-C₅ alkyl. In embodiments, R⁴ is fluoro-substituted or unsubstituted C₃-C₅ alkyl. In embodiments, R⁴ is independently substituted C₂-C₆ alkenyl. In embodiments, R⁴ is substituted C₃-C₅ alkenyl. In embodiments, R⁴ is independently unsubstituted C₂-C₆ alkenyl. In embodiments, R⁴ is unsubstituted C₃-C₅ alkyl. In embodiments, R⁴ is independently fluoro-substituted C₂-C₆ alkenyl. In embodiments, R⁴ is fluoro-substituted C₃-C₅ alkenyl. In embodiments, R⁴ is fluoro-substituted or unsubstituted C₃-C₅ alkenyl.

In embodiments, R⁴ is independently substituted or unsubstituted 2 to 6 membered heteroalkyl. In embodiments, R⁴ is independently substituted 2 to 6 membered heteroalkyl. In embodiments, R⁴ is independently unsubstituted 2 to 6 membered heteroalkyl. In embodiments, R⁴ is independently fluoro-substituted 2 to 6 membered heteroalkyl. In embodiments, R⁴ is independently substituted 3 to 6 membered heteroalkyl. In embodiments, R⁴ is independently unsubstituted 3 to 6 membered heteroalkyl. In embodiments, R⁴ is independently fluoro-substituted 3 to 6 membered heteroalkyl. In embodiments, R⁴ is independently substituted 2 to 6 membered heteroalkenyl. In embodiments, R⁴ is independently unsubstituted 2 to 6 membered heteroalkenyl. In embodiments, R⁴ is independently fluoro-substituted 2 to 6 membered heteroalkenyl. In embodiments, R⁴ is independently substituted 3 to 6 membered heteroalkenyl. In embodiments, R⁴ is independently unsubstituted 3 to 6 membered heteroalkenyl. In embodiments, R⁴ is independently fluoro-substituted 3 to 6 membered heteroalkenyl.

In embodiments, R⁴ is

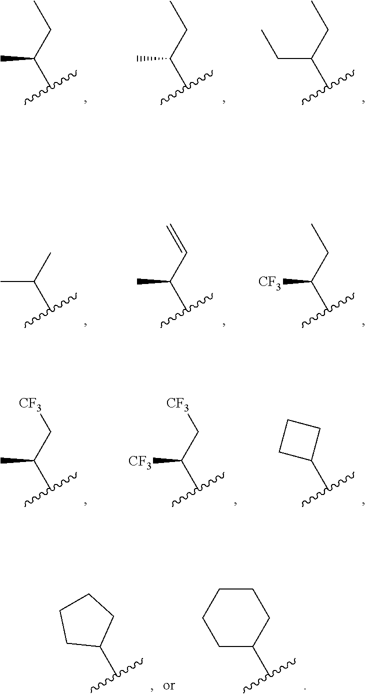

In embodiments, R⁴ is

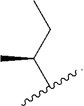 [SMILES: *[C@@H](C)CC]

In embodiments, R⁴ is

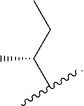 [SMILES: *[C@H](C)CC]

In embodiments, R⁴ is

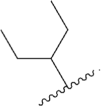 [SMILES: *C(CC)CC]

In embodiments, R⁴ is

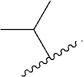 [SMILES: *C(C)C]

In embodiments, R⁴ is

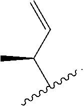 [SMILES: *[C@@H](C)C=C]

In embodiments, R⁴ is

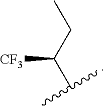 [SMILES: *[C@H](CC)C(F)(F)F]

In embodiments, R⁴ is

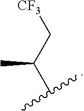 [SMILES: *[C@@H](C)CC(F)(F)F]

In embodiments, R⁴ is

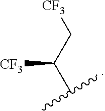 [SMILES: *[C@H](CC(F)(F)F)C(F)(F)F]

In embodiments, R⁴ is

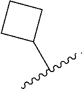 [SMILES: *C1CCC1]

In embodiments, R⁴ is

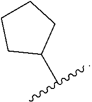 [SMILES: *C1CCCC1]

In embodiments, R⁴ is

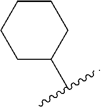 [SMILES: *C1CCCCC1]

In embodiments, R⁴ is not

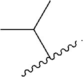 [SMILES: *C(C)C]

In embodiments, R⁵ is halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁵ is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁵ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R⁵ is independently hydrogen, —CCl₃, —CF₃, —CHF₂, —CH₂F, or substituted or unsubstituted alkyl.

In embodiments, R⁵ is independently hydrogen, substituted or unsubstituted C₁-C₄ alkyl, or substituted or unsubstituted 2 to 4 membered heteroalkyl. In embodiments, R⁵ is independently hydrogen. In embodiments, R⁵ is independently substituted or unsubstituted C₁-C₄ alkyl. In embodiments, R⁵ is independently substituted or unsubstituted 2 to 4 membered heteroalkyl. In embodiments, R⁵ is independently unsubstituted C₁-C₄ alkyl. In embodiments, R⁵ is independently unsubstituted 2 to 4 membered heteroalkyl. In embodiments, R⁵ is independently substituted or unsubstituted C₁-C₆ alkyl or substituted or unsubstituted 2 to 6 membered heteroalkyl. In embodiments, R⁵ is independently —CH₃. In embodiments, R⁵ is independently —CCl₃. In embodiments, R⁵ is independently —CBr₃. In embodiments, R⁵ is independently —CF₃. In embodiments, R⁵ is independently —CI₃. In embodiments, R⁵ is independently —CHCl₂. In embodiments, R⁵ is independently —CHBr₂. In embodiments, R⁵ is independently —CHF₂. In embodiments, R⁵ is independently —CHI₂. In embodiments, R⁵ is independently —CH₂Cl. In embodiments, R⁵ is independently —CH₂Br. In embodiments, R⁵ is independently —CH₂F. In embodiments, R⁵ is independently —CH₂I. In embodiments, R⁵ is independently —CN. In embodiments, R⁵ is independently —OCH₃. In embodiments, R⁵ is independently —NH₂. In embodiments, R⁵ is independently —COOH. In embodiments, R⁵ is independently —COCH₃. In embodiments, R⁵ is independently —CONH₂. In embodiments, R⁵ is independently —OCCl₃. In embodiments, R⁵ is independently —OCF₃. In embodiments, R⁵ is independently —OCBr₃. In embodiments, R⁵ is independently —OCI₃. In embodiments, R⁵ is independently —OCHCl₂. In embodiments, R⁵ is independently —OCHBr₂. In embodiments, R⁵ is independently —OCHI₂. In embodiments, R⁵ is independently —OCHF₂. In embodiments, R⁵ is independently —OCH₂Cl. In embodiments, R⁵ is independently —OCH₂Br. In embodiments, R⁵ is independently —OCH₂I. In embodiments, R⁵ is independently —OCH₂F. In embodiments, R⁵ is independently unsubstituted methyl. In embodiments, R⁵ is independently —OCH₃. In embodiments, R⁵ is independently —OCH₂CH₃. In embodiments, R⁵ is independently —OCH(CH₃)₂. In embodiments, R⁵ is independently —OC(CH₃)₃. In embodiments, R⁵ is independently —CH₃. In embodiments, R⁵ is independently —CH₂CH₃. In embodiments, R⁵ is independently —CH(CH₃)₂. In embodiments, R⁵ is independently —C(CH₃)₃.

In embodiments, R⁵ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁵ is independently substituted or unsubstituted alkyl. In embodiments, R⁵ is independently unsubstituted alkyl. In embodiments, R⁵ is independently unsubstituted methyl. In embodiments, R⁵ is independently unsubstituted ethyl. In embodiments, R⁵ is independently unsubstituted propyl. In embodiments, R⁵ is independently substituted or unsubstituted heteroalkyl. In embodiments, R⁵ is independently unsubstituted heteroalkyl. In embodiments, R⁵ is independently substituted or unsubstituted cycloalkyl. In embodiments, R⁵ is independently unsubstituted cycloalkyl. In embodiments, R⁵ is independently substituted or unsubstituted heterocycloalkyl. In embodiments, R⁵ is independently unsubstituted heterocycloalkyl. In embodiments, R⁵ is independently substituted or unsubstituted aryl. In embodiments, R⁵ is independently unsubstituted phenyl. In embodiments, R⁵ is independently substituted or unsubstituted heteroaryl. In embodiments, R⁵ is independently unsubstituted heteroaryl. In embodiments, R⁵ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R⁵ is independently substituted or unsubstituted C₁-C₆ alkyl. In embodiments, R⁵ is independently unsubstituted C₁-C₆ alkyl. In embodiments, R⁵ is independently unsubstituted methyl. In embodiments, R⁵ is independently unsubstituted ethyl. In embodiments, R⁵ is independently unsubstituted propyl. In embodiments, R⁵ is independently substituted or unsubstituted 2 to 6 membered heteroalkyl. In embodiments, R⁵ is independently unsubstituted 2 to 6 membered heteroalkyl. In embodiments, R⁵ is independently substituted or unsubstituted C₃-C₆ cycloalkyl. In embodiments, R⁵ is independently unsubstituted C₃-C₆ cycloalkyl. In embodiments, R⁵ is independently substituted or unsubstituted 3 to 6 membered heterocycloalkyl. In embodiments, R⁵ is independently unsubstituted 3 to 6 membered heterocycloalkyl. In embodiments, R⁵ is independently substituted or unsubstituted C₆-C₁₀ aryl. In embodiments, R⁵ is independently unsubstituted C₆-C₁₀ aryl. In embodiments, R⁵ is independently substituted phenyl. In embodiments, R⁵ is independently unsubstituted phenyl. In embodiments, R⁵ is independently substituted or unsubstituted 5 to 10 membered heteroaryl. In embodiments, R⁵ is independently substituted or unsubstituted 5 to 6 membered heteroaryl. In embodiments, R⁵ is independently unsubstituted 5 to 10 membered heteroaryl. In embodiments, R⁵ is independently unsubstituted 5 to 6 membered heteroaryl.

In embodiments, R⁵ is independently substituted or unsubstituted C₁-C₆ alkyl. In embodiments, R⁵ is substituted or unsubstituted C₃-C₅ alkyl. In embodiments, R⁵ is independently substituted C₁-C₆ alkyl. In embodiments, R⁵ is substituted C₃-C₅ alkyl. In embodiments, R⁵ is independently unsubstituted C₁-C₆ alkyl. In embodiments, R⁵ is unsubstituted C₃-C₅ alkyl. In embodiments, R⁵ is independently fluoro-substituted C₁-C₆ alkyl. In embodiments, R⁵ is fluoro-substituted C₃-C₅ alkyl. In embodiments, R⁵ is fluoro-substituted or unsubstituted C₃-C₅ alkyl. In embodiments, R⁵ is independently substituted C₂-C₆ alkenyl. In embodiments, R⁵ is substituted C₃-C₅ alkenyl. In embodiments, R⁵ is independently unsubstituted C₂-C₆ alkenyl. In embodiments, R⁵ is unsubstituted C₃-C₅ alkyl. In embodiments, R⁵ is independently fluoro-substituted C₂-C₆ alkenyl. In embodiments, R⁵ is fluoro-substituted C₃-C₅ alkenyl. In embodiments, R⁵ is fluoro-substituted or unsubstituted C₃-C₅ alkenyl.

In embodiments, R⁵ is independently substituted or unsubstituted 2 to 6 membered heteroalkyl. In embodiments, R⁵ is independently substituted 2 to 6 membered heteroalkyl. In embodiments, R⁵ is independently unsubstituted 2 to 6 membered heteroalkyl. In embodiments, R⁵ is independently fluoro-substituted 2 to 6 membered heteroalkyl. In embodiments, R⁵ is independently substituted 3 to 6 membered heteroalkyl. In embodiments, R⁵ is independently unsubstituted 3 to 6 membered heteroalkyl. In embodiments, R⁵ is independently fluoro-substituted 3 to 6 membered heteroalkyl. In embodiments, R⁵ is independently substituted 2 to 6 membered heteroalkenyl. In embodiments, R⁵ is independently unsubstituted 2 to 6 membered heteroalkenyl. In embodiments, R⁵ is independently fluoro-substituted 2 to 6 membered heteroalkenyl. In embodiments, R⁵ is independently substituted 3 to 6 membered heteroalkenyl. In embodiments, R⁵ is independently unsubstituted 3 to 6 membered heteroalkenyl. In embodiments, R⁵ is independently fluoro-substituted 3 to 6 membered heteroalkenyl.

In embodiments, R⁶ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl. In embodiments, R⁶ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R⁶ is independently hydrogen, —CCl₃, —CF₃, —CHF₂, —CH₂F, substituted or unsubstituted alkyl, or substituted or unsubstituted heteroalkyl. In embodiments, R⁶ is independently hydrogen, —CCl₃, —CF₃, —CHF₂, —CH₂F, substituted or unsubstituted C₁-C₄ alkyl, or substituted or unsubstituted 2 to 4 membered heteroalkyl. In embodiments, R⁶ is independently fluoro-substituted or unsubstituted C₁-C₄ alkyl, or fluoro-substituted or unsubstituted 2 to 4 membered heteroalkyl. In embodiments, R⁶ is independently fluoro-substituted or unsubstituted C₁-C₄ alkyl. In embodiments, R⁶ is independently fluoro-substituted or unsubstituted 2 to 4 membered heteroalkyl.

In embodiments, R⁶ is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁶ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R⁶ is independently hydrogen, —CCl₃, —CF₃, —CHF₂, —CH₂F, or substituted or unsubstituted alkyl.

In embodiments, R⁶ is independently hydrogen, substituted or unsubstituted C₁-C₄ alkyl, or substituted or unsubstituted 2 to 4 membered heteroalkyl. In embodiments, R⁶ is independently hydrogen. In embodiments, R⁶ is independently substituted or unsubstituted C₁-C₄ alkyl. In embodiments, R⁶ is independently substituted or unsubstituted 2 to 4 membered heteroalkyl. In embodiments, R⁶ is independently unsubstituted C₁-C₄ alkyl. In embodiments, R⁶ is independently unsubstituted 2 to 4 membered heteroalkyl. In embodiments, R⁶ is independently substituted or unsubstituted C₁-C₆ alkyl or substituted or unsubstituted 2 to 6 membered heteroalkyl. In embodiments, R⁶ is independently —CH₃. In embodiments, R⁶ is independently —CCl₃. In embodiments, R⁶ is independently —CBr₃. In embodiments, R⁶ is independently —CF₃. In embodiments, R⁶ is independently —CI₃. In embodiments, R⁶ is independently —CHCl₂. In embodiments, R⁶ is independently —CHBr₂. In embodiments, R⁶ is independently —CHF₂. In embodiments, R⁶ is independently —CHI₂. In embodiments, R⁶ is independently —CH₂Cl. In embodiments, R⁶ is independently —CH₂Br. In embodiments, R⁶ is independently —CH₂F. In embodiments, R⁶ is independently —CH₂I. In embodiments, R⁶ is independently —COOH. In embodiments, R⁶ is independently —COCH₃. In embodiments, R⁶ is independently —CONH₂. In embodiments, R⁶ is independently —CH₃. In embodiments, R⁶ is independently —CH₂CH₃. In embodiments, R⁶ is independently —CH(CH₃)₂. In embodiments, R⁶ is independently —C(CH₃)₃.

In embodiments, R⁶ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R⁶ is independently substituted or unsubstituted C₁-C₆ alkyl. In embodiments, R⁶ is independently unsubstituted C₁-C₆ alkyl. In embodiments, R⁶ is independently unsubstituted methyl. In embodiments, R⁶ is independently unsubstituted ethyl. In embodiments, R⁶ is independently unsubstituted propyl. In embodiments, R⁶ is independently substituted or unsubstituted 2 to 6 membered heteroalkyl. In embodiments, R⁶ is independently unsubstituted 2 to 6 membered heteroalkyl. In embodiments, R⁶ is independently substituted or unsubstituted C₃-C₆ cycloalkyl. In embodiments, R⁶ is independently unsubstituted C₃-C₆ cycloalkyl. In embodiments, R⁶ is independently substituted or unsubstituted 3 to 6 membered heterocycloalkyl. In embodiments, R⁶ is independently unsubstituted 3 to 6 membered heterocycloalkyl. In embodiments, R⁶ is independently substituted or unsubstituted C₆-C₁₀ aryl. In embodiments, R⁶ is independently unsubstituted C₆-C₁₀ aryl. In embodiments, R⁶ is independently substituted phenyl. In embodiments, R⁶ is independently unsubstituted phenyl. In embodiments, R⁶ is independently substituted or unsubstituted 5 to 10 membered heteroaryl. In embodiments, R⁶ is independently substituted or unsubstituted 5 to 6 membered heteroaryl. In embodiments, R⁶ is independently unsubstituted 5 to 10 membered heteroaryl. In embodiments, R⁶ is independently unsubstituted 5 to 6 membered heteroaryl.

In embodiments, R⁶ is independently substituted or unsubstituted C₁-C₆ alkyl. In embodiments, R⁶ is substituted or unsubstituted C₃-C₅ alkyl. In embodiments, R⁶ is independently substituted C₁-C₆ alkyl. In embodiments, R⁶ is substituted C₃-C₅ alkyl. In embodiments, R⁶ is independently unsubstituted C₁-C₆ alkyl. In embodiments, R⁶ is unsubstituted C₃-C₅ alkyl. In embodiments, R⁶ is independently fluoro-substituted C₁-C₆ alkyl. In embodiments, R⁶ is fluoro-substituted C₃-C₅ alkyl. In embodiments, R⁶ is fluoro-substituted or unsubstituted C₃-C₅ alkyl. In embodiments, R⁶ is independently substituted C₂-C₆ alkenyl. In embodiments, R⁶ is substituted C₃-C₅ alkenyl. In embodiments, R⁶ is independently unsubstituted C₂-C₆ alkenyl. In embodiments, R⁶ is unsubstituted C₃-C₅ alkyl. In embodiments, R⁶ is independently fluoro-substituted C₂-C₆ alkenyl. In embodiments, R⁶ is fluoro-substituted C₃-C₅ alkenyl. In embodiments, R⁶ is fluoro-substituted or unsubstituted C₃-C₅ alkenyl.

In embodiments, R⁶ is independently substituted or unsubstituted 2 to 6 membered heteroalkyl. In embodiments, R⁶ is independently substituted 2 to 6 membered heteroalkyl. In embodiments, R⁶ is independently unsubstituted 2 to 6 membered heteroalkyl. In embodiments, R⁶ is independently fluoro-substituted 2 to 6 membered heteroalkyl. In embodiments, R⁶ is independently substituted 3 to 6 membered heteroalkyl. In embodiments, R⁶ is independently unsubstituted 3 to 6 membered heteroalkyl. In embodiments, R⁶ is independently fluoro-substituted 3 to 6 membered heteroalkyl. In embodiments, R⁶ is independently substituted 2 to 6 membered heteroalkenyl. In embodiments, R⁶ is independently unsubstituted 2 to 6 membered heteroalkenyl. In embodiments, R⁶ is independently fluoro-substituted 2 to 6 membered heteroalkenyl. In embodiments, R⁶ is independently substituted 3 to 6 membered heteroalkenyl. In embodiments, R⁶ is independently unsubstituted 3 to 6 membered heteroalkenyl. In embodiments, R⁶ is independently fluoro-substituted 3 to 6 membered heteroalkenyl.

In embodiments, R⁶ is

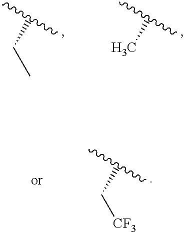

In embodiments, R⁶ is

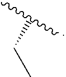

In embodiments, R⁶ is

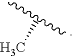

In embodiments, R⁶ is

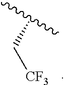

In embodiments, R⁷ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl. In embodiments, R⁷ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R⁷ is independently hydrogen, —CCl₃, —CF₃, —CHF₂, —CH₂F, substituted or unsubstituted alkyl, or substituted or unsubstituted heteroalkyl. In embodiments, R⁷ is independently hydrogen, —CCl₃, —CF₃, —CHF₂, —CH₂F, substituted or unsubstituted C₁-C₄ alkyl, or substituted or unsubstituted 2 to 4 membered heteroalkyl. In embodiments, R⁷ is independently fluoro-substituted or unsubstituted C₁-C₄ alkyl, or fluoro-substituted or unsubstituted 2 to 4 membered heteroalkyl. In embodiments, R⁷ is independently fluoro-substituted or unsubstituted C₁-C₄ alkyl. In embodiments, R⁷ is independently fluoro-substituted or unsubstituted 2 to 4 membered heteroalkyl. In embodiments, R⁷ is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁷ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R⁷ is independently hydrogen, —CCl₃, —CF₃, —CHF₂, —CH₂F, or substituted or unsubstituted alkyl.

In embodiments, R⁷ is independently hydrogen, substituted or unsubstituted C₁-C₄ alkyl, or substituted or unsubstituted 2 to 4 membered heteroalkyl. In embodiments, R⁷ is independently hydrogen. In embodiments, R⁷ is independently substituted or unsubstituted C₁-C₄ alkyl. In embodiments, R⁷ is independently substituted or unsubstituted 2 to 4 membered heteroalkyl. In embodiments, R⁷ is independently unsubstituted C₁-C₄ alkyl. In embodiments, R⁷ is independently unsubstituted 2 to 4 membered heteroalkyl. In embodiments, R⁷ is independently substituted or unsubstituted C₁-C₆ alkyl or substituted or unsubstituted 2 to 6 membered heteroalkyl. In embodiments, R⁷ is independently hydrogen. In embodiments, R⁷ is independently —CH₃. In embodiments, R⁷ is independently —CCl₃. In embodiments, R⁷ is independently —CBr₃. In embodiments, R⁷ is independently —CF₃. In embodiments, R⁷ is independently —CI₃. In embodiments, R⁷ is independently —CHCl₂. In embodiments, R⁷ is independently —CHBr₂. In embodiments, R⁷ is independently —CHF₂. In embodiments, R⁷ is independently —CHI₂. In embodiments, R⁷ is independently —CH₂Cl. In embodiments, R⁷ is independently —CH₂Br. In embodiments, R⁷ is independently —CH₂F. In embodiments, R⁷ is independently —CH₂I. In embodiments, R⁷ is independently —COOH. In embodiments, R⁷ is independently —COCH₃. In embodiments, R⁷ is independently —CONH₂. In embodiments, R⁷ is independently —CH₃. In embodiments, R⁷ is independently —CH₂CH₃. In embodiments, R⁷ is independently —CH(CH₃)₂. In embodiments, R⁷ is independently —C(CH₃)₃.

In embodiments, R⁷ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R⁷ is independently substituted or unsubstituted C₁-C₆ alkyl. In embodiments, R⁷ is independently unsubstituted C₁-C₆ alkyl. In embodiments, R⁷ is independently unsubstituted methyl. In embodiments, R⁷ is independently unsubstituted ethyl. In embodiments, R⁷ is independently unsubstituted propyl. In embodiments, R⁷ is independently substituted or unsubstituted 2 to 6 membered heteroalkyl. In embodiments, R⁷ is independently unsubstituted 2 to 6 membered heteroalkyl. In embodiments, R⁷ is independently substituted or unsubstituted C₃-C₆ cycloalkyl. In embodiments, R⁷ is independently unsubstituted C₃-C₆ cycloalkyl. In embodiments, R⁷ is independently substituted or unsubstituted 3 to 6 membered heterocycloalkyl. In embodiments, R⁷ is independently unsubstituted 3 to 6 membered heterocycloalkyl. In embodiments, R⁷ is independently substituted or unsubstituted C₆-C₁₀ aryl. In embodiments, R⁷ is independently unsubstituted C₆-C₁₀ aryl. In embodiments, R⁷ is independently substituted phenyl. In embodiments, R⁷ is independently unsubstituted phenyl. In embodiments, R⁷ is independently substituted or unsubstituted 5 to 10 membered heteroaryl. In embodiments, R⁷ is independently substituted or unsubstituted 5 to 6 membered heteroaryl. In embodiments, R⁷ is independently unsubstituted 5 to 10 membered heteroaryl. In embodiments, R⁷ is independently unsubstituted 5 to 6 membered heteroaryl.

In embodiments, R⁷ is independently substituted or unsubstituted C₁-C₆ alkyl. In embodiments, R⁷ is substituted or unsubstituted C₃-C₅ alkyl. In embodiments, R⁷ is independently substituted C₁-C₆ alkyl. In embodiments, R⁷ is substituted C₃-C₅ alkyl. In embodiments, R⁷ is independently unsubstituted C₁-C₆ alkyl. In embodiments, R⁷ is unsubstituted C₃-C₅ alkyl. In embodiments, R⁷ is independently fluoro-substituted C₁-C₆ alkyl. In embodiments, R⁷ is fluoro-substituted C₃-C₅ alkyl. In embodiments, R⁷ is fluoro-substituted or unsubstituted C₃-C₅ alkyl. In embodiments, R⁷ is independently substituted C₂-C₆ alkenyl. In embodiments, R⁷ is substituted C₃-C₅ alkenyl. In embodiments, R⁷ is independently unsubstituted C₂-C₆ alkenyl. In embodiments, R⁷ is unsubstituted C₃-C₅ alkyl. In embodiments, R⁷ is independently fluoro-substituted C₂-C₆ alkenyl. In embodiments, R⁷ is fluoro-substituted C₃-C₅ alkenyl. In embodiments, R⁷ is fluoro-substituted or unsubstituted C₃-C₅ alkenyl.

In embodiments, R⁷ is

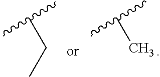

In embodiments, R⁷ is

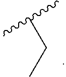 [SMILES: *CC]

In embodiments, R⁷ is

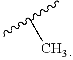

In embodiments, R⁶ and R⁷ substituents are joined to form, in combination with the —CHN— connecting the two substituents, a substituted or unsubstituted heterocycloalkyl. In embodiments, R⁶ and R⁷ substituents are joined to form, in combination with the —CHN— connecting the two substituents, a substituted or unsubstituted 6 to 8 membered heterocycloalkyl. In embodiments, R⁶ and R⁷ substituents are joined to form, in combination with the —CHN— connecting the two substituents, a fluoro-substituted or unsubstituted 6 to 8 membered heterocycloalkyl.

In embodiments, R⁶ and R⁷ substituents are joined to form, in combination with the —CHN— connecting the two substituents, a substituted heterocycloalkyl. In embodiments, R⁶ and R⁷ substituents are joined to form, in combination with the —CHN— connecting the two substituents, an unsubstituted heterocycloalkyl. In embodiments, R⁶ and R⁷ substituents are joined to form, in combination with the —CHN— connecting the two substituents, a substituted or unsubstituted 6 to 8 membered heterocycloalkyl. In embodiments, R⁶ and R⁷ substituents are joined to form, in combination with the —CHN— connecting the two substituents, a substituted 6 to 8 membered heterocycloalkyl. In embodiments, R⁶ and R⁷ substituents are joined to form, in combination with the —CHN— connecting the two substituents, an unsubstituted 6 to 8 membered heterocycloalkyl. In embodiments, R⁶ and R⁷ substituents are joined to form, in combination with the —CHN— connecting the two substituents, a substituted or unsubstituted 5 to 8 membered heterocycloalkyl. In embodiments, R⁶ and R⁷ substituents are joined to form, in combination with the —CHN— connecting the two substituents, a substituted 5 to 8 membered heterocycloalkyl. In embodiments, R⁶ and R⁷ substituents are joined to form, in combination with the —CHN— connecting the two substituents, an unsubstituted 5 to 8 membered heterocycloalkyl. In embodiments, R⁶ and R⁷ substituents are joined to form, in combination with the —CHN— connecting the two substituents, a substituted or unsubstituted 4 to 10 membered heterocycloalkyl. In embodiments, R⁶ and R⁷ substituents are joined to form, in combination with the —CHN— connecting the two substituents, a substituted 4 to 10 membered heterocycloalkyl. In embodiments, R⁶ and R⁷ substituents are joined to form, in combination with the —CHN— connecting the two substituents, an unsubstituted 4 to 10 membered heterocycloalkyl.

In embodiments, R⁶ and R⁷ substituents are joined to form, in combination with the —CHN— connecting the two substituents, a fluoro-substituted or unsubstituted 6 to 8 membered heterocycloalkyl. In embodiments, R⁶ and R⁷ substituents are joined to form, in combination with the —CHN— connecting the two substituents, a fluoro-substituted 6 to 8 membered heterocycloalkyl. In embodiments, R⁶ and R⁷ substituents are joined to form, in combination with the —CHN— connecting the two substituents, an unsubstituted 6 to 8 membered heterocycloalkyl.

In embodiments, R⁶ and R⁷ substituents are joined to form, in combination with the —CHN— connecting the two substituents,

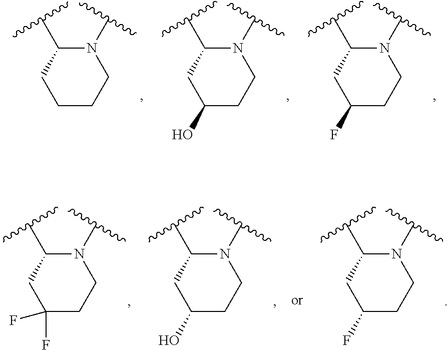 [SMILES: *[C@H]1CCCCN1* ; *[C@H]1C[C@H](O)CCN1* ; *[C@H]1C[C@H](F)CCN1* ; *[C@H]1CC(F)(F)CCN1* ; *[C@H]1C[C@@H](O)CCN1* ; *[C@H]1C[C@@H](F)CCN1*]

In embodiments, R⁶ and R⁷ substituents are joined to form, in combination with the —CHN— connecting the two substituents,

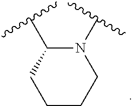 [SMILES: *[C@H]1CCCCN1*]

In embodiments, R⁶ and R⁷ substituents are joined to form, in combination with the —CHN— connecting the two substituents,

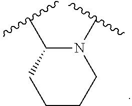 [SMILES: *[C@H]1CCCCN1*]

In embodiments, R⁶ and R⁷ substituents are joined to form, in combination with the —CHN— connecting the two substituents

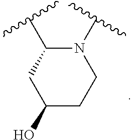 [SMILES: *[C@H]1C[C@H](O)CCN1*]

In embodiments, R⁶ and R⁷ substituents are joined to form, in combination with the —CHN— connecting the two substituents,

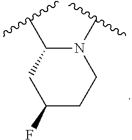 [SMILES: *[C@H]1C[C@H](F)CCN1*]

In embodiments, R⁶ and R⁷ substituents are joined to form, in combination with the —CHN— connecting the two substituents

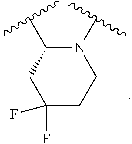 [SMILES: *[C@H]1CC(F)(F)CCN1*]

In embodiments, R⁶ and R⁷ substituents are joined to form, in combination with the —CHN— connecting the two substituents

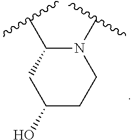 [SMILES: *[C@H]1C[C@@H](O)CCN1*]

In embodiments, R⁶ and R⁷ substituents are joined to form, in combination with the —CHN— connecting the two substituents

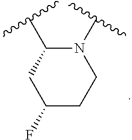 [SMILES: *[C@H]1C[C@@H](F)CCN1*]

In embodiments, R⁸ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R⁸ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl. In embodiments, R⁸ is hydrogen, —CCl₃, —CF₃, —CHF₂, —CH₂F, or substituted or unsubstituted alkyl. In embodiments, R⁸ is hydrogen, —CCl₃, —CF₃, —CHF₂, —CH₂F, or substituted or unsubstituted C₁-C₆ alkyl. In embodiments, R⁸ is fluoro-substituted or unsubstituted C₁-C₆ alkyl.

In embodiments, R⁸ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl. In embodiments, R⁸ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R⁸ is independently hydrogen, —CCl₃, —CF₃, —CHF₂, —CH₂F, substituted or unsubstituted alkyl, or substituted or unsubstituted heteroalkyl. In embodiments, R⁸ is independently hydrogen, —CCl₃, —CF₃, —CHF₂, —CH₂F, substituted or unsubstituted C₁-C₄ alkyl, or substituted or unsubstituted 2 to 4 membered heteroalkyl. In embodiments, R⁸ is independently fluoro-substituted or unsubstituted C₁-C₄ alkyl, or fluoro-substituted or unsubstituted 2 to 4 membered heteroalkyl. In embodiments, R⁸ is independently fluoro-substituted or unsubstituted C₁-C₄ alkyl. In embodiments, R⁸ is independently fluoro-substituted or unsubstituted 2 to 4 membered heteroalkyl. In embodiments, R⁸ is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁸ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R⁸ is independently hydrogen, —CCl₃, —CF₃, —CHF₂, —CH₂F, or substituted or unsubstituted alkyl.

In embodiments, R⁸ is independently hydrogen, substituted or unsubstituted C₁-C₄ alkyl, or substituted or unsubstituted 2 to 4 membered heteroalkyl. In embodiments, R⁸ is independently hydrogen. In embodiments, R⁸ is independently substituted or unsubstituted C₁-C₄ alkyl. In embodiments, R⁸ is independently substituted or unsubstituted 2 to 4 membered heteroalkyl. In embodiments, R⁸ is independently unsubstituted C₁-C₄ alkyl. In embodiments, R⁸ is independently unsubstituted 2 to 4 membered heteroalkyl. In embodiments, R⁸ is independently substituted or unsubstituted C₁-C₆ alkyl or substituted or unsubstituted 2 to 6 membered heteroalkyl. In embodiments, R⁸ is independently —CH₃. In embodiments, R⁸ is independently —CCl₃. In embodiments, R⁸ is independently —CBr₃. In embodiments, R⁸ is independently —CF₃. In embodiments, R⁸ is independently —CI₃. In embodiments, R⁸ is independently —CHCl₂. In embodiments, R⁸ is independently —CHBr₂. In embodiments, R⁸ is independently —CHF₂. In embodiments, R⁸ is independently —CHI₂. In embodiments, R⁸ is independently —CH₂Cl. In embodiments, R⁸ is independently —CH₂Br. In embodiments, R⁸ is independently —CH₂F. In embodiments, R⁸ is independently —CH₂I. In embodiments, R⁸ is independently —COOH. In embodiments, R⁸ is independently —COCH₃. In embodiments, R⁸ is independently —CONH₂. In embodiments, R⁸ is independently —CH₃. In embodiments, R⁸ is independently —CH₂CH₃. In embodiments, R⁸ is independently —CH(CH₃)₂. In embodiments, R⁸ is independently —C(CH₃)₃.

In embodiments, R⁸ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R⁸ is independently substituted or unsubstituted C₁-C₆ alkyl. In embodiments, R⁸ is independently unsubstituted C₁-C₆ alkyl. In embodiments, R⁸ is independently unsubstituted methyl. In embodiments, R⁸ is independently unsubstituted ethyl. In embodiments, R⁸ is independently unsubstituted propyl. In embodiments, R⁸ is independently substituted or unsubstituted 2 to 6 membered heteroalkyl. In embodiments, R⁸ is independently unsubstituted 2 to 6 membered heteroalkyl. In embodiments, R⁸ is independently substituted or unsubstituted C₃-C₆ cycloalkyl. In embodiments, R⁸ is independently unsubstituted C₃-C₆ cycloalkyl. In embodiments, R⁸ is independently substituted or unsubstituted 3 to 6 membered heterocycloalkyl. In embodiments, R⁸ is independently unsubstituted 3 to 6 membered heterocycloalkyl. In embodiments, R⁸ is independently substituted or unsubstituted C₆-C₁₀ aryl. In embodiments, R⁸ is independently unsubstituted C₆-C₁₀ aryl. In embodiments, R⁸ is independently substituted phenyl. In embodiments, R⁸ is independently unsubstituted phenyl. In embodiments, R⁸ is independently substituted or unsubstituted 5 to 10 membered heteroaryl. In embodiments, R⁸ is independently substituted or unsubstituted 5 to 6 membered heteroaryl. In embodiments, R⁸ is independently unsubstituted 5 to 10 membered heteroaryl. In embodiments, R⁸ is independently unsubstituted 5 to 6 membered heteroaryl.

In embodiments, R⁸ is independently substituted or unsubstituted C₁-C₆ alkyl. In embodiments, R⁸ is substituted or unsubstituted C₃-C₅ alkyl. In embodiments, R⁸ is independently substituted C₁-C₆ alkyl. In embodiments, R⁸ is substituted C₃-C₅ alkyl. In embodiments, R⁸ is independently unsubstituted C₁-C₆ alkyl. In embodiments, R⁸ is unsubstituted C₃-C₅ alkyl. In embodiments, R⁸ is independently fluoro-substituted C₁-C₆ alkyl. In embodiments, R⁸ is fluoro-substituted C₃-C₅ alkyl. In embodiments, R⁸ is fluoro-substituted or unsubstituted C₃-C₅ alkyl. In embodiments, R⁸ is independently substituted C₂-C₆ alkenyl. In embodiments, R⁸ is substituted C₃-C₅ alkenyl. In embodiments, R⁸ is independently unsubstituted C₂-C₆ alkenyl. In embodiments, R⁸ is unsubstituted C₃-C₅ alkyl. In embodiments, R⁸ is independently fluoro-substituted C₂-C₆ alkenyl. In embodiments, R⁸ is fluoro-substituted C₃-C₅ alkenyl. In embodiments, R⁸ is fluoro-substituted or unsubstituted C₃-C₅ alkenyl.

In embodiments, R⁸ is independently substituted or unsubstituted 2 to 6 membered heteroalkyl. In embodiments, R⁸ is independently substituted 2 to 6 membered heteroalkyl. In embodiments, R⁸ is independently unsubstituted 2 to 6 membered heteroalkyl. In embodiments, R′ is independently fluoro-substituted 2 to 6 membered heteroalkyl. In embodiments, R⁸ is independently substituted 3 to 6 membered heteroalkyl. In embodiments, R⁸ is independently unsubstituted 3 to 6 membered heteroalkyl. In embodiments, R⁸ is independently fluoro-substituted 3 to 6 membered heteroalkyl. In embodiments, R⁸ is independently substituted 2 to 6 membered heteroalkenyl. In embodiments, R⁸ is independently unsubstituted 2 to 6 membered heteroalkenyl. In embodiments, R⁸ is independently fluoro-substituted 2 to 6 membered heteroalkenyl. In embodiments, R⁸ is independently substituted 3 to 6 membered heteroalkenyl. In embodiments, R⁸ is independently unsubstituted 3 to 6 membered heteroalkenyl. In embodiments, R⁸ is independently fluoro-substituted 3 to 6 membered heteroalkenyl.

In embodiments, R⁹ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R⁹ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl. In embodiments, R⁹ is hydrogen, —CCl₃, —CF₃, —CHF₂, —CH₂F, or substituted or unsubstituted alkyl. In embodiments, R⁹ is hydrogen, —CCl₃, —CF₃, —CHF₂, —CH₂F, or substituted or unsubstituted C₁-C₆ alkyl. In embodiments, R⁹ is fluoro-substituted or unsubstituted C₁-C₆ alkyl.

In embodiments, R⁹ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl. In embodiments, R⁹ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R⁹ is independently hydrogen, —CCl₃, —CF₃, —CHF₂, —CH₂F, substituted or unsubstituted alkyl, or substituted or unsubstituted heteroalkyl. In embodiments, R⁹ is independently hydrogen, —CCl₃, —CF₃, —CHF₂, —CH₂F, substituted or unsubstituted C₁-C₄ alkyl, or substituted or unsubstituted 2 to 4 membered heteroalkyl. In embodiments, R⁹ is independently fluoro-substituted or unsubstituted C₁-C₄ alkyl, or fluoro-substituted or unsubstituted 2 to 4 membered heteroalkyl. In embodiments, R⁹ is independently fluoro-substituted or unsubstituted C₁-C₄ alkyl. In embodiments, R⁹ is independently fluoro-substituted or unsubstituted 2 to 4 membered heteroalkyl. In embodiments, R⁹ is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁹ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R⁹ is independently hydrogen, —CCl₃, —CF₃, —CHF₂, —CH₂F, or substituted or unsubstituted alkyl.

In embodiments, R⁹ is independently hydrogen, substituted or unsubstituted C₁-C₄ alkyl, or substituted or unsubstituted 2 to 4 membered heteroalkyl. In embodiments, R⁹ is independently hydrogen. In embodiments, R⁹ is independently substituted or unsubstituted C₁-C₄ alkyl. In embodiments, R⁹ is independently substituted or unsubstituted 2 to 4 membered heteroalkyl. In embodiments, R⁹ is independently unsubstituted C₁-C₄ alkyl. In embodiments, R⁹ is independently unsubstituted 2 to 4 membered heteroalkyl. In embodiments, R⁹ is independently substituted or unsubstituted C₁-C₆ alkyl or substituted or unsubstituted 2 to 6 membered heteroalkyl. In embodiments, R⁹ is independently —CH₃. In embodiments, R⁹ is independently —CCl₃. In embodiments, R⁹ is independently —CBr₃. In embodiments, R⁹ is independently —CF₃. In embodiments, R⁹ is independently —CI₃. In embodiments, R⁹ is independently —CHCl₂. In embodiments, R⁹ is independently —CHBr₂. In embodiments, R⁹ is independently —CHF₂. In embodiments, R⁹ is independently —CHI₂. In embodiments, R⁹ is independently —CH₂Cl. In embodiments, R⁹ is independently —CH₂Br. In embodiments, R⁹ is independently —CH₂F. In embodiments, R⁹ is independently —CH₂I. In embodiments, R⁹ is independently —COOH. In embodiments, R⁹ is independently —COCH₃. In embodiments, R⁹ is independently —CONH₂. In embodiments, R⁹ is independently —CH₃. In embodiments, R⁹ is independently —CH₂CH₃. In embodiments, R⁹ is independently —CH(CH₃)₂. In embodiments, R⁹ is independently —C(CH₃)₃.

In embodiments, R⁹ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R⁹ is independently substituted or unsubstituted C₁-C₆ alkyl. In embodiments, R⁹ is independently unsubstituted C₁-C₆ alkyl. In embodiments, R⁹ is independently unsubstituted methyl. In embodiments, R⁹ is independently unsubstituted ethyl. In embodiments, R⁹ is independently unsubstituted propyl. In embodiments, R⁹ is independently substituted or unsubstituted 2 to 6 membered heteroalkyl. In embodiments, R⁹ is independently unsubstituted 2 to 6 membered heteroalkyl. In embodiments, R⁹ is independently substituted or unsubstituted C₃-C₆ cycloalkyl. In embodiments, R⁹ is independently unsubstituted C₃-C₆ cycloalkyl. In embodiments, R⁹ is independently substituted or unsubstituted 3 to 6 membered heterocycloalkyl. In embodiments, R⁹ is independently unsubstituted 3 to 6 membered heterocycloalkyl. In embodiments, R⁹ is independently substituted or unsubstituted C₆-C₁₀ aryl. In embodiments, R⁹ is independently unsubstituted C₆-C₁₀ aryl. In embodiments, R⁹ is independently substituted phenyl. In embodiments, R⁹ is independently unsubstituted phenyl. In embodiments, R⁹ is independently substituted or unsubstituted 5 to 10 membered heteroaryl. In embodiments, R⁹ is independently substituted or unsubstituted 5 to 6 membered heteroaryl. In embodiments, R⁹ is independently unsubstituted 5 to 10 membered heteroaryl. In embodiments, R⁹ is independently unsubstituted 5 to 6 membered heteroaryl.

In embodiments, R⁹ is independently substituted or unsubstituted C₁-C₆ alkyl. In embodiments, R⁹ is substituted or unsubstituted C₃-C₅ alkyl. In embodiments, R⁹ is independently substituted C₁-C₆ alkyl. In embodiments, R⁹ is substituted C₃-C₅ alkyl. In embodiments, R⁹ is independently unsubstituted C₁-C₆ alkyl. In embodiments, R⁹ is unsubstituted C₃-C₅ alkyl. In embodiments, R⁹ is independently fluoro-substituted C₁-C₆ alkyl. In embodiments, R⁹ is fluoro-substituted C₃-C₅ alkyl. In embodiments, R⁹ is fluoro-substituted or unsubstituted C₃-C₅ alkyl. In embodiments, R⁹ is independently substituted C₂-C₆ alkenyl. In embodiments, R⁹ is substituted C₃-C₅ alkenyl. In embodiments, R⁹ is independently unsubstituted C₂-C₆ alkenyl. In embodiments, R⁹ is unsubstituted C₃-C₅ alkyl. In embodiments, R⁹ is independently fluoro-substituted C₂-C₆ alkenyl. In embodiments, R⁹ is fluoro-substituted C₃-C₅ alkenyl. In embodiments, R⁹ is fluoro-substituted or unsubstituted C₃-C₅ alkenyl.

In embodiments, R⁹ is independently substituted or unsubstituted 2 to 6 membered heteroalkyl. In embodiments, R⁹ is independently substituted 2 to 6 membered heteroalkyl. In embodiments, R⁹ is independently unsubstituted 2 to 6 membered heteroalkyl. In embodiments, R⁹ is independently fluoro-substituted 2 to 6 membered heteroalkyl. In embodiments, R⁹ is independently substituted 3 to 6 membered heteroalkyl. In embodiments, R⁹ is independently unsubstituted 3 to 6 membered heteroalkyl. In embodiments, R⁹ is independently fluoro-substituted 3 to 6 membered heteroalkyl. In embodiments, R⁹ is independently substituted 2 to 6 membered heteroalkenyl. In embodiments, R⁹ is independently unsubstituted 2 to 6 membered heteroalkenyl. In embodiments, R⁹ is independently fluoro-substituted 2 to 6 membered heteroalkenyl. In embodiments, R⁹ is independently substituted 3 to 6 membered heteroalkenyl. In embodiments, R⁹ is independently unsubstituted 3 to 6 membered heteroalkenyl. In embodiments, R⁹ is independently fluoro-substituted 3 to 6 membered heteroalkenyl.

In embodiments, R¹⁰ is

hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹⁰ is
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl. In embodiments, R¹⁰ is
hydrogen, —CCl₃, —CF₃, —CHF₂, —CH₂F, or substituted or unsubstituted alkyl. In embodiments, R¹⁰ is hydrogen, —CCl₃, —CF₃, —CHF₂, —CH₂F, or substituted or unsubstituted C₁-C₆ alkyl. In embodiments, R¹⁰ is fluoro-substituted or unsubstituted C₁-C₆ alkyl. In embodiments, R¹⁰ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl. In embodiments, R¹⁰ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R¹⁰ is independently hydrogen, —CCl₃, —CF₃, —CHF₂, —CH₂F, substituted or unsubstituted alkyl, or substituted or unsubstituted heteroalkyl. In embodiments, R¹⁰ is independently
hydrogen, —CCl₃, —CF₃, —CHF₂, —CH₂F, substituted or unsubstituted C₁-C₄ alkyl, or substituted or unsubstituted 2 to 4 membered heteroalkyl. In embodiments, R¹⁰ is independently fluoro-substituted or unsubstituted C₁-C₄ alkyl, or fluoro-substituted or unsubstituted 2 to 4 membered heteroalkyl. In embodiments, R¹⁰ is independently fluoro-substituted or unsubstituted C₁-C₄ alkyl. In embodiments, R¹⁰ is independently fluoro-substituted or unsubstituted 2 to 4 membered heteroalkyl. In embodiments, R¹⁰ is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁰ is independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹⁰ is independently
hydrogen, —CCl₃, —CF₃, —CHF₂, —CH₂F, or substituted or unsubstituted alkyl. In embodiments, R¹⁰ is independently hydrogen, substituted or unsubstituted C₁-C₄ alkyl, or substituted or unsubstituted 2 to 4 membered heteroalkyl. In embodiments, R¹⁰ is independently hydrogen. In embodiments, R¹⁰ is independently substituted or unsubstituted C₁-C₄ alkyl. In embodiments, R¹⁰ is independently substituted or unsubstituted 2 to 4 membered heteroalkyl. In embodiments, R¹⁰ is independently unsubstituted C₁-C₄ alkyl. In embodiments, R¹⁰ is independently unsubstituted 2 to 4 membered heteroalkyl. In embodiments, R¹⁰ is independently —CH₃. In embodiments, R¹⁰ is independently —CCl₃. In embodiments, R¹⁰ is independently —CBr₃. In embodiments, R¹⁰ is independently —CF₃. In embodiments, R¹⁰ is independently —CI₃. In embodiments, R¹⁰ is independently —CHCl₂. In embodiments, R¹⁰ is independently —CHBr₂. In embodiments, R¹⁰ is independently —CHF₂. In embodiments, R¹⁰ is independently —CHI₂. In embodiments, R¹⁰ is independently —CH₂Cl. In embodiments, R¹⁰ is independently —CH₂Br. In embodiments, R¹⁰ is independently —CH₂F. In embodiments, R¹⁰ is independently —CH₂I. In embodiments, R¹⁰ is independently —CN. In embodiments, R¹⁰ is independently —COOH. In embodiments, R¹⁰ is independently —COCH₃. In embodiments, R¹⁰ is independently —CONH₂. In embodiments, R¹⁰ is independently —CH₃. In embodiments, R¹⁰ is independently —CH₂CH₃. In embodiments, R¹⁰ is independently —CH(CH₃)₂. In embodiments, R¹⁰ is independently —C(CH₃)₃. In embodiments, R¹⁰ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹⁰ is independently substituted or unsubstituted C₁-C₆ alkyl. In embodiments, R¹⁰ is independently unsubstituted C₁-C₆ alkyl. In embodiments, R¹⁰ is independently unsubstituted methyl. In embodiments, R¹⁰ is independently unsubstituted ethyl. In embodiments, R¹⁰ is independently unsubstituted propyl. In embodiments, R¹⁰ is independently substituted or unsubstituted 2 to 6 membered heteroalkyl. In embodiments, R¹⁰ is independently unsubstituted 2 to 6 membered heteroalkyl. In embodiments, R¹⁰ is independently substituted or unsubstituted C₃-C₆ cycloalkyl. In embodiments, R¹⁰ is independently unsubstituted C₃-C₆ cycloalkyl. In embodiments, R¹⁰ is independently substituted or unsubstituted 3 to 6 membered heterocycloalkyl. In embodiments, R¹⁰ is independently unsubstituted 3 to 6 membered heterocycloalkyl. In embodiments, R¹⁰ is independently substituted or unsubstituted C₆-C₁₀ aryl. In embodiments, R¹⁰ is independently unsubstituted C₆-C₁₀ aryl. In embodiments, R¹⁰ is independently substituted phenyl. In embodiments, R¹⁰ is independently unsubstituted phenyl. In embodiments, R¹⁰ is independently substituted or unsubstituted 5 to 10 membered heteroaryl. In embodiments, R¹⁰ is independently substituted or unsubstituted 5 to 6 membered heteroaryl. In embodiments, R¹⁰ is independently unsubstituted 5 to 10 membered heteroaryl. In embodiments, R¹⁰ is independently unsubstituted 5 to 6 membered heteroaryl.

In embodiments, R¹¹ is

hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹¹ is
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl. In embodiments, R¹¹ is
hydrogen, —CCl₃, —CF₃, —CHF₂, —CH₂F, or substituted or unsubstituted alkyl. In embodiments, R¹¹ is hydrogen, —CCl₃, —CF₃, —CHF₂, —CH₂F, or substituted or unsubstituted C₁-C₆ alkyl. In embodiments, R¹¹ is fluoro-substituted or unsubstituted C₁-C₆ alkyl. In embodiments, R¹¹ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl. In embodiments, R¹¹ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R¹¹ is independently
hydrogen, —CCl₃, —CF₃, —CHF₂, —CH₂F, substituted or unsubstituted alkyl, or substituted or unsubstituted heteroalkyl. In embodiments, R¹¹ is independently
hydrogen, —CCl₃, —CF₃, —CHF₂, —CH₂F, substituted or unsubstituted C₁-C₄ alkyl, or substituted or unsubstituted 2 to 4 membered heteroalkyl. In embodiments, R¹¹ is independently fluoro-substituted or unsubstituted C₁-C₄ alkyl, or fluoro-substituted or unsubstituted 2 to 4 membered heteroalkyl. In embodiments, R¹¹ is independently fluoro-substituted or unsubstituted C₁-C₄ alkyl. In embodiments, R¹¹ is independently fluoro-substituted or unsubstituted 2 to 4 membered heteroalkyl. In embodiments, R¹¹ is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹¹ is independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹¹ is independently
hydrogen, —CCl₃, —CF₃, —CHF₂, —CH₂F, or substituted or unsubstituted alkyl. In embodiments, R¹¹ is independently hydrogen, substituted or unsubstituted C₁-C₄ alkyl, or substituted or unsubstituted 2 to 4 membered heteroalkyl. In embodiments, R¹¹ is independently hydrogen. In embodiments, R¹¹ is independently substituted or unsubstituted C₁-C₄ alkyl. In embodiments, R¹¹ is independently substituted or unsubstituted 2 to 4 membered heteroalkyl. In embodiments, R¹¹ is independently unsubstituted C₁-C₄ alkyl. In embodiments, R¹¹ is independently unsubstituted 2 to 4 membered heteroalkyl. In embodiments, R¹¹ is independently —CH₃. In embodiments, R¹¹ is independently —CCl₃. In embodiments, R¹¹ is independently —CBr₃. In embodiments, R¹¹ is independently —CF₃. In embodiments, R¹¹ is independently —CI₃. In embodiments, R¹¹ is independently —CHCl₂. In embodiments, R¹¹ is independently —CHBr₂. In embodiments, R¹¹ is independently —CHF₂. In embodiments, R¹¹ is independently —CHI₂. In embodiments, R¹¹ is independently —CH₂Cl. In embodiments, R¹¹ is independently —CH₂Br. In embodiments, R¹¹ is independently —CH₂F. In embodiments, R¹¹ is independently —CH₂I. In embodiments, R¹¹ is independently —CN. In embodiments, R¹¹ is independently —COOH. In embodiments, R¹¹ is independently —COCH₃. In embodiments, R¹¹ is independently —CONH₂. In embodiments, R¹¹ is independently —CH₃. In embodiments, R¹¹ is independently —CH₂CH₃. In embodiments, R¹¹ is independently —CH(CH₃)₂. In embodiments, R¹¹ is independently —C(CH₃)₃. In embodiments, R″ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹¹ is independently substituted or unsubstituted C₁-C₆ alkyl. In embodiments, R¹¹ is independently unsubstituted C₁-C₆ alkyl. In embodiments, R¹¹ is independently unsubstituted methyl. In embodiments, R¹¹ is independently unsubstituted ethyl. In embodiments, R¹¹ is independently unsubstituted propyl. In embodiments, R¹¹ is independently substituted or unsubstituted 2 to 6 membered heteroalkyl. In embodiments, R″ is independently unsubstituted 2 to 6 membered heteroalkyl. In embodiments, R¹¹ is independently substituted or unsubstituted C₃-C₆ cycloalkyl. In embodiments, R¹¹ is independently unsubstituted C₃-C₆ cycloalkyl. In embodiments, R¹¹ is independently substituted or unsubstituted 3 to 6 membered heterocycloalkyl. In embodiments, R¹¹ is independently unsubstituted 3 to 6 membered heterocycloalkyl. In embodiments, R¹¹ is independently substituted or unsubstituted C₆-C₁₀ aryl. In embodiments, R¹¹ is independently unsubstituted C₆-C₁₀ aryl. In embodiments, R¹¹ is independently substituted phenyl. In embodiments, R¹¹ is independently unsubstituted phenyl. In embodiments, R¹¹ is independently substituted or unsubstituted 5 to 10 membered heteroaryl. In embodiments, R¹¹ is independently substituted or unsubstituted 5 to 6 membered heteroaryl. In embodiments, R¹¹ is independently unsubstituted 5 to 10 membered heteroaryl. In embodiments, R¹¹ is independently unsubstituted 5 to 6 membered heteroaryl.

In embodiments, R¹² is

hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹² is
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl. In embodiments, R¹² is
hydrogen, —CCl₃, —CF₃, —CHF₂, —CH₂F, or substituted or unsubstituted alkyl. In embodiments, R¹² is hydrogen, —CCl₃, —CF₃, —CHF₂, —CH₂F, or substituted or unsubstituted C₁-C₆ alkyl. In embodiments, R¹² is fluoro-substituted or unsubstituted C₁-C₆ alkyl. In embodiments, R¹² is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl. In embodiments, R¹² is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R¹² is independently
hydrogen, —CCl₃, —CF₃, —CHF₂, —CH₂F, substituted or unsubstituted alkyl, or substituted or unsubstituted heteroalkyl. In embodiments, R¹² is independently
hydrogen, —CCl₃, —CF₃, —CHF₂, —CH₂F, substituted or unsubstituted C₁-C₄ alkyl, or substituted or unsubstituted 2 to 4 membered heteroalkyl. In embodiments, R¹² is independently fluoro-substituted or unsubstituted C₁-C₄ alkyl, or fluoro-substituted or unsubstituted 2 to 4 membered heteroalkyl. In embodiments, R¹² is independently fluoro-substituted or unsubstituted C₁-C₄ alkyl. In embodiments, R¹² is independently fluoro-substituted or unsubstituted 2 to 4 membered heteroalkyl. In embodiments, R¹² is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹² is independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹² is independently
hydrogen, —CCl₃, —CF₃, —CHF₂, —CH₂F, or substituted or unsubstituted alkyl. In embodiments, R¹² is independently hydrogen, substituted or unsubstituted C₁-C₄ alkyl, or substituted or unsubstituted 2 to 4 membered heteroalkyl. In embodiments, R¹² is independently hydrogen. In embodiments, R¹² is independently substituted or unsubstituted C₁-C₄ alkyl. In embodiments, R¹² is independently substituted or unsubstituted 2 to 4 membered heteroalkyl. In embodiments, R¹² is independently unsubstituted C₁-C₄ alkyl. In embodiments, R¹² is independently unsubstituted 2 to 4 membered heteroalkyl. In embodiments, R¹² is independently —CH₃. In embodiments, R¹² is independently —CCl₃. In embodiments, R¹² is independently —CBr₃. In embodiments, R¹² is independently —CF₃. In embodiments, R¹² is independently —CI₃. In embodiments, R¹² is independently —CHCl₂. In embodiments, R¹² is independently —CHBr₂. In embodiments, R¹² is independently —CHF₂. In embodiments, R¹² is independently —CHI₂. In embodiments, R¹² is independently —CH₂Cl. In embodiments, R¹² is independently —CH₂Br. In embodiments, R¹² is independently —CH₂F. In embodiments, R¹² is independently —CH₂I. In embodiments, R¹² is independently —CN. In embodiments, R¹² is independently —COOH. In embodiments, R¹² is independently —COCH₃. In embodiments, R¹² is independently —CONH₂. In embodiments, R¹² is independently —CH₃. In embodiments, R¹² is independently —CH₂CH₃. In embodiments, R¹² is independently —CH(CH₃)₂. In embodiments, R¹² is independently —C(CH₃)₃. In embodiments, R¹² is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹² is independently substituted or unsubstituted C₁-C₆ alkyl. In embodiments, R¹² is independently unsubstituted C₁-C₆ alkyl. In embodiments, R¹² is independently unsubstituted methyl. In embodiments, R¹² is independently unsubstituted ethyl. In embodiments, R¹² is independently unsubstituted propyl. In embodiments, R¹² is independently substituted or unsubstituted 2 to 6 membered heteroalkyl. In embodiments, R¹² is independently unsubstituted 2 to 6 membered heteroalkyl. In embodiments, R¹² is independently substituted or unsubstituted C₃-C₆ cycloalkyl. In embodiments, R¹² is independently unsubstituted C₃-C₆ cycloalkyl. In embodiments, R¹² is independently substituted or unsubstituted 3 to 6 membered heterocycloalkyl. In embodiments, R¹² is independently unsubstituted 3 to 6 membered heterocycloalkyl. In embodiments, R¹² is independently substituted or unsubstituted C₆-C₁₀ aryl. In embodiments, R¹² is independently unsubstituted C₆-C₁₀ aryl. In embodiments, R¹² is independently substituted phenyl. In embodiments, R¹² is independently unsubstituted phenyl. In embodiments, R¹² is independently substituted or unsubstituted 5 to 10 membered heteroaryl. In embodiments, R¹² is independently substituted or unsubstituted 5 to 6 membered heteroaryl. In embodiments, R¹² is independently unsubstituted 5 to 10 membered heteroaryl. In embodiments, R¹² is independently unsubstituted 5 to 6 membered heteroaryl.

In embodiments, R¹³ is

hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹³ is
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl. In embodiments, R¹³ is
hydrogen, —CCl₃, —CF₃, —CHF₂, —CH₂F, or substituted or unsubstituted alkyl. In embodiments, R¹³ is hydrogen, —CCl₃, —CF₃, —CHF₂, —CH₂F, or substituted or unsubstituted C₁-C₆ alkyl. In embodiments, R¹³ is fluoro-substituted or unsubstituted C₁-C₆ alkyl. In embodiments, R¹³ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl. In embodiments, R¹³ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R¹³ is independently
hydrogen, —CCl₃, —CF₃, —CHF₂, —CH₂F, substituted or unsubstituted alkyl, or substituted or unsubstituted heteroalkyl. In embodiments, R¹³ is independently
hydrogen, —CCl₃, —CF₃, —CHF₂, —CH₂F, substituted or unsubstituted C₁-C₄ alkyl, or substituted or unsubstituted 2 to 4 membered heteroalkyl. In embodiments, R¹³ is independently fluoro-substituted or unsubstituted C₁-C₄ alkyl, or fluoro-substituted or unsubstituted 2 to 4 membered heteroalkyl. In embodiments, R¹³ is independently fluoro-substituted or unsubstituted C₁-C₄ alkyl. In embodiments, R¹³ is independently fluoro-substituted or unsubstituted 2 to 4 membered heteroalkyl. In embodiments, R¹³ is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹³ is independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹³ is independently hydrogen, —CCl₃, —CF₃, —CHF₂, —CH₂F, or substituted or unsubstituted alkyl. In embodiments, R¹³ is independently hydrogen, substituted or unsubstituted C₁-C₄ alkyl, or substituted or unsubstituted 2 to 4 membered heteroalkyl. In embodiments, R¹³ is independently hydrogen. In embodiments, R¹³ is independently substituted or unsubstituted C₁-C₄ alkyl. In embodiments, R¹³ is independently substituted or unsubstituted 2 to 4 membered heteroalkyl. In embodiments, R¹³ is independently unsubstituted C₁-C₄ alkyl. In embodiments, R¹³ is independently unsubstituted 2 to 4 membered heteroalkyl. In embodiments, R¹³ is independently —CH₃. In embodiments, R¹³ is independently —CCl₃. In embodiments, R¹³ is independently —CBr₃. In embodiments, R¹³ is independently —CF₃. In embodiments, R¹³ is independently —CI₃. In embodiments, R¹³ is independently —CHCl₂. In embodiments, R¹³ is independently —CHBr₂. In embodiments, R¹³ is independently —CHF₂. In embodiments, R¹³ is independently —CHI₂. In embodiments, R¹³ is independently —CH₂Cl. In embodiments, R¹³ is independently —CH₂Br. In embodiments, R¹³ is independently —CH₂F. In embodiments, R¹³ is independently —CH₂I. In embodiments, R¹³ is independently —CN. In embodiments, R¹³ is independently —COOH. In embodiments, R¹³ is independently —COCH₃. In embodiments, R¹³ is independently —CONH₂. In embodiments, R¹³ is independently —CH₃. In embodiments, R¹³ is independently —CH₂CH₃. In embodiments, R¹³ is independently —CH(CH₃)₂. In embodiments, R¹³ is independently —C(CH₃)₃. In embodiments, R¹³ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹³ is independently substituted or unsubstituted C₁-C₆ alkyl. In embodiments, R¹³ is independently unsubstituted C₁-C₆ alkyl. In embodiments, R¹³ is independently unsubstituted methyl. In embodiments, R¹³ is independently unsubstituted ethyl. In embodiments, R¹³ is independently unsubstituted propyl. In embodiments, R¹³ is independently substituted or unsubstituted 2 to 6 membered heteroalkyl. In embodiments, R¹³ is independently unsubstituted 2 to 6 membered heteroalkyl. In embodiments, R¹³ is independently substituted or unsubstituted C₃-C₆ cycloalkyl. In embodiments, R¹³ is independently unsubstituted C₃-C₆ cycloalkyl. In embodiments, R¹³ is independently substituted or unsubstituted 3 to 6 membered heterocycloalkyl. In embodiments, R¹³ is independently unsubstituted 3 to 6 membered heterocycloalkyl. In embodiments, R¹³ is independently substituted or unsubstituted C₆-C₁₀ aryl. In embodiments, R¹³ is independently unsubstituted C₆-C₁₀ aryl. In embodiments, R¹³ is independently substituted phenyl. In embodiments, R¹³ is independently unsubstituted phenyl. In embodiments, R¹³ is independently substituted or unsubstituted 5 to 10 membered heteroaryl. In embodiments, R¹³ is independently substituted or unsubstituted 5 to 6 membered heteroaryl. In embodiments, R¹³ is independently unsubstituted 5 to 10 membered heteroaryl. In embodiments, R¹³ is independently unsubstituted 5 to 6 membered heteroaryl.

In embodiments, R¹⁴ is

hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹⁴ is
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl. In embodiments, R¹⁴ is
hydrogen, —CCl₃, —CF₃, —CHF₂, —CH₂F, or substituted or unsubstituted alkyl. In embodiments, R¹⁴ is hydrogen, —CCl₃, —CF₃, —CHF₂, —CH₂F, or substituted or unsubstituted C₁-C₆ alkyl. In embodiments, R¹⁴ is fluoro-substituted or unsubstituted C₁-C₆ alkyl. In embodiments, R¹⁴ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl. In embodiments, R¹⁴ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R¹⁴ is independently hydrogen, —CCl₃, —CF₃, —CHF₂, —CH₂F, substituted or unsubstituted alkyl, or substituted or unsubstituted heteroalkyl. In embodiments, R¹⁴ is independently
hydrogen, —CCl₃, —CF₃, —CHF₂, —CH₂F, substituted or unsubstituted C₁-C₄ alkyl, or substituted or unsubstituted 2 to 4 membered heteroalkyl. In embodiments, R¹⁴ is independently fluoro-substituted or unsubstituted C₁-C₄ alkyl, or fluoro-substituted or unsubstituted 2 to 4 membered heteroalkyl. In embodiments, R¹⁴ is independently fluoro-substituted or unsubstituted C₁-C₄ alkyl. In embodiments, R¹⁴ is independently fluoro-substituted or unsubstituted 2 to 4 membered heteroalkyl. In embodiments, R¹⁴ is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁴ is independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹⁴ is independently
hydrogen, —CCl₃, —CF₃, —CHF₂, —CH₂F, or substituted or unsubstituted alkyl. In embodiments, R¹⁴ is independently hydrogen, substituted or unsubstituted C₁-C₄ alkyl, or substituted or unsubstituted 2 to 4 membered heteroalkyl. In embodiments, R¹⁴ is independently hydrogen. In embodiments, R¹⁴ is independently substituted or unsubstituted C₁-C₄ alkyl. In embodiments, R¹⁴ is independently substituted or unsubstituted 2 to 4 membered heteroalkyl. In embodiments, R¹⁴ is independently unsubstituted C₁-C₄ alkyl. In embodiments, R¹⁴ is independently unsubstituted 2 to 4 membered heteroalkyl. In embodiments, R¹⁴ is independently —CH₃. In embodiments, R¹⁴ is independently —CCl₃. In embodiments, R¹⁴ is independently —CBr₃. In embodiments, R¹⁴ is independently —CF₃. In embodiments, R¹⁴ is independently —CI₃. In embodiments, R¹⁴ is independently —CHCl₂. In embodiments, R¹⁴ is independently —CHBr₂. In embodiments, R¹⁴ is independently —CHF₂. In embodiments, R¹⁴ is independently —CHI₂. In embodiments, R¹⁴ is independently —CH₂Cl. In embodiments, R¹⁴ is independently —CH₂Br. In embodiments, R¹⁴ is independently —CH₂F. In embodiments, R¹⁴ is independently —CH₂I. In embodiments, R¹⁴ is independently —CN. In embodiments, R¹⁴ is independently —COOH. In embodiments, R¹⁴ is independently —COCH₃. In embodiments, R¹⁴ is independently —CONH₂. In embodiments, R¹⁴ is independently —CH₃. In embodiments, R¹⁴ is independently —CH₂CH₃. In embodiments, R¹⁴ is independently —CH(CH₃)₂. In embodiments, R¹⁴ is independently —C(CH₃)₃. In embodiments, R¹⁴ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹⁴ is independently substituted or unsubstituted C₁-C₆ alkyl. In embodiments, R¹⁴ is independently unsubstituted C₁-C₆ alkyl. In embodiments, R¹⁴ is independently unsubstituted methyl. In embodiments, R¹⁴ is independently unsubstituted ethyl. In embodiments, R¹⁴ is independently unsubstituted propyl. In embodiments, R¹⁴ is independently substituted or unsubstituted 2 to 6 membered heteroalkyl. In embodiments, R¹⁴ is independently unsubstituted 2 to 6 membered heteroalkyl. In embodiments, R¹⁴ is independently substituted or unsubstituted C₃-C₆ cycloalkyl. In embodiments, R¹⁴ is independently unsubstituted C₃-C₆ cycloalkyl. In embodiments, R¹⁴ is independently substituted or unsubstituted 3 to 6 membered heterocycloalkyl. In embodiments, R¹⁴ is independently unsubstituted 3 to 6 membered heterocycloalkyl. In embodiments, R¹⁴ is independently substituted or unsubstituted C₆-C₁₀ aryl. In embodiments, R¹⁴ is independently unsubstituted C₆-C₁₀ aryl. In embodiments, R¹⁴ is independently substituted phenyl. In embodiments, R¹⁴ is independently unsubstituted phenyl. In embodiments, R¹⁴ is independently substituted or unsubstituted 5 to 10 membered heteroaryl. In embodiments, R¹⁴ is independently substituted or unsubstituted 5 to 6 membered heteroaryl. In embodiments, R¹⁴ is independently unsubstituted 5 to 10 membered heteroaryl. In embodiments, R¹⁴ is independently unsubstituted 5 to 6 membered heteroaryl.

In embodiments, R¹⁵ is

hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹⁵ is
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl. In embodiments, R¹⁵ is
hydrogen, —CCl₃, —CF₃, —CHF₂, —CH₂F, or substituted or unsubstituted alkyl. In embodiments, R¹⁵ is hydrogen, —CCl₃, —CF₃, —CHF₂, —CH₂F, or substituted or unsubstituted C₁-C₆ alkyl. In embodiments, R¹⁵ is fluoro-substituted or unsubstituted C₁-C₆ alkyl. In embodiments, R¹⁵ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl. In embodiments, R¹⁵ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R¹⁵ is independently hydrogen, —CCl₃, —CF₃, —CHF₂, —CH₂F, substituted or unsubstituted alkyl, or substituted or unsubstituted heteroalkyl. In embodiments, R¹⁵ is independently
hydrogen, —CCl₃, —CF₃, —CHF₂, —CH₂F, substituted or unsubstituted C₁-C₄ alkyl, or substituted or unsubstituted 2 to 4 membered heteroalkyl. In embodiments, R¹⁵ is independently fluoro-substituted or unsubstituted C₁-C₄ alkyl, or fluoro-substituted or unsubstituted 2 to 4 membered heteroalkyl. In embodiments, R¹⁵ is independently fluoro-substituted or unsubstituted C₁-C₄ alkyl. In embodiments, R¹⁵ is independently fluoro-substituted or unsubstituted 2 to 4 membered heteroalkyl. In embodiments, R¹⁵ is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁵ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹⁵ is independently hydrogen, —CCl₃, —CF₃, —CHF₂, —CH₂F, or substituted or unsubstituted alkyl. In embodiments, R¹⁵ is independently hydrogen, substituted or unsubstituted C₁-C₄ alkyl, or substituted or unsubstituted 2 to 4 membered heteroalkyl. In embodiments, R¹⁵ is independently hydrogen. In embodiments, R″ is independently substituted or unsubstituted C₁-C₄ alkyl. In embodiments, R¹⁵ is independently substituted or unsubstituted 2 to 4 membered heteroalkyl. In embodiments, R¹⁵ is independently unsubstituted C₁-C₄ alkyl. In embodiments, R¹⁵ is independently unsubstituted 2 to 4 membered heteroalkyl. In embodiments, R¹⁵ is independently —CH₃. In embodiments, R¹⁵ is independently —CCl₃. In embodiments, R¹⁵ is independently —CBr₃. In embodiments, R¹⁵ is independently —CF₃. In embodiments, R¹⁵ is independently —CI₃. In embodiments, R¹⁵ is independently —CHCl₂. In embodiments, R¹⁵ is independently —CHBr₂. In embodiments, R¹⁵ is independently —CHF₂. In embodiments, R¹⁵ is independently —CHI₂. In embodiments, R¹⁵ is independently —CH₂Cl. In embodiments, R¹⁵ is independently —CH₂Br. In embodiments, R¹⁵ is independently —CH₂F. In embodiments, R¹⁵ is independently —CH₂I. In embodiments, R¹⁵ is independently —CN. In embodiments, R¹⁵ is independently —COOH. In embodiments, R¹⁵ is independently —COCH₃. In embodiments, R¹⁵ is independently —CONH₂. In embodiments, R¹⁵ is independently —CH₃. In embodiments, R¹⁵ is independently —CH₂CH₃. In embodiments, R¹⁵ is independently —CH(CH₃)₂. In embodiments, R¹⁵ is independently —C(CH₃)₃. In embodiments, R″ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹⁵ is independently substituted or unsubstituted C₁-C₆ alkyl. In embodiments, R¹⁵ is independently unsubstituted C₁-C₆ alkyl. In embodiments, R¹⁵ is independently unsubstituted methyl. In embodiments, R¹⁵ is independently unsubstituted ethyl. In embodiments, R¹⁵ is independently unsubstituted propyl. In embodiments, R¹⁵ is independently substituted or unsubstituted 2 to 6 membered heteroalkyl. In embodiments, R¹⁵ is independently unsubstituted 2 to 6 membered heteroalkyl. In embodiments, R¹⁵ is independently substituted or unsubstituted C₃-C₆ cycloalkyl. In embodiments, R¹⁵ is independently unsubstituted C₃-C₆ cycloalkyl. In embodiments, R¹⁵ is independently substituted or unsubstituted 3 to 6 membered heterocycloalkyl. In embodiments, R¹⁵ is independently unsubstituted 3 to 6 membered heterocycloalkyl. In embodiments, R¹⁵ is independently substituted or unsubstituted C₆-C₁₀ aryl. In embodiments, R¹⁵ is independently unsubstituted C₆-C₁₀ aryl. In embodiments, R¹⁵ is independently substituted phenyl. In embodiments, R¹⁵ is independently unsubstituted phenyl. In embodiments, R¹⁵ is independently substituted or unsubstituted 5 to 10 membered heteroaryl. In embodiments, R¹⁵ is independently substituted or unsubstituted 5 to 6 membered heteroaryl. In embodiments, R¹⁵ is independently unsubstituted 5 to 10 membered heteroaryl. In embodiments, R¹⁵ is independently unsubstituted 5 to 6 membered heteroaryl.

In embodiments, R¹⁶ is —NR^{16A}R^{16B} or —OR^{16B}. In embodiments, R¹⁶ is —OR^{16B} In embodiments, R¹⁶ is —OH. In embodiments, R¹⁶ is —NH₂.

In embodiments, R¹⁶ is —OCX¹⁶₃. In embodiments, R¹⁶ is —OCH₂X¹⁶. In embodiments, R¹⁶ is —OCHX¹⁶₂. In embodiments, R¹⁶ is —SR^{16B}. In embodiments, R¹⁶ is —NR^{16A}R^{16B}.

In embodiments, X¹⁶ is independently —F. In embodiments, X¹⁶ is independently —Cl. In embodiments, X¹⁶ is independently —Br. In embodiments, X¹⁶ is independently —I.

In embodiments, R^{16A} and R^{16B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{16A} and R^{16B} substituents bonded to the same nitrogen atom are joined to form a substituted or unsubstituted heterocycloalkyl or substituted or unsubstituted heteroaryl. In embodiments, R^{16A} and R^{16B} are independently hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{16A} and R^{16B} are independently hydrogen, —CCl₃, —CF₃, —CHF₂, —CH₂F, —CN, —OH, —NH₂, —COOH, —CONH₂, —OCCl₃, —OCF₃, —OCHF 2, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{16A} is independently

hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{16A} is independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl. In embodiments, R^{16A} is independently
hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl. In embodiments, R^{16A} is independently hydrogen, —CCl₃, —CF₃, —CHF₂, —CH₂F, —CN, —OH, —NH₂, —COOH, —CONH₂, —OCCl₃, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{16A} is independently hydrogen, —CCl₃, —CF₃, —CHF₂, —CH₂F, —CN, —OH, —NH₂, —COOH, —CONH₂, —OCCl₃, —OCF₃, —OCHF₂, —OCH₂F, substituted or unsubstituted alkyl.

In embodiments, R^{16A} is independently hydrogen, substituted or unsubstituted C₁-C₄ alkyl, or substituted or unsubstituted 2 to 4 membered heteroalkyl. In embodiments, R^{16A} is independently hydrogen. In embodiments, R^{16A} is independently substituted or unsubstituted C₁-C₄ alkyl. In embodiments, R^{16A} is independently substituted or unsubstituted 2 to 4 membered heteroalkyl. In embodiments, R^{16A} is independently unsubstituted C₁-C₄ alkyl. In embodiments, R^{16A} is independently unsubstituted 2 to 4 membered heteroalkyl. In embodiments, R^{16A} is independently substituted or unsubstituted C₁-C₆ alkyl or substituted or unsubstituted 2 to 6 membered heteroalkyl. In embodiments, R^{16A} is independently —CH₃. In embodiments, R^{16A} is independently —CCl₃. In embodiments, R^{16A} is independently —CBr₃. In embodiments, R^{16A} is independently —CF₃. In embodiments, R^{16A} is independently —CI₃. In embodiments, R^{16A} is independently —CHCl₂. In embodiments, R^{16A} is independently —CHBr₂. In embodiments, R^{16A} is independently —CHF₂. In embodiments, R^{16A} is independently —CHI₂. In embodiments, R^{16A} is independently —CH₂Cl. In embodiments, R^{16A} is independently —CH₂Br. In embodiments, R^{16A} is independently —CH₂F. In embodiments, R^{16A} is independently —CH₂I. In embodiments, R^{16A} is independently —CN. In embodiments, R^{16A} is independently —OCH₃. In embodiments, R^{16A} is independently —NH₂. In embodiments, R^{16A} is independently —COOH. In embodiments, R^{16A} is independently —COCH₃. In embodiments, R^{16A} is independently —CONH₂. In embodiments, R^{16A} is independently —OCCl₃. In embodiments, R^{16A} is independently —OCF₃. In embodiments, R^{16A} is independently —OCBr₃. In embodiments, R^{16A} is independently —OCI₃. In embodiments, R^{16A} is independently —OCHCl₂. In embodiments, R^{16A} is independently —OCHBr₂. In embodiments, R^{16A} is independently —OCHI₂. In embodiments, R^{16A} is independently —OCHF₂. In embodiments, R^{16A} is independently —OCH₂Cl. In embodiments, R^{16A} is independently —OCH₂Br. In embodiments, R^{16A} is independently —OCH₂I. In embodiments, R^{16A} is independently —OCH₂F. In embodiments, R^{16A} is independently unsubstituted methyl. In embodiments, R^{16A} is independently —OCH₃. In embodiments, R^{16A} is independently —OCH₂CH₃. In embodiments, R^{16A} is independently —OCH(CH₃)₂. In embodiments, R^{16A} is independently —OC(CH₃)₃. In embodiments, R^{16A} is independently —CH₃. In embodiments, R^{16A} is independently —CH₂CH₃. In embodiments, R^{16A} is independently —CH(CH₃)₂. In embodiments, R^{16A} is independently —C(CH₃)₃.

In embodiments, R^{16A} is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R^{16A} is independently substituted or unsubstituted C₁-C₆ alkyl. In embodiments, R^{16A} is independently unsubstituted C₁-C₆ alkyl. In embodiments, R^{16A} is independently unsubstituted methyl. In embodiments, R^{16A} is independently unsubstituted ethyl. In embodiments, R^{16A} is independently unsubstituted propyl. In embodiments, R^{16A} is independently substituted or unsubstituted 2 to 6 membered heteroalkyl. In embodiments, R^{16A} is independently unsubstituted 2 to 6 membered heteroalkyl. In embodiments, R^{16A} is independently substituted or unsubstituted C₃-C₆ cycloalkyl. In embodiments, R^{16A} is independently unsubstituted C₃-C₆ cycloalkyl. In embodiments, R^{16A} is independently substituted or unsubstituted 3 to 6 membered heterocycloalkyl. In embodiments, R^{16A} is independently unsubstituted 3 to 6 membered heterocycloalkyl. In embodiments, R^{16A} is independently substituted or unsubstituted C₆-C₁₀ aryl. In embodiments, R^{16A} is independently unsubstituted C₆-C₁₀ aryl. In embodiments, R^{16A} is independently substituted phenyl. In embodiments, R^{16A} is independently unsubstituted phenyl. In embodiments, R^{16A} is independently substituted or unsubstituted 5 to 10 membered heteroaryl. In embodiments, R^{16A} is independently substituted or unsubstituted 5 to 6 membered heteroaryl. In embodiments, R^{16A} is independently unsubstituted 5 to 10 membered heteroaryl. In embodiments, R^{16A} is independently unsubstituted 5 to 6 membered heteroaryl.

In embodiments, R^{16B} is independently

hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{16B} is independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl. In embodiments, R^{16B} is independently
hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl. In embodiments, R^{16B} is independently
hydrogen, —CCl₃, —CF₃, —CHF₂, —CH₂F, —CN, —OH, —NH₂, —COOH, —CONH₂, —OCCl₃, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{16B} is independently hydrogen, —CCl₃, —CF₃, —CHF₂, —CH₂F, —CN, —OH, —NH₂, —COOH, —CONH₂, —OCCl₃, —OCF₃, —OCHF₂, —OCH₂F, substituted or unsubstituted alkyl.

In embodiments, R^{16B} is independently hydrogen, substituted or unsubstituted C₁-C₄ alkyl, or substituted or unsubstituted 2 to 4 membered heteroalkyl. In embodiments, R^{16B} is independently hydrogen. In embodiments, R^{16B} is independently substituted or unsubstituted C₁-C₄ alkyl. In embodiments, R^{16B} is independently substituted or unsubstituted 2 to 4 membered heteroalkyl. In embodiments, R^{16B} is independently unsubstituted C₁-C₄ alkyl. In embodiments, R^{16B} is independently unsubstituted 2 to 4 membered heteroalkyl. In embodiments, R^{16B} is independently substituted or unsubstituted C₁-C₆ alkyl or substituted or unsubstituted 2 to 6 membered heteroalkyl. In embodiments, R^{16B} is independently —CH₃. In embodiments, R^{16B} is independently —CCl₃. In embodiments, R^{16B} is independently —CBr₃. In embodiments, R^{16B} is independently —CF₃. In embodiments, R^{16B} is independently —CI₃. In embodiments, R^{16B} is independently —CHCl₂. In embodiments, R^{16B} is independently —CHBr₂. In embodiments, R^{16B} is independently —CHF₂. In embodiments, R^{16B} is independently —CHI₂. In embodiments, R^{16B} is independently —CH₂Cl. In embodiments, R^{16B} is independently —CH₂Br. In embodiments, R^{16B} is independently —CH₂F. In embodiments, R^{16B} is independently —CH₂I. In embodiments, R^{16B} is independently —CN. In embodiments, R^{16B} is independently —OCH₃. In embodiments, R^{16B} is independently —NH₂. In embodiments, R^{16B} is independently —COOH. In embodiments, R^{16B} is independently —COCH₃. In embodiments, R^{16B} is independently —CONH₂. In embodiments, R^{16B} is independently —OCCl₃. In embodiments, R^{16B} is independently —OCF₃. In embodiments, R^{16B} is independently —OCBr₃. In embodiments, R^{16B} is independently —OCl₃. In embodiments, R^{16B} is independently —OCHCl₂. In embodiments, R^{16B} is independently —OCHBr₂. In embodiments, R^{16B} is independently —OCHI₂. In embodiments, R^{16B} is independently —OCHF₂. In embodiments, R^{16B} is independently —OCH₂Cl. In embodiments, R^{16B} is independently —OCH₂Br. In embodiments, R^{16B} is independently —OCH₂I. In embodiments, R^{16B} is independently —OCH₂F. In embodiments, R^{16B} is independently unsubstituted methyl. In embodiments, R^{16B} is independently —OCH₃. In embodiments, R^{16B} is independently —OCH₂CH₃. In embodiments, R^{16B} is independently —OCH(CH₃)₂. In embodiments, R^{16B} is independently —OC(CH₃)₃. In embodiments, R^{16B} is independently —CH₃. In embodiments, R^{16B} is independently —CH₂CH₃. In embodiments, R^{16B} is independently —CH(CH₃)₂. In embodiments, R^{16B} is independently —C(CH₃)₃.

In embodiments, R^{16B} is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R^{16B} is independently substituted or unsubstituted C₁-C₆ alkyl. In embodiments, R^{16B} is independently unsubstituted C₁-C₆ alkyl. In embodiments, R^{16B} is independently unsubstituted methyl. In embodiments, R^{16B} is independently unsubstituted ethyl. In embodiments, R^{16B} is independently unsubstituted propyl. In embodiments, R^{16B} is independently substituted or unsubstituted 2 to 6 membered heteroalkyl. In embodiments, R^{16B} is independently unsubstituted 2 to 6 membered heteroalkyl. In embodiments, R^{16B} is independently substituted or unsubstituted C₃-C₆ cycloalkyl. In embodiments, R^{16B} is independently unsubstituted C₃-C₆ cycloalkyl. In embodiments, R^{16B} is independently substituted or unsubstituted 3 to 6 membered heterocycloalkyl. In embodiments, R^{16B} is independently unsubstituted 3 to 6 membered heterocycloalkyl. In embodiments, R^{16B} is independently substituted or unsubstituted C₆-C₁₀ aryl. In embodiments, R^{16B} is independently unsubstituted C₆-C₁₀ aryl. In embodiments, R^{16B} is independently substituted phenyl. In embodiments, R^{16B} is independently unsubstituted phenyl. In embodiments, R^{16B} is independently substituted or unsubstituted 5 to 10 membered heteroaryl. In embodiments, R^{16B} is independently substituted or unsubstituted 5 to 6 membered heteroaryl. In embodiments, R^{16B} is independently unsubstituted 5 to 10 membered heteroaryl. In embodiments, R^{16B} is independently unsubstituted 5 to 6 membered heteroaryl.

In embodiments, R^{16A} and R^{16B} substituents bonded to the same nitrogen atom are joined to form a substituted or unsubstituted heterocycloalkyl or substituted or unsubstituted heteroaryl.

In embodiments, R^{16A} and R^{16B} substituents bonded to the same nitrogen atom are joined to form a substituted or unsubstituted heterocycloalkyl. In embodiments, R^{16A} and R^{16B} substituents bonded to the same nitrogen atom are joined to form an unsubstituted heterocycloalkyl. In embodiments, R^{16A} and R^{16B} substituents bonded to the same nitrogen atom are joined to form a substituted or unsubstituted heteroaryl. In embodiments, R^{16A} and R^{16B} substituents bonded to the same nitrogen atom are joined to form an unsubstituted heteroaryl. In embodiments, R^{16A} and R^{16B} substituents bonded to the same nitrogen atom are joined to form a substituted or unsubstituted 3 to 6 membered heterocycloalkyl or substituted or unsubstituted 5 to 10 membered heteroaryl. In embodiments, R^{16A} and R^{16B} substituents bonded to the same nitrogen atom are joined to form a substituted or unsubstituted 3 to 6 membered heterocycloalkyl. In embodiments, R^{16A} and R^{16B} substituents bonded to the same nitrogen atom are joined to form an unsubstituted 3 to 6 membered heterocycloalkyl. In embodiments, R^{16A} and R^{16B} substituents bonded to the same nitrogen atom are joined to form a substituted or unsubstituted 5 to 10 membered heteroaryl. In embodiments, R^{16A} and R^{16B} substituents bonded to the same nitrogen atom are joined to form a substituted or unsubstituted 5 to 6 membered heteroaryl. In embodiments, R^{16A} and R^{16B} substituents bonded to the same nitrogen atom are joined to form an unsubstituted 5 to 10 membered heteroaryl. In embodiments, R^{16A} and R^{16B} substituents bonded to the same nitrogen atom are joined to form an unsubstituted 5 to 6 membered heteroaryl.

In embodiments, R¹⁷ is substituted or unsubstituted alkyl or substituted or unsubstituted cycloalkyl. In embodiments, R¹⁷ is substituted or unsubstituted C₁-C₆ alkyl or substituted or unsubstituted C₃-C₆ cycloalkyl. In embodiments, R¹⁷ is fluoro-substituted or unsubstituted C₁-C₆ alkyl or fluoro-substituted or unsubstituted C₃-C₆ cycloalkyl.

In embodiments, R¹⁷ is independently hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁷ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCF₃, —OCBr₃, —OCI₃, —OCHC 12, —OCHBr₂, —OCHI₂, —OCHF₂, —OCH₂Cl, —OCH₂Br, —OCH₂I, —OCH₂F, substituted or unsubstituted alkyl. In embodiments, R¹⁷ is independently hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl.

In embodiments, R¹⁷ is independently hydrogen, substituted or unsubstituted C₁-C₄ alkyl, or substituted or unsubstituted 2 to 4 membered heteroalkyl. In embodiments, R¹⁷ is independently hydrogen. In embodiments, R¹⁷ is independently substituted or unsubstituted C₁-C₄ alkyl. In embodiments, R¹⁷ is independently substituted or unsubstituted 2 to 4 membered heteroalkyl. In embodiments, R¹⁷ is independently unsubstituted C₁-C₄ alkyl. In embodiments, R¹⁷ is independently unsubstituted 2 to 4 membered heteroalkyl. In embodiments, R¹⁷ is independently substituted or unsubstituted C₁-C₆ alkyl or substituted or unsubstituted 2 to 6 membered heteroalkyl. In embodiments, R¹⁷ is independently —CH₃. In embodiments, R¹⁷ is independently —CCl₃. In embodiments, R¹⁷ is independently —CBr₃. In embodiments, R¹⁷ is independently —CF₃. In embodiments, R¹⁷ is independently —CI₃. In embodiments, R¹⁷ is independently —CHCl₂. In embodiments, R¹⁷ is independently —CHBr₂. In embodiments, R¹⁷ is independently —CHF₂. In embodiments, R¹⁷ is independently —CHI₂. In embodiments, R¹⁷ is independently —CH₂Cl. In embodiments, R¹⁷ is independently —CH₂Br. In embodiments, R¹⁷ is independently —CH₂F. In embodiments, R¹⁷ is independently —CH₂I. In embodiments, R¹⁷ is independently —CN. In embodiments, R¹⁷ is independently —OCH₃. In embodiments, R¹⁷ is independently —NH₂. In embodiments, R¹⁷ is independently —COOH. In embodiments, R¹⁷ is independently —COCH₃. In embodiments, R¹⁷ is independently —CONH₂. In embodiments, R¹⁷ is independently —OCCl₃. In embodiments, R¹⁷ is independently —OCF₃. In embodiments, R¹⁷ is independently —OCBr₃. In embodiments, R¹⁷ is independently —OCI₃. In embodiments, R¹⁷ is independently —OCHCl₂. In embodiments, R¹⁷ is independently —OCHBr₂. In embodiments, R¹⁷ is independently —OCHI₂. In embodiments, R¹⁷ is independently —OCHF₂. In embodiments, R¹⁷ is independently —OCH₂Cl. In embodiments, R¹⁷ is independently —OCH₂Br. In embodiments, R¹⁷ is independently —OCH₂I. In embodiments, R¹⁷ is independently —OCH₂F. In embodiments, R¹⁷ is independently unsubstituted methyl. In embodiments, R¹⁷ is independently —OCH₃. In embodiments, R¹⁷ is independently —OCH₂CH₃. In embodiments, R¹⁷ is independently —OCH(CH₃)₂. In embodiments, R¹⁷ is independently —OC(CH₃)₃. In embodiments, R¹⁷ is independently —CH₃. In embodiments, R¹⁷ is independently —CH₂CH₃. In embodiments, R¹⁷ is independently —CH(CH₃)₂. In embodiments, R¹⁷ is independently —C(CH₃)₃.

In embodiments, R¹⁷ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹⁷ is independently substituted or unsubstituted C₁-C₆ alkyl. In embodiments, R¹⁷ is independently unsubstituted C₁-C₆ alkyl. In embodiments, R¹⁷ is independently unsubstituted methyl. In embodiments, R¹⁷ is independently unsubstituted ethyl. In embodiments, R¹⁷ is independently unsubstituted propyl. In embodiments, R¹⁷ is independently substituted or unsubstituted 2 to 6 membered heteroalkyl. In embodiments, R¹⁷ is independently unsubstituted 2 to 6 membered heteroalkyl. In embodiments, R¹⁷ is independently substituted or unsubstituted C₃-C₆ cycloalkyl. In embodiments, R¹⁷ is independently unsubstituted C₃-C₆ cycloalkyl. In embodiments, R¹⁷ is independently substituted or unsubstituted 3 to 6 membered heterocycloalkyl. In embodiments, R¹⁷ is independently unsubstituted 3 to 6 membered heterocycloalkyl. In embodiments, R¹⁷ is independently substituted or unsubstituted C₆-C₁₀ aryl. In embodiments, R¹⁷ is independently unsubstituted C₆-C₁₀ aryl. In embodiments, R¹⁷ is independently substituted phenyl. In embodiments, R¹⁷ is independently unsubstituted phenyl. In embodiments, R¹⁷ is independently substituted or unsubstituted 5 to 10 membered heteroaryl. In embodiments, R¹⁷ is independently substituted or unsubstituted 5 to 6 membered heteroaryl. In embodiments, R¹⁷ is independently unsubstituted 5 to 10 membered heteroaryl. In embodiments, R¹⁷ is independently unsubstituted 5 to 6 membered heteroaryl. In embodiments, R¹⁷ is independently substituted or unsubstituted C₃-C₈ cycloalkyl. In embodiments, R¹⁷ is independently unsubstituted C₃-C₈ cycloalkyl. In embodiments, R¹⁷ is independently substituted C₃-C₈ cycloalkyl. In embodiments, R¹⁷ is independently substituted or unsubstituted C₄-C₈ cycloalkyl. In embodiments, R¹⁷ is independently unsubstituted C₄-C₈ cycloalkyl. In embodiments, R¹⁷ is independently substituted C₄-C₈ cycloalkyl. In embodiments, R¹⁷ is independently substituted or unsubstituted C₅-C₈ cycloalkyl. In embodiments, R¹⁷ is independently unsubstituted C₅-C₈ cycloalkyl. In embodiments, R¹⁷ is independently substituted C₅-C₅ cycloalkyl. In embodiments, R¹⁷ is independently substituted or unsubstituted C₄-C₆ cycloalkyl. In embodiments, R¹⁷ is independently unsubstituted C₄-C₆ cycloalkyl. In embodiments, R¹⁷ is independently substituted C₄-C₆ cycloalkyl. In embodiments, R¹⁷ is independently substituted or unsubstituted C₅-C₆ cycloalkyl. In embodiments, R¹⁷ is independently unsubstituted C₅-C₆ cycloalkyl. In embodiments, R¹⁷ is independently substituted C₅-C₆ cycloalkyl.

In embodiments, R¹⁷ is independently substituted or unsubstituted C₁-C₆ alkyl. In embodiments, R¹⁷ is substituted or unsubstituted C₃-C₅ alkyl. In embodiments, R¹⁷ is independently substituted C₁-C₆ alkyl. In embodiments, R¹⁷ is substituted C₃-C₅ alkyl. In embodiments, R¹⁷ is independently unsubstituted C₁-C₆ alkyl. In embodiments, R¹⁷ is unsubstituted C₃-C₅ alkyl. In embodiments, R¹⁷ is independently fluoro-substituted C₁-C₆ alkyl. In embodiments, R¹⁷ is fluoro-substituted C₃-C₅ alkyl. In embodiments, R¹⁷ is fluoro-substituted or unsubstituted C₃-C₅ alkyl. In embodiments, R¹⁷ is independently substituted C₂-C₆ alkenyl. In embodiments, R¹⁷ is substituted C₃-C₅ alkenyl. In embodiments, R¹⁷ is independently unsubstituted C₂-C₆ alkenyl. In embodiments, R¹⁷ is unsubstituted C₃-C₅ alkyl. In embodiments, R¹⁷ is independently fluoro-substituted C₂-C₆ alkenyl. In embodiments, R¹⁷ is fluoro-substituted C₃-C₅ alkenyl. In embodiments, R¹⁷ is fluoro-substituted or unsubstituted C₃-C₅ alkenyl.

In embodiments, R¹⁷ is independently substituted or unsubstituted 2 to 6 membered heteroalkyl. In embodiments, R¹⁷ is independently substituted 2 to 6 membered heteroalkyl. In embodiments, R¹⁷ is independently unsubstituted 2 to 6 membered heteroalkyl. In embodiments, R¹⁷ is independently fluoro-substituted 2 to 6 membered heteroalkyl. In embodiments, R¹⁷ is independently substituted 3 to 6 membered heteroalkyl. In embodiments, R¹⁷ is independently unsubstituted 3 to 6 membered heteroalkyl. In embodiments, R¹⁷ is independently fluoro-substituted 3 to 6 membered heteroalkyl. In embodiments, R¹⁷ is independently substituted 2 to 6 membered heteroalkenyl. In embodiments, R¹⁷ is independently unsubstituted 2 to 6 membered heteroalkenyl. In embodiments, R¹⁷ is independently fluoro-substituted 2 to 6 membered heteroalkenyl. In embodiments, R¹⁷ is independently substituted 3 to 6 membered heteroalkenyl. In embodiments, R¹⁷ is independently unsubstituted 3 to 6 membered heteroalkenyl. In embodiments, R¹⁷ is independently fluoro-substituted 3 to 6 membered heteroalkenyl. P In embodiments, R¹⁷ is

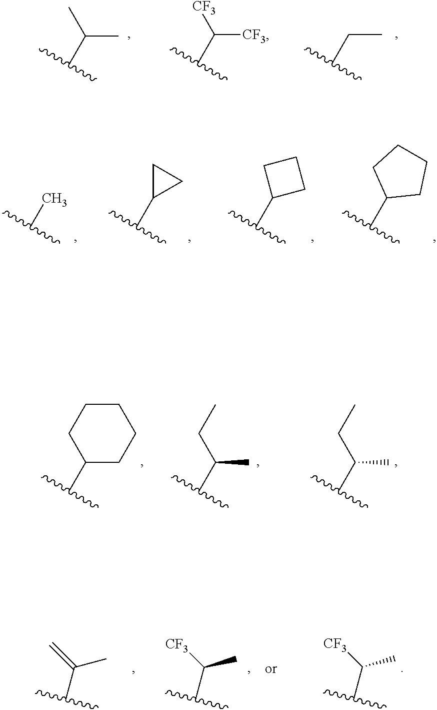

In embodiments, R¹⁷ is

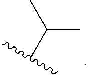 [SMILES: *C(C)C]

In embodiments, R¹⁷ is

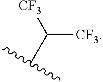 [SMILES: *C(C(F)(F)F)C(F)(F)F]

In embodiments, R¹⁷ is

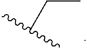 [SMILES: *CC]

In embodiments, R¹⁷ is

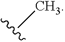

In embodiments, R¹⁷ is

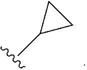

In embodiments, R¹⁷ is

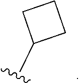

In embodiments, R¹⁷ is

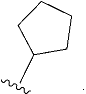

In embodiments, R¹⁷ is

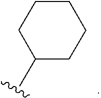

In embodiments, R¹⁷ is

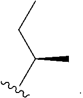

In embodiments, R¹⁷ is

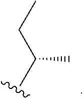

In embodiments, R¹⁷ is

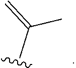

In embodiments, R¹⁷ is

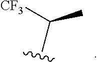

In embodiments, R¹⁷ is

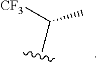

In embodiments, R¹⁸ is

hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹⁸ is
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl. In embodiments, R¹⁸ is
hydrogen, —CCl₃, —CF₃, —CHF₂, —CH₂F, or substituted or unsubstituted alkyl. In embodiments, R¹⁸ is hydrogen, —CCl₃, —CF₃, —CHF₂, —CH₂F, or substituted or unsubstituted C₁-C₆ alkyl. In embodiments, R¹⁸ is fluoro-substituted or unsubstituted C₁-C₆ alkyl. In embodiments, R¹⁸ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl. In embodiments, R¹⁸ is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R¹⁸ is independently
hydrogen, —CCl₃, —CF₃, —CHF₂, —CH₂F, substituted or unsubstituted alkyl, or substituted or unsubstituted heteroalkyl. In embodiments, R¹⁸ is independently
hydrogen, —CCl₃, —CF₃, —CHF₂, —CH₂F, substituted or unsubstituted C₁-C₄ alkyl, or substituted or unsubstituted 2 to 4 membered heteroalkyl. In embodiments, R¹⁸ is independently fluoro-substituted or unsubstituted C₁-C₄ alkyl, or fluoro-substituted or unsubstituted 2 to 4 membered heteroalkyl. In embodiments, R¹⁸ is independently fluoro-substituted or unsubstituted C₁-C₄ alkyl. In embodiments, R¹⁸ is independently fluoro-substituted or unsubstituted 2 to 4 membered heteroalkyl. In embodiments, R¹⁸ is independently hydrogen, —CCl₃, —CF₃, —CHF₂, —CH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁸ is independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl. In embodiments, R¹⁸ is independently
hydrogen, —CCl₃, —CF₃, —CHF₂, —CH₂F, or substituted or unsubstituted alkyl. In embodiments, R¹⁸ is independently hydrogen, substituted or unsubstituted C₁-C₄ alkyl, or substituted or unsubstituted 2 to 4 membered heteroalkyl. In embodiments, R¹⁸ is independently hydrogen. In embodiments, R¹⁸ is independently substituted or unsubstituted C₁-C₄ alkyl. In embodiments, R¹⁸ is independently substituted or unsubstituted 2 to 4 membered heteroalkyl. In embodiments, R¹⁸ is independently unsubstituted C₁-C₄ alkyl. In embodiments, R¹⁸ is independently unsubstituted 2 to 4 membered heteroalkyl. In embodiments, R¹⁸ is independently —CH₃. In embodiments, R¹⁸ is independently —OCH₃. In embodiments, R¹⁸ is independently —CCl₃. In embodiments, R¹⁸ is independently —CBr₃. In embodiments, R¹⁸ is independently —CF₃. In embodiments, R¹⁸ is independently —CI₃. In embodiments, R¹⁸ is independently —CHCl₂. In embodiments, R¹⁸ is independently —CHBr₂. In embodiments, R¹⁸ is independently —CHF₂. In embodiments, R¹⁸ is independently —CHI₂. In embodiments, R¹⁸ is independently —CH₂Cl. In embodiments, R¹⁸ is independently —CH₂Br. In embodiments, R¹⁸ is independently —CH₂F. In embodiments, R¹⁸ is independently —CH₂I. In embodiments, R¹⁸ is independently —CN. In embodiments, R¹⁸ is independently —COOH. In embodiments, R¹⁸ is independently —COCH₃. In embodiments, R¹⁸ is independently —CONH₂. In embodiments, R¹⁸ is independently —CH₃. In embodiments, R¹⁸ is independently —CH₂CH₃. In embodiments, R¹⁸ is independently —CH(CH₃)₂. In embodiments, R¹⁸ is independently —C(CH₃)₃. In embodiments, R¹⁸ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹⁸ is independently substituted or unsubstituted C₁-C₆ alkyl. In embodiments, R¹⁸ is independently unsubstituted C₁-C₆ alkyl. In embodiments, R¹⁸ is independently unsubstituted methyl. In embodiments, R¹⁸ is independently unsubstituted ethyl. In embodiments, R¹⁸ is independently unsubstituted propyl. In embodiments, R¹⁸ is independently substituted or unsubstituted 2 to 6 membered heteroalkyl. In embodiments, R¹⁸ is independently unsubstituted 2 to 6 membered heteroalkyl. In embodiments, R¹⁸ is independently substituted or unsubstituted C₃-C₆ cycloalkyl. In embodiments, R¹⁸ is independently unsubstituted C₃-C₆ cycloalkyl. In embodiments, R¹⁸ is independently substituted or unsubstituted 3 to 6 membered heterocycloalkyl. In embodiments, R¹⁸ is independently unsubstituted 3 to 6 membered heterocycloalkyl. In embodiments, R¹⁸ is independently substituted or unsubstituted C₆-C₁₀ aryl. In embodiments, R¹⁸ is independently unsubstituted C₆-C₁₀ aryl. In embodiments, R¹⁸ is independently substituted phenyl. In embodiments, R¹⁸ is independently unsubstituted phenyl. In embodiments, R¹⁸ is independently substituted or unsubstituted 5 to 10 membered heteroaryl. In embodiments, R¹⁸ is independently substituted or unsubstituted 5 to 6 membered heteroaryl. In embodiments, R¹⁸ is independently unsubstituted 5 to 10 membered heteroaryl. In embodiments, R¹⁸ is independently unsubstituted 5 to 6 membered heteroaryl.

In embodiments, R¹⁸ is hydrogen. In embodiments, R¹⁸ is unsubstituted C₁-C₄ alkyl. In embodiments, R¹⁸ is unsubstituted methyl. In embodiments, R¹⁸ is a resin moiety. In embodiments, the resin moiety is a moiety of TGR A resin, Oxime resin, 2-Chlorotrityl chloride resin, Wang resin, TGA resin, Merrifield resin, TGT alcohol resin, HMBA resin, HMPB resin, HMPA resin, Rink Acid resin, Hydrazinobenzoyl AM resin. In embodiments, the resin moiety is a moiety of TGR A resin. In embodiments, the resin moiety is a moiety of Oxime resin. In embodiments, the resin moiety is a moiety of 2-Chlorotrityl chloride resin. In embodiments, the resin moiety is a moiety of Wang resin. In embodiments, the resin moiety is a moiety of TGA resin. In embodiments, the resin moiety is a moiety of Merrifield resin. In embodiments, the resin moiety is a moiety of TGT alcohol resin. In embodiments, the resin moiety is a moiety of HMBA resin. In embodiments, the resin moiety is a moiety of HMPB resin. In embodiments, the resin moiety is a moiety of HMPA resin. In embodiments, the resin moiety is a moiety of Rink Acid resin. In embodiments, the resin moiety is a moiety of Hydrazinobenzoyl AM resin. In embodiments R¹⁸ is a moiety of a solid substrate useful for compound synthesis. In embodiments R¹⁸ is a moiety of a solid substrate. In embodiments R¹⁸ is a moiety of a bead, gel, polymer, particle, or grain. In embodiments, a resin is a polymer. In embodiments, a resin is a polymer including substituents capable of forming a bond with a compound described herein, wherein the resin is a monovalent form when bonded to a compound described herein as an R¹⁸ substituent.

In embodiments, the compound has the formula

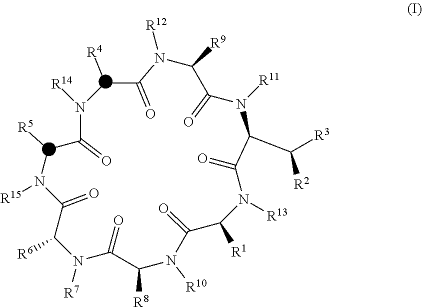

and R¹, R², R³, R⁴, R⁵, R⁶, R⁷, R⁸, R⁹, R¹⁰, R¹¹, R¹², R¹³, R¹⁴, R¹⁵, R¹⁶, and R¹⁷ are as described herein including in embodiments. In embodiments, the compound has the formula

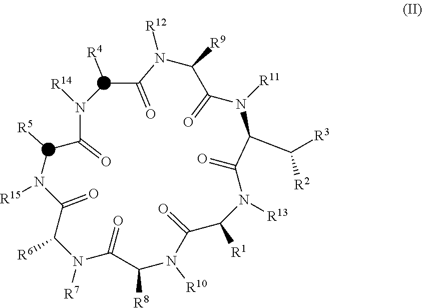

and R¹, R², R³, R⁴, R⁵, R⁶, R⁷, R⁸, R⁹, R¹⁰, R¹¹, R¹², R¹³, R¹⁴, R¹⁵, R¹⁶, and R¹⁷ are as described herein, including in embodiments. In embodiments, the compound has the formula:

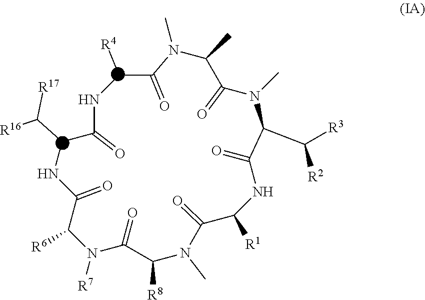

and R¹, R², R³, R⁴, R⁶, R⁷, R⁸, R¹⁶, and R¹⁷ are as described herein, including in embodiments. In embodiments, the compound has the formula

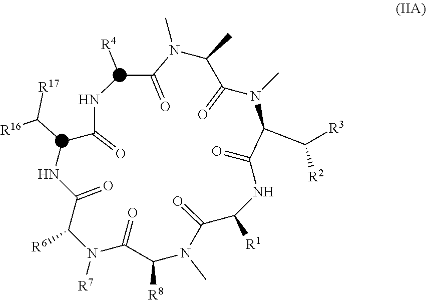

and R¹, R², R³, R⁴, R⁶, R⁷, R⁸, R¹⁶, and R¹⁷ are as described herein, including in embodiments. In embodiments, the compound has the formula:

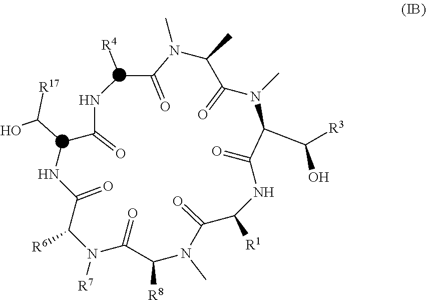

and R¹, R³, R⁴, R⁶, R⁷, R⁸ and R¹⁷ are as described herein, including in embodiments. In embodiments, the compound has the formula

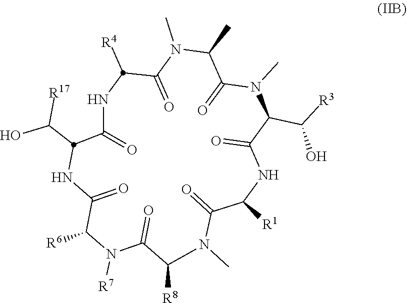

and R¹, R³, R⁴, R⁶, R⁷, R⁸ and R¹⁷ are as described herein, including in embodiments. In embodiments, the compound has the formula:

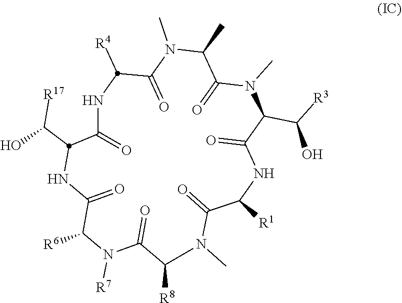

and R¹, R³, R⁴, R⁶, R⁷, R⁸ and R¹⁷ are as described herein, including in embodiments. In embodiments, the compound has the formula

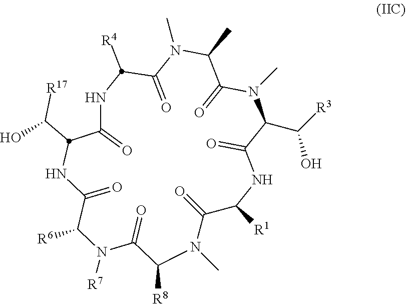

and R¹, R³, R⁴, R⁶, R⁷, R⁸ and R¹⁷ are as described herein, including in embodiments. In embodiments, the compound has the formula

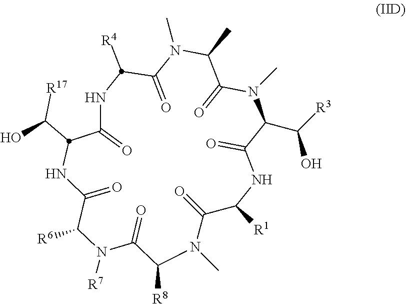

and R¹, R³, R⁴, R⁶, R⁷, R⁸ and R¹⁷ are as described herein, including in embodiments. In embodiments, the compound has the formula

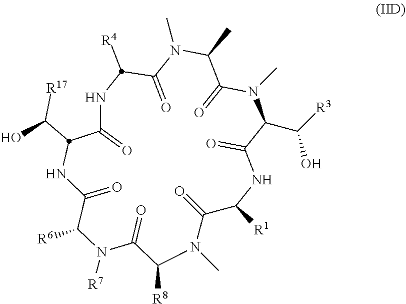

and R¹, R³, R⁴, R⁶, R⁷, R⁸ and R¹⁷ are as described herein,
including in embodiments. In embodiments, the compound is:

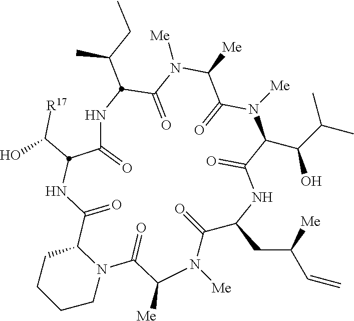

and R¹⁷ is as described herein, including in embodiments. In embodiments, the compound is:

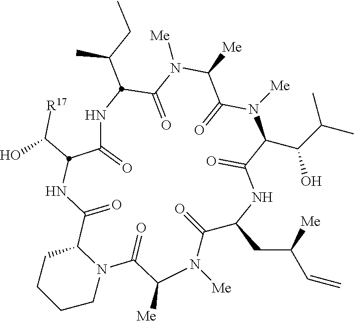

and R¹⁷ is as described herein, including in embodiments. In embodiments, the compound is:

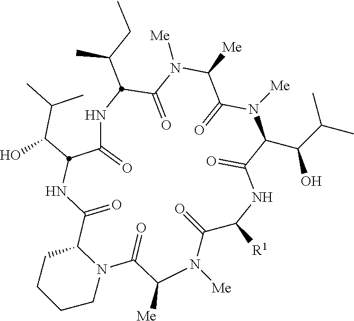

and R¹ is as described herein, including in embodiments. In embodiments, the compound is:

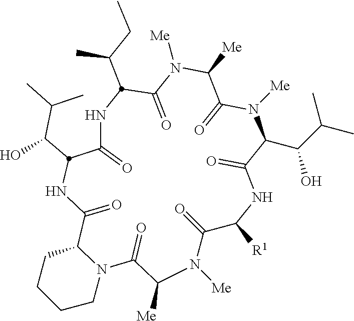

and R¹ is as described herein, including in embodiments. In embodiments, the compound is:

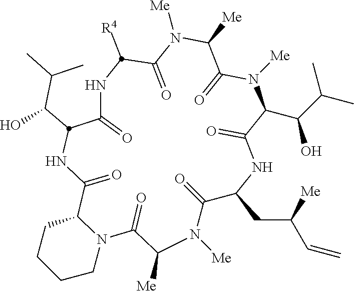

and R⁴ is as described herein, including in embodiments. In embodiments, the compound is:

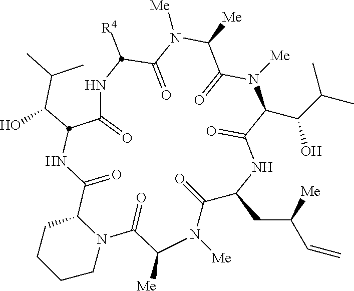

and R⁴ is as described herein,
including in embodiments. In embodiments, the compound is:

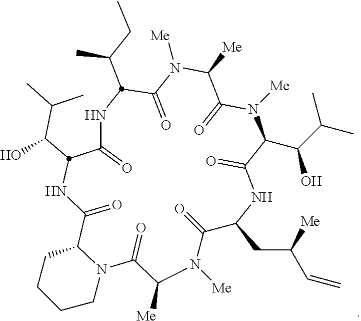 [SMILES: C=C[C@H](C)C[C@@H]1NC(=O)[C@H]([C@H](O)C(C)C)N(C)C(=O)[C@H](C)N(C)C(=O)[C@@H]([C@@H](C)CC)NC(=O)[C@@H]([C@H](O)C(C)C)NC(=O)[C@H]2CCCCN2C(=O)[C@H](C)N(C)C1=O]

In embodiments, the compound is:

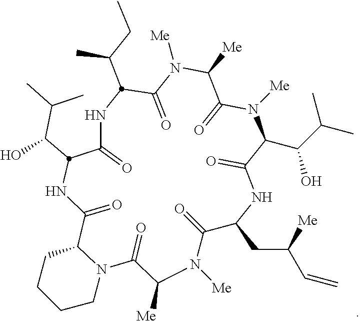 [SMILES: C=C[C@H](C)C[C@@H]1NC(=O)[C@H]([C@@H](O)C(C)C)N(C)C(=O)[C@H](C)N(C)C(=O)[C@@H]([C@@H](C)CC)NC(=O)[C@@H]([C@H](O)C(C)C)NC(=O)[C@H]2CCCCN2C(=O)[C@H](C)N(C)C1=O]

In embodiments, the compound is:

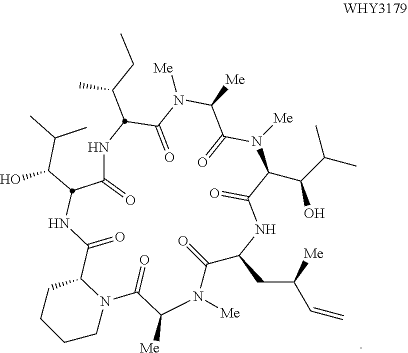 [SMILES: C=C[C@H](C)C[C@@H]1NC(=O)[C@H]([C@H](O)C(C)C)N(C)C(=O)[C@H](C)N(C)C(=O)[C@@H]([C@H](C)CC)NC(=O)[C@@H]([C@H](O)C(C)C)NC(=O)[C@H]2CCCCN2C(=O)[C@H](C)N(C)C1=O]

In embodiments, the compound is:

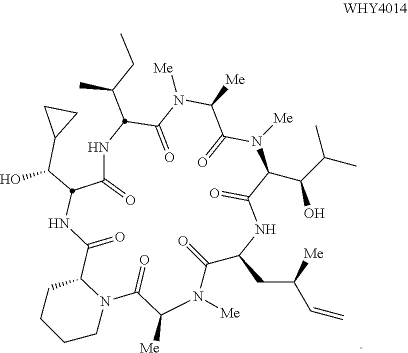 [SMILES: C=C[C@H](C)C[C@@H]1NC(=O)[C@H]([C@H](O)C(C)C)N(C)C(=O)[C@H](C)N(C)C(=O)[C@@H]([C@@H](C)CC)NC(=O)[C@@H]([C@H](O)C2CC2)NC(=O)[C@H]2CCCCN2C(=O)[C@H](C)N(C)C1=O]

In embodiments, the compound has the formula

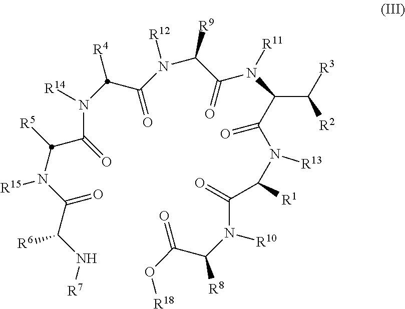

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

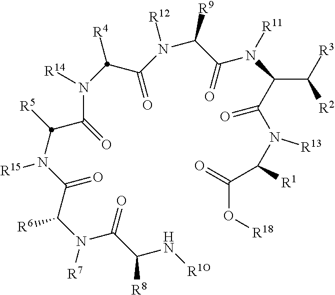

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

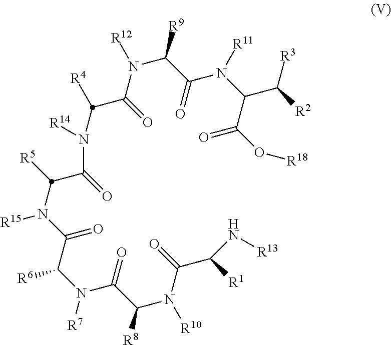

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

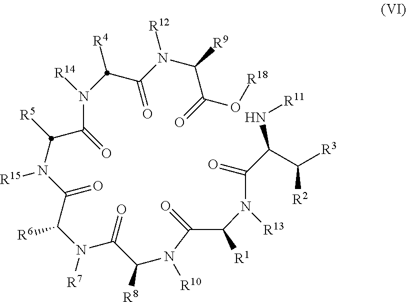

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

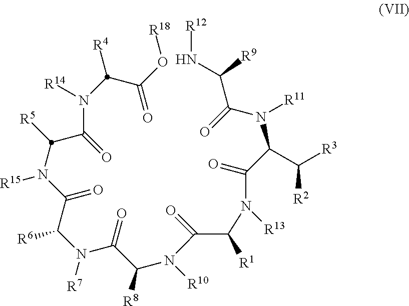

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

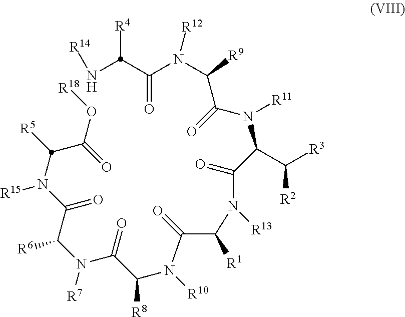

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

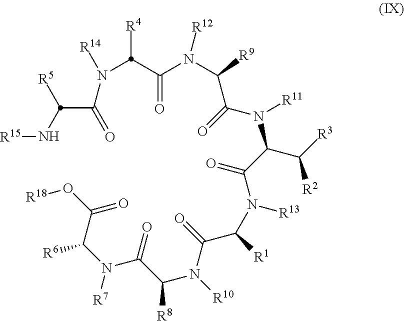

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

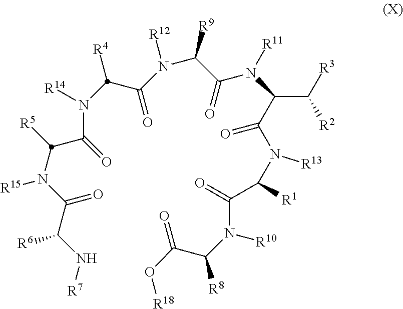

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

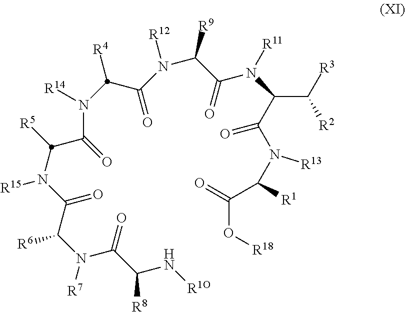

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

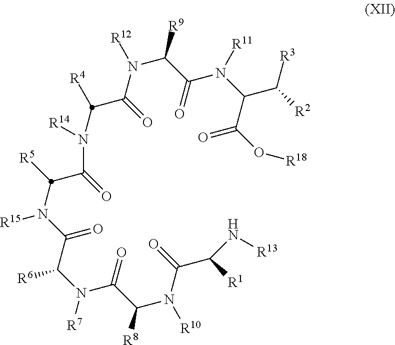

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

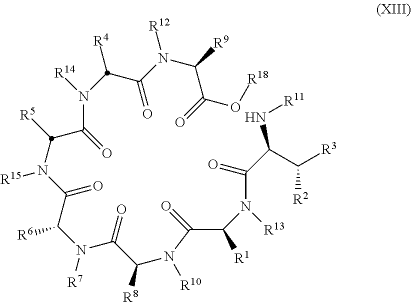

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

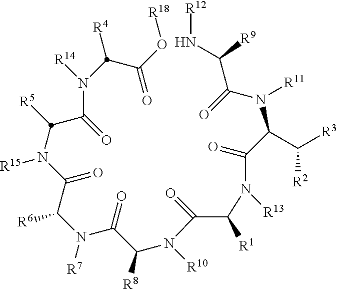

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

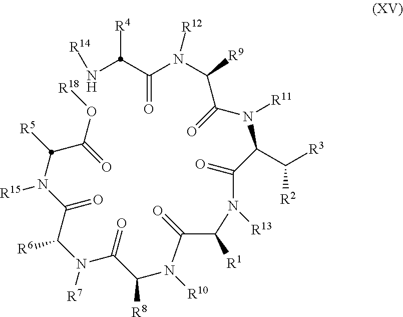

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula

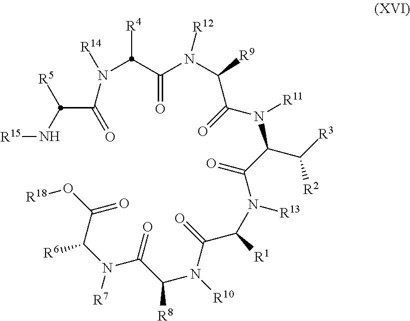

and R¹, R², R³, R⁴, R⁵, R⁶, R⁷, R⁸, R⁹, R¹⁰, R¹¹, R¹², R¹³, R¹⁴, R¹⁵, and R¹⁸ are as described herein, including in embodiments. In embodiments, the compound has the formula:

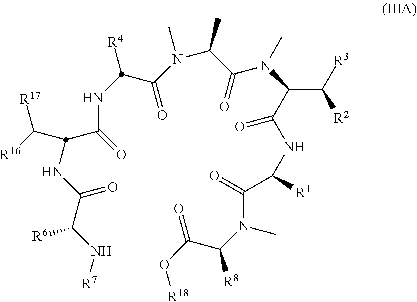

and R¹, R², R³, R⁴, R⁶, R⁷, R⁸, R¹⁶, R¹⁷, and R¹⁸ are as described herein, including in embodiments. In embodiments, the compound has the formula

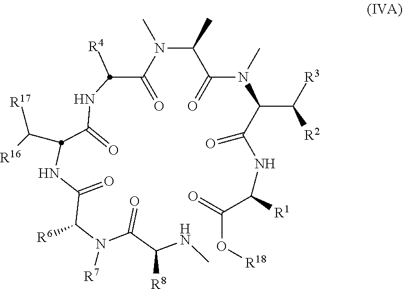

and R¹, R², R³, R⁴, R⁶, R⁷, R⁸, R¹⁶, R¹⁷, and R¹⁸ are as described herein, including in embodiments. In embodiments, the compound has the formula

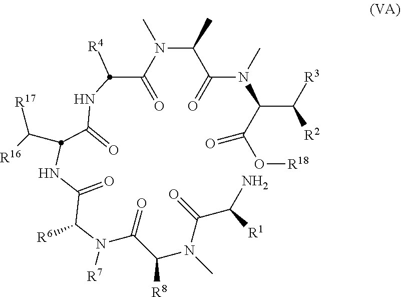

and R¹, R², R³, R⁴, R⁶, R⁷, R⁸, R¹⁶, R¹⁷, and R¹⁸ are as described herein, including in embodiments. In embodiments, the compound has the formula

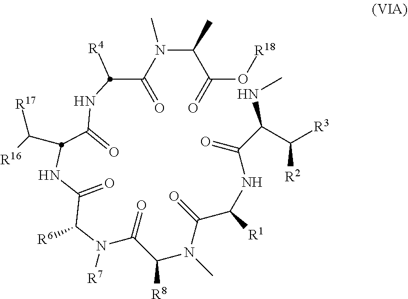

and R¹, R², R³, R⁴, R⁶, R⁷, R⁸, R¹⁶, R¹⁷, and R¹⁸ are as described herein, including in embodiments. In embodiments, the compound has the formula

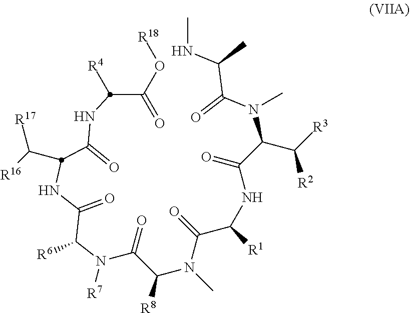

and R¹, R², R³, R⁴, R⁶, R⁷, R⁸, R¹⁶, R¹⁷, and R¹⁸ are as described herein, including in embodiments. In embodiments, the compound has the formula

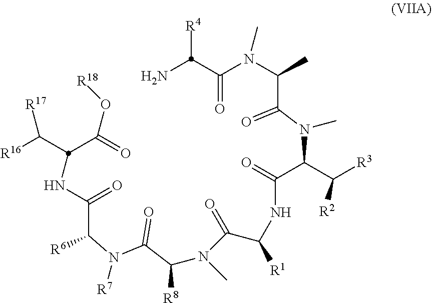

and R¹, R², R³, R⁴, R⁶, R⁷, R⁸, R¹⁶, R¹⁷, and R¹⁸ are as described herein, including in embodiments. In embodiments, the compound has the formula

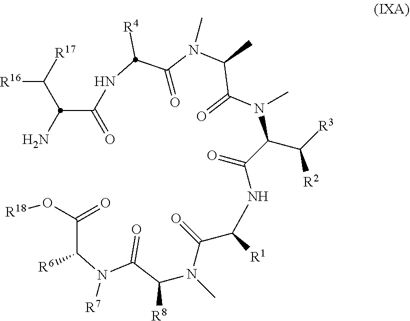

and R¹, R², R³, R⁴, R⁶, R⁷, R⁸, R¹⁶, R¹⁷, and R¹⁸ are as described herein, including in embodiments. In embodiments, the compound has the formula

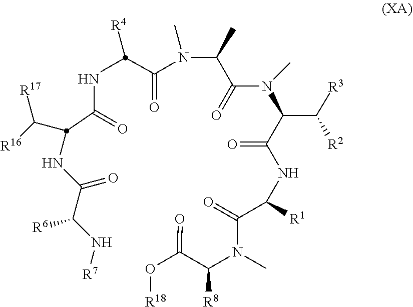

and R¹, R², R³, R⁴, R⁶, R⁷, R⁸, R¹⁶, R¹⁷, and R¹⁸ are as described herein, including in embodiments. In embodiments, the compound has the formula

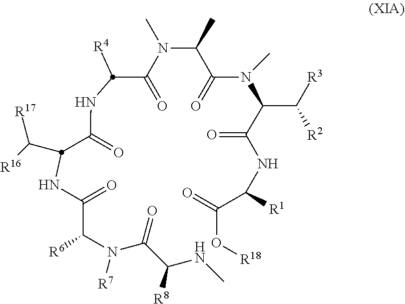

and R¹, R², R³, R⁴, R⁶, R⁷, R⁸, R¹⁶, R¹⁷, and R¹⁸ are as described herein, including in embodiments. In embodiments, the compound has the formula

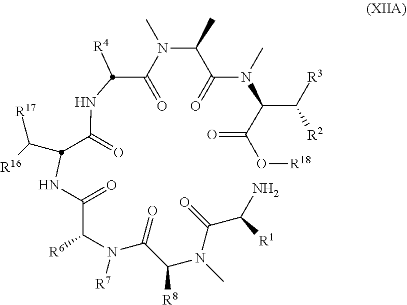

and R¹, R², R³, R⁴, R⁶, R⁷, R⁸, R¹⁶, R¹⁷, and R¹⁸ are as described herein, including in embodiments. In embodiments, the compound has the formula

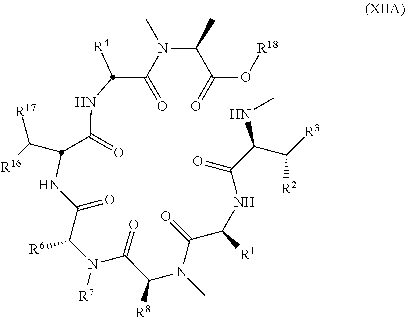

and R¹, R², R³, R⁴, R⁶, R⁷, R⁸, R¹⁶, R¹⁷, and R¹⁸ are as described herein, including in embodiments. In embodiments, the compound has the formula

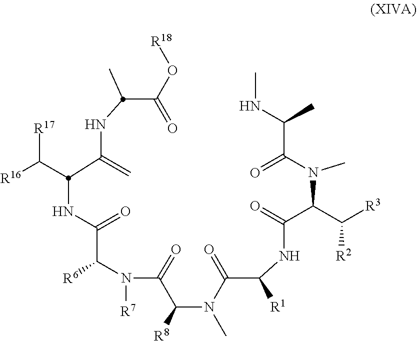

and R¹, R², R³, R⁴, R⁶, R⁷, R⁸, R¹⁶, R¹⁷, and R¹⁸ are as described herein, including in embodiments. In embodiments, the compound has the formula

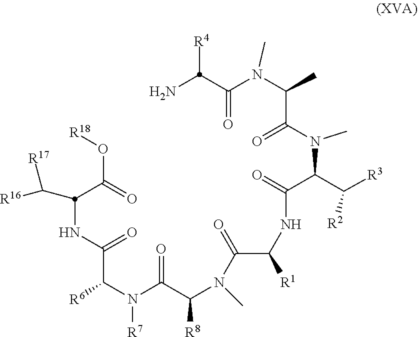

and R¹, R², R³, R⁴, R⁶, R⁷, R⁸, R¹⁶, R¹⁷, and R¹⁸ are as described herein, including in embodiments. In embodiments, the compound has the formula

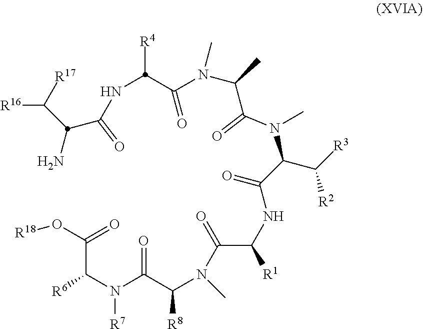

and R¹, R², R³, R⁴, R⁶, R⁷, R⁸, R¹⁶, R¹⁷, and R¹⁸ are as described herein, including in embodiments. In embodiments, the compound has the formula:

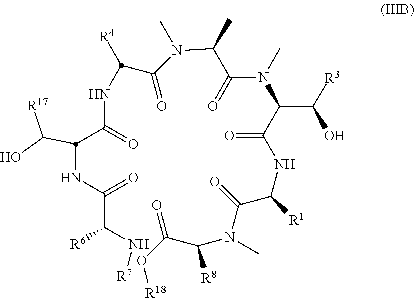

and R¹, R³, R⁴, R⁶, R⁷, R⁸, R¹⁷, and R¹⁸ are as described herein, including in embodiments. In embodiments, the compound has the formula

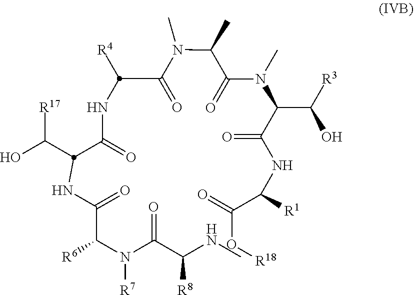

and R¹, R³, R⁴, R⁶, R⁷, R⁸, R¹⁷, and R¹⁸ are as described herein, including in embodiments. In embodiments, the compound has the formula

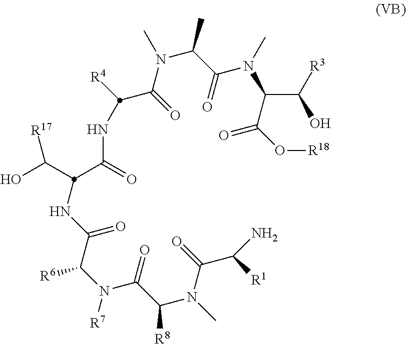

and R¹, R³, R⁴, R⁶, R⁷, R⁸, R¹⁷, and R¹⁸ are as described herein, including in embodiments. In embodiments, the compound has the formula

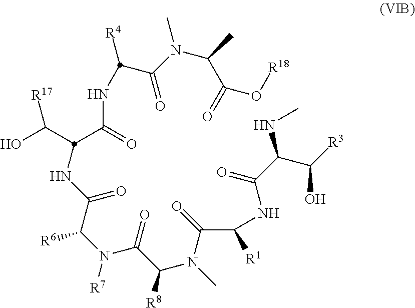

and R¹, R³, R⁴, R⁶, R⁷, R⁸, R¹⁷, and R¹⁸ are as described herein, including in embodiments. In embodiments, the compound has the formula

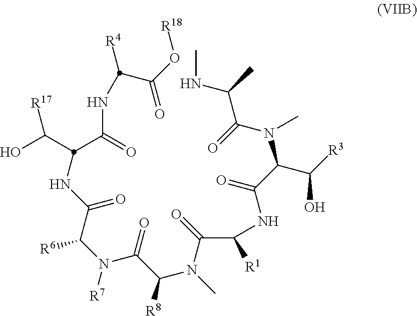

and R¹, R³, R⁴, R⁶, R⁷, R⁸, R¹⁷, and R¹⁸ are as described herein, including in embodiments. In embodiments, the compound has the formula

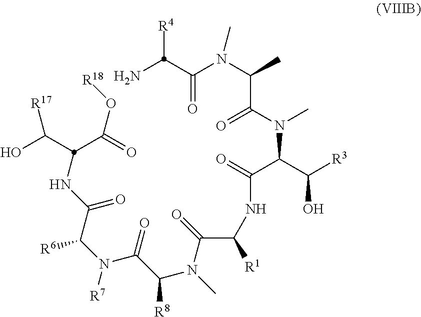

and R¹, R³, R⁴, R⁶, R⁷, R⁸, R¹⁷, and R¹⁸ are as described herein, including in embodiments. In embodiments, the compound has the formula

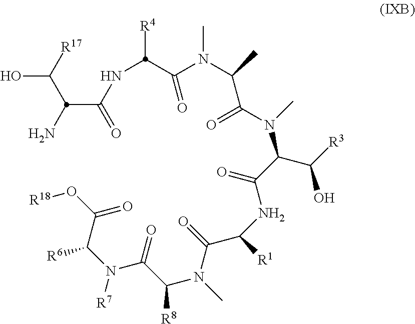

and R¹, R³, R⁴, R⁶, R⁷, R⁸, R¹⁷, and R¹⁸ are as described herein, including in embodiments. In embodiments, the compound has the formula

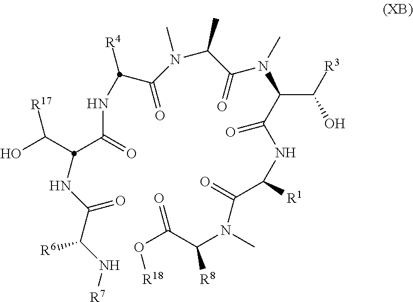

and R¹, R³, R⁴, R⁶, R⁷, R⁸, R¹⁷, and R¹⁸ are as described herein, including in embodiments. In embodiments, the compound has the formula

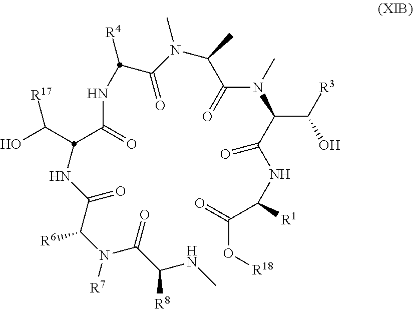

and R¹, R³, R⁴, R⁶, R⁷, R⁸, R¹⁷, and R¹⁸ are as described herein, including in embodiments. In embodiments, the compound has the formula

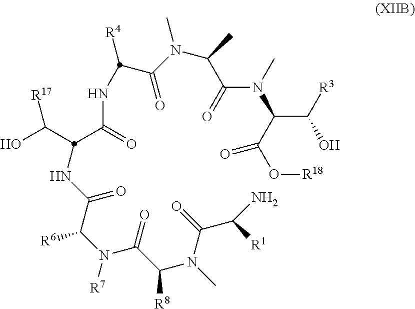

and R¹, R³, R⁴, R⁶, R⁷, R⁸, R¹⁷, and R¹⁸ are as described herein, including in embodiments. In embodiments, the compound has the formula

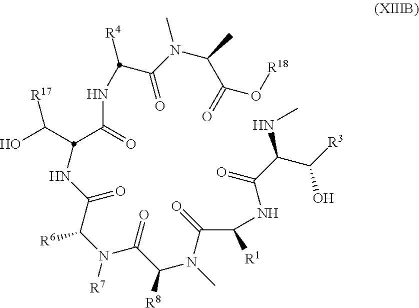

and R¹, R³, R⁴, R⁶, R⁷, R⁸, R¹⁷, and R¹⁸ are as described herein, including in embodiments. In embodiments, the compound has the formula

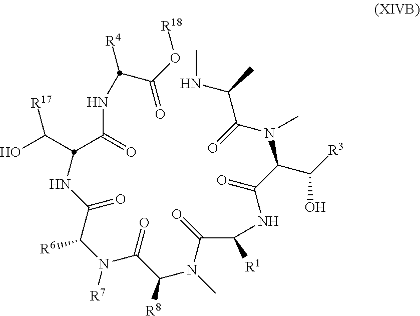

and R¹, R³, R⁴, R⁶, R⁷, R⁸, R¹⁷, and R¹⁸ are as described herein, including in embodiments. In embodiments, the compound has the formula

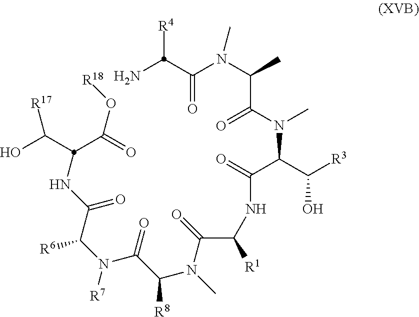

and R¹, R³, R⁴, R⁶, R⁷, R⁸, R¹⁷, and R¹⁸ are as described herein, including in embodiments. In embodiments, the compound has the formula

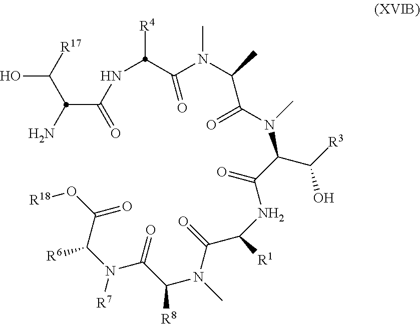

and R¹, R³, R⁴, R⁶, R⁷, R⁸, R¹⁷, and R¹⁸ are as described herein, including in embodiments. In embodiments, the compound has the formula:

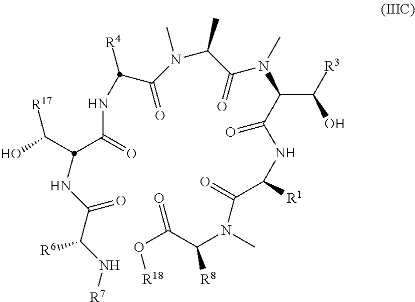

and R¹, R³, R⁴, R⁶, R⁷, R⁸, R¹⁷, and R¹⁸ are as described herein, including in embodiments. In embodiments, the compound has the formula

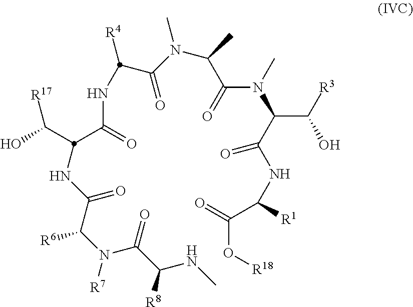

and R¹, R³, R⁴, R⁶, R⁷, R⁸, R¹⁷, and R¹⁸ are as described herein, including in embodiments. In embodiments, the compound has the formula

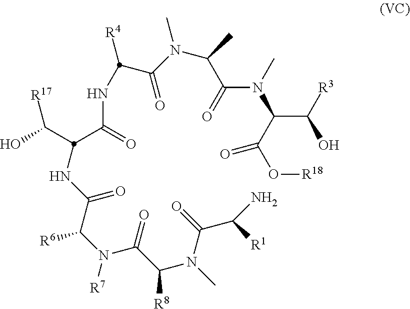

and R¹, R³, R⁴, R⁶, R⁷, R⁸, R¹⁷, and R¹⁸ are as described herein, including in embodiments. In embodiments, the compound has the formula

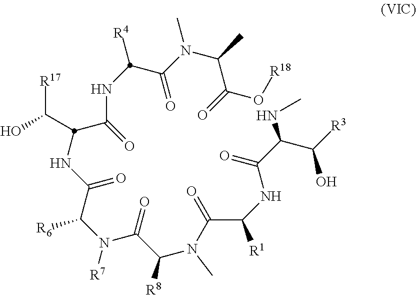

and R¹, R³, R⁴, R⁶, R⁷, R⁸, R¹⁷, and R¹⁸ are as described herein, including in embodiments. In embodiments, the compound has the formula

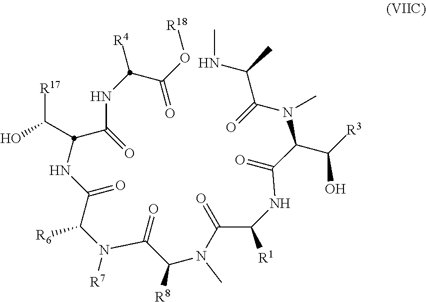

and R¹, R³, R⁴, R⁶, R⁷, R⁸, R¹⁷, and R¹⁸ are as described herein, including in embodiments. In embodiments, the compound has the formula

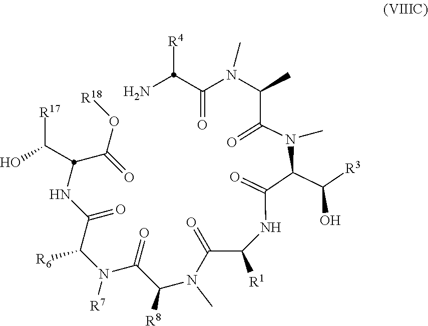

and R¹, R³, R⁴, R⁶, R⁷, R⁸, R¹⁷, and R¹⁸ are as described herein, including in embodiments. In embodiments, the compound has the formula

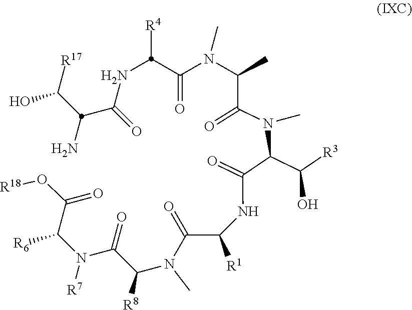

and R¹, R³, R⁴, R⁶, R⁷, R⁸, R¹⁷, and R¹⁸ are as described herein, including in embodiments. In embodiments, the compound has the formula

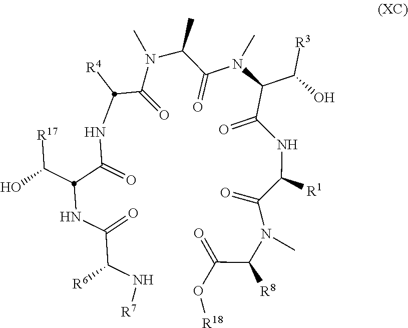

and R¹, R³, R⁴, R⁶, R⁷, R⁸, R¹⁷, and R¹⁸ are as described herein, including in embodiments. In embodiments, the compound has the formula

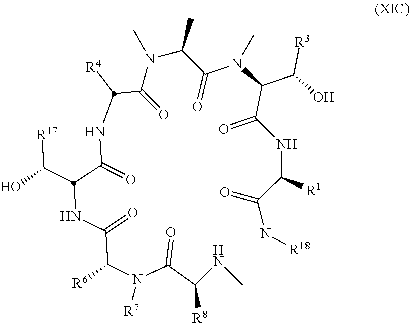

and R¹, R³, R⁴, R⁶, R⁷, R⁸, R¹⁷, and R¹⁸ are as described herein, including in embodiments. In embodiments, the compound has the formula

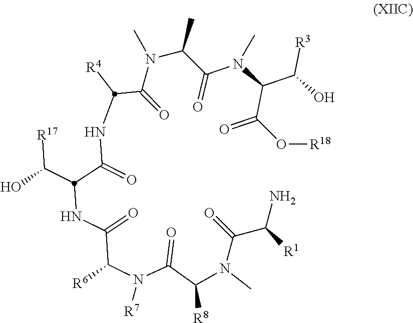

and R¹, R³, R⁴, R⁶, R⁷, R⁸, R¹⁷, and R¹⁸ are as described herein, including in embodiments. In embodiments, the compound has the formula

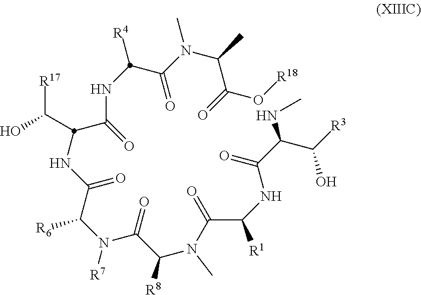

and R¹, R³, R⁴, R⁶, R⁷, R⁸, R¹⁷, and R¹⁸ are as described herein, including in embodiments. In embodiments, the compound has the formula

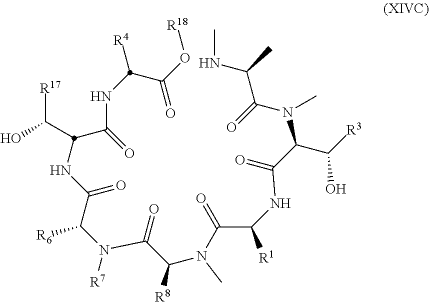

and R¹, R³, R⁴, R⁶, R⁷, R⁸, R¹⁷, and R¹⁸ are as described herein, including in embodiments. In embodiments, the compound has the formula

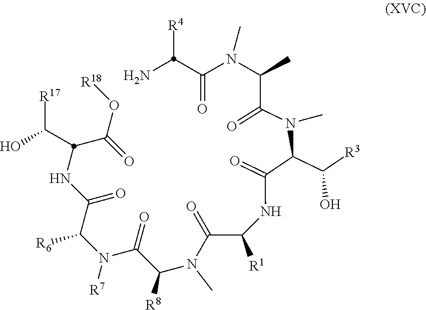

and R¹, R³, R⁴, R⁶, R⁷, R⁸, R¹⁷, and R¹⁸ are as described herein, including in embodiments. In embodiments, the compound has the formula

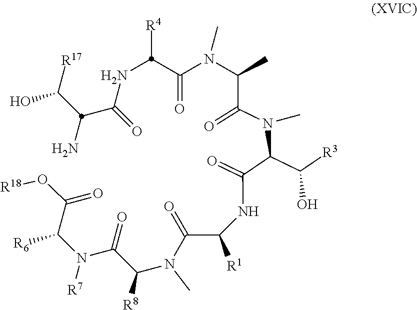

and R¹, R³, R⁴, R⁶, R⁷, R⁸, R¹⁷, and R¹⁸ are as described herein, including in embodiments. In embodiments, the compound has the formula

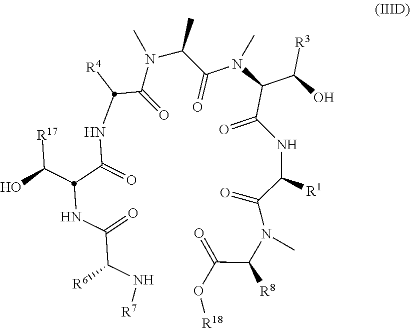

and R¹, R³, R⁴, R⁶, R⁷, R⁸, R¹⁷, and R¹⁸ are as described herein, including in embodiments. In embodiments, the compound has the formula

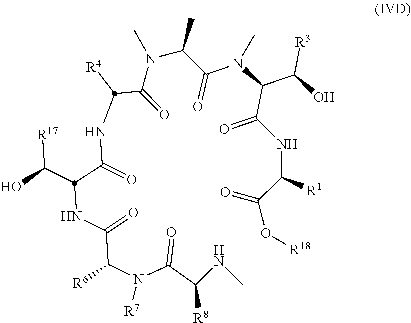

and R¹, R³, R⁴, R⁶, R⁷, R⁸, R¹⁷, and R¹⁸ are as described herein, including in embodiments. In embodiments, the compound has the formula

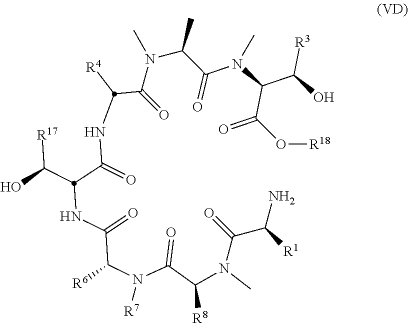

and R¹, R³, R⁴, R⁶, R⁷, R⁸, R¹⁷, and R¹⁸ are as described herein, including in embodiments. In embodiments, the compound has the formula

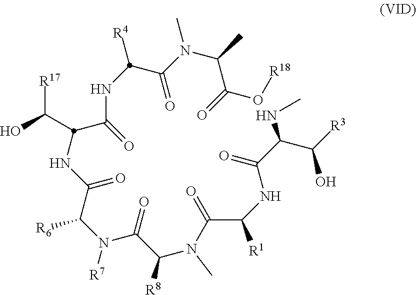

and R¹, R³, R⁴, R⁶, R⁷, R⁸, R¹⁷, and R¹⁸ are as described herein, including in embodiments. In embodiments, the compound has the formula

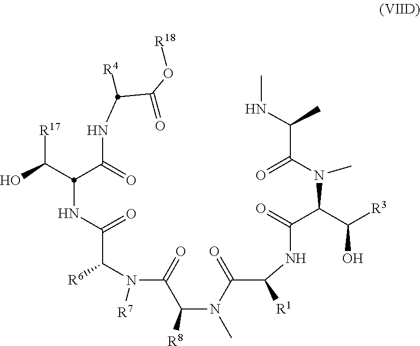

and R¹, R³, R⁴, R⁶, R⁷, R⁸, R¹⁷, and R¹⁸ are as described herein, including in embodiments. In embodiments, the compound has the formula

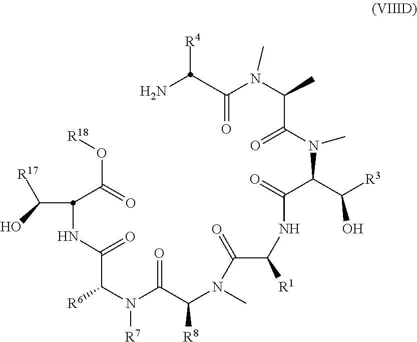

and R¹, R³, R⁴, R⁶, R⁷, R⁸, R¹⁷, and R¹⁸ are as described herein, including in embodiments. In embodiments, the compound has the formula

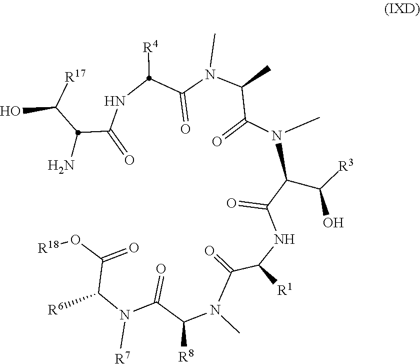

and R¹, R³, R⁴, R⁶, R⁷, R⁸, R¹⁷, and R¹⁸ are as described herein, including in embodiments. In embodiments, the compound has the formula

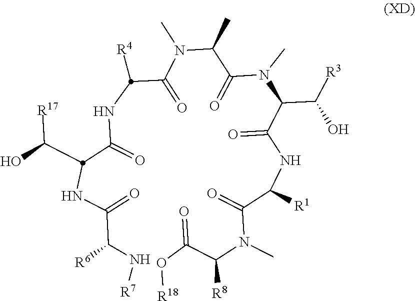

and R¹, R³, R⁴, R⁶, R⁷, R⁸, R¹⁷, and R¹⁸ are as described herein, including in embodiments. In embodiments, the compound has the formula

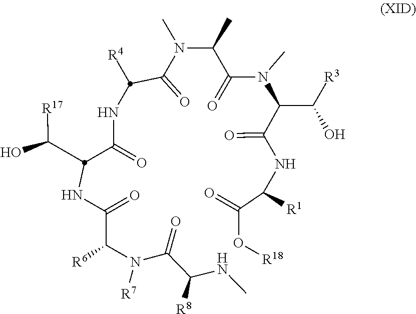

and R¹, R³, R⁴, R⁶, R⁷, R⁸, R¹⁷, and R¹⁸ are as described herein, including in embodiments. In embodiments, the compound has the formula

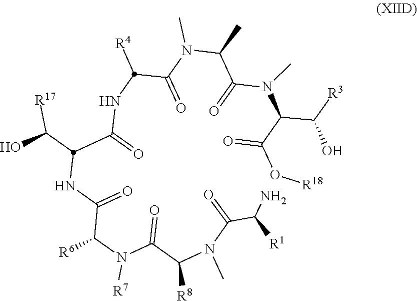

and R¹, R³, R⁴, R⁶, R⁷, R⁸, R¹⁷, and R¹⁸ are as described herein, including in embodiments. In embodiments, the compound has the formula

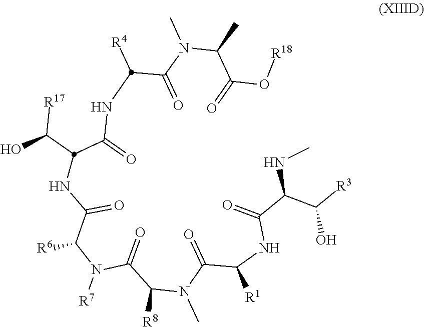

and R¹, R³, R⁴, R⁶, R⁷, R⁸, R¹⁷, and R¹⁸ are as described herein, including in embodiments. In embodiments, the compound has the formula

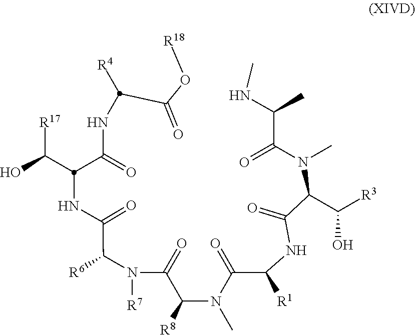

and R¹, R³, R⁴, R⁶, R⁷, R⁸, R¹⁷, and R¹⁸ are as described herein, including in embodiments. In embodiments, the compound has the formula

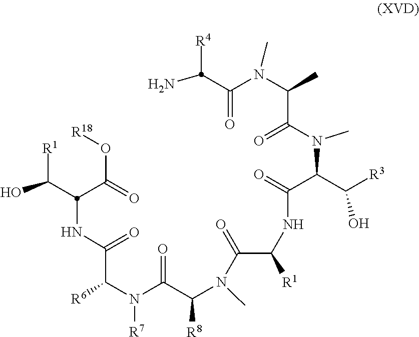

and R¹, R³, R⁴, R⁶, R⁷, R⁸, R¹⁷, and R¹⁸ are as described herein, including in embodiments. In embodiments, the compound has the formula

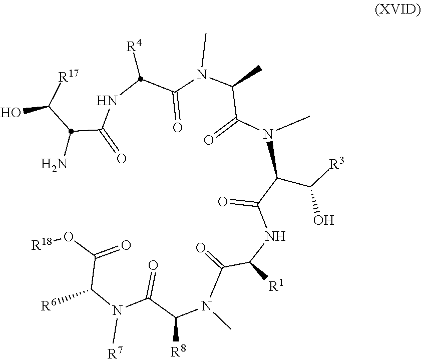

and R¹, R³, R⁴, R⁶, R⁷, R⁸, R¹⁷, and R¹⁸ are as described herein, including in embodiments. In embodiments, the compound has the formula:

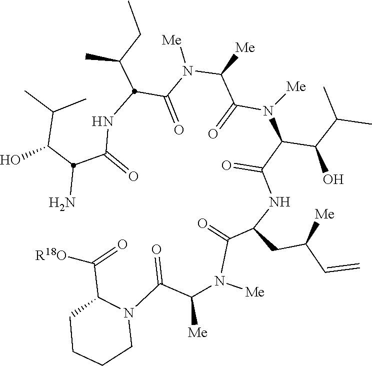

and R¹⁸ is as described herein, including in embodiments. In embodiments, the compound has the formula

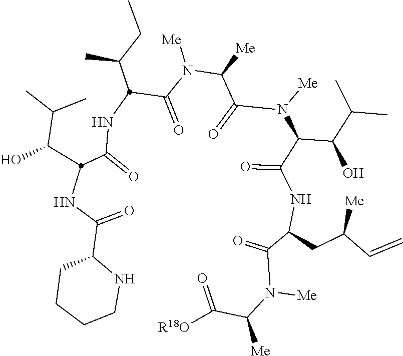

and R¹⁸ is as described herein, including in embodiments. In embodiments, the compound has the formula

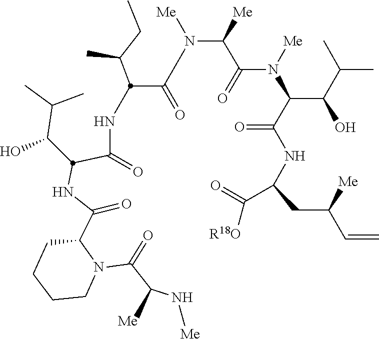

and R¹⁸ is as described herein, including in embodiments. In embodiments, the compound has the formula

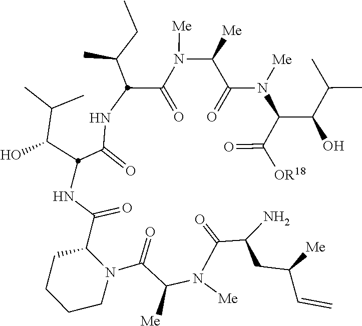

and R¹⁸ is as described herein, including in embodiments. In embodiments, the compound has the formula

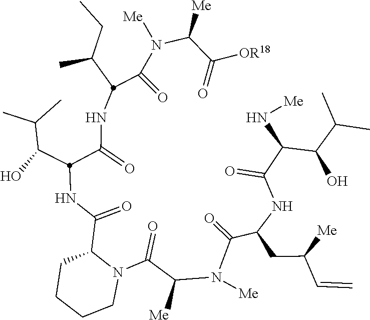

and R¹⁸ is as described herein, including in embodiments. In embodiments, the compound has the formula

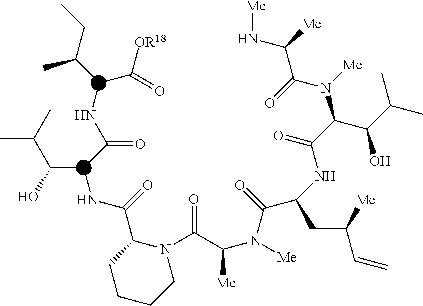

and R¹⁸ is as described herein, including in embodiments. In embodiments, the compound has the formula

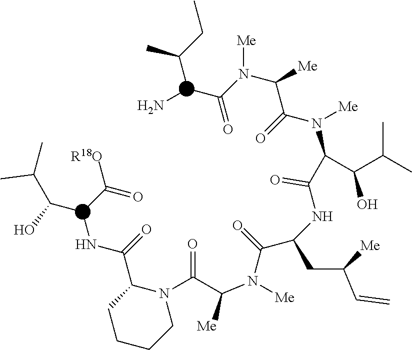

and R¹⁸ is as described herein, including in embodiments. In embodiments, the compound has the formula

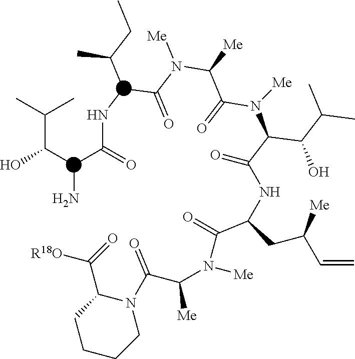

and R¹⁸ is as described herein, including in embodiments. In embodiments, the compound has the formula

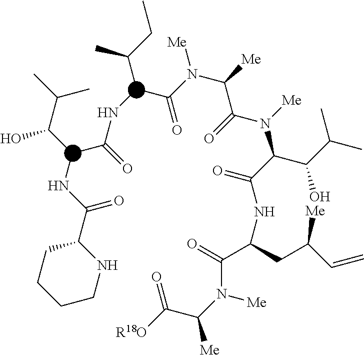

and R¹⁸ is as described herein, including in embodiments. In embodiments, the compound has the formula

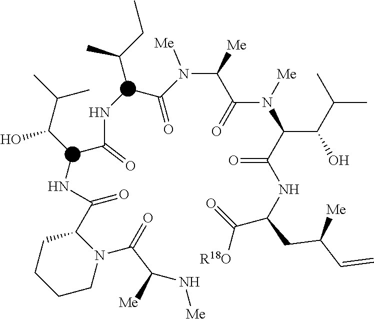

and R¹⁸ is as described herein, including in embodiments. In embodiments, the compound has the formula

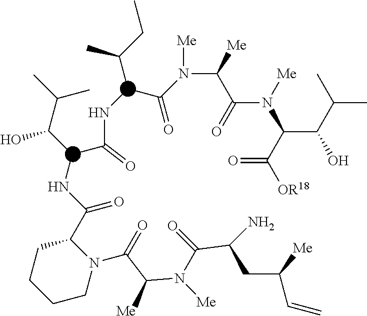

and R¹⁸ is as described herein, including in embodiments. In embodiments, the compound has the formula

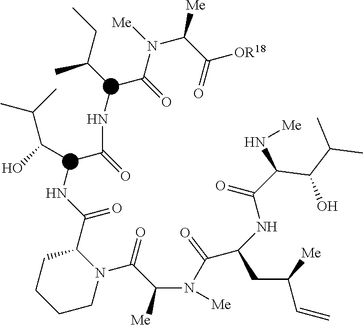

and R¹⁸ is as described herein, including in embodiments. In embodiments, the compound has the formula

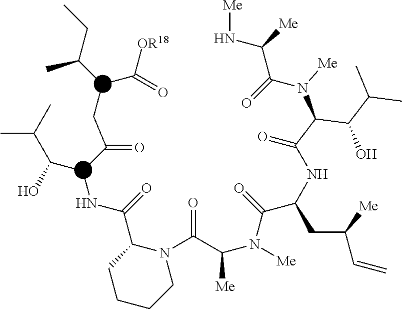

and R¹⁸ is as described herein, including in embodiments. In embodiments, the compound has the formula

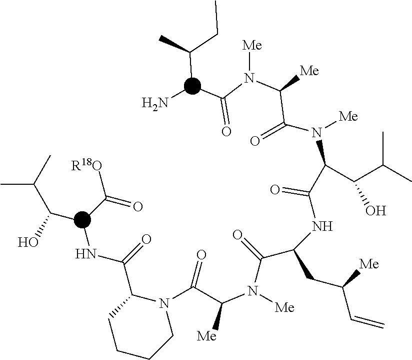

and R¹⁸ is as described herein, including in embodiments.

In embodiments, the compound has the formula:

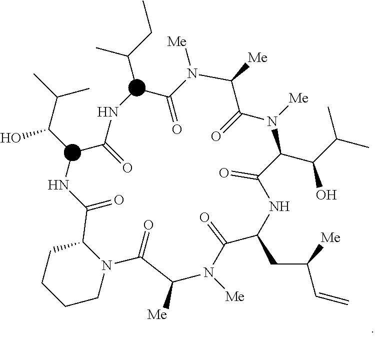 [SMILES: C=C[C@H](C)C[C@@H]1NC(=O)[C@H]([C@H](O)C(C)C)N(C)C(=O)[C@H](C)N(C)C(=O)C(C(C)CC)NC(=O)C([C@H](O)C(C)C)NC(=O)[C@H]2CCCCN2C(=O)[C@H](C)N(C)C1=O]

In embodiments, the compound has the formula:

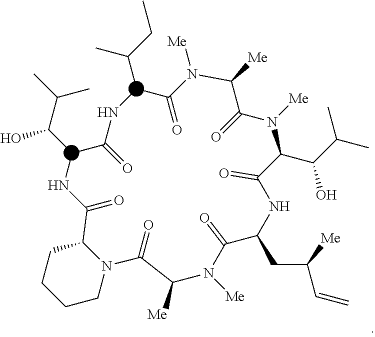 [SMILES: C=C[C@H](C)C[C@@H]1NC(=O)[C@H]([C@@H](O)C(C)C)N(C)C(=O)[C@H](C)N(C)C(=O)C(C(C)CC)NC(=O)C([C@H](O)C(C)C)NC(=O)[C@H]2CCCCN2C(=O)[C@H](C)N(C)C1=O]

In an aspect is provided a protected amino acid. In embodiments, the protected amino acid is an Fmoc-protected amino acid. In embodiments, the protected amino acid is a Boc-protected amino acid.

In embodiments, the protected amino acid is

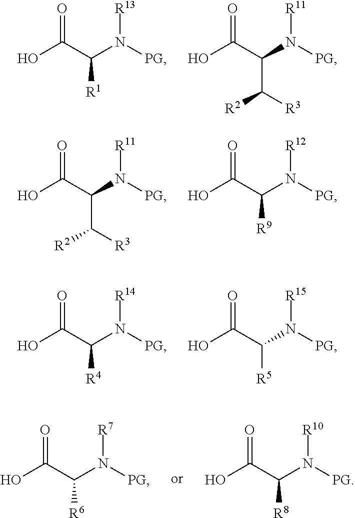

R¹, R², R³, R⁴, R⁵, R⁶, R⁷, R⁸, R⁹, R¹⁰, R¹¹, R¹², R¹³, R¹⁴, and R¹⁵ are as described herein, including in embodiments. PG is a protecting group.

In embodiments, the protected amino acid is

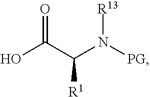

wherein R¹, R¹³, and PG are as described herein, including in embodiments. In embodiments, the protected amino acid is

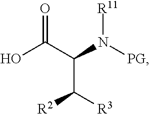

wherein R², R³, R¹¹, and PG are as described herein, including in embodiments. In embodiments, the protected amino acid is

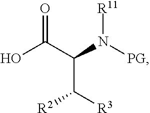

wherein R², R³, R¹¹, and PG are as described herein, including in embodiments. In embodiments, the protected amino acid is

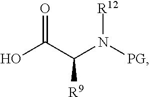

wherein R⁹, R¹², and PG are as described herein, including in embodiments. In embodiments, the protected amino acid is

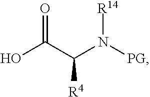

wherein R⁴, R¹⁴, and PG are as described herein, including in embodiments. In embodiments, the protected amino acid is

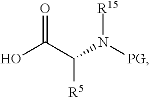

wherein R⁵, R¹⁵, and PG are as described herein, including in embodiments. In embodiments, the protected amino acid is

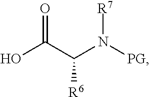

wherein R⁶, R⁷, and PG are as described herein, including in embodiments. In embodiments, the protected amino acid is

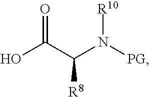

wherein R⁸, R¹⁰, and PG are as described herein, including in embodiments.

In embodiments, PG is independently 9-fluorenylmethyloxycarbonyl (Fmoc). In embodiments, PG is independently

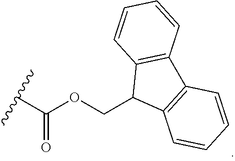 [SMILES: *C(=O)OCC1c2ccccc2-c2ccccc21]

In embodiments, PG is independently tert-butyloxycarbonyl (Boc). In embodiments, PG is independently

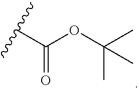 [SMILES: *C(=O)OC(C)(C)C]

In embodiments, the protected amino acid is

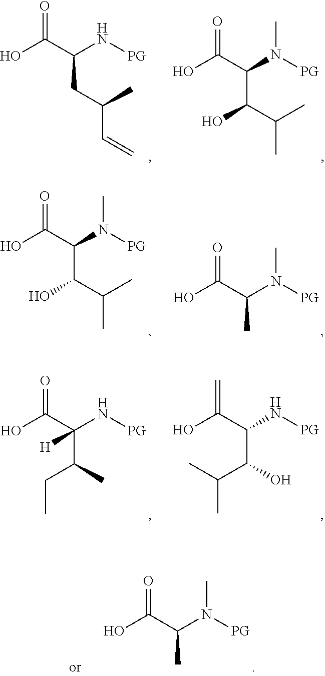

PG is as described herein, including in embodiments.

In embodiments, the protected amino acid is

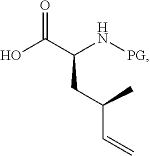

wherein PG is as described herein, including in embodiments. In embodiments, the protected amino acid is

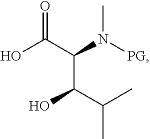

wherein PG is as described herein, including in embodiments. In embodiments, the protected amino acid is

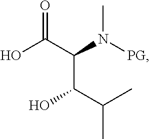

wherein PG is as described herein, including in embodiments. In embodiments, the protected amino acid is

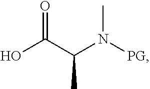

wherein PG is as described herein, including in embodiments. In embodiments, the protected amino acid is

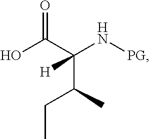

wherein PG is as described herein, including in embodiments. In embodiments, the protected amino acid is

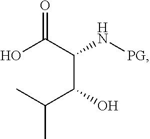

wherein PG is as described herein, including in embodiments. In embodiments, the protected amino acid is

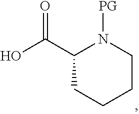

wherein PG is as described herein, including in embodiments. In embodiments, the protected amino acid is

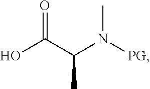

wherein PG is as described herein, including in embodiments.

In embodiments, the protected amino acid is

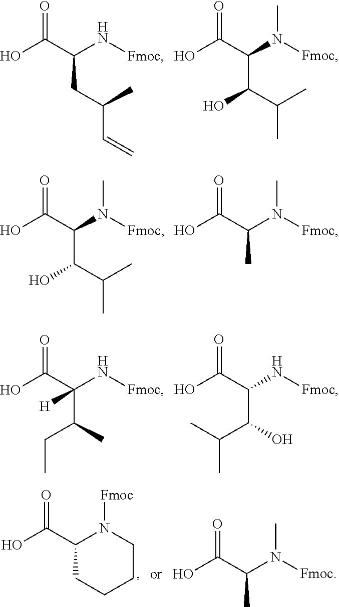

In embodiments, the protected amino acid is

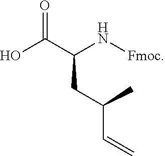

In embodiments, the protected amino acid is

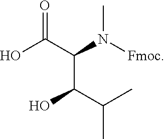

In embodiments, the protected amino acid is

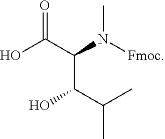

In embodiments, the protected amino acid is

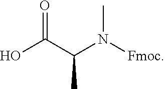

In embodiments, the protected amino acid is

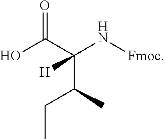

In embodiments, the protected amino acid is

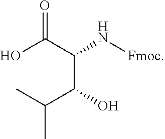

In embodiments, the protected amino acid is

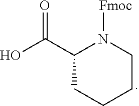

In embodiments, the protected amino acid is

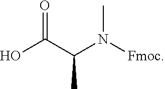

In embodiments, the protected amino acid is.

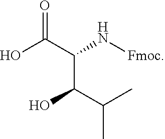

In embodiments, the protected amino acid is

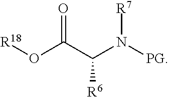

R⁶, R⁷, R¹⁸, and PG are as described herein, including in embodiments.

In embodiments, the protected amino acid is

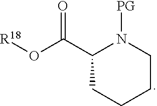

R¹⁸ and PG are as described herein, including in embodiments.

In embodiments, the protected amino acid is

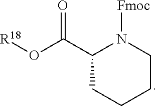

R¹⁸ is as described herein, including in embodiments.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3} respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents that are bonded to the same nitrogen atom are joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW}, R^{WW.2}, and R^{WW.3}, respectively, as explained in the definitions section above in the description of “first substituent group(s)”, wherein R^{WW.1}, R^{WW.2} and R^{WW.3} correspond to R^{2A.1}, R^{2A.2}, and R^{2A.3}, respectively.

In embodiments, when R^{2A} and R^{2B} substituents that are bonded to the same nitrogen atom are joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW}, R^{WW.2}, and R^{WW.3}, respectively, as explained in the definitions section above in the description of “first substituent group(s)”, wherein R^{WW.1}, R^{WW.2} and R^{WW.3} correspond to R^{2B.1}, R^{2B.2}, and R^{2B.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2} and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2} and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R⁵¹ substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R^{5.2}, and R^{5.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R⁶, R^{6.1}, R^{6.2} and R^{6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁶, R^{6.1}, R^{6.2}, and R^{6.3}, respectively.

In embodiments, when R⁷ is substituted, R⁷ is substituted with one or more first substituent groups denoted by R^{7.1} as explained in the definitions section above in the description of “first substituent group(s)”. In embodiments, when an R^{7.1} substituent group is substituted, the R^{7.1} substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R⁷, R^{7.1}, R^{7.2} and R^{7.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁷, R^{7.1}, R^{7.2}, and R^{7.3}, respectively.

In embodiments, when R⁶ and R⁷ substituents are joined to form, in combination with the —CHN— connecting the two substituents, a moiety that is substituted (e.g., a substituted heterocycloalkyl), the moiety is substituted with one or more first substituent groups denoted by R^{6.1} as explained in the definitions section above in the description of “first substituent group(s)”. In embodiments, when an R⁶¹ substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R^{6.1}, R^{6.2}, and R^{6.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{6.1}, R^{6.2}, and R^{6.3}, respectively.

In embodiments, when R⁶ and R⁷ substituents are joined to form, in combination with the —CHN— connecting the two substituents, a moiety that is substituted (e.g., a substituted heterocycloalkyl), the moiety is substituted with one or more first substituent groups denoted by R^{7.1} as explained in the definitions section above in the description of “first substituent group(s)”. In embodiments, when an R^{7.1} substituent group is substituted, the R^{7.1} substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R^{7.1}, R^{7.2}, and R^{7.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{7.1}, R^{7.2}, and R^{7.3}, respectively.

In embodiments, when R⁸ is substituted, R⁸ is substituted with one or more first substituent groups denoted by R^{8.1} as explained in the definitions section above in the description of “first substituent group(s)”. In embodiments, when an R^{8.1} substituent group is substituted, the R^{8.1} substituent group is substituted with one or more second substituent groups denoted by R^{8.2} as explained in the definitions section above in the description of “first substituent group(s)”. In embodiments, when an R^{8.2} substituent group is substituted, the R^{8.2} substituent group is substituted with one or more third substituent groups denoted by R^{8.3} as explained in the definitions section above in the description of “first substituent group(s)”. In the above embodiments, R⁸, R^{8.1}, R^{8.2} and R^{8.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁸, R^{8.1}, R^{8.2}, and R^{8.3}, respectively.

In embodiments, when R⁹ is substituted, R⁹ is substituted with one or more first substituent groups denoted by R^{9.1} as explained in the definitions section above in the description of “first substituent group(s)”. In embodiments, when an R^{9.1} substituent group is substituted, the R^{9.1} substituent group is substituted with one or more second substituent groups denoted by R^{9.2} as explained in the definitions section above in the description of “first substituent group(s)”. In embodiments, when an R^{9.2} substituent group is substituted, the R^{9.2} substituent group is substituted with one or more third substituent groups denoted by R^{9.3} as explained in the definitions section above in the description of “first substituent group(s)”. In the above embodiments, R⁹, R^{9.1}, R^{9.2} and R^{9.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁹, R^{9.1}, R^{9.2}, and R^{9.3}, respectively.

In embodiments, when R¹⁰ is substituted, R¹⁰ is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R^{10.1}, R^{10.2}, and R^{10.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰, R^{10.1}, R^{10.2}, and R^{10.3}, respectively.

In embodiments, when R¹¹ is substituted, R¹¹ is substituted with one or more first substituent groups denoted by R^{11.1} as explained in the definitions section above in the description of “first substituent group(s)”. In embodiments, when an R^{11.1} substituent group is substituted, the R^{11.1} substituent group is substituted with one or more second substituent groups denoted by R^{11.2} as explained in the definitions section above in the description of “first substituent group(s)”. In embodiments, when an R^{11.2} substituent group is substituted, the R^{11.2} substituent group is substituted with one or more third substituent groups denoted by R^{11.3} as explained in the definitions section above in the description of “first substituent group(s)”. In the above embodiments, R¹¹, R^{11.1}, R^{11.2}, and R^{11.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹¹, R^{11.1}, R^{11.2}, and R^{11.3}, respectively.

In embodiments, when R¹² is substituted, R¹² is substituted with one or more first substituent groups denoted by R^{12.1} as explained in the definitions section above in the description of “first substituent group(s)”. In embodiments, when an R^{12.1} substituent group is substituted, the R^{12.1} substituent group is substituted with one or more second substituent groups denoted by R^{12.2} as explained in the definitions section above in the description of “first substituent group(s)”. In embodiments, when an R^{12.2} substituent group is substituted, the R^{12.2} substituent group is substituted with one or more third substituent groups denoted by R¹²³ as explained in the definitions section above in the description of “first substituent group(s)”. In the above embodiments, R¹², R^{12.1}, R^{12.2}, and R^{12.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹², R^{12.1}, R^{12.2}, and R^{12.3} respectively.

In embodiments, when R¹³ is substituted, R¹³ is substituted with one or more first substituent groups denoted by R^{13.1} as explained in the definitions section above in the description of “first substituent group(s)”. In embodiments, when an R^{13.1} substituent group is substituted, the R^{13.1} substituent group is substituted with one or more second substituent groups denoted by R^{13.2} as explained in the definitions section above in the description of “first substituent group(s)”. In embodiments, when an R^{13.2} substituent group is substituted, the R^{13.2} substituent group is substituted with one or more third substituent groups denoted by R^{13.3} as explained in the definitions section above in the description of “first substituent group(s)”. In the above embodiments, R¹³, R^{13.1}, R^{13.2}, and R^{13.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹³, R^{13.1}, R^{13.2}, and R^{13.3}, respectively.

In embodiments, when R¹⁴ is substituted, R¹⁴ is substituted with one or more first substituent groups denoted by R^{14.1} as explained in the definitions section above in the description of “first substituent group(s)”. In embodiments, when an R^{14.1} substituent group is substituted, the R^{14.1} substituent group is substituted with one or more second substituent groups denoted by R^{14.2} as explained in the definitions section above in the description of “first substituent group(s)”. In embodiments, when an R^{14.2} substituent group is substituted, the R^{14.2} substituent group is substituted with one or more third substituent groups denoted by R^{14.3} as explained in the definitions section above in the description of “first substituent group(s)”. In the above embodiments, R¹⁴, R^{14.1}, R^{14.2}, and R^{14.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁴, R^{14.1}, R^{14.2}, and R^{14.3}, respectively.

In embodiments, when R¹⁵ is substituted, R¹⁵ is substituted with one or more first substituent groups denoted by R^{15.1} as explained in the definitions section above in the description of “first substituent group(s)”. In embodiments, when an R^{15.1} substituent group is substituted, the R^{15.1} substituent group is substituted with one or more second substituent groups denoted by R^{15.2} as explained in the definitions section above in the description of “first substituent group(s)”. In embodiments, when an R^{15.2} substituent group is substituted, the R^{15.2} substituent group is substituted with one or more third substituent groups denoted by R^{15.3} as explained in the definitions section above in the description of “first substituent group(s)”. In the above embodiments, R¹⁵, R^{15.1}, R^{15.2}, and R^{15.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁵, R^{15.1}, R^{15.2}, and R^{15.3}, respectively.

In embodiments, when R¹⁶ is substituted, R¹⁶ is substituted with one or more first substituent groups denoted by R^{16.1} as explained in the definitions section above in the description of “first substituent group(s)”. In embodiments, when an R^{16.1} substituent group is substituted, the R^{16.1} substituent group is substituted with one or more second substituent groups denoted by R^{16.2} as explained in the definitions section above in the description of “first substituent group(s)”. In embodiments, when an R^{16.2} substituent group is substituted, the R^{16.2} substituent group is substituted with one or more third substituent groups denoted by R^{16.3} as explained in the definitions section above in the description of “first substituent group(s)”. In the above embodiments, R¹⁶, R^{16.1}, R^{16.2}, and R^{16.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁶, R^{16.1}, R^{16.2}, and R^{16.3}, respectively.

In embodiments, when R^{16A} is substituted, R^{16A} is substituted with one or more first substituent groups denoted by R^{16A.1} as explained in the definitions section above in the description of “first substituent group(s)”. In embodiments, when an R^{16A.1} substituent group is substituted, the R^{16A.1} substituent group is substituted with one or more second substituent groups denoted by R^{16A.2} as explained in the definitions section above in the description of “first substituent group(s)”. In embodiments, when an R^{16A.2} substituent group is substituted, the R^{16A.2} substituent group is substituted with one or more third substituent groups denoted by R^{16A.3} as explained in the definitions section above in the description of “first substituent group(s)”. In the above embodiments, R^{16A}, R^{16A.1}, R^{16A.2}, and R^{16A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{16A}, R^{16A.1}, R^{16A.2}, and R^{16A.3}, respectively.

In embodiments, when R^{16B} is substituted, R^{16B} is substituted with one or more first substituent groups denoted by R^{16B.1} as explained in the definitions section above in the description of “first substituent group(s)”. In embodiments, when an R^{16B.1} substituent group is substituted, the R^{16B.1} substituent group is substituted with one or more second substituent groups denoted by R^{16B.2} as explained in the definitions section above in the description of “first substituent group(s)”. In embodiments, when an R^{16B.2} substituent group is substituted, the R^{16B.2} substituent group is substituted with one or more third substituent groups denoted by R^{16B.3} as explained in the definitions section above in the description of “first substituent group(s)”. In the above embodiments, R^{16B}, R^{16B.1}, R^{16B.2}, and R^{16B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{16B}, R^{16B.1}, R^{16B.2}, and R^{16B.3}, respectively.

In embodiments, when R^{16A} and R^{16B} substituents that are bonded to the same nitrogen atom are joined to form a moiety that is substituted (e.g., a substituted heterocycloalkyl or substituted heteroaryl), the moiety is substituted with one or more first substituent groups denoted by R^{16A.1} as explained in the definitions section above in the description of “first substituent group(s)”. In embodiments, when an R^{16A.1} substituent group is substituted, the R^{16A.1} substituent group is substituted with one or more second substituent groups denoted by R^{16A.2} as explained in the definitions section above in the description of “first substituent group(s)”. In embodiments, when an R^{16A.2} substituent group is substituted, the R^{16A.2} substituent group is substituted with one or more third substituent groups denoted by R^{16A.3} as explained in the definitions section above in the description of “first substituent group(s)”. In the above embodiments, R^{16A.1}, R^{16A.2}, and R^{16A.3} have values corresponding to the values of R^{WW}, R^{WW.2}, and R^{WW.3}, respectively, as explained in the definitions section above in the description of “first substituent group(s)”, wherein R^{WW.1}, R^{WW.2} and R^{WW.3} correspond to R^{16A.1}, R^{16A.2}, and R^{16A.3}, respectively.

In embodiments, when R^{16A} and R^{16B} substituents that are bonded to the same nitrogen atom are joined to form a moiety that is substituted (e.g., a substituted heterocycloalkyl or substituted heteroaryl), the moiety is substituted with one or more first substituent groups denoted by R^{16B.1} as explained in the definitions section above in the description of “first substituent group(s)”. In embodiments, when an R^{16B.1} substituent group is substituted, the R^{16B}1 substituent group is substituted with one or more second substituent groups denoted by R^{16B.2} as explained in the definitions section above in the description of “first substituent group(s)”. In embodiments, when an R^{16B.2} substituent group is substituted, the R^{16B.2} substituent group is substituted with one or more third substituent groups denoted by R^{16B.3} as explained in the definitions section above in the description of “first substituent group(s)”. In the above embodiments, R^{16B.1}, R^{16B.2}, and R^{16B.3} have values corresponding to the values of R^{WW}, R^{WW.2}, and R^{WW.3}, respectively, as explained in the definitions section above in the description of “first substituent group(s)”, wherein R^{WW.1}, R^{WW.2} and R^{WW.3} correspond to R^{16B.1}, R^{16B.2}, and R^{16B.3}, respectively.

In embodiments, when R¹⁷ is substituted, R¹⁷ is substituted with one or more first substituent groups denoted by R^{17.1} as explained in the definitions section above in the description of “first substituent group(s)”. In embodiments, when an R^{17.1} substituent group is substituted, the R^{17.1} substituent group is substituted with one or more second substituent groups denoted by R^{17.2} as explained in the definitions section above in the description of “first substituent group(s)”. In embodiments, when an R^{17.2} substituent group is substituted, the R^{17.2} substituent group is substituted with one or more third substituent groups denoted by R^{17.3} as explained in the definitions section above in the description of “first substituent group(s)”. In the above embodiments, R¹⁷, R^{17.1}, R^{17.2}, and R^{17.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁷, R^{17.1}, R^{17.2}, and R^{17.3}, respectively.

In embodiments, when R¹⁸ is substituted, R¹⁸ is substituted with one or more first substituent groups denoted by R^{18.1} as explained in the definitions section above in the description of “first substituent group(s)”. In embodiments, when an R^{18.1} substituent group is substituted, the R^{18.1} substituent group is substituted with one or more second substituent groups denoted by R^{18.2} as explained in the definitions section above in the description of “first substituent group(s)”. In embodiments, when an R^{18.2} substituent group is substituted, the R^{18.2} substituent group is substituted with one or more third substituent groups denoted by R^{18.3} as explained in the definitions section above in the description of “first substituent group(s)”. In the above embodiments, R¹⁸, R^{18.1}, R^{18.2}, and R^{18.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁸, R^{18.1}, R^{18.2}, and R^{18.3}, respectively.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, a substituted ring formed when R⁶ and R⁷ substituents, in combination with the —CHN— connecting the two substituents, are joined (e.g., substituted heterocycloalkyl) is substituted with at least one substituent group, size-limited substituent group, or lower substituent group; wherein if the substituted ring formed when R⁶ and R⁷ substituents, in combination with the —CHN— connecting the two substituents, are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R⁶ and R⁷ substituents, in combination with the —CHN— connecting the two substituents, are joined is substituted, it is substituted with at least one substituent group. In embodiments, when the substituted ring formed when R⁶ and R⁷ substituents, in combination with the —CHN— connecting the two substituents, are joined is substituted, it is substituted with at least one size-limited substituent group. In embodiments, when the substituted ring formed when R⁶ and R⁷ substituents, in combination with the —CHN— connecting the two substituents, are joined is substituted, it is substituted with at least one lower substituent group.

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, a substituted R⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁹ is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R⁹ is substituted, it is substituted with at least one size-limited substituent group. In embodiments, when R⁹ is substituted, it is substituted with at least one lower substituent group.

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, a substituted R¹¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹¹ is substituted with a plurality of groups selected from substituent groups, size-limited substituent groups, and lower substituent groups; each substituent group, size-limited substituent group, and/or lower substituent group may optionally be different. In embodiments, when R¹¹ is substituted, it is substituted with at least one substituent group. In embodiments, when R¹¹ is substituted, it is substituted with at least one size-limited substituent group. In embodiments, when R¹¹ is substituted, it is substituted with at least one lower substituent group.

In embodiments, a substituted R¹² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹² is substituted with a plurality of groups selected from substituent groups, size-limited substituent groups, and lower substituent groups; each substituent group, size-limited substituent group, and/or lower substituent group may optionally be different. In embodiments, when R¹² is substituted, it is substituted with at least one substituent group. In embodiments, when R¹² is substituted, it is substituted with at least one size-limited substituent group. In embodiments, when R¹² is substituted, it is substituted with at least one lower substituent group.

In embodiments, a substituted R¹³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹³ is substituted with a plurality of groups selected from substituent groups, size-limited substituent groups, and lower substituent groups; each substituent group, size-limited substituent group, and/or lower substituent group may optionally be different. In embodiments, when R¹³ is substituted, it is substituted with at least one substituent group. In embodiments, when R¹³ is substituted, it is substituted with at least one size-limited substituent group. In embodiments, when R¹³ is substituted, it is substituted with at least one lower substituent group.

In embodiments, a substituted R¹⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁴ is substituted with a plurality of groups selected from substituent groups, size-limited substituent groups, and lower substituent groups; each substituent group, size-limited substituent group, and/or lower substituent group may optionally be different. In embodiments, when R¹⁴ is substituted, it is substituted with at least one substituent group. In embodiments, when R¹⁴ is substituted, it is substituted with at least one size-limited substituent group. In embodiments, when R¹⁴ is substituted, it is substituted with at least one lower substituent group.

In embodiments, a substituted R¹⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁵ is substituted with a plurality of groups selected from substituent groups, size-limited substituent groups, and lower substituent groups; each substituent group, size-limited substituent group, and/or lower substituent group may optionally be different. In embodiments, when R¹⁵ is substituted, it is substituted with at least one substituent group. In embodiments, when R¹⁵ is substituted, it is substituted with at least one size-limited substituent group. In embodiments, when R¹⁵ is substituted, it is substituted with at least one lower substituent group.

In embodiments, a substituted R¹⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁶ is substituted with a plurality of groups selected from substituent groups, size-limited substituent groups, and lower substituent groups; each substituent group, size-limited substituent group, and/or lower substituent group may optionally be different. In embodiments, when R¹⁶ is substituted, it is substituted with at least one substituent group. In embodiments, when R¹⁶ is substituted, it is substituted with at least one size-limited substituent group. In embodiments, when R¹⁶ is substituted, it is substituted with at least one lower substituent group.

In embodiments, a substituted R^{16A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16A} is substituted with a plurality of groups selected from substituent groups, size-limited substituent groups, and lower substituent groups; each substituent group, size-limited substituent group, and/or lower substituent group may optionally be different. In embodiments, when R^{16A} is substituted, it is substituted with at least one substituent group. In embodiments, when R^{16A} is substituted, it is substituted with at least one size-limited substituent group. In embodiments, when R^{16A} is substituted, it is substituted with at least one lower substituent group.

In embodiments, a substituted R^{16B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16B} is substituted with a plurality of groups selected from substituent groups, size-limited substituent groups, and lower substituent groups; each substituent group, size-limited substituent group, and/or lower substituent group may optionally be different. In embodiments, when R^{16B} is substituted, it is substituted with at least one substituent group. In embodiments, when R^{16B} is substituted, it is substituted with at least one size-limited substituent group. In embodiments, when R^{16B} is substituted, it is substituted with at least one lower substituent group.

In embodiments, a substituted ring formed when R^{16A} and R^{16B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{16A} and R^{16B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{16A} and R^{16B} substituents bonded to the same nitrogen atom are joined is substituted, it is substituted with at least one substituent group. In embodiments, when the substituted ring formed when R^{16A} and R^{16B} substituents bonded to the same nitrogen atom are joined is substituted, it is substituted with at least one size-limited substituent group. In embodiments, when the substituted ring formed when R^{16A} and R^{16B} substituents bonded to the same nitrogen atom are joined is substituted, it is substituted with at least one lower substituent group.

In embodiments, a substituted R¹⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁷ is substituted with a plurality of groups selected from substituent groups, size-limited substituent groups, and lower substituent groups; each substituent group, size-limited substituent group, and/or lower substituent group may optionally be different. In embodiments, when R¹⁷ is substituted, it is substituted with at least one substituent group. In embodiments, when R¹⁷ is substituted, it is substituted with at least one size-limited substituent group. In embodiments, when R¹⁷ is substituted, it is substituted with at least one lower substituent group.

In embodiments, a substituted R¹⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁸ is substituted with a plurality of groups selected from substituent groups, size-limited substituent groups, and lower substituent groups; each substituent group, size-limited substituent group, and/or lower substituent group may optionally be different. In embodiments, when R¹⁸ is substituted, it is substituted with at least one substituent group. In embodiments, when R¹⁸ is substituted, it is substituted with at least one size-limited substituent group. In embodiments, when R¹⁸ is substituted, it is substituted with at least one lower substituent group.

In embodiments, the compound is a compound described herein. In embodiments, the compound, or a pharmaceutically acceptable salt thereof, is the compound. In embodiments, the compound, or a pharmaceutically acceptable salt thereof, is the pharmaceutically acceptable salt of the compound. In embodiments, the compound has a slow off rate following binding to Elongation Factor 1-alpha (e.g., EEF1A1, EEF1A2, or (EEF1A1 and EEF1A2)). In embodiments, the compound irreversibly binds to Elongation Factor 1-alpha (e.g., EEF1A1, EEF1A2, or (EEF1A1 and EEF1A2)). In embodiments, the compound irreversibly binds to Elongation Factor 1-alpha (e.g., EEF1A1, EEF1A2, or (EEF1A1 and EEF1A2)). In embodiments, the Elongation Factor 1-alpha is EEF1A1. In embodiments, the Elongation Factor 1-alpha is EEF1A2. In embodiments, the Elongation Factor 1-alpha is EEF1A1 and EEF1A2. In embodiments, the Elongation Factor 1-alpha is EEF1A1 or EEF1A2. In embodiments, the compound has a slow off rate following binding to Elongation Factor 1-alpha 1. In embodiments, the compound irreversibly binds to Elongation Factor 1-alpha 1. In embodiments, the compound irreversibly binds to Elongation Factor 1-alpha 1. In embodiments, the compound has a slow off rate following binding to Elongation Factor 1-alpha 2. In embodiments, the compound irreversibly binds to Elongation Factor 1-alpha 2. In embodiments, the compound irreversibly binds to Elongation Factor 1-alpha 2. In embodiments, the compound is not A1, A2, A3, A4, or A5 as described in WO2010062159 and US20110201642A1, which are incorporated by reference in their entirety for all purposes. In embodiments, the compound is not A1 as described in WO2010062159 and US20110201642A1, which are incorporated by reference in their entirety for all purposes. In embodiments, the compound is not A2 as described in WO2010062159 and US20110201642A1, which are incorporated by reference in their entirety for all purposes. In embodiments, the compound is not A3 as described in WO2010062159 and US20110201642A1, which are incorporated by reference in their entirety for all purposes. In embodiments, the compound is not A4 as described in WO2010062159 and US20110201642A1, which are incorporated by reference in their entirety for all purposes. In embodiments, the compound is not A5 as described in WO2010062159 and US20110201642A1, which are incorporated by reference in their entirety for all purposes.

In embodiments, the compound is not:

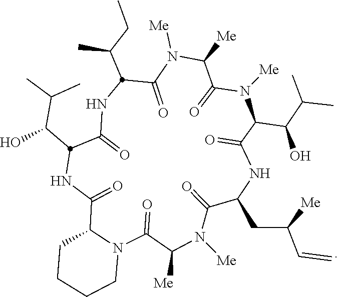 [SMILES: C=C[C@H](C)C[C@@H]1NC(=O)[C@H]([C@H](O)C(C)C)N(C)C(=O)[C@H](C)N(C)C(=O)[C@@H]([C@@H](C)CC)NC(=O)[C@@H]([C@H](O)C(C)C)NC(=O)[C@H]2CCCCN2C(=O)[C@H](C)N(C)C1=O]

In embodiments, the compound is not:

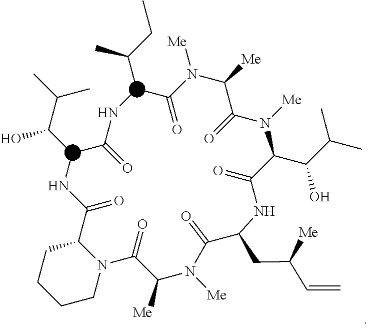 [SMILES: C=C[C@H](C)C[C@@H]1NC(=O)[C@H]([C@@H](O)C(C)C)N(C)C(=O)[C@H](C)N(C)C(=O)C([C@@H](C)CC)NC(=O)C([C@H](O)C(C)C)NC(=O)[C@H]2CCCCN2C(=O)[C@H](C)N(C)C1=O]

In embodiments, the compound (e.g., SRA3) is metabolicly stable. In embodiments, the compound has a long half-life. In embodiments, the compound has a long in vivo half-life (e.g., greater than about 1, 2, 3, 4, 5, 6, 7, 8, 9, 10, 20, 30, 40, 50, 60, 70, 80, 90, 100, 200, 300, 400, 500, 600, 700, 800, 900, or 1000) hours. In embodiments, the compound has an in vivo half-life greater than about 1 (e.g., 2, 3, 4, 5, 6, 7, 8, 9, 10, 20, 30, 40, 50, 60, 70, 80, 90, 100, 200, 300, 400, 500, 600, 700, 800, 900, or 1000) hours. In embodiments, the compound has a long in vivo half-life (e.g., greater than 1, 2, 3, 4, 5, 6, 7, 8, 9, 10, 20, 30, 40, 50, 60, 70, 80, 90, 100, 200, 300, 400, 500, 600, 700, 800, 900, or 1000) hours. In embodiments, the compound has an in vivo half-life greater than 1 (e.g., 2, 3, 4, 5, 6, 7, 8, 9, 10, 20, 30, 40, 50, 60, 70, 80, 90, 100, 200, 300, 400, 500, 600, 700, 800, 900, or 1000) hours. In embodiments, the compounds inhibits cell proliferation at a lower concentration then the compound inhibits protein synthesis (e.g., at a concentration at least 1.1, 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 fold less). In embodiments, the compound is administered by IV. In embodiments, the compound binds eEF1A.

In embodiments, the compound is useful as a comparator compound. In embodiments, the comparator compound can be used to assess the activity of a test compound in an assay (e.g., an assay as described herein, for example in the examples section, figures, or tables).

In embodiments, the compound is a compound described herein (e.g., in an aspect, embodiment, example, table, figure, or claim).

### III. Pharmaceutical Compositions

In an aspect is provided a pharmaceutical composition including a compound as described herein, including embodiments, and a pharmaceutically acceptable excipient. In embodiments, the compound as described herein is included in a therapeutically effective amount.

In embodiments of the pharmaceutical compositions, the compound, or pharmaceutically acceptable salt thereof, is included in a therapeutically effective amount. In embodiments, the compound is administered in a unit dose amount from 5 mg/m² to 200 mg/m². In embodiments, the compound is administered in a unit dose amount from 1 mg/m² to 500 mg/m². In embodiments, the compound is administered in a unit dose amount from 1 mg/m² to 300 mg/m². In embodiments, the compound is administered in a unit dose amount from 5 mg/m² to 100 mg/m². In embodiments, the compound is administered in a unit dose amount from 100 mg/m² to 200 mg/m². In embodiments, the compound is administered in a unit dose amount of about 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308,309,310,311,312,313,314,315,316,317,318,319,320,321,322,323,324,325,326,327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 mg/m2. In embodiments, the compound is administered in a unit dose amount of at least 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309,310,311,312,313,314,315,316,317,318,319,320,321,322,323,324,325,326,327,328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 mg/m2. In embodiments, the compound is administered in a unit dose amount equal to or less than 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306,307,308,309,310,311,312,313,314,315,316,317,318,319,320,321,322,323,324,325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 mg/m². In embodiments, the pharmaceutical composition is administered by IV.

In embodiments of the pharmaceutical compositions, the pharmaceutical composition includes a second agent (e.g. therapeutic agent). In embodiments of the pharmaceutical compositions, the pharmaceutical composition includes a second agent (e.g. therapeutic agent) in a therapeutically effective amount. In embodiments of the pharmaceutical compositions, the second agent is an agent for treating cancer. In embodiments, the administering does not include administration of any active agent other than the recited active agent (e.g., a compound described herein). In embodiments of the pharmaceutical compositions, the second agent is an agent for treating a virus infection (e.g., RNA virus infection, single stranded RNA virus infection, positive-sense single stranded RNA virus infection, coronavirus infection, SARS coronavirus infection, or SARS-CoV-2 infection). In embodiments of the pharmaceutical compositions, the second agent is an agent for treating arrhythmia. In embodiments of the pharmaceutical compositions, the second agent is an agent for treating acute respiratory distress syndrome (ARDS). In embodiments of the pharmaceutical compositions, the second agent is an agent for treating a SARS-CoV-2 associated disease.

### IV. Methods of Use

In an aspect is provided a method of decreasing the level of Elongation Factor 1-alpha (e.g., EEF1A1, EEF1A2, or (EEF1A1 and EEF1A2)) protein activity in a subject, the method including administering a compound described herein to the subject. In embodiments, the Elongation Factor 1-alpha is EEF1A1. In embodiments, the Elongation Factor 1-alpha is EEF1A2. In embodiments, the Elongation Factor 1-alpha is EEF1A1 and EEF1A2. In embodiments, the Elongation Factor 1-alpha is EEF1A1 or EEF1A2. In embodiments, the method includes an increased level of degradation of Elongation Factor 1-alpha (e.g., EEF1A1, EEF1A2, or (EEF1A1 and EEF1A2)) compared to absence of the compound described herein. In embodiments, the method includes a decreased level of Elongation Factor 1-alpha (e.g., EEF1A1, EEF1A2, or (EEF1A1 and EEF1A2)) protein compared to absence of the compound described herein. In embodiments, the compound inhibits translation elongation.

In an aspect is provided a method of inhibiting cancer growth in a subject in need thereof, the method including administering to the subject in need thereof an effective amount of a compound described herein, or a pharmaceutically acceptable salt thereof. In embodiments, the effective amount is a therapeutically effective amount.

In an aspect is provided a method of inhibiting cancer cell growth, the method including contacting the cancer cell with an effective amount of a compound described herein. In embodiments, the effective amount is a therapeutically effective amount.

In an aspect is provided a method of treating cancer in a subject in need thereof, the method including administering to the subject in need thereof an effective amount of a compound described herein. In embodiments, the effective amount is a therapeutically effective amount.

In embodiments, the cancer is a hematological cancer. In embodiments, the cancer is acute lymphoblastic leukemia, acute myeloid leukemia, chronic myelogenous leukemia, or multiple myeloma. In embodiments, the cancer is acute lymphoblastic leukemia. In embodiments, the cancer is acute myeloid leukemia. In embodiments, the cancer is chronic myelogenous leukemia. In embodiments, the cancer is multiple myeloma. In embodiments, the cancer is relapsed multiple myeloma. In embodiments, the cancer is refractory multiple myeloma. In embodiments, the cancer is relapsed and refractory multiple myeloma. In embodiments, the cancer is resistant to treatment with a tyrosine kinase inhibitor. In embodiments, the tyrosine kinase inhibitor is bosutinib, crizotinib, dasatinib, erlotinib, gefitinib, imatinib, afatinib, neratinib, lapatinib, nilotinib, ponatinib, midostaurin, gilteritinib, osimertinib, ibrutinib, or acalabrutinib. In embodiments, the tyrosine kinase inhibitor is bosutinib. In embodiments, the tyrosine kinase inhibitor is crizotinib. In embodiments, the tyrosine kinase inhibitor is dasatinib. In embodiments, the tyrosine kinase inhibitor is erlotinib. In embodiments, the tyrosine kinase inhibitor is gefitinib. In embodiments, the tyrosine kinase inhibitor is imatinib. In embodiments, the tyrosine kinase inhibitor is afatinib. In embodiments, the tyrosine kinase inhibitor is neratinib. In embodiments, the tyrosine kinase inhibitor is lapatinib. In embodiments, the tyrosine kinase inhibitor is nilotinib. In embodiments, the tyrosine kinase inhibitor is ponatinib. In embodiments, the tyrosine kinase inhibitor is midostaurin. In embodiments, the tyrosine kinase inhibitor is gilteritinib. In embodiments, the tyrosine kinase inhibitor is osimertinib. In embodiments, the tyrosine kinase inhibitor is ibrutinib. In embodiments, the tyrosine kinase inhibitor is acalabrutinib. In embodiments, the method does not include myalgia.

In embodiments, the cancer is lymphoma. In embodiments, the cancer is associated with MYC activity (e.g., aberrant MYC activity; increased MYC activity compared to non-cancer cells of the same tissue; genomic translocation of MYC and increased MYC activity compared to control or non-cancer cell, tissue, or patient; a or MYC amplication and increased MYC activity compared to control or non-cancer cell, tissue, or patient). In embodiments, the cancer is associated with aberrant MYC activity. In embodiments, the lymphoma is associated with MYC activity (e.g., aberrant MYC activity). In embodiments, the cancer is pancreatic cancer. In embodiments, the cancer is pancreatic ductal adenocarcinoma. In embodiments, the pancreatic cancer is associated with MYC activity (e.g., aberrant MYC activity). In embodiments, the cancer is diffuse large B-Cell lymphoma (DLBCL). In embodiments, the cancer is diffuse large B-Cell lymphoma (DLBCL) with a MYC-associated mutation. In embodiments, the cancer is diffuse large B-Cell lymphoma (DLBCL) with a MYC genetic alteration (e.g., translocation such as a translocation involving an immunoglobulin enhancer). In embodiments, the cancer is pancreatic cancer. In embodiments, the cancer is pancreatic cancer associated with KRAS mutation. In embodiments, the cancer is lung cancer. In embodiments, the cancer is lung cancer associated with KRAS mutation. In embodiments, the cancer is colorectal carcinoma. In embodiments, the cancer is Burkitt's lymphoma. In embodiments, the cancer is breast cancer. In embodiments, the cancer is colon cancer. In embodiments, the cancer is sarcoma. In embodiments, the cancer is colon adenocarcinoma. In embodiments, the cancer is colorectal adenocarcinoma. In embodiments, the cancer is prostate cancer. In embodiments, the cancer is triple negative breast cancer. In embodiments, the cancer is multiple myeloma. In embodiments, the cancer is T-cell leukemia. In embodiments, the cancer is epithelial ovarian cancer. In embodiments, the cancer is gastric cancer. In embodiments, the cancer is uterine cancer. In embodiments, the cancer is relapsed/refractory multiple myeloma.

In embodiments, the method further includes co-administering an anti-cancer agent to the subject in need. In embodiments, the anti-cancer agent is dexamethasone. In embodiments, the anti-cancer agent is administered in an effective amount. In embodiments, the anti-cancer agent is administered in a therapeutically effective amount.

In an aspect is provided a method of treating a viral infection in a subject in need thereof, the method including administering to the subject in need thereof an effective amount of a compound described herein. In embodiments, the effective amount is a therapeutically effective amount.

In embodiments, the viral infection is an RNA virus infection. In embodiments, the viral infection is a single stranded RNA virus infection. In embodiments, the viral infection is a positive-sense single stranded RNA virus infection. In embodiments, the viral infection is a coronavirus infection. In embodiments, the viral infection is a SARS coronavirus (i.e., Severe acute respiratory syndrome-related coronavirus, SARS-CoV, or SARSr-Cov) infection. In embodiments, the viral infection is a SARS-CoV-2 (i.e., Severe acute respiratory syndrome coronavirus 2) infection.

In an aspect is provided a method of treating acute respiratory distress syndrome (ARDS) in a subject in need thereof, the method including administering to the subject in need thereof an effective amount of a compound described herein. In embodiments, the effective amount is a therapeutically effective amount. In embodiments, the acute respiratory distress syndrome (ARDS) is associated with a virus infection (e.g., RNA virus infection, single stranded RNA virus infection, positive-sense single stranded RNA virus infection, coronavirus infection, SARS coronavirus infection, or SARS-CoV-2 infection). In embodiments, the method further includes co-administering an agent for treating acute respiratory distress syndrome (ARDS) to the subject in need. In embodiments, the agent for treating acute respiratory distress syndrome (ARDS) is administered in an effective amount. In embodiments, the agent for treating acute respiratory distress syndrome (ARDS) is administered in a therapeutically effective amount.

In an aspect is provided a method of treating a coronavirus disease in a subject in need thereof, the method including administering to the subject in need thereof an effective amount of a compound described herein. In embodiments, the effective amount is a therapeutically effective amount. In embodiments, the coronavirus disease is associated with a virus infection (e.g., RNA virus infection, single stranded RNA virus infection, positive-sense single stranded RNA virus infection, coronavirus infection, SARS coronavirus infection, or SARS-CoV-2 infection).

In an aspect is provided a method of treating a SARS-CoV-2 infection in a subject in need thereof, the method including administering to the subject in need thereof an effective amount of a compound described herein.

In an aspect is provided a method of treating a SARS-CoV-2 associated disease in a subject in need thereof, the method including administering to the subject in need thereof an effective amount of a compound described herein.

In an aspect is provided a method of treating arrhythmia in a subject in need thereof, the method including administering to the subject in need thereof an effective amount of a compound described herein. In embodiments, the effective amount is a therapeutically effective amount. In embodiments, the arrhythmia is associated with a virus infection (e.g., RNA virus infection, single stranded RNA virus infection, positive-sense single stranded RNA virus infection, coronavirus infection, SARS coronavirus infection, or SARS-CoV-2 infection). In embodiments, the method further includes co-administering an agent for treating arrhythmia to the subject in need. In embodiments, the agent for treating arrhythmia is administered in an effective amount. In embodiments, the agent for treating arrhythmia is administered in a therapeutically effective amount.

In embodiments, the method includes inhibiting cap-dependent translation. In embodiments, the method includes inhibiting cap-dependent RNA translation. In embodiments, the method includes inhibiting the rate of translation elongation (e.g., compared to absence of the compound). In embodiments, the method includes inhibiting viral infectivity. In embodiments, the method includes inhibiting protein biogenesis. In embodiments, the compound is cytostatic. In embodiments, the compound is cytotoxic. In embodiments, the compound has a superior therapeutic index compared to plitidepsin. In embodiments, the compound has less side effects (e.g., less number of different side effects, a less severe side effect) compared to plitidepsin. In embodiments, the compound is administered by IV. In embodiments, the method further includes co-administering an agent for treating a virus infection (e.g., RNA virus infection, single stranded RNA virus infection, positive-sense single stranded RNA virus infection, coronavirus infection, SARS coronavirus infection, or SARS-CoV-2 infection) to the subject in need. In embodiments, the agent for treating a virus infection (e.g., RNA virus infection, single stranded RNA virus infection, positive-sense single stranded RNA virus infection, coronavirus infection, SARS coronavirus infection, or SARS-CoV-2 infection) is administered in an effective amount. In embodiments, the agent for treating a virus infection (e.g., RNA virus infection, single stranded RNA virus infection, positive-sense single stranded RNA virus infection, coronavirus infection, SARS coronavirus infection, or SARS-CoV-2 infection) is administered in a therapeutically effective amount.

### V. Additional Embodiments

Embodiment 1. A compound having the formula:

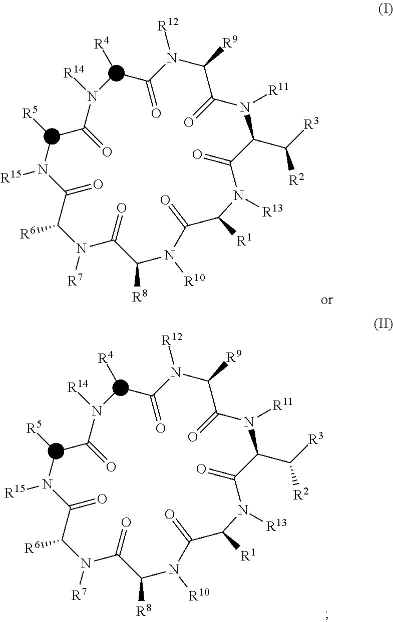

R¹ is substituted or unsubstituted alkyl or substituted or unsubstituted heteroalkyl; R² is —OCX²₃, —OCH₂X², —OCHX²₂, —SR^{2B}, —NR^{2A}R^{2B}, or —OR^{2B}; R^{2A} and R^{2B} are independently hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R³ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ substituents may optionally be joined to form, in combination with the —CHN— connecting the two substituents, a substituted or unsubstituted heterocycloalkyl; R⁸ and R⁹ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCCl₃, —OCF₃, —OCBr₃, —OCI₃, —OC HCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁰, R¹¹, R¹², R¹³, R¹⁴, and R¹⁵ are independently
hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X² is independently —F, —Cl, —Br, or —I; or a pharmaceutically acceptable salt thereof.

Embodiment 2. The compound of embodiment 1, wherein R² is —NR^{2A}R^{2B} or —OR^{2B}; R^{2A} and R^{2B} are independently

hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{2A} and R^{2B} substituents bonded to the same nitrogen atom may optionally be joined to form a substituted or unsubstituted heterocycloalkyl or substituted or unsubstituted heteroaryl.

Embodiment 3. The compound of embodiment 1, wherein R² is —OR^{2B}; R^{2B} is independently

hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl.

Embodiment 4. The compound of embodiment 1, wherein R² is —OH.

Embodiment 5. The compound of embodiment 1, wherein R² is —NH₂.

Embodiment 6. The compound of one of embodiments 1 to 5, having the formula:

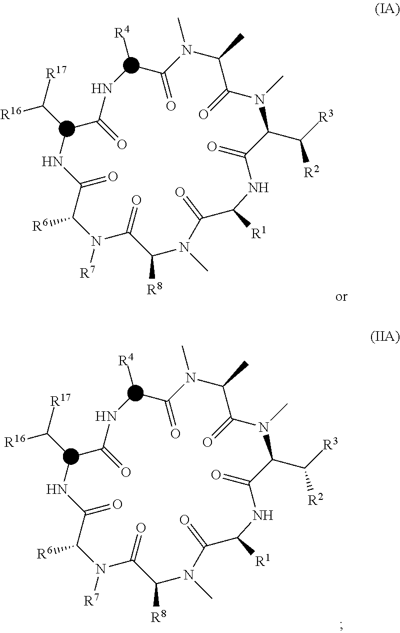

wherein R¹ is substituted or unsubstituted alkyl or substituted or unsubstituted heteroalkyl; R³ is substituted or unsubstituted alkyl or substituted or unsubstituted cycloalkyl; R⁴ is substituted or unsubstituted alkyl or substituted or unsubstituted cycloalkyl; R⁶ and R⁷ are independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl; R⁶ and R⁷ substituents may optionally be joined to form, in combination with the —CHN— connecting the two substituents, a substituted or unsubstituted heterocycloalkyl; R⁸ is
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl; R¹⁶ is —OCX¹⁶₃, —OCH₂X¹⁶, —OCHX¹⁶₂, —SR^{16B}, —NR^{16A}R^{16B} or —OR^{16B}; R^{16A} and R^{16B} are independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R¹⁷ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X¹⁶ is independently —F, —Cl, —Br, or —I; or a pharmaceutically acceptable salt thereof.

Embodiment 7. The compound of embodiment 1, having the formula:

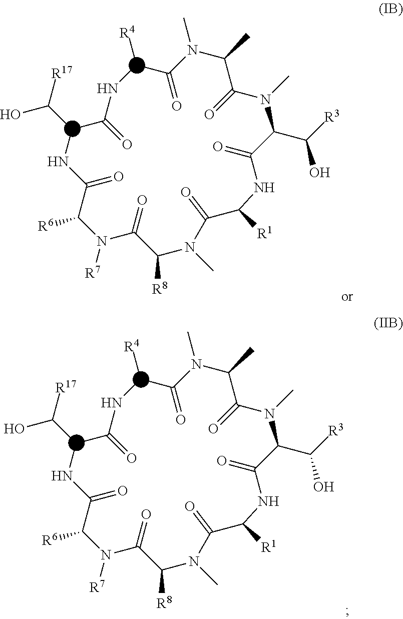

wherein R¹ is substituted or unsubstituted C₃-C₅ alkyl; R³ is substituted or unsubstituted C₁-C₆ alkyl or substituted or unsubstituted C₃-C₆ cycloalkyl; R⁴ is substituted or unsubstituted C₁-C₆ alkyl or substituted or unsubstituted C₃-C₆ cycloalkyl; R⁶ and R⁷ are independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl; R⁶ and R⁷ substituents may optionally be joined to form, in combination with the —CHN— connecting the two substituents, a substituted or unsubstituted 6 to 8 membered heterocycloalkyl; R⁸ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl; R¹⁷ is substituted or unsubstituted C₁-C₆ alkyl or substituted or unsubstituted C₃-C₆ cycloalkyl; or a pharmaceutically acceptable salt thereof.

Embodiment 8. The compound of embodiment 7, having the formula:

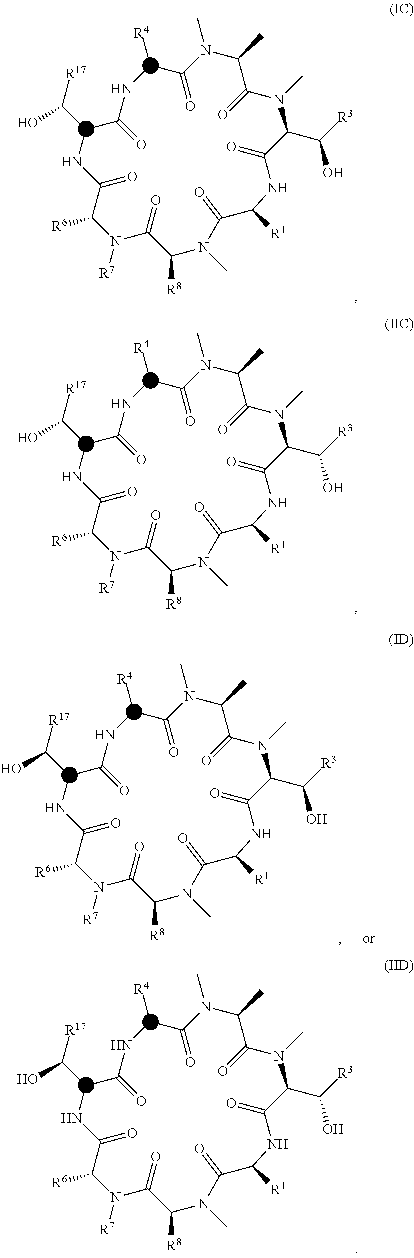

Embodiment 9. The compound of one of embodiments 6 to 8, wherein R¹ is fluoro-substituted or unsubstituted C₃-C₅ alkyl; R³ is fluoro-substituted or unsubstituted C₁-C₆ alkyl or fluoro-substituted or unsubstituted C₃-C₆ cycloalkyl; R⁴ is fluoro-substituted or unsubstituted C₁-C₆ alkyl or fluoro-substituted or unsubstituted C₃-C₆ cycloalkyl;

R⁶ and R⁷ are independently fluoro-substituted or unsubstituted C₁-C₄ alkyl, or fluoro-substituted or unsubstituted 2 to 4 membered heteroalkyl; R⁶ and R⁷ substituents may optionally be joined to form, in combination with the —CHN— connecting the two substituents, a fluoro-substituted or unsubstituted 6 to 8 membered heterocycloalkyl; R⁸ is fluoro-substituted or unsubstituted C₁-C₆ alkyl; R¹⁷ is fluoro-substituted or unsubstituted C₁-C₆ alkyl or fluoro-substituted or unsubstituted C₃-C₆ cycloalkyl; or a pharmaceutically acceptable salt thereof.

Embodiment 10. The compound of one of embodiments 1 to 9, wherein R¹ is

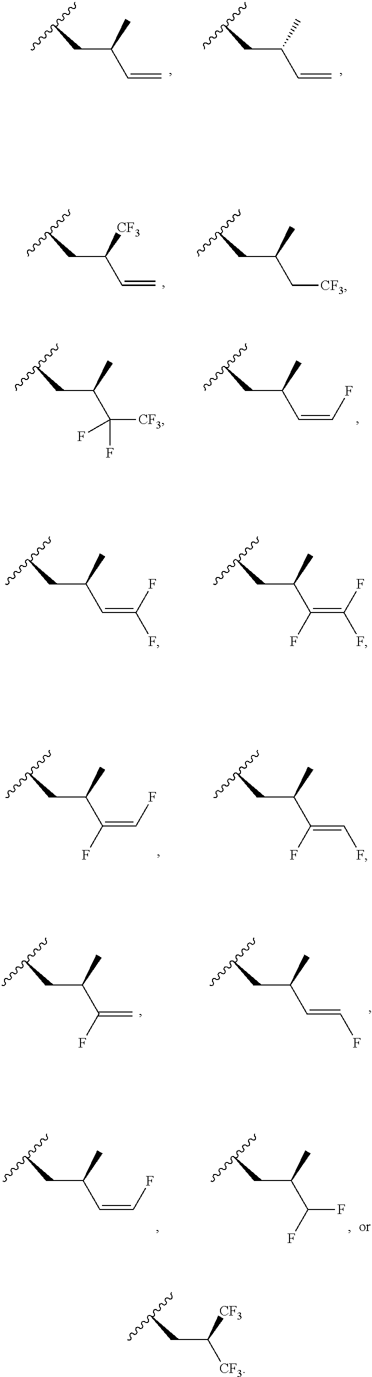

Embodiment 11. The compound of one of embodiments 1 to 9, wherein R¹ is

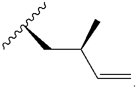

Embodiment 12. The compound of one of embodiments 1 to 11, wherein R³ is

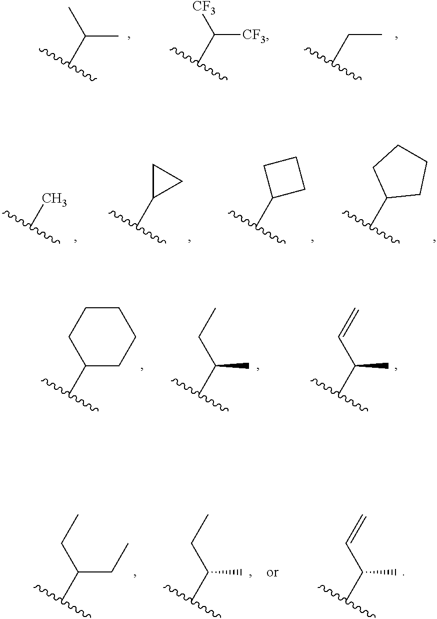

Embodiment 13. The compound of one of embodiments 1 to 11, wherein R³ is

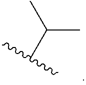 [SMILES: *C(C)C]

Embodiment 14. The compound of one of embodiments 1 to 13, wherein R⁴ is

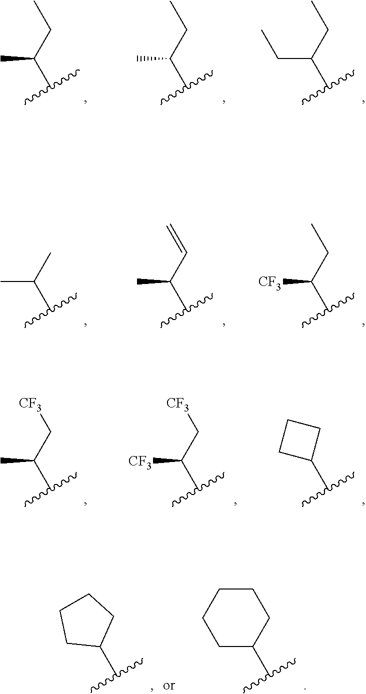

Embodiment 15. The compound of one of embodiments 1 to 13, wherein R⁴ is

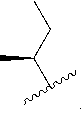 [SMILES: *[C@@H](C)CC]

Embodiment 16. The compound of one of embodiments 6 to 15, wherein R¹⁷ is

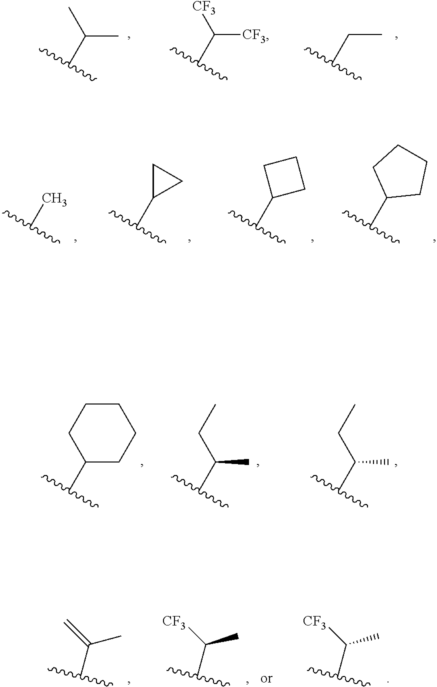

Embodiment 17. The compound of one of embodiments 6 to 15, wherein R¹⁷ is

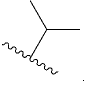 [SMILES: *C(C)C]

Embodiment 18. The compound of one of embodiments 1 to 17, wherein R⁶ is

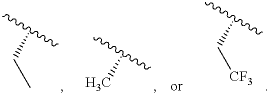

Embodiment 19. The compound of one of embodiments 1 to 18, wherein R⁷ is

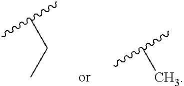

Embodiment 20. The compound of one of embodiments 1 to 17, wherein R⁶ and R⁷ substituents are joined to form, in combination with the —CHN— connecting the two substituents,

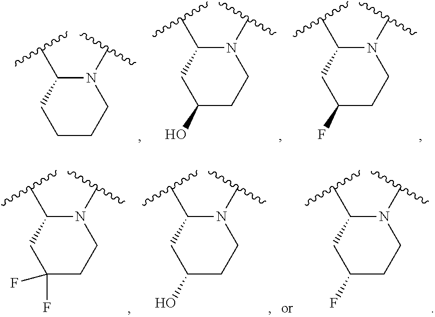 [SMILES: *[C@H]1CCCCN1* ; *[C@H]1C[C@H](O)CCN1* ; *[C@H]1C[C@H](F)CCN1* ; *[C@H]1CC(F)(F)CCN1* ; *[C@H]1C[C@@H](O)CCN1* ; *[C@H]1C[C@@H](F)CCN1*]

Embodiment 21. The compound of one of embodiments 1 to 17, wherein R⁶ and R⁷ substituents are joined to form, in combination with the —CHN— connecting the two substituents,

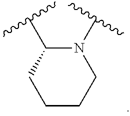 [SMILES: *[C@H]1CCCCN1*]

Embodiment 22. The compound of embodiment 1, having the formula:

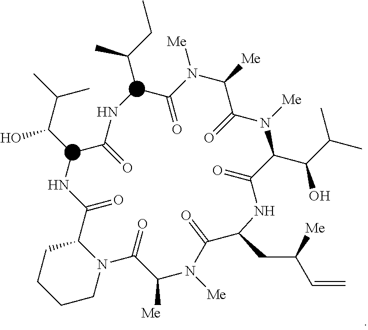 [SMILES: C=C[C@H](C)C[C@@H]1NC(=O)[C@H]([C@H](O)C(C)C)N(C)C(=O)[C@H](C)N(C)C(=O)C([C@@H](C)CC)NC(=O)C([C@H](O)C(C)C)NC(=O)[C@H]2CCCCN2C(=O)[C@H](C)N(C)C1=O]

Embodiment 23. The compound of embodiment 1, having the formula:

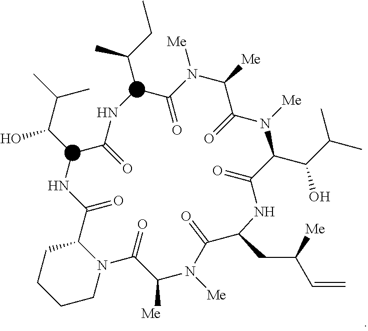 [SMILES: C=C[C@H](C)C[C@@H]1NC(=O)[C@H]([C@@H](O)C(C)C)N(C)C(=O)[C@H](C)N(C)C(=O)C([C@@H](C)CC)NC(=O)C([C@H](O)C(C)C)NC(=O)[C@H]2CCCCN2C(=O)[C@H](C)N(C)C1=O]

Embodiment 24. A pharmaceutical composition comprising the compound of any one of embodiments 1 to 23, or a pharmaceutically acceptable salt thereof, and a pharmaceutically acceptable excipient.

Embodiment 25. A method of decreasing the level of Elongation Factor 1-alpha protein activity in a subject, said method comprising administering a compound of one of embodiments 1 to 23 to said subject.

Embodiment 26. A method of inhibiting cancer growth in a subject in need thereof, said method comprising administering to the subject in need thereof an effective amount of a compound of one of embodiments 1 to 23, or a pharmaceutically acceptable salt thereof.

Embodiment 27. A method of inhibiting cancer cell growth, said method comprising contacting the cancer cell with an effective amount of a compound of one of embodiments 1 to 23.

Embodiment 28. A method of treating a cancer in a subject in need thereof, said method comprising administering to the subject in need thereof an effective amount of a compound of one of embodiments 1 to 23.

Embodiment 29. The method of embodiment 28, wherein the cancer is a hematological cancer.

Embodiment 30. The method of embodiment 28, wherein the cancer is acute lymphoblastic leukemia, acute myeloid leukemia, chronic myelogenous leukemia, or multiple myeloma.

Embodiment 31. The method of one of embodiments 28 to 30, wherein the cancer is resistant to treatment with a tyrosine kinase inhibitor.

Embodiment 32. The method of embodiment 31, wherein the tyrosine kinase inhibitor is bosutinib, crizotinib, dasatinib, erlotinib, gefitinib, imatinib, afatinib, neratinib, lapatinib, nilotinib, ponatinib, midostaurin, gilteritinib, osimertinib, ibrutinib, or acalabrutinib.

Embodiment 33. The method of one of embodiments 28 to 32, further comprising co-administering an anti-cancer agent to said subject in need.

Embodiment 34. A compound having the formula:

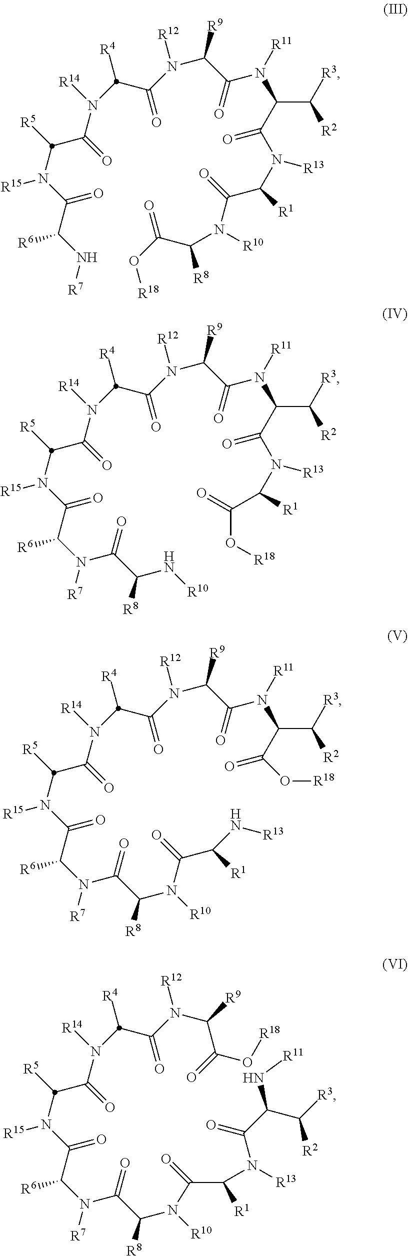

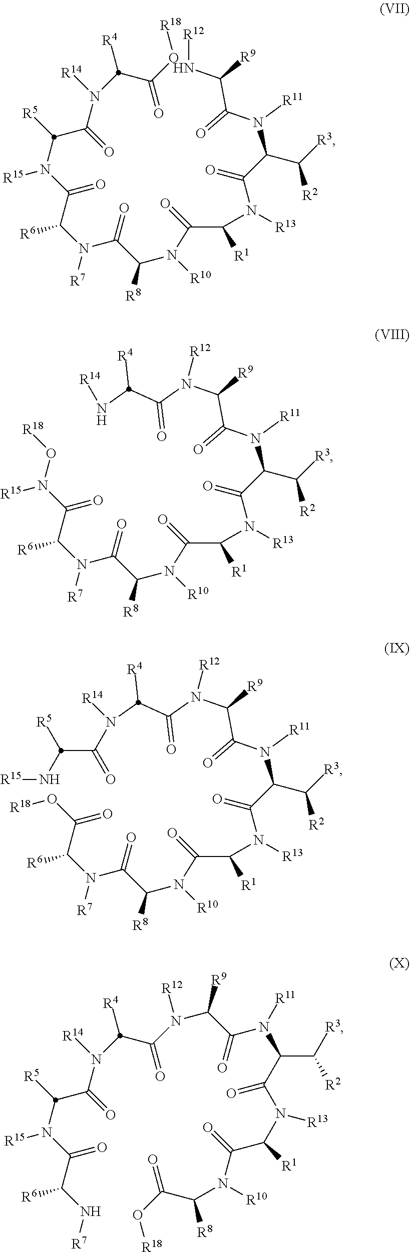

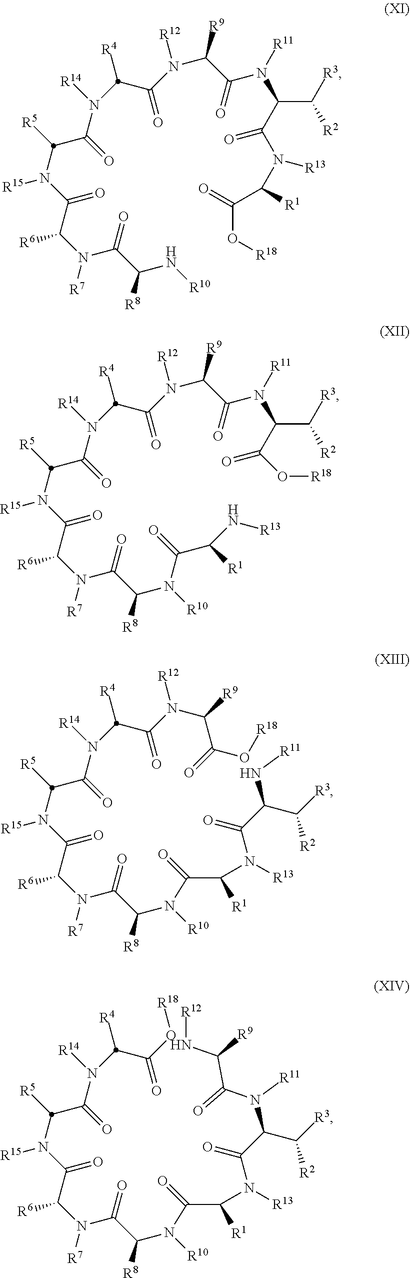

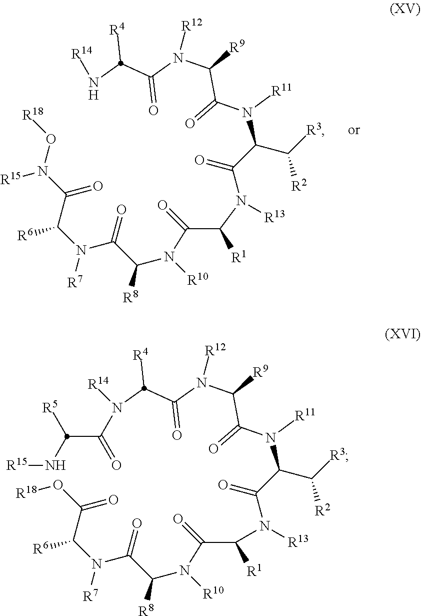

R¹ is substituted or unsubstituted alkyl or substituted or unsubstituted heteroalkyl; R² is —OCX²₃, —OCH₂X², —OCHX²₂, —SR^{2B}, —N^{2A}R^{2B}, or —OR^{2B}; R^{2A} and R^{2B} are independently hydrogen, —CCl₃, —CF₃, —CHF₂, —CH₂F, —CN, —OH, —NH₂, —COOH, —CONH₂, —OCCl₃, —OCF₃, —OCHF 2, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R³ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ is —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —O CH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ substituents may optionally be joined to form, in combination with the —CHN— connecting the two substituents, a substituted or unsubstituted heterocycloalkyl; R⁸ and R⁹ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCCl₃, —OCF₃, —OCBr₃, —OCl₃, —OC HCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁰, R¹¹, R¹², R¹³, R¹⁴, and R¹⁵ are independently
hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X² is independently —F, —Cl, —Br, or —I; R¹⁸ is independently
hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a resin moiety; or a pharmaceutically acceptable salt thereof.

Embodiment 35. The compound of embodiment 34, wherein R² is —NR^{2A}R^{2B} or —OR^{2B}; R^{2A} and R^{2B} are independently

hydrogen, —CCl₃, —CF₃, —CHF₂, —CH₂F, —CN, —OH, —NH₂, —COOH, —CONH₂, —OCCl₃, —OCF₃, —OCHF 2, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{2A} and R^{2B} substituents bonded to the same nitrogen atom may optionally be joined to form a substituted or unsubstituted heterocycloalkyl or substituted or unsubstituted heteroaryl.

Embodiment 36. The compound of embodiment 34, wherein R² is —OR^{2B}; R^{2B} is independently hydrogen, —CCl₃, —CF₃, —CHF₂, —CH₂F, —CN, —OH, —NH₂, —COOH, —CONH₂, —OCCl₃, —OCF₃, —OCHF 2, —OCH₂F, substituted or unsubstituted alkyl.

Embodiment 37. The compound of embodiment 34, wherein R² is —OH.

Embodiment 38. The compound of embodiment 34, wherein R² is —NH₂.

Embodiment 39. The compound of one of embodiments 34 to 38, having the formula:

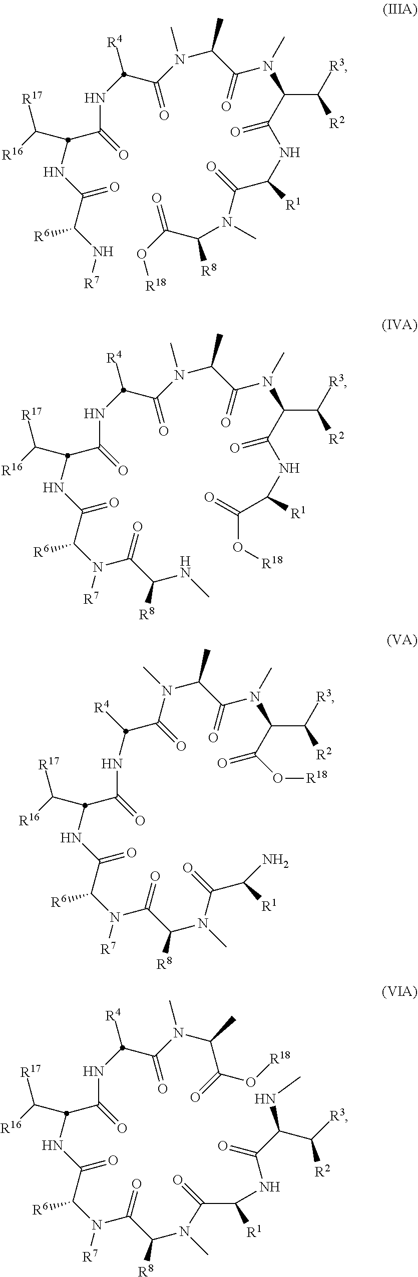

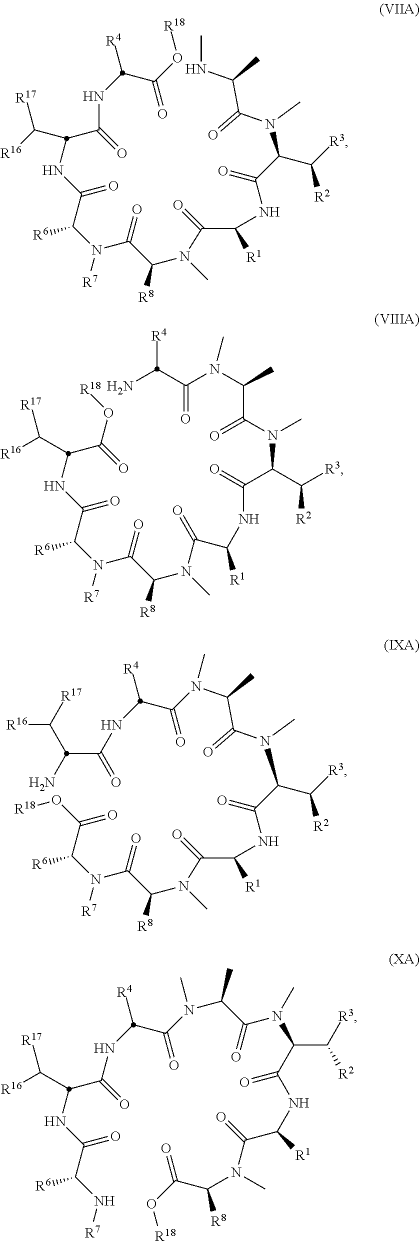

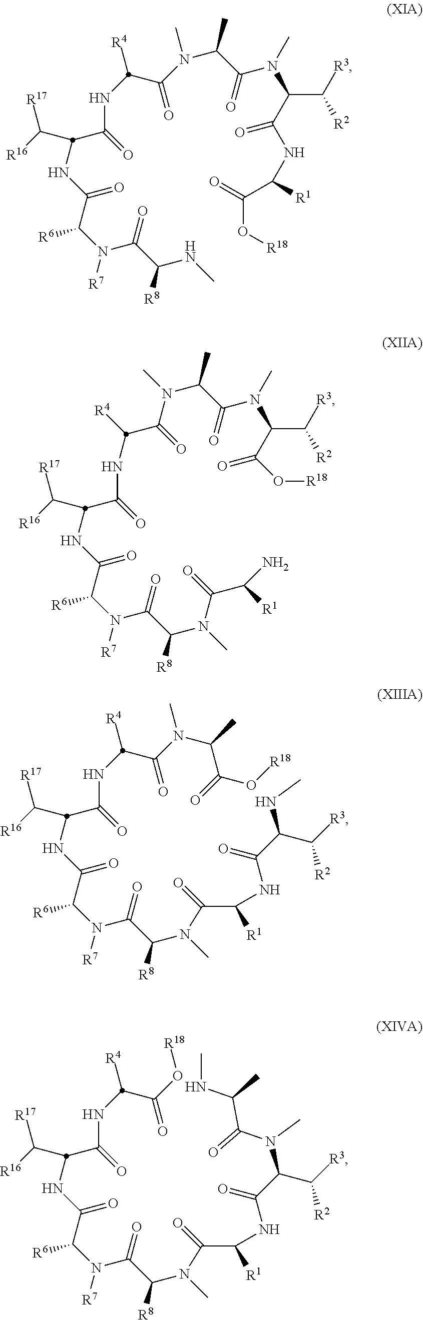

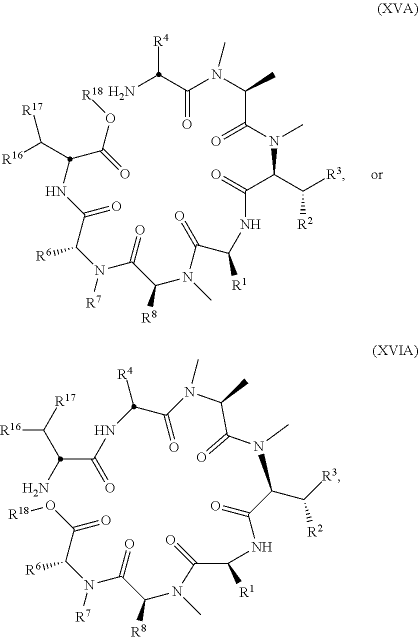

wherein R¹ is substituted or unsubstituted alkyl or substituted or unsubstituted heteroalkyl; R³ is substituted or unsubstituted alkyl or substituted or unsubstituted cycloalkyl; R⁴ is substituted or unsubstituted alkyl or substituted or unsubstituted cycloalkyl; R⁶ and R⁷ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl; R⁶ and R⁷ substituents may optionally be joined to form, in combination with the —CHN— connecting the two substituents, a substituted or unsubstituted heterocycloalkyl;
R⁸ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl; R¹⁶ is —OCX¹⁶₃, —OCH₂X¹⁶, —OCHX¹⁶₂, —SR^{16B}, —NR^{16A}R^{16B}, or —OR^{16B}; R^{16A} and R^{16B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R¹⁷ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X¹⁶ is independently —F, —Cl, —Br, or —I; or a pharmaceutically acceptable salt thereof.

Embodiment 40. The compound of embodiment 34, having the formula:

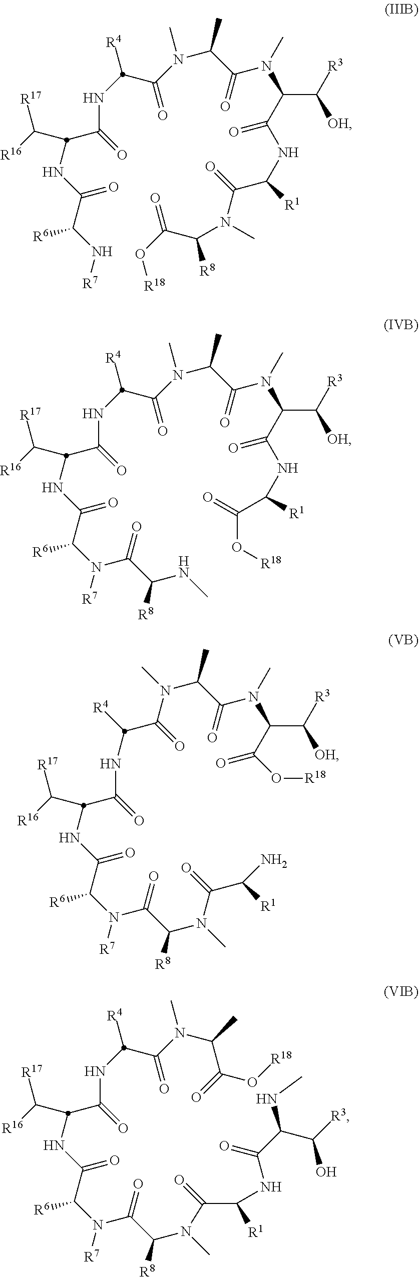

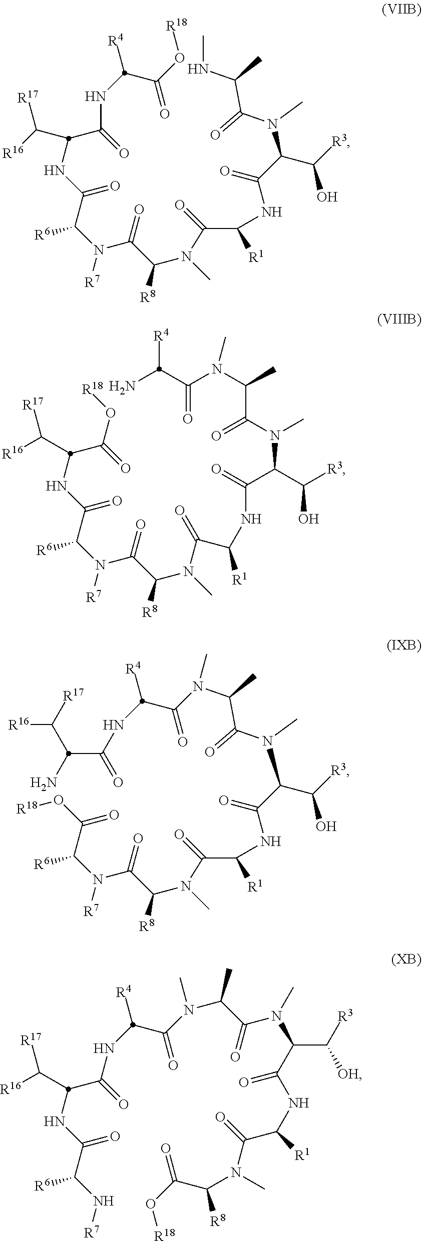

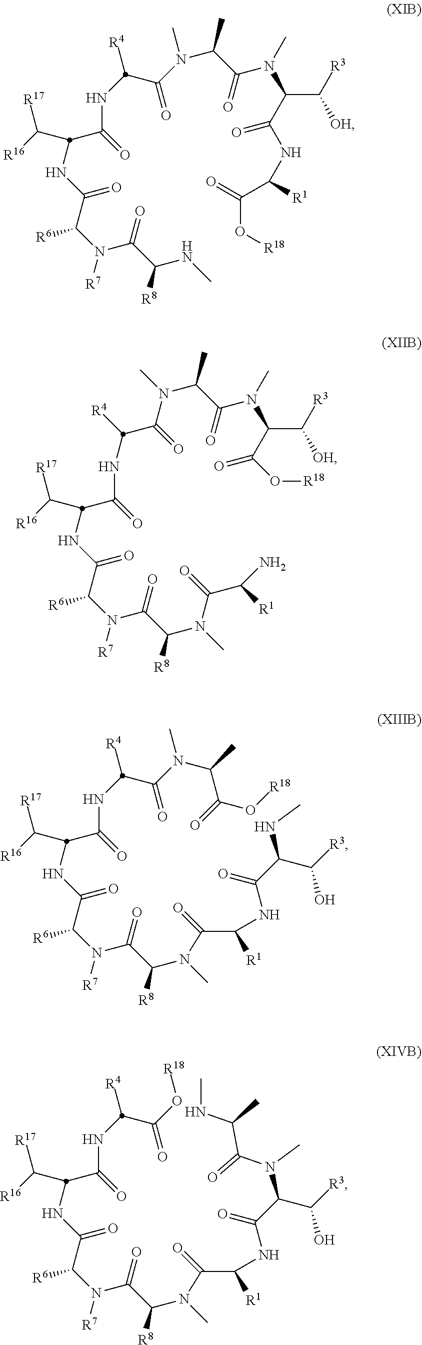

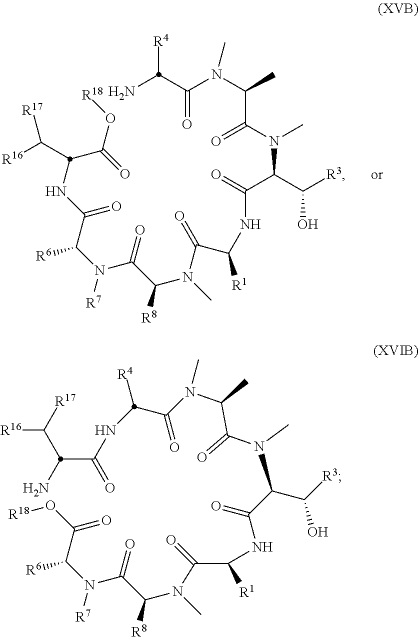

wherein R¹ is substituted or unsubstituted C₃-C₅ alkyl; R³ is substituted or unsubstituted C₁-C₆ alkyl or substituted or unsubstituted C₃-C₆ cycloalkyl; R⁴ is substituted or unsubstituted C₁-C₆ alkyl or substituted or unsubstituted C₃-C₆ cycloalkyl; R⁶ and R⁷ are independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl; R⁶ and R⁷ substituents may optionally be joined to form, in combination with the —CHN— connecting the two substituents, a substituted or unsubstituted 6 to 8 membered heterocycloalkyl; R⁸ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl; R¹⁷ is substituted or unsubstituted C₁-C₆ alkyl or substituted or unsubstituted C₃-C₆ cycloalkyl; or a pharmaceutically acceptable salt thereof.

Embodiment 41. The compound of embodiment 40, having the formula:

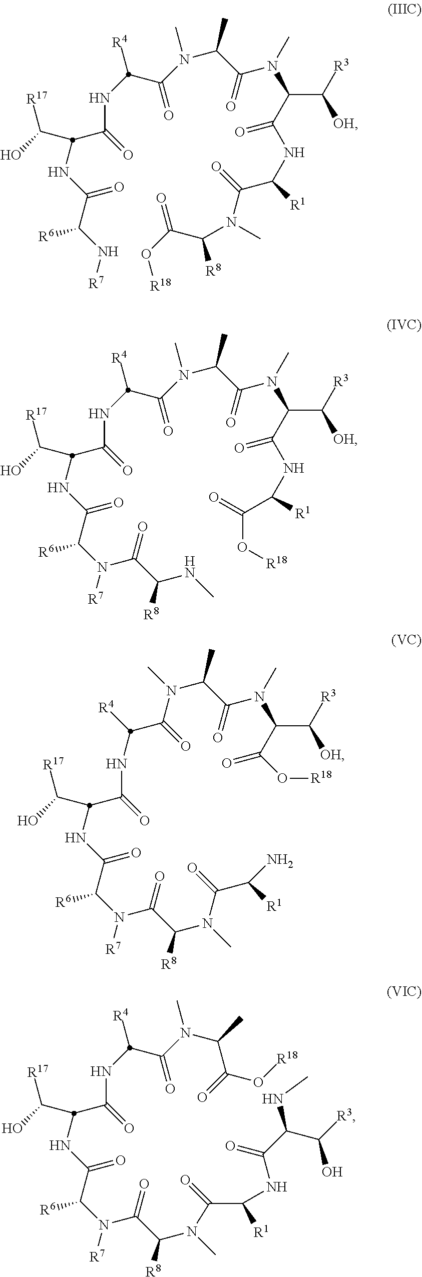

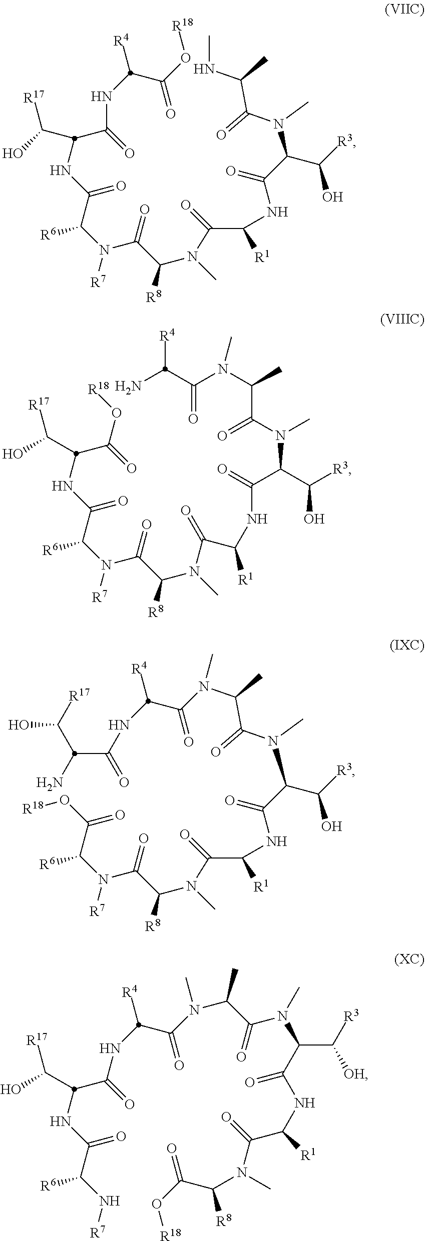

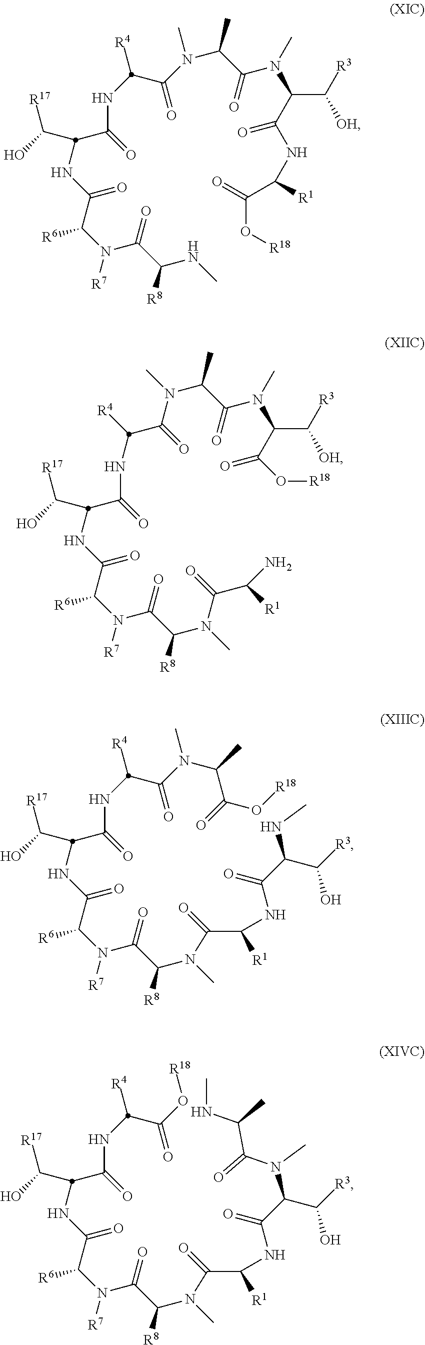

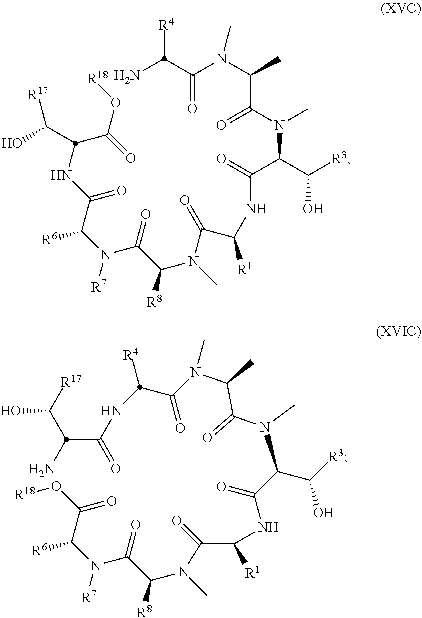

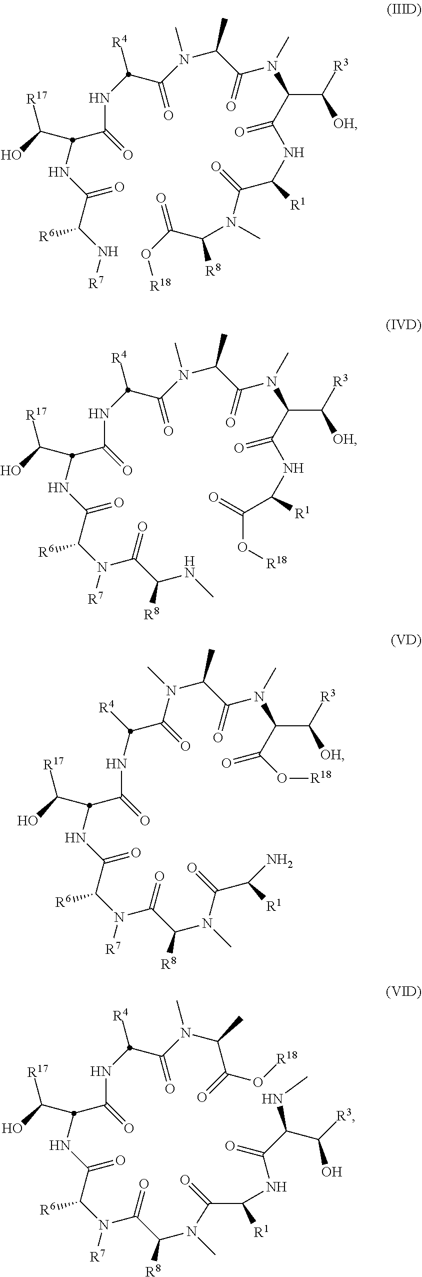

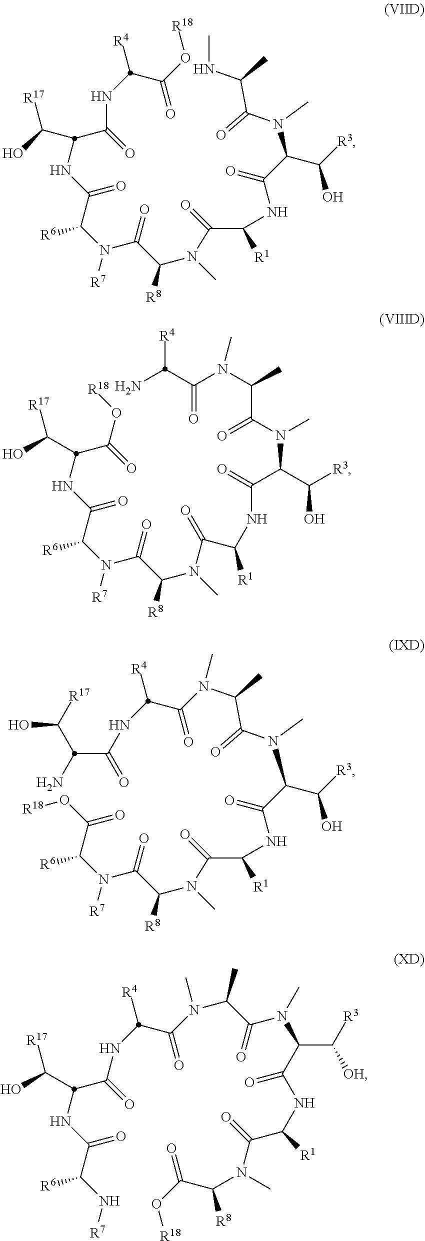

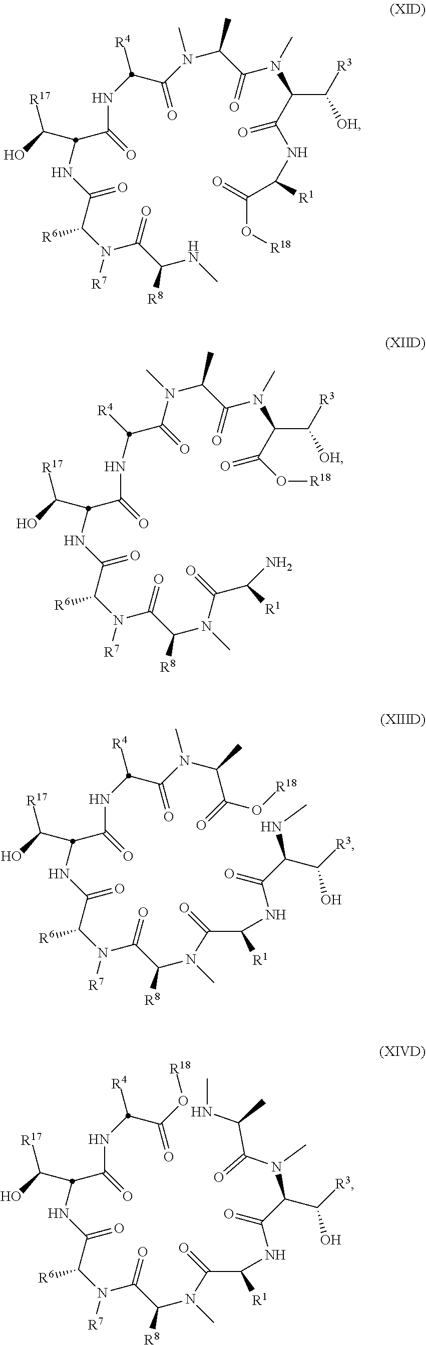

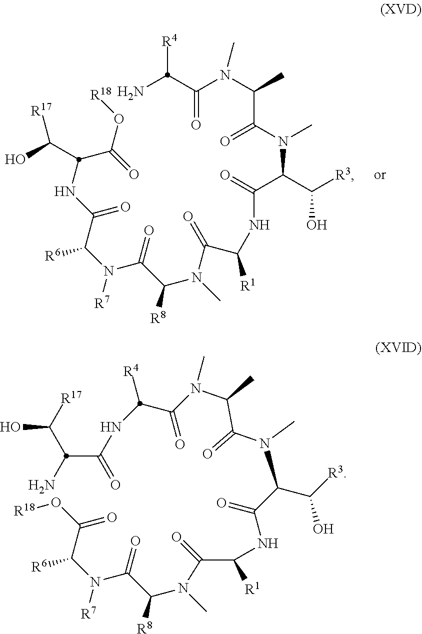

Embodiment 42. The compound of one of embodiments 39 to 41, wherein R¹ is fluoro-substituted or unsubstituted C₃-C₅ alkyl; R³ is fluoro-substituted or unsubstituted C₁-C₆ alkyl or fluoro-substituted or unsubstituted C₃-C₆ cycloalkyl; R⁴ is fluoro-substituted or unsubstituted C₁-C₆ alkyl or fluoro-substituted or unsubstituted C₃-C₆ cycloalkyl; R⁶ and R⁷ are independently fluoro-substituted or unsubstituted C₁-C₄ alkyl, or fluoro-substituted or unsubstituted 2 to 4 membered heteroalkyl; R⁶ and R⁷ substituents may optionally be joined to form, in combination with the —CHN— connecting the two substituents, a fluoro-substituted or unsubstituted 6 to 8 membered heterocycloalkyl; R⁸ is fluoro-substituted or unsubstituted C₁-C₆ alkyl; R¹⁷ is fluoro-substituted or unsubstituted C₁-C₆ alkyl or fluoro-substituted or unsubstituted C₃-C₆ cycloalkyl; or a pharmaceutically acceptable salt thereof.

Embodiment 43. The compound of one of embodiments 34 to 42, wherein R¹ is

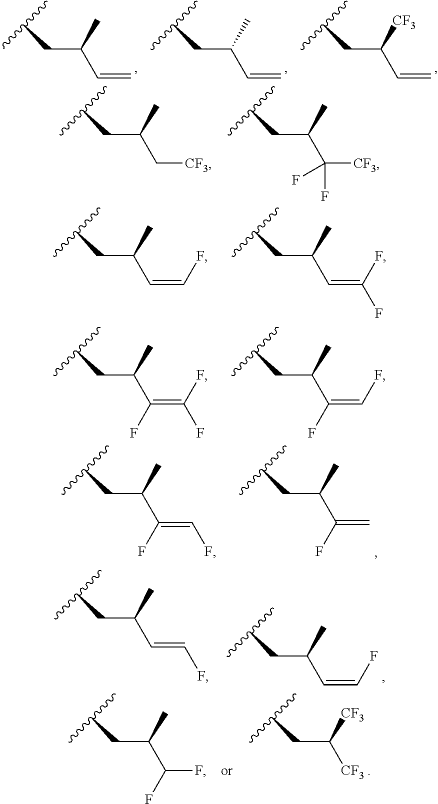

Embodiment 44. The compound of one of embodiments 34 to 42, wherein R¹ is

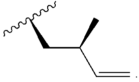

Embodiment 45. The compound of one of embodiments 34 to 44, wherein R³ is

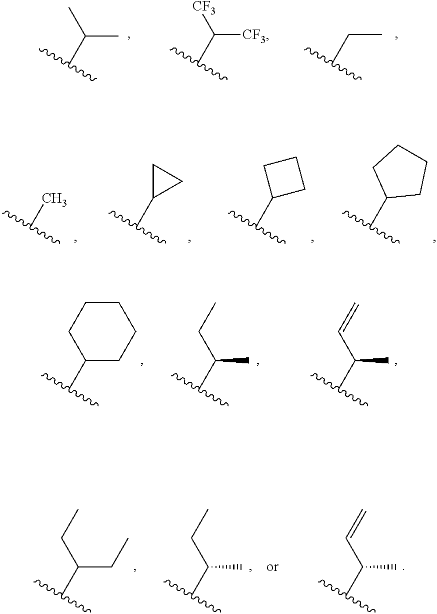

Embodiment 46. The compound of one of embodiments 34 to 44, wherein R³ is

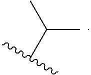 [SMILES: *C(C)C]

Embodiment 47. The compound of one of embodiments 34 to 46, wherein R⁴ is

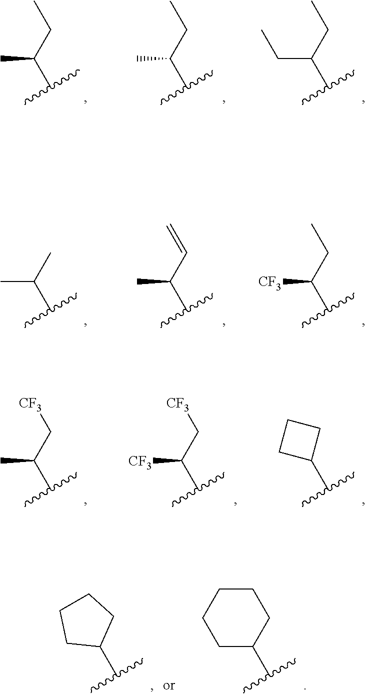

Embodiment 48. The compound of one of embodiments 34 to 46, wherein R⁴ is

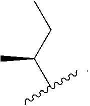 [SMILES: *[C@@H](C)CC]

Embodiment 49. The compound of one of embodiments 39 to 48, wherein R¹⁷ is

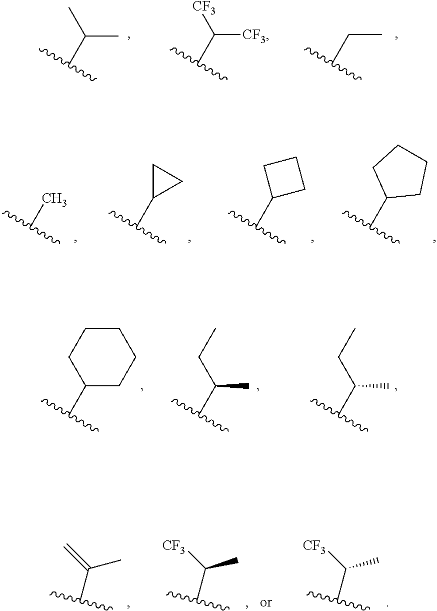

Embodiment 50. The compound of one of embodiments 39 to 48, wherein R¹⁷ is

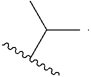 [SMILES: *C(C)C]

Embodiment 51. The compound of one of embodiments 34 to 50, wherein R⁶ is

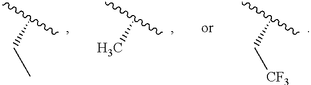

Embodiment 52. The compound of one of embodiments 34 to 51, wherein R⁷ is or

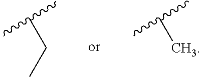

Embodiment 53. The compound of one of embodiments 34 to 50, wherein R⁶ and R⁷ substituents are joined to form, in combination with the —CHN— connecting the two substituents,

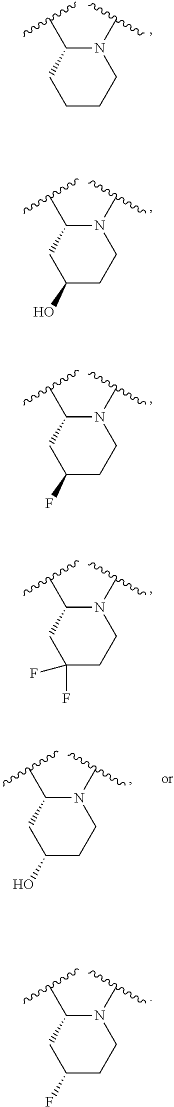 [SMILES: *[C@H]1CCCCN1* ; *[C@H]1C[C@H](O)CCN1* ; *[C@H]1C[C@H](F)CCN1* ; *[C@H]1CC(F)(F)CCN1* ; *[C@H]1C[C@@H](O)CCN1* ; *[C@H]1C[C@@H](F)CCN1*]

Embodiment 54. The compound of one of embodiments 34 to 50, wherein R⁶ and R⁷ substituents are joined to form, in combination with the —CHN— connecting the two substituents,

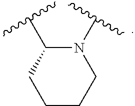 [SMILES: *[C@H]1CCCCN1*]

Embodiment 55. The compound of embodiment 34, having the formula:

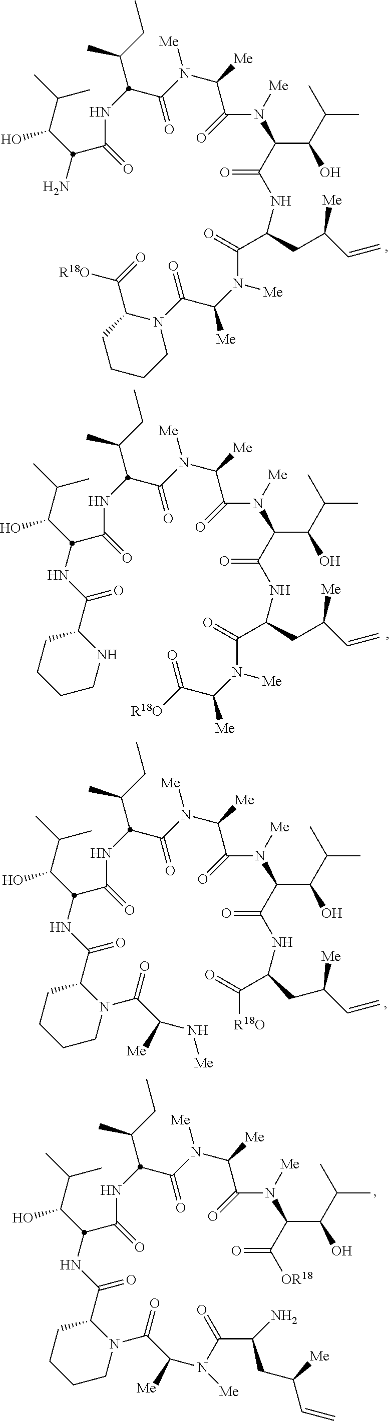

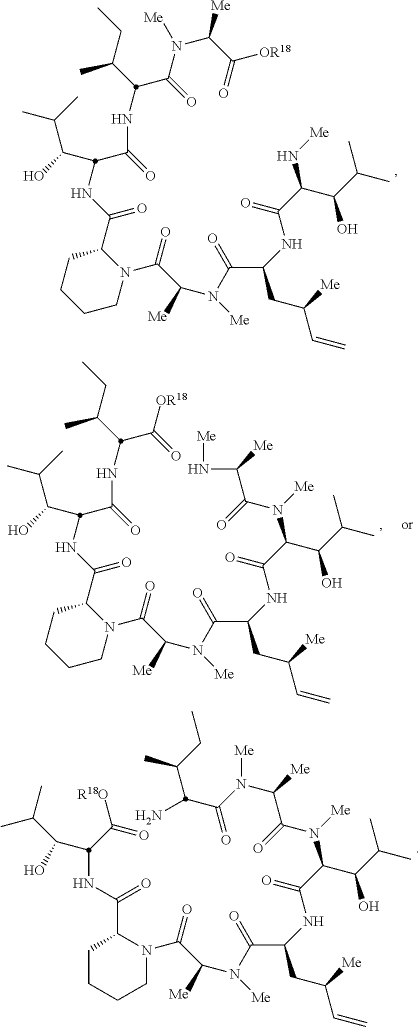

Embodiment 56. The compound of embodiment 34, having the formula:

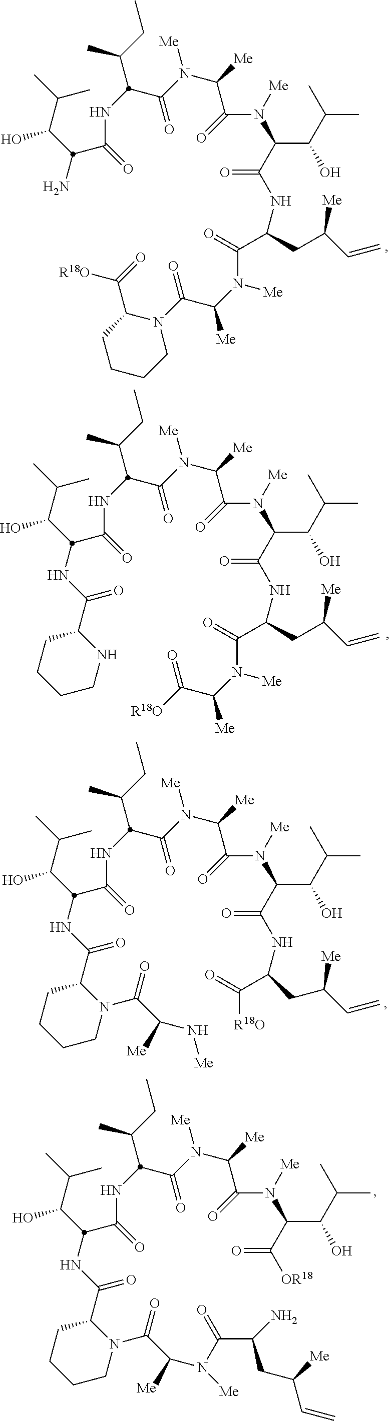

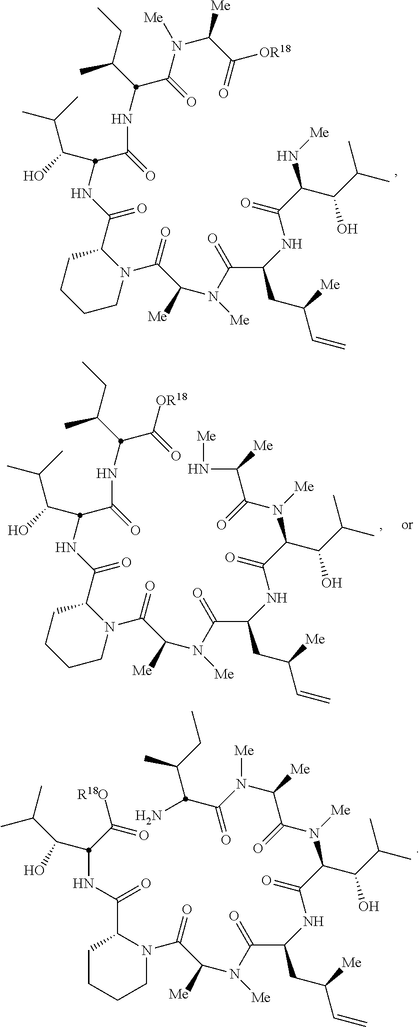

Embodiment 57. The compound of one of embodiments 34 to 56, wherein R¹⁸ is hydrogen.

Embodiment 58. The compound of one of embodiments 34 to 56, wherein R¹⁸ is unsubstituted C₁-C₄ alkyl.

Embodiment 59. The compound of one of embodiments 34 to 56, wherein R¹⁸ is unsubstituted methyl.

Embodiment 60. The compound of one of embodiments 34 to 56, wherein R¹⁸ is a resin moiety.

Embodiment 61. The compound of embodiment 60, wherein the resin moiety is a moiety of TGR A resin, Oxime resin, 2-Chlorotrityl chloride resin, Wang resin, TGA resin, Merrifield resin, TGT alcohol resin, HMBA resin, HMPB resin, HMPA resin, Rink Acid resin, or Hydrazinobenzoyl AM resin.

Embodiment 62. A method of treating a viral infection in a subject in need thereof, the method comprising administering to the subject in need thereof an effective amount of a compound of one of embodiments 1 to 23, or a pharmaceutically acceptable salt thereof.

Embodiment 63. The method of embodiment 62, wherein the viral infection is an RNA virus infection.

Embodiment 64. The method of embodiment 62, wherein the viral infection is a single stranded RNA virus infection.

Embodiment 65. The method of embodiment 62, wherein the viral infection is a positive-sense single stranded RNA virus infection.

Embodiment 66. The method of embodiment 62, wherein the viral infection is a coronavirus infection.

Embodiment 67. The method of embodiment 62, wherein the viral infection is a SARS coronavirus infection.

Embodiment 68. The method of embodiment 62, wherein the viral infection is a SARS-CoV-2 infection.

Embodiment 69. A method of treating acute respiratory distress syndrome (ARDS) in a subject in need thereof, the method comprising administering to the subject in need thereof an effective amount of a compound of one of embodiments 1 to 23, or a pharmaceutically acceptable salt thereof.

Embodiment 70. A method of treating a coronavirus disease in a subject in need thereof, the method comprising administering to the subject in need thereof an effective amount of a compound of one of embodiments 1 to 23, or a pharmaceutically acceptable salt thereof.

Embodiment 71. A method of treating a SARS-CoV-2 infection in a subject in need thereof, the method comprising administering to the subject in need thereof an effective amount of a compound of one of embodiments 1 to 23, or a pharmaceutically acceptable salt thereof.

Embodiment 72. A method of treating a SARS-CoV-2 associated disease in a subject in need thereof, the method comprising administering to the subject in need thereof an effective amount of a compound of one of embodiments 1 to 23, or a pharmaceutically acceptable salt thereof.

Embodiment 73. A method of treating arrhythmia in a subject in need thereof, the method comprising administering to the subject in need thereof an effective amount of a compound of one of embodiments 1 to 23, or a pharmaceutically acceptable salt thereof.

Embodiment N1. A compound having the formula:

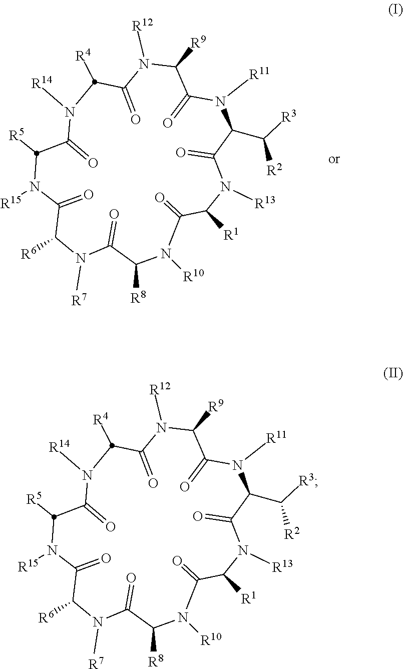

R¹ is substituted or unsubstituted alkyl or substituted or unsubstituted heteroalkyl; R² is —OCX²₃, —OCH₂X², —OCHX²₂, —SR^{2B}, —NR^{2A}R^{2B}, or —OR^{2B}; R^{2A} and R^{2B} are independently
hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R³ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ substituents may optionally be joined to form, in combination with the —CHN— connecting the two substituents, a substituted or unsubstituted heterocycloalkyl; R⁸ and R⁹ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCCl₃, —OCF₃, —OCBr₃, —OCl₃, —OC HCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁰, R¹¹, R¹², R¹³, R¹⁴, and R¹⁵ are independently
hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X² is independently —F, —Cl, —Br, or —I; or a pharmaceutically acceptable salt thereof.

Embodiment N2. The compound of embodiment N1, wherein R² is —NR^{2A}R^{2B} or —OR^{2B}; R^{2A} and R^{2B} are independently

hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{2A} and R^{2B} substituents bonded to the same nitrogen atom may optionally be joined to form a substituted or unsubstituted heterocycloalkyl or substituted or unsubstituted heteroaryl.

Embodiment N3. The compound of embodiment N1, wherein R² is —OR^{2B}; R^{2B} is independently

hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl.

Embodiment N4. The compound of embodiment N1, wherein R² is —OH.

Embodiment N5. The compound of embodiment N1, wherein R² is —NH₂.

Embodiment N6. The compound of one of embodiments N1 to N5, having the formula:

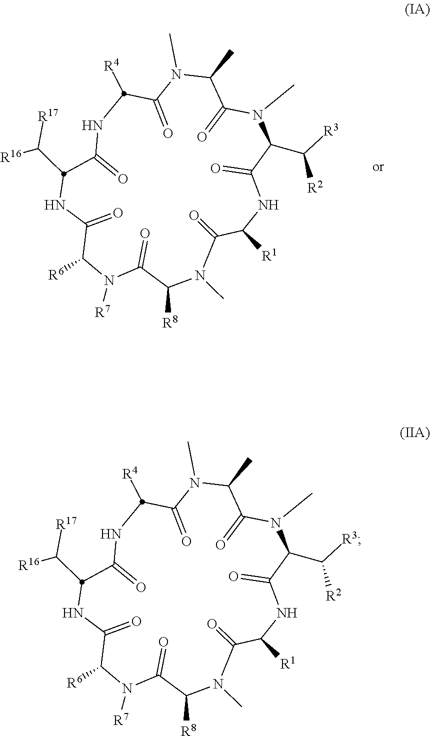

wherein R⁸ is substituted or unsubstituted alkyl or substituted or unsubstituted heteroalkyl; R³ is substituted or unsubstituted alkyl or substituted or unsubstituted cycloalkyl; R⁴ is substituted or unsubstituted alkyl or substituted or unsubstituted cycloalkyl; R⁶ and R⁷ are independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl; R⁶ and R⁷ substituents may optionally be joined to form, in combination with the —CHN— connecting the two substituents, a substituted or unsubstituted heterocycloalkyl; R⁸ is
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl; R¹⁶ is —OCX¹⁶³, —OCH₂X¹⁶, —OCHX¹⁶₂, —SR^{16B}, —NR^{16A}R^{16B} or —OR^{16B}; R^{16A} and R^{16B} are independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R¹⁷ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X¹⁶ is independently —F, —Cl, —Br, or —I; or a pharmaceutically acceptable salt thereof.

Embodiment N7. The compound of embodiment N1, having the formula:

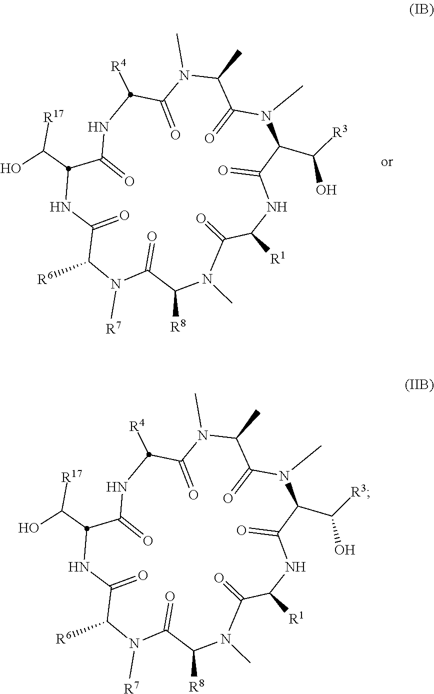

wherein R¹ is substituted or unsubstituted C₃-C₅ alkyl; R³ is substituted or unsubstituted C₁-C₆ alkyl or substituted or unsubstituted C₃-C₆ cycloalkyl; R⁴ is substituted or unsubstituted C₁-C₆ alkyl or substituted or unsubstituted C₃-C₆ cycloalkyl; R⁶ and R⁷ are independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl; R⁶ and R⁷ substituents may optionally be joined to form, in combination with the —CHN— connecting the two substituents, a substituted or unsubstituted 6 to 8 membered heterocycloalkyl; R⁸ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl; R¹⁷ is substituted or unsubstituted C₁-C₆ alkyl or substituted or unsubstituted C₃-C₆ cycloalkyl; or a pharmaceutically acceptable salt thereof.

Embodiment N8. The compound of embodiment N7, having the formula:

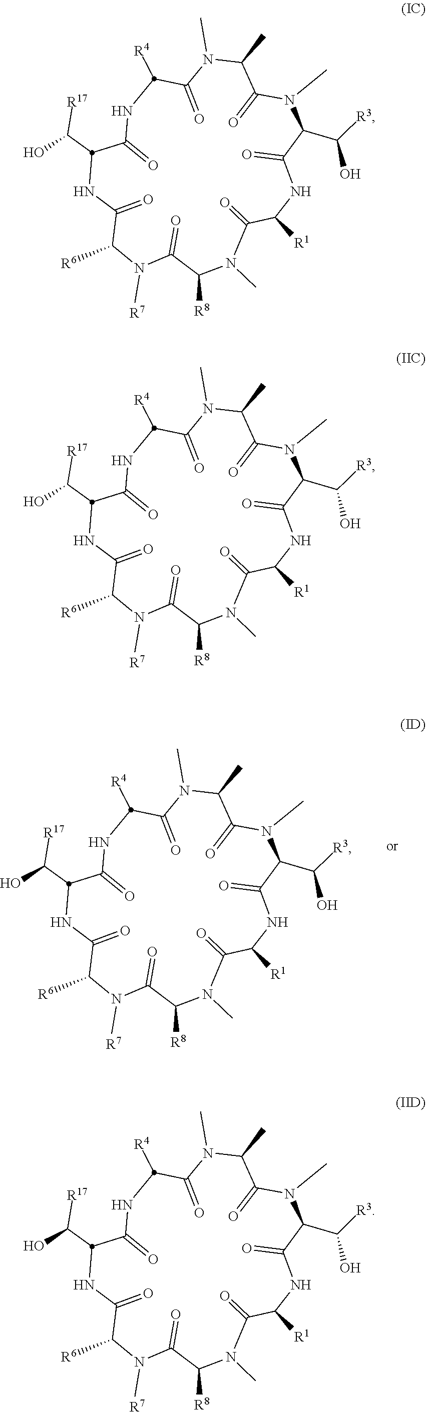

Embodiment N9. The compound of one of embodiments N6 to N8, wherein R¹ is fluoro-substituted or unsubstituted C₃-C₅ alkyl; R³ is fluoro-substituted or unsubstituted C₁-C₆ alkyl or fluoro-substituted or unsubstituted C₃-C₆ cycloalkyl; R⁴ is fluoro-substituted or unsubstituted C₁-C₆ alkyl or fluoro-substituted or unsubstituted C₃-C₆ cycloalkyl; R⁶ and R⁷ are independently fluoro-substituted or unsubstituted C₁-C₄ alkyl, or fluoro-substituted or unsubstituted 2 to 4 membered heteroalkyl; R⁶ and R⁷ substituents may optionally be joined to form, in combination with the —CHN— connecting the two substituents, a fluoro-substituted or unsubstituted 6 to 8 membered heterocycloalkyl; R⁸ is fluoro-substituted or unsubstituted C₁-C₆ alkyl; R¹⁷ is fluoro-substituted or unsubstituted C₁-C₆ alkyl or fluoro-substituted or unsubstituted C₃-C₆ cycloalkyl; or a pharmaceutically acceptable salt thereof.

Embodiment N10. The compound of one of embodiments N1 to N9, wherein R¹ is

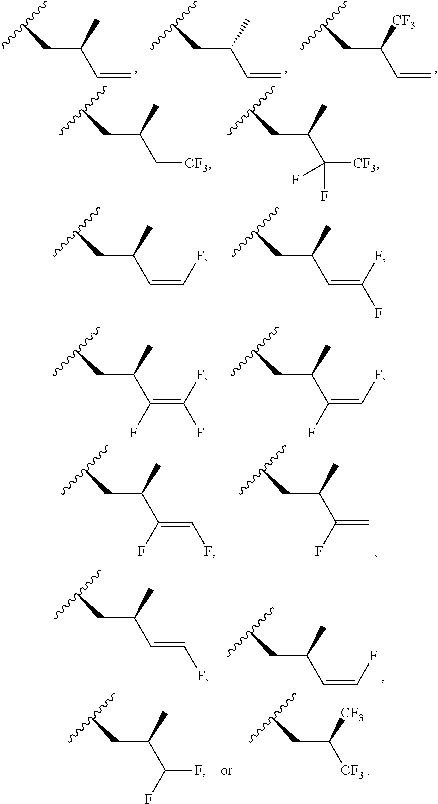

Embodiment N11. The compound of one of embodiments N1 to N9, wherein R¹ is

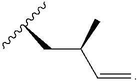

Embodiment N12. The compound of one of embodiments N1 to N11, wherein R³ is

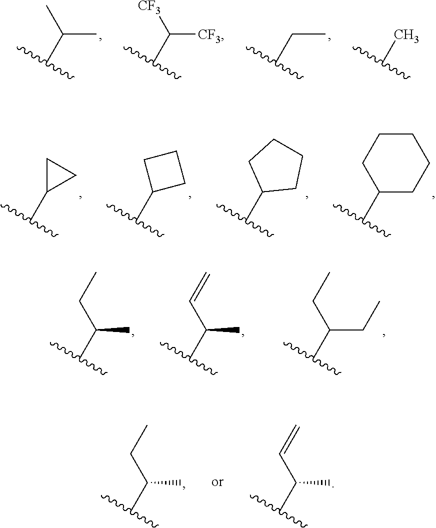

Embodiment N13. The compound of one of embodiments N1 to N11, wherein R³ is

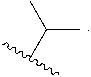 [SMILES: *C(C)C]

Embodiment N14. The compound of one of embodiments N1 to N13, wherein R⁴ is

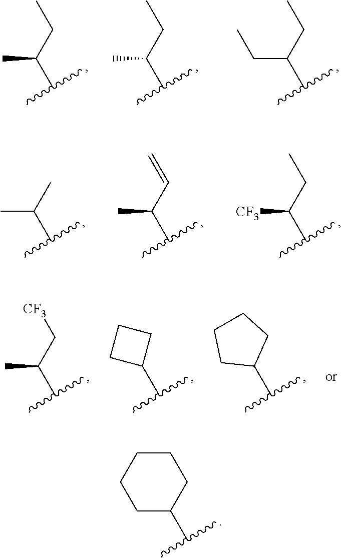

Embodiment N15. The compound of one of embodiments N1 to N13, wherein R⁴ is

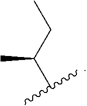 [SMILES: *[C@@H](C)CC]

Embodiment N16. The compound of one of embodiments N6 to N16, wherein R¹⁷ is

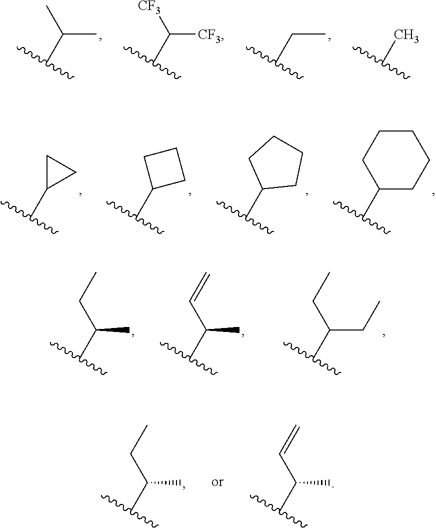

Embodiment N17. The compound of one of embodiments N6 to N15, wherein R¹⁷ is

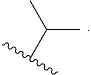 [SMILES: *C(C)C]

Embodiment N18. The compound of one of embodiments N1 to N17, wherein R⁶ is

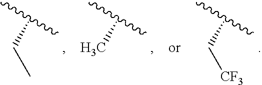

Embodiment N19. The compound of one of embodiments N1 to N18, wherein R⁷ is

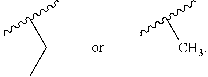

Embodiment N20. The compound of one of embodiments N1 to N17, wherein R⁶ and R⁷ substituents are joined to form, in combination with the —CHN— connecting the two substituents,

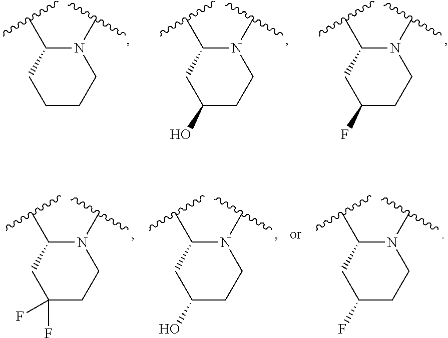 [SMILES: *[C@H]1CCCCN1* ; *[C@H]1C[C@H](O)CCN1* ; *[C@H]1C[C@H](F)CCN1* ; *[C@H]1CC(F)(F)CCN1* ; *[C@H]1C[C@@H](O)CCN1* ; *[C@H]1C[C@@H](F)CCN1*]

Embodiment N21. The compound of one of embodiments N1 to N17, wherein R⁶ and R⁷ substituents are joined to form, in combination with the —CHN— connecting the two substituents,

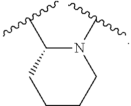 [SMILES: *[C@H]1CCCCN1*]

Embodiment N22. The compound of embodiment N1, having the formula:

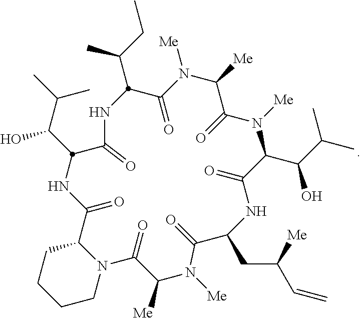 [SMILES: C=C[C@H](C)C[C@@H]1NC(=O)[C@H]([C@H](O)C(C)C)N(C)C(=O)[C@H](C)N(C)C(=O)[C@@H]([C@@H](C)CC)NC(=O)[C@@H]([C@H](O)C(C)C)NC(=O)[C@H]2CCCCN2C(=O)[C@H](C)N(C)C1=O]

Embodiment N23. The compound of embodiment N1, having the formula:

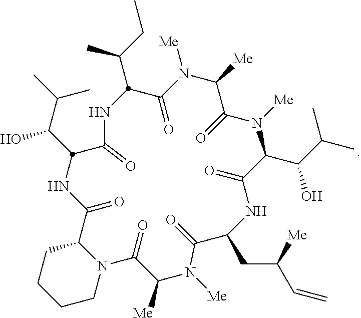 [SMILES: C=C[C@H](C)C[C@@H]1NC(=O)[C@H]([C@@H](O)C(C)C)N(C)C(=O)[C@H](C)N(C)C(=O)[C@@H]([C@@H](C)CC)NC(=O)[C@@H]([C@H](O)C(C)C)NC(=O)[C@H]2CCCCN2C(=O)[C@H](C)N(C)C1=O]

Embodiment N24. The compound of embodiment N1, having the formula:

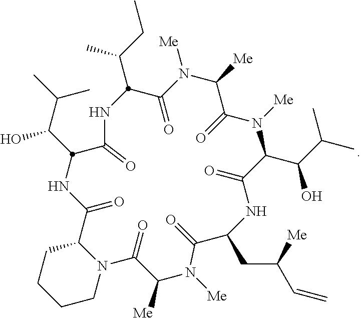 [SMILES: C=C[C@H](C)C[C@@H]1NC(=O)[C@H]([C@H](O)C(C)C)N(C)C(=O)[C@H](C)N(C)C(=O)[C@@H]([C@H](C)CC)NC(=O)[C@@H]([C@H](O)C(C)C)NC(=O)[C@H]2CCCCN2C(=O)[C@H](C)N(C)C1=O]

Embodiment N25. The compound of embodiment N1, having the formula:

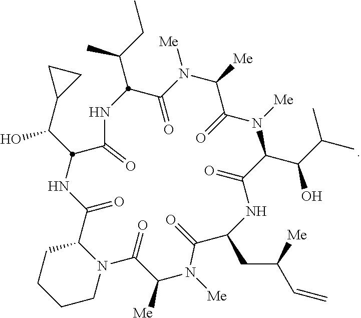 [SMILES: C=C[C@H](C)C[C@@H]1NC(=O)[C@H]([C@H](O)C(C)C)N(C)C(=O)[C@H](C)N(C)C(=O)[C@@H]([C@@H](C)CC)NC(=O)[C@@H]([C@H](O)C2CC2)NC(=O)[C@H]2CCCCN2C(=O)[C@H](C)N(C)C1=O]

Embodiment N26. A pharmaceutical composition comprising the compound of any one of embodiments N1 to N25, or a pharmaceutically acceptable salt thereof, and a pharmaceutically acceptable excipient.

Embodiment N27. A method of decreasing the level of Elongation Factor 1-alpha protein activity in a subject, said method comprising administering a compound of one of embodiments N1 to N25 to said subject.

Embodiment N28. A method of inhibiting cancer growth in a subject in need thereof, said method comprising administering to the subject in need thereof an effective amount of a compound of one of embodiments N1 to N25, or a pharmaceutically acceptable salt thereof.

Embodiment N29. A method of inhibiting cancer cell growth, said method comprising contacting the cancer cell with an effective amount of a compound of one of embodiments N1 to N25.

Embodiment N30. A method of treating a cancer in a subject in need thereof, said method comprising administering to the subject in need thereof an effective amount of a compound of one of embodiments N1 to N25.

Embodiment N31. The method of embodiment N30, wherein the cancer is a hematological cancer.

Embodiment N32. The method of embodiment N30, wherein the cancer is acute lymphoblastic leukemia, acute myeloid leukemia, chronic myelogenous leukemia, or multiple myeloma.

Embodiment N33. The method of one of embodiments N30 to N32, wherein the cancer is resistant to treatment with a tyrosine kinase inhibitor.

Embodiment N34. The method of embodiment N33, wherein the tyrosine kinase inhibitor is bosutinib, crizotinib, dasatinib, erlotinib, gefitinib, imatinib, afatinib, neratinib, lapatinib, nilotinib, ponatinib, midostaurin, gilteritinib, osimertinib, ibrutinib, or acalabrutinib.

Embodiment N35. The method of one of embodiments N30 to N34, further comprising co-administering an anti-cancer agent to said subject in need.

Embodiment N36. A compound having the formula:

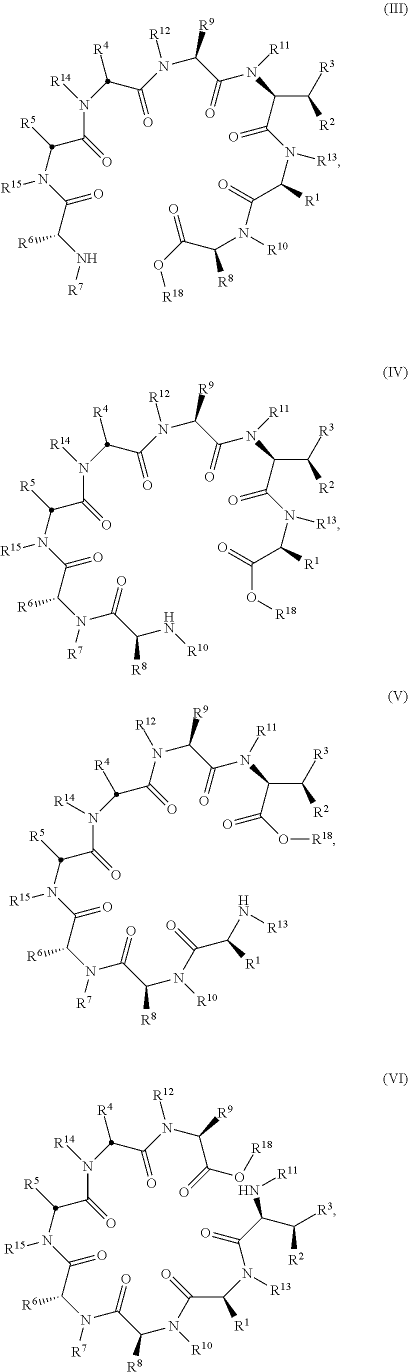

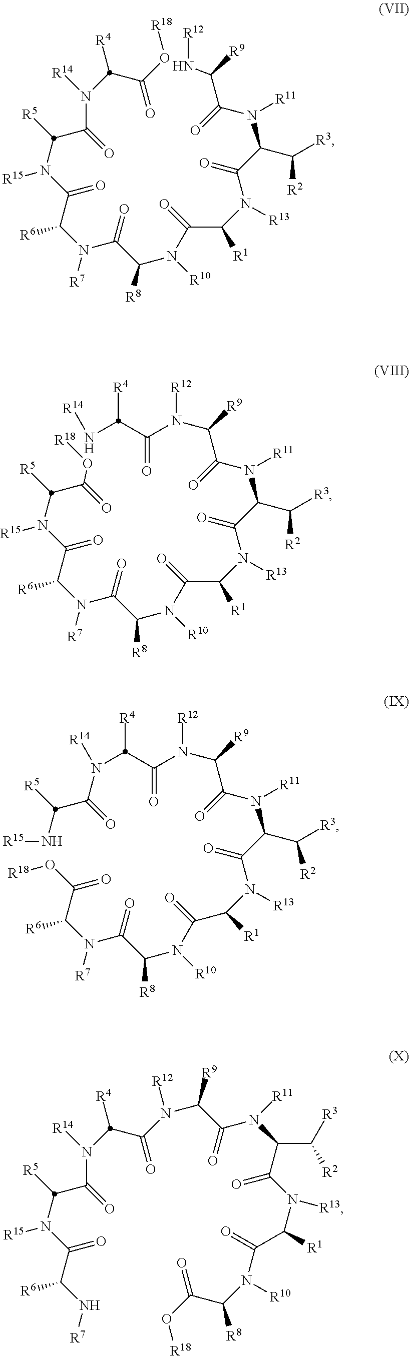

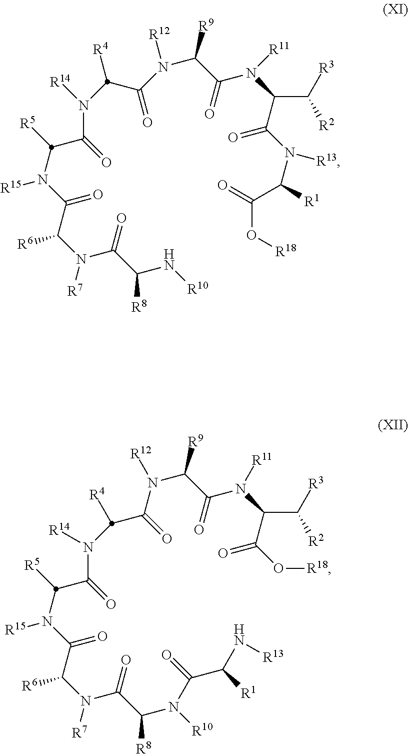

R¹ is substituted or unsubstituted alkyl or substituted or unsubstituted heteroalkyl; R² is —OCX², —OCH₂X², —OCHX²₂, —SR^{2B}, —NR^{2A}R^{2B}, or —OR^{2B}; R^{2A} and R^{2B} are independently
hydrogen, —CCl₃, —CF₃, —CHF₂, —CH₂F, —CN, —OH, —NH₂, —COOH, —CONH₂, —OCCl₃, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} Substituents bonded to the same nitrogen atom may optionally be joined to form a substituted or unsubstituted heterocycloalkyl or substituted or unsubstituted heteroaryl; R³ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ is —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ substituents may optionally be joined to form, in combination with the —CHN— connecting the two substituents, a substituted or unsubstituted heterocycloalkyl; R⁸ and R⁹ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCCl₃, —OCF₃, —OCBr₃, —OCl₃, —OC HCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁰, R¹¹, R¹², R¹³, R¹⁴, and R¹⁵ are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X² is independently —F, —Cl, —Br, or —I; R¹⁸ is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a resin moiety; or a pharmaceutically acceptable salt thereof.

Embodiment N37. The compound of embodiment N36, wherein R² is —NR^{2A}R^{2B} or —OR^{2B}; R^{2A} and R^{2B} are independently

hydrogen, —CCl₃, —CF₃, —CHF₂, —CH₂F, —CN, —OH, —NH₂, —COOH, —CONH₂, —OCCl₃, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{2A} and R^{2B} substituents bonded to the same nitrogen atom may optionally be joined to form a substituted or unsubstituted heterocycloalkyl or substituted or unsubstituted heteroaryl.

Embodiment N38. The compound of embodiment N36, wherein R² is —OR^{2B}; R^{2B} is independently

hydrogen, —CCl₃, —CF₃, —CHF₂, —CH₂F, —CN, —OH, —NH₂, —COOH, —CONH₂, —OCCl₃, —OCF₃, —OCHF 2, —OCH₂F, substituted or unsubstituted alkyl.

Embodiment N39. The compound of embodiment N36, wherein R² is —OH.

Embodiment N40. The compound of embodiment N36, wherein R² is —NH₂.

Embodiment N41. The compound of one of embodiments N36 to N40, having the formula:

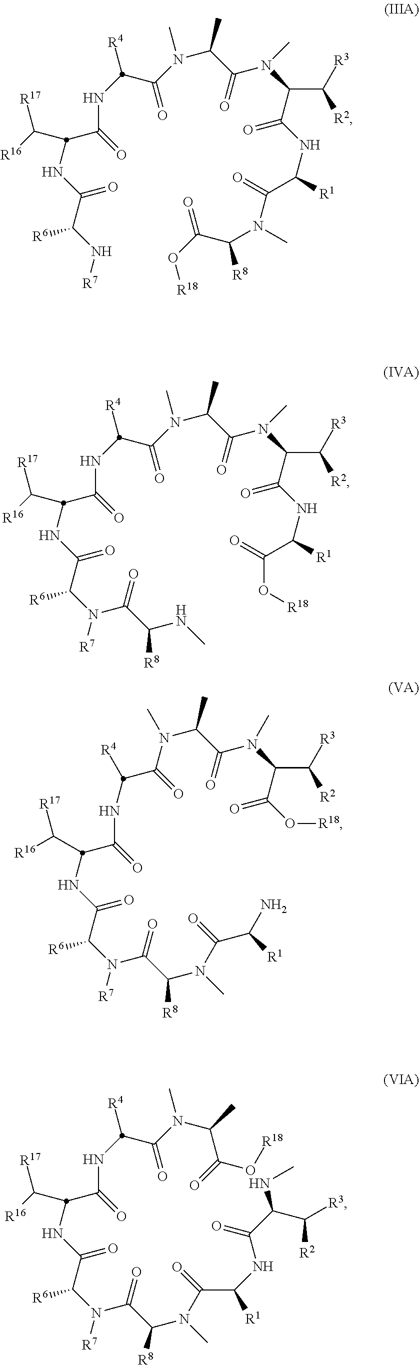

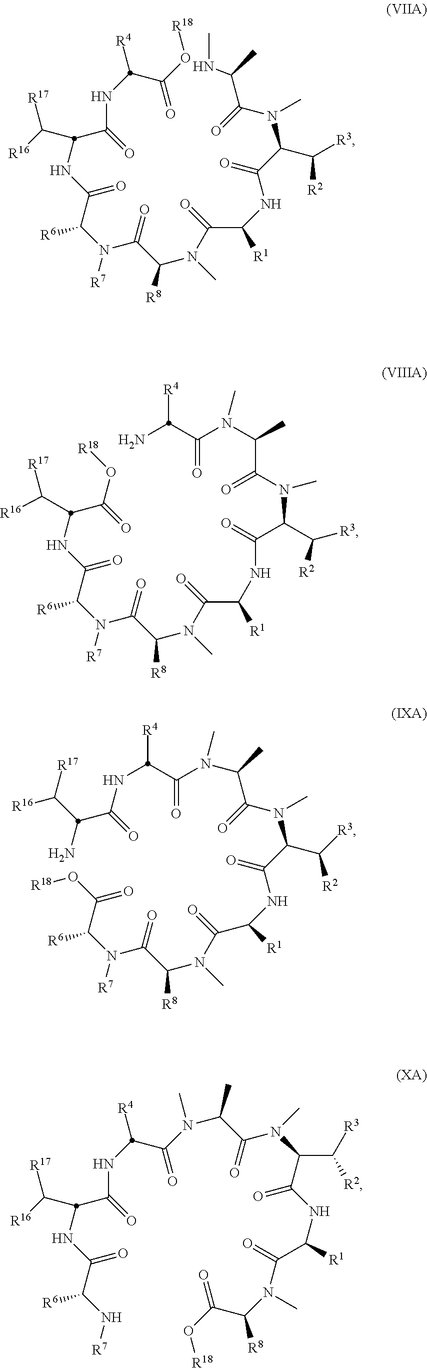

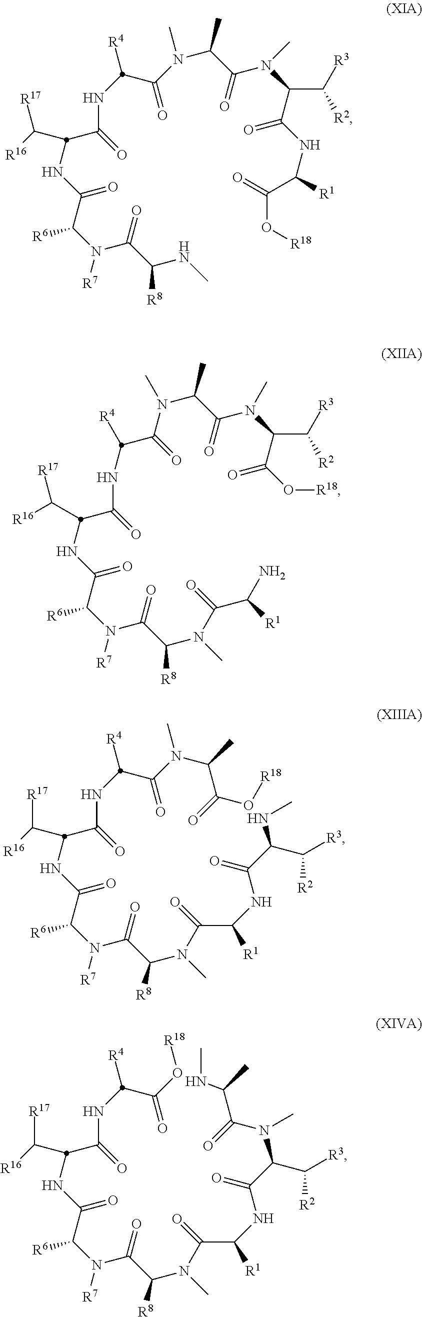

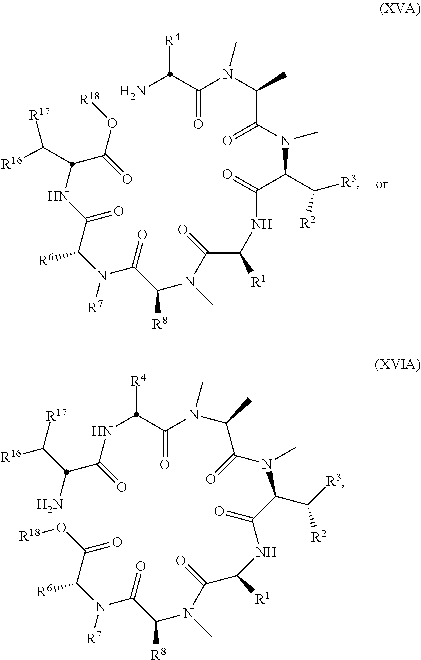

wherein
R¹ is substituted or unsubstituted alkyl or substituted or unsubstituted heteroalkyl; R³ is substituted or unsubstituted alkyl or substituted or unsubstituted cycloalkyl; R⁴ is substituted or unsubstituted alkyl or substituted or unsubstituted cycloalkyl; R⁶ and R⁷ are independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl; R⁶ and R⁷ substituents may optionally be joined to form, in combination with the —CHN— connecting the two substituents, a substituted or unsubstituted heterocycloalkyl; R′ is
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl; R¹⁶ is —OCX¹⁶₃, —OCH₂X¹⁶, —OCHX¹⁶₂, —SR^{16B}, —NR^{16A}R^{16B} or —OR^{16B}; R^{16A} and R^{16B} are independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R¹⁷ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X¹⁶ is independently —F, —Cl, —Br, or —I; or a pharmaceutically acceptable salt thereof.

Embodiment N42. The compound of embodiment N36, having the formula:

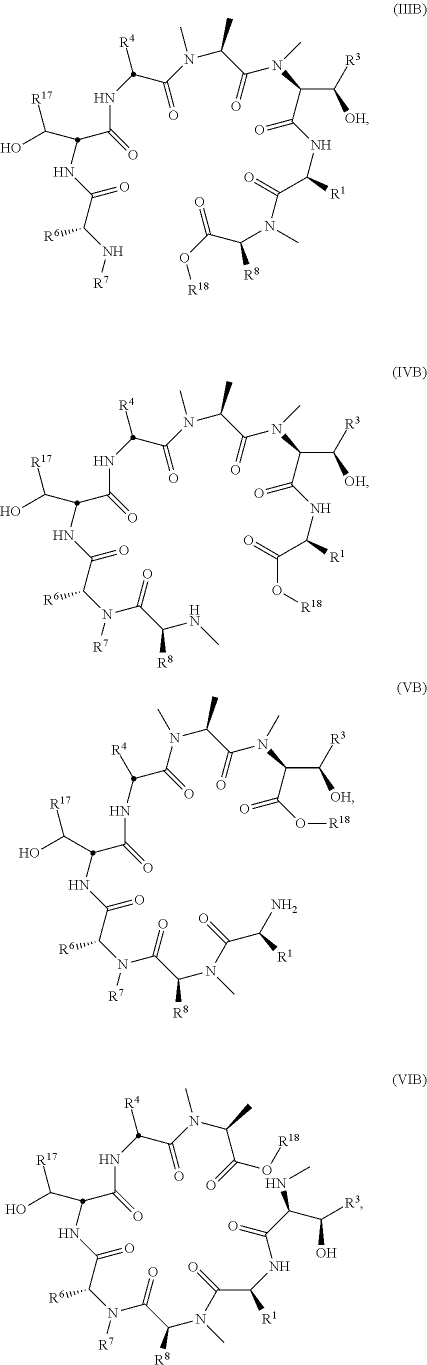

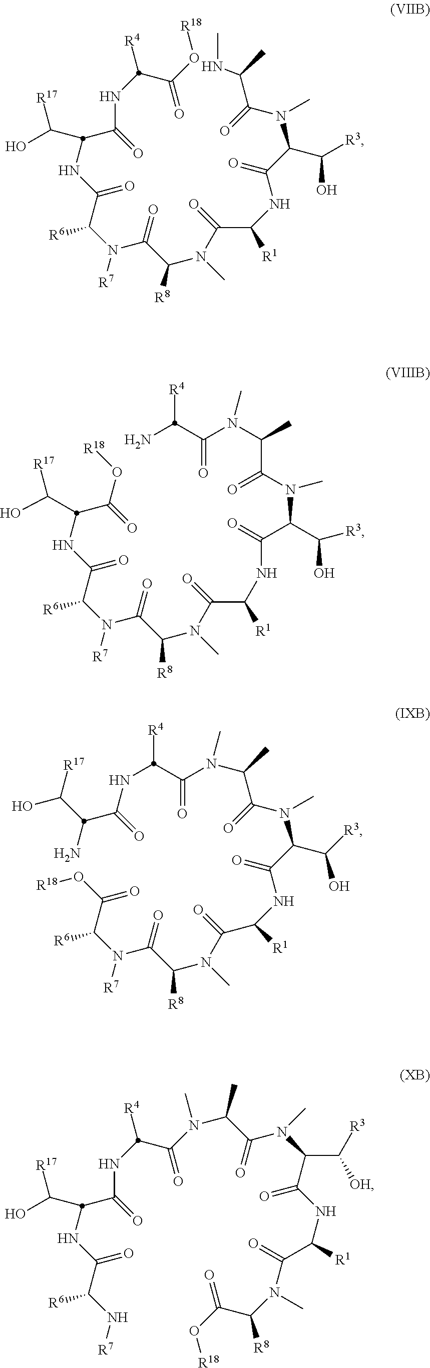

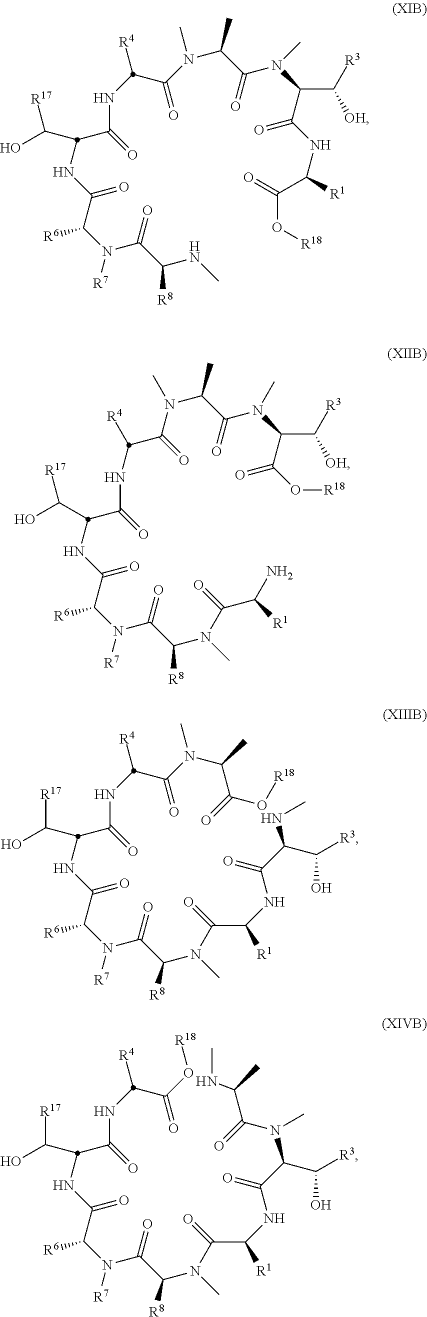

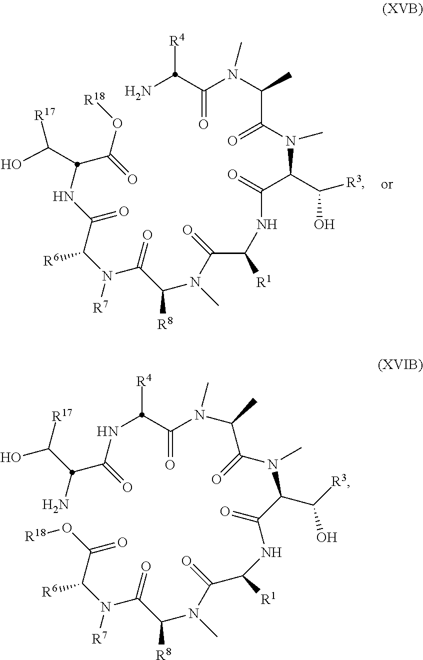

wherein
R¹ is substituted or unsubstituted C₃-C₅ alkyl; R³ is substituted or unsubstituted C₁-C₆ alkyl or substituted or unsubstituted C₃-C₆ cycloalkyl; R⁴ is substituted or unsubstituted C₁-C₆ alkyl or substituted or unsubstituted C₃-C₆ cycloalkyl; R⁶ and R⁷ are independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl; R⁶ and R⁷ substituents may optionally be joined to form, in combination with the —CHN— connecting the two substituents, a substituted or unsubstituted 6 to 8 membered heterocycloalkyl; R⁸ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted C₁-C₆ alkyl; R¹⁷ is substituted or unsubstituted C₁-C₆ alkyl or substituted or unsubstituted C₃-C₆ cycloalkyl; or a pharmaceutically acceptable salt thereof.

Embodiment N43. The compound of embodiment N42, having the formula:

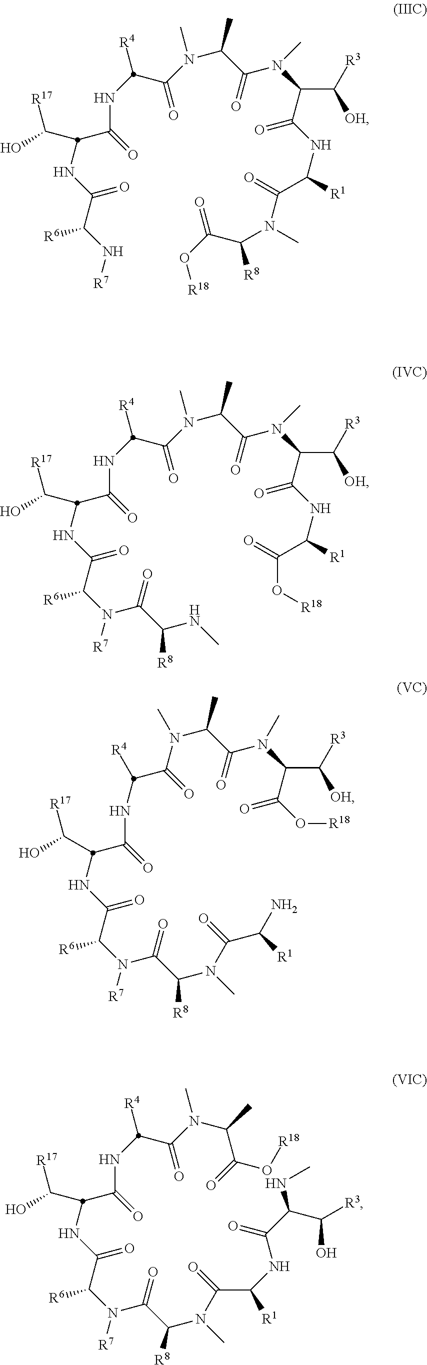

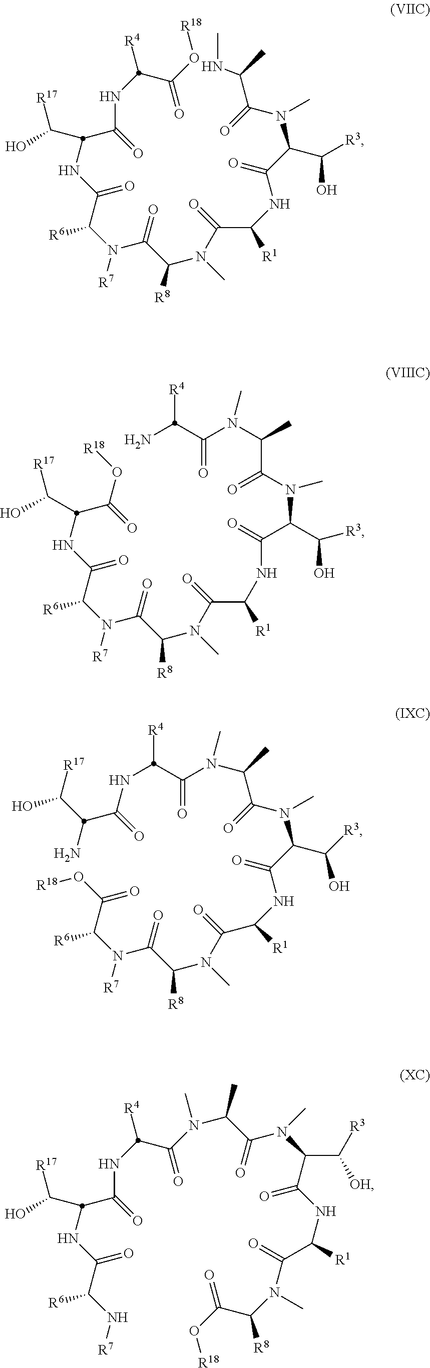

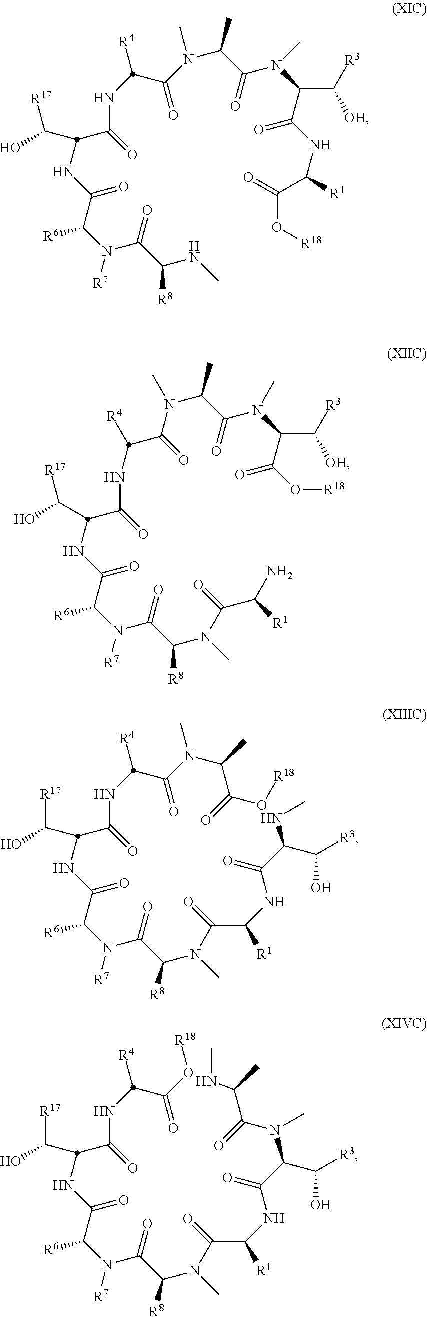

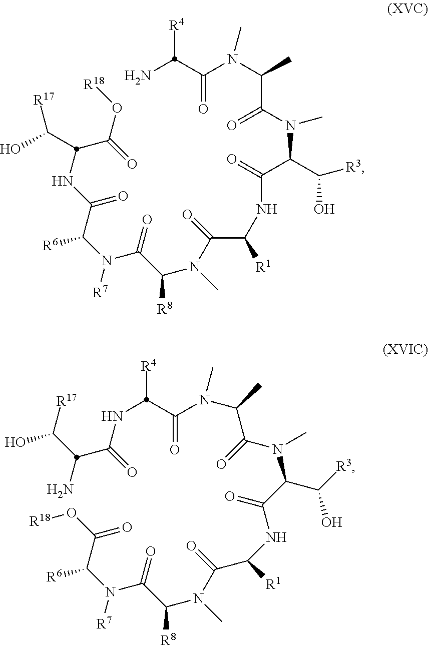

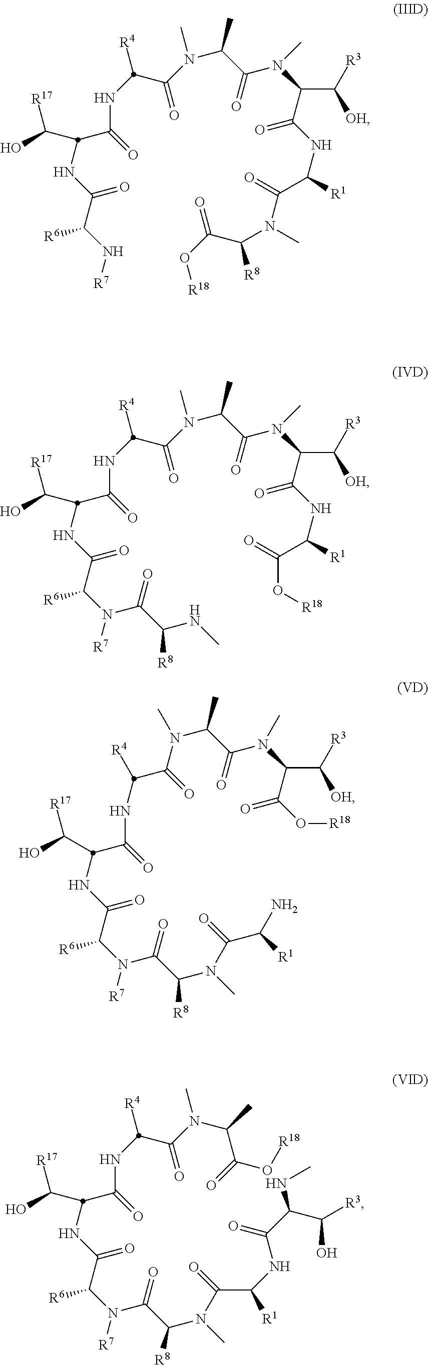

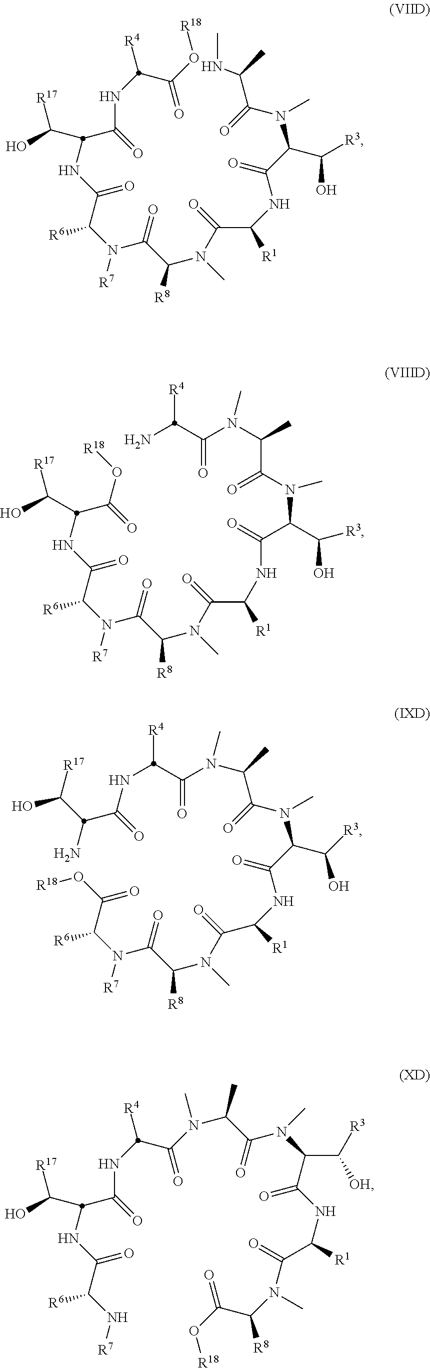

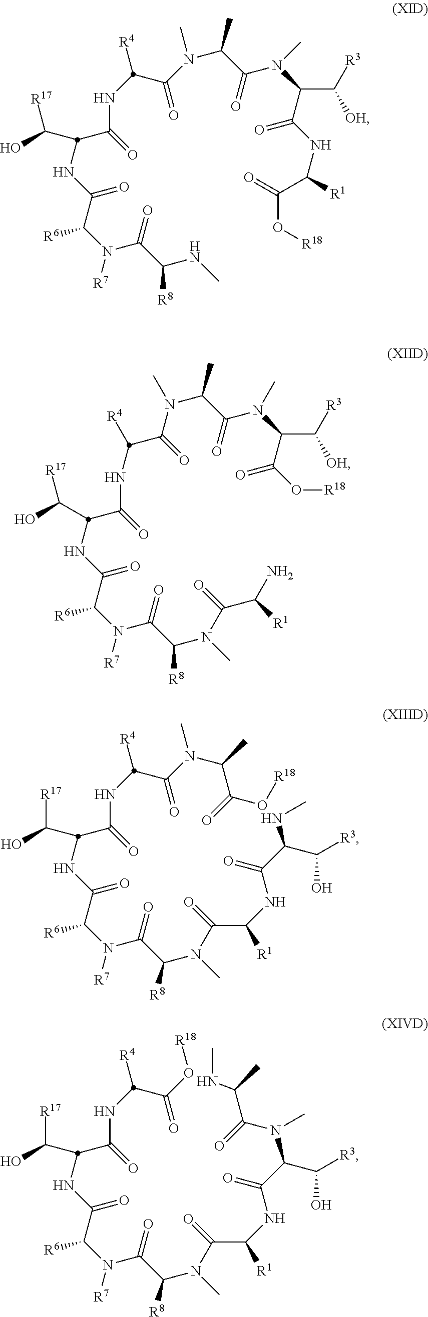

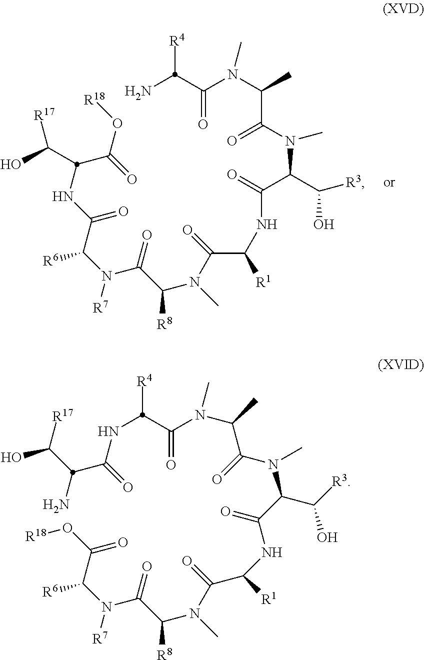

Embodiment N44. The compound of one of embodiments N41 to N43, wherein R¹ is fluoro-substituted or unsubstituted C₃-C₅ alkyl; R³ is fluoro-substituted or unsubstituted C₁-C₆ alkyl or fluoro-substituted or unsubstituted C₃-C₆ cycloalkyl; R⁴ is fluoro-substituted or unsubstituted C₁-C₆ alkyl or fluoro-substituted or unsubstituted C₃-C₆ cycloalkyl; R⁶ and R⁷ are independently fluoro-substituted or unsubstituted C₁-C₄ alkyl, or fluoro-substituted or unsubstituted 2 to 4 membered heteroalkyl; R⁶ and R⁷ substituents may optionally be joined to form, in combination with the —CHN— connecting the two substituents, a fluoro-substituted or unsubstituted 6 to 8 membered heterocycloalkyl; R⁸ is fluoro-substituted or unsubstituted C₁-C₆ alkyl; R¹⁷ is fluoro-substituted or unsubstituted C₁-C₆ alkyl or fluoro-substituted or unsubstituted C₃-C₆ cycloalkyl; or a pharmaceutically acceptable salt thereof.

Embodiment N45. The compound of one of embodiments N36 to N44, wherein R¹ is

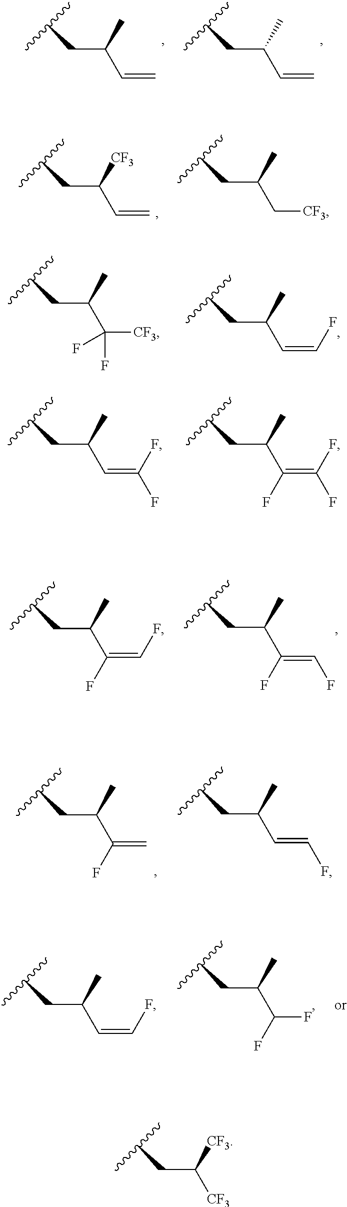

Embodiment N46. The compound of one of embodiments N36 to N44, wherein R¹ is

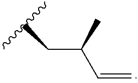

Embodiment N47. The compound of one of embodiments N36 to N46, wherein R³ is

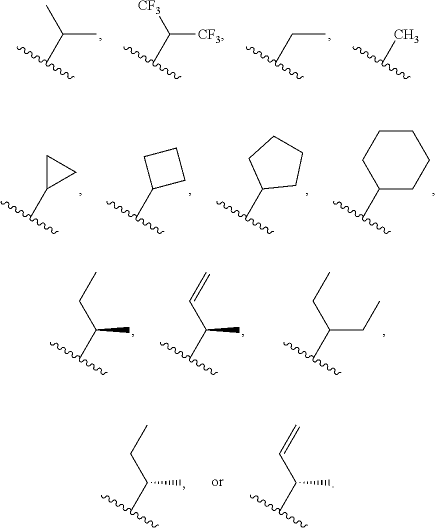

Embodiment N48. The compound of one of embodiments N36 to N46, wherein R³ is

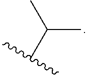 [SMILES: *C(C)C]

Embodiment N49. The compound of one of embodiments N36 to N48, wherein R⁴ is

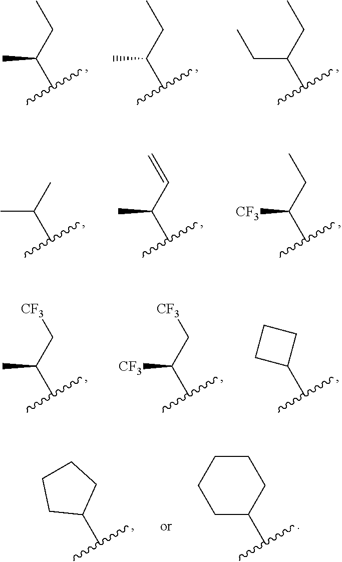

Embodiment N50. The compound of one of embodiments N36 to N48, wherein R⁴ is

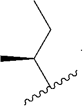 [SMILES: *[C@@H](C)CC]

Embodiment N51. The compound of one of embodiments N41 to N50, wherein R¹⁷ is

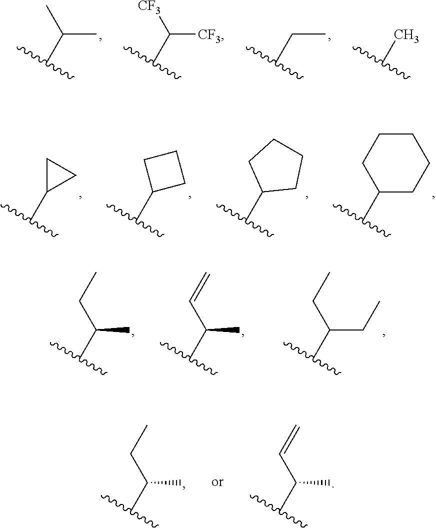

Embodiment N52. The compound of one of embodiments N41 to N50, wherein R¹⁷ is

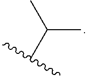 [SMILES: *C(C)C]

Embodiment N53. The compound of one of embodiments N36 to N52, wherein R⁶ is

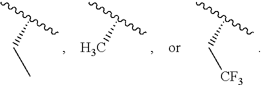

Embodiment N54. The compound of one of embodiments N36 to N53, wherein R⁷ is

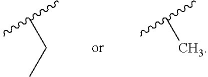

Embodiment N55. The compound of one of embodiments N36 to N52, wherein R⁶ and R⁷ substituents are joined to form, in combination with the —CHN— connecting the two substituents,

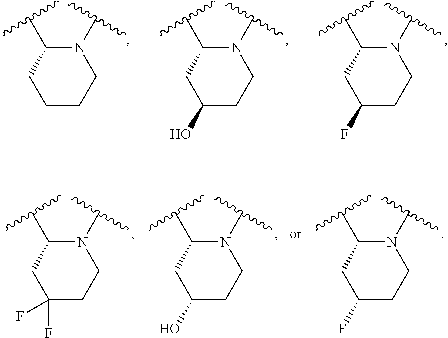 [SMILES: *[C@H]1CCCCN1* ; *[C@H]1C[C@H](O)CCN1* ; *[C@H]1C[C@H](F)CCN1* ; *[C@H]1CC(F)(F)CCN1* ; *[C@H]1C[C@@H](O)CCN1* ; *[C@H]1C[C@@H](F)CCN1*]

Embodiment N56. The compound of one of embodiments N36 to N52, wherein R⁶ and R⁷ substituents are joined to form, in combination with the —CHN— connecting the two substituents,

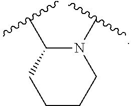 [SMILES: *[C@H]1CCCCN1*]

Embodiment N57. The compound of embodiment N36, having the formula:

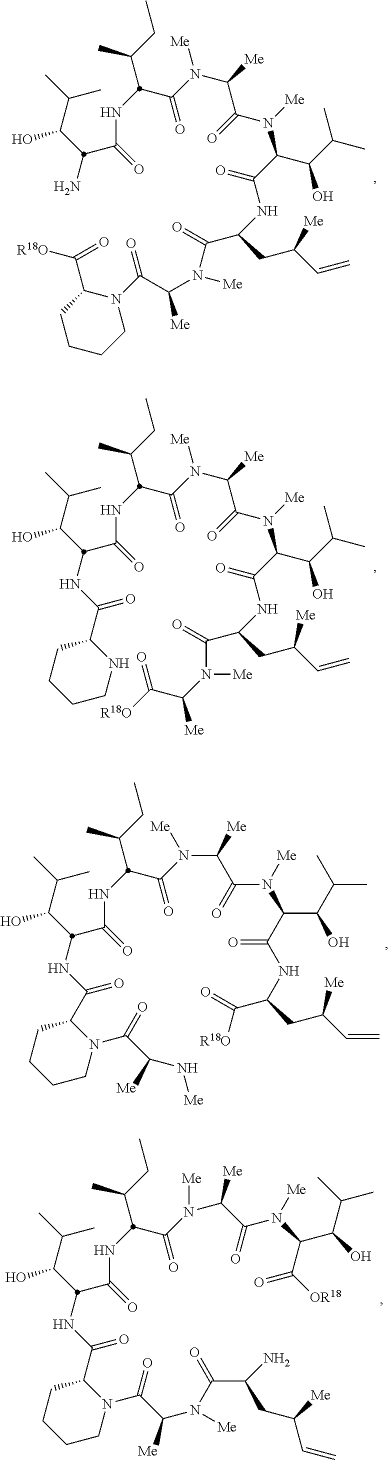

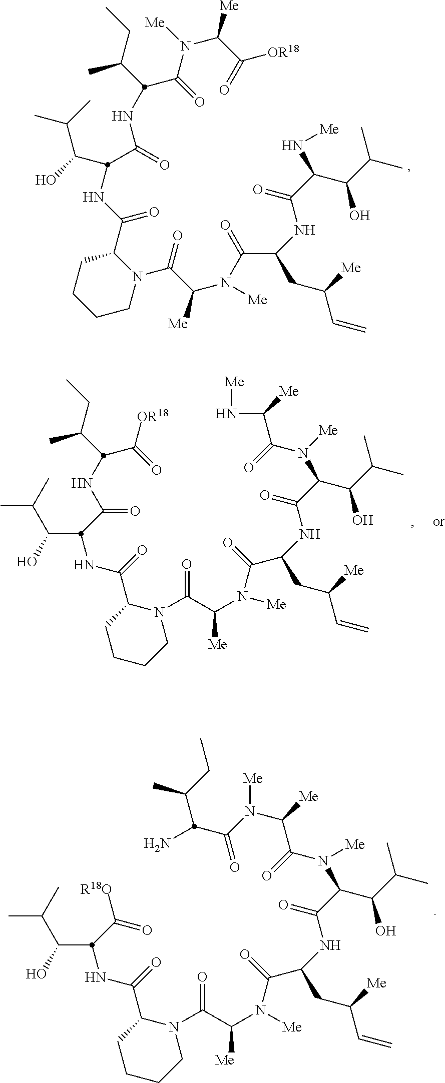

Embodiment N58. The compound of embodiment N36, having the formula:

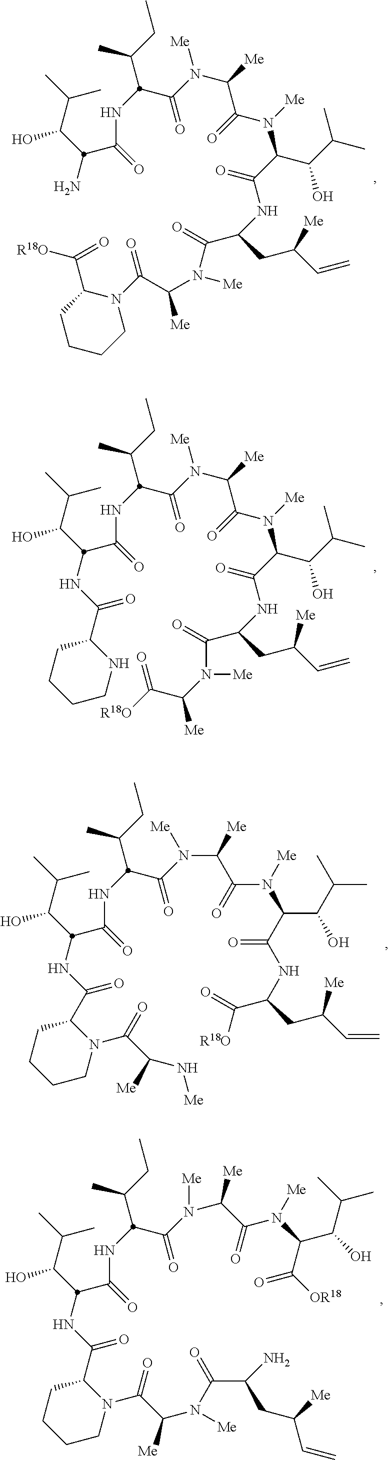

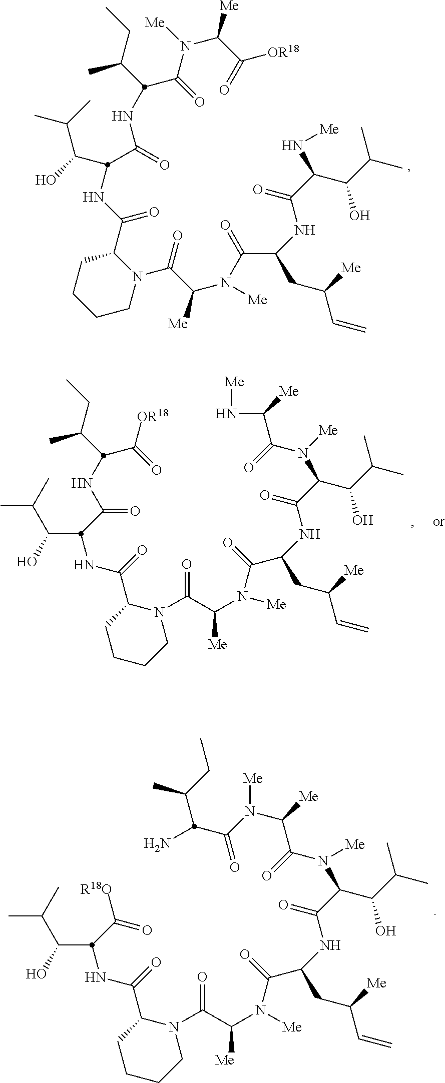

Embodiment N59. The compound of one of embodiments N36 to N58, wherein R¹⁸ is hydrogen.

Embodiment N60. The compound of one of embodiments N36 to N58, wherein R¹⁸ is unsubstituted C₁-C₄ alkyl.

Embodiment N61. The compound of one of embodiments N36 to N58, wherein R¹⁸ is unsubstituted methyl.

Embodiment N62. The compound of one of embodiments N36 to N58, wherein R¹⁸ is a resin moiety.

Embodiment N63. The compound of embodiment N62, wherein the resin moiety is a moiety of TGR A resin, Oxime resin, 2-Chlorotrityl chloride resin, Wang resin, TGA resin, Merrifield resin, TGT alcohol resin, HMBA resin, HMPB resin, HMPA resin, Rink Acid resin, or Hydrazinobenzoyl AM resin.

Embodiment N64. A method of treating a viral infection in a subject in need thereof, the method comprising administering to the subject in need thereof an effective amount of a compound of one of embodiments N1 to N25, or a pharmaceutically acceptable salt thereof.

Embodiment N65. The method of embodiment N64, wherein the viral infection is an RNA virus infection.

Embodiment N66. The method of embodiment N64, wherein the viral infection is a single stranded RNA virus infection.

Embodiment N67. The method of embodiment N64, wherein the viral infection is a positive-sense single stranded RNA virus infection.

Embodiment N68. The method of embodiment N64, wherein the viral infection is a coronavirus infection.

Embodiment N69. The method of embodiment N64, wherein the viral infection is a SARS coronavirus infection.

Embodiment N70. The method of embodiment N64, wherein the viral infection is a SARS-CoV-2 infection.

Embodiment N71. A method of treating acute respiratory distress syndrome (ARDS) in a subject in need thereof, the method comprising administering to the subject in need thereof an effective amount of a compound of one of embodiments N1 to N25, or a pharmaceutically acceptable salt thereof.

Embodiment N72. A method of treating a coronavirus disease in a subject in need thereof, the method comprising administering to the subject in need thereof an effective amount of a compound of one of embodiments N1 to N25, or a pharmaceutically acceptable salt thereof.

Embodiment N73. A method of treating a SARS-CoV-2 infection in a subject in need thereof, the method comprising administering to the subject in need thereof an effective amount of a compound of one of embodiments N1 to N25, or a pharmaceutically acceptable salt thereof.

Embodiment N74. A method of treating a SARS-CoV-2 associated disease in a subject in need thereof, the method comprising administering to the subject in need thereof an effective amount of a compound of one of embodiments N1 to N25, or a pharmaceutically acceptable salt thereof.

Embodiment N75. A method of treating arrhythmia in a subject in need thereof, the method comprising administering to the subject in need thereof an effective amount of a compound of one of embodiments N1 to N25, or a pharmaceutically acceptable salt thereof.

### EXAMPLES

Described herein is the total synthesis, structure determination, and biological characterization of SRA3, epi-SRA3, and deshydroxyl-A3 (dA3). SRA3 and epi-SRA3 have the opposite stereochemical configuration for the same side-chain hydroxyl group, while dA3 lacks one of two side-chain hydroxyls.

### Example 1: Assay Methods

Cell Culture

HCT116 cells (ATCC, Manassas, Va.) were maintained in McCoy's 5A media (Gibco, Grand Island, N.Y.) supplemented with 10% fetal bovine serum (Axenia Biologix, Dixon, Calif.), 100 units/mL penicillin, and 100 ug/mL streptomycin (Gibco). H929 cells (ATCC) were maintained in advanced RPMI 1640 media (Gibco) supplemented with 6% fetal bovine serum, 2 mM glutamine, 100 units/mL penicillin, and 100 mg/mL streptomycin. MM1S cells (ATCC) were maintained in RPMI 1640 media (Gibco) supplemented with 10% fetal bovine serum, 100 units/mL penicillin, and 100 mg/mL streptomycin. All cells were cultured at 37° C. in a 5% CO₂ atmosphere.

Proliferation Assay (Continuous Exposure)

Adherent cells were briefly trypsinized and repeatedly pipetted to produce a homogeneous cell suspension. 2,500 cells were seeded in 100 uL complete growth media per well in 96-well clear-bottom plates. Suspension cells were repeatedly pipetted to produce a homogeneous cell suspension. 10,000 cells were seeded in 100 uL complete growth media per well in 96-well clear-bottom plates. After allowing cells to grow/adhere overnight, cells were treated with 25 uL/well 5×drug stocks (0.1% DMSO final) and incubated for 72 hr. AlamarBlue (Life Technologies, Grand Island, N.Y.) was used to assess cell viability per the manufacturer's instructions. Briefly, 12.5 uL alamarBlue reagent was added to each well, and plates were incubated at 37° C. Fluorescence intensity was measured every 30 min to determine the linear range for each assay (Ex 545 nm, Em 590 nm, SPARK, Tecan Austria GmbH, Austria). Proliferation curves were generated by first normalizing fluorescence intensity in each well to the DMSO-treated plate average. Normalized fluorescence intensities were plotted in GraphPad Prism (GraphPad, La Jolla, Calif.), and IC50 values calculated from nonlinear regression curves. The reported IC50 values represent the average of at least three independent determinations (±SD).

Proliferation Assay (Transient Exposure, Followed by Washout)

Adherent cells were briefly trypsinized and repeatedly pipetted to produce a homogeneous cell suspension. 2,500 cells were seeded in 100 uL complete growth media per well in 96-well clear-bottom plates. Suspension cells were repeatedly pipetted to produce a homogeneous cell suspension. 0.6×10⁶ cells were seeded in 3 mL complete growth media per well in 6-well plates. After allowing cells to grow/adhere overnight, cells were treated with compounds (100 nM or 200 nM as indicated, 0.1% DMSO final) and incubated for the indicated time. For adherent cells, growth media was carefully removed, cells were washed with warm PBS twice (2×short wash), and followed by 5 min incubation in warm media at 37° C. (long wash). After repeating the “short-long” washing cycle 3 times, cells were resuspended in warm media and plated in flat-bottom 96-well plates (100 uL/well) at equal cell number. For suspension cells, cells were collected and spun down at 300×g for 3 min. Supernatant was carefully removed, cells were then washed with warm PBS twice (2× short wash), followed by 5 min incubation in warm media at 37° C. (long wash). After repeating the “short-long” washing cycle 3 times, cells were resuspended in warm media and plated in flat-bottom 96-well plates (100 uL/well) at equal cell number. After the indicated times post-washout, CellTiter Glo (Promega, Madison, Wis.) was used to asses the cell viability per the manufacturer's instructions. Briefly, after adding 100 uL CellTiter Glo reagent to each well, the plate was rocked at room temperature for 5-10 min, and luminescence intensity was measured. Proliferation curves were generated by first normalizing luminescence intensity in each well to the average T=0 value for each compound. Normalized luminescence intensity was plotted in GraphPad Prism (triplicate values, mean±SD).

Protein Synthesis Inhibition Assay (Continuous Exposure)

HCT116 cells at 80% confluency in 12-well plates were incubated with compounds with varying concentrations for 10 min or 24 hours at 37° C. After the indicated time, O-propargyl-puromycin (30 μM final concentration) was added, and the cells were incubated for 1 hour at 37° C. Subsequently, media was removed, and cells were trypsinized, collected, and washed twice with ice-cold PBS before transferring to a 96-well V bottom plate. 100 uL Zombie Red (BioLegend, San Diego, Calif.) solution was added to each well and incubated for 30 min at RT in dark. Cells were then washed with 2% FBS in PBS buffer before fixed with 200 uL of 4% PFA in PBS for 15 min on ice in dark. After washing the cells with 2% FBS in PBS buffer, 200 uL permeabilization buffer (3% FBS, 0.1% saponin in PBS) was added to each well, and the cells were incubated for 5 min at RT in the dark. Cells were then washed and resuspended in 25 uL permeabilization buffer. 100 uL click chemistry mix (50 mM HEPES pH=7.5, 150 mM NaCl, 400 uM TCEP, 250 uM TBTA, 5 uM CF405M-Azide (Biotium, Fremont, Calif.), 200 uM Cu₂SO₄) was added to each well, and cells were incubated at RT in the dark. After the overnight incubation, the cells were washed with permeabilization buffer followed by FACS buffer (2% FBS, 1% P/S, 2 mM EDTA, in PBS w/o Ca/Mg). Cells were then resuspended in 200 uL FACS buffer and filtered before FACS analysis (FACSCanto II, Becton Dickinson, Franklin Lakes, N.J.). Only single, live cells were analyzed (FlowJo), as judged by forward/side scattering and the absence of Zombie Red staining. Protein synthesis inhibition curves were generated by first normalizing intensity to cycloheximide-treated (50 μg/mL for 4 h, maximum inhibition) and DMSO-treated control samples. Normalized intensity was plotted in GraphPad Prism.

Protein Synthesis Inhibition Assay (Transient Exposure, Followed by Washout)

HCT116 cells at 80% confluency in 24-well plates were incubated with compounds with at 100 nM for 4 hours at 37° C. O-propargyl-puromycin (30 uM final concentration) was then added, and the cells were incubated for 1 hour at 37° C. Media was carefully removed, cells were washed with warm PBS twice (2× short wash), followed by 5 min incubation in warm medium at 37° C. (long wash). After repeating the “short-long” washing cycle for 3 times, cells were then resuspended in warm media and incubated at 37° C. After the indicated time post-washout, media was removed, and cells were trypsinized, collected, and washed twice with ice-cold PBS before transferring to a 96-well V bottom plate. 100 uL Zombie Red (BioLegend, San Diego, Calif.) solution was added to each well and incubated for 30 min at RT in the dark. Cells were then washed with 2% FBS in PBS buffer before fixed with 200 uL of 4% PFA in PBS for 15 min on ice in dark. After washing the cells with 2% FBS in PBS buffer, 200 uL permeabilization buffer (3% FBS, 0.1% Saponin in PBS) was added to each well, and cells were incubated for 5 min at RT in the dark. Cells were then washed and resuspended in 25 uL permeabilization buffer. 100 uL click chemistry mix (50 mM HEPES pH=7.5, 150 mM NaCl, 400 uM TCEP, 250 uM TBTA, 5 uM CF405M-Azide (Biotium, Fremont, Calif.), 200 uM Cu₂SO₄) was added to each well, and cells were incubated at RT in dark. After the overnight incubation, cells were washed with permeabilization buffer followed by FACS buffer (2% FBS, 1% P/S, 2 mM EDTA, in PBS w/o Ca/Mg). Cells were then resuspended in 200 uL FACS buffer and filtered before FACS analysis (FACSCanto II, Becton Dickinson, Franklin Lakes, N.J.). Only single, live cells were analyzed (FlowJo), as judged by forward/side scattering and the absence of Zombie Red staining. Protein synthesis inhibition curves were generated by first normalizing intensity to the cycloheximide-treated (50 μg/mL for 1 h, maximum inhibition) and DMSO-treated control samples. Normalized intensity was plotted in GraphPad Prism.

Ep-Myc Mouse Lymphoma Model

Mouse studies were approved by the University of California, San Francisco Institutional Animal Care and Use Committee (IACUC). Eμ-Myc/+ mice were purchased from the Jackson Laboratory. For the Eμ-Myc tumor model, allograft mice were generated using spontaneous lymph node tumors derived from Eμ-Myc mice. 5×10⁵ Eμ-Myc/+ lymphoma cells were injected via the tail vein into eight-week-old male C57BL/6J mice. Seven days after lymphoma cell injection, mice were randomized to receive vehicle (10% EtOH/Kolliphor EL in water, n=5 mice) or (S,R)-A3 (n=5 mice) by intraperitoneal injection every other day (3 times per week) until the endpoint based on the IACUC-approved protocol. Eμ-Myc mice in the (S,R)-A3 treatment arm received (S,R)-A3 at a dose of 2 mg/kg for the first 21 days and 1-2 mg/kg after treatment day 21 according to their body weights (BW): BW>26 g, 2 mg/kg; BW 25-26 g, 1.5 mg/kg; BW 24-25 g, 1 mg/kg).

### Example 2: Synthetic Methods

Chemical Synthesis

All reactions in non-aqueous media were conducted under a positive pressure of dry argon in glassware that had been dried in oven prior to use unless noted otherwise. Anhydrous solutions of reaction mixtures were transferred via an oven dried syringe or cannula. All solvents were dried prior to use unless noted otherwise. Thin layer chromatography was performed using precoated silica gel plates (EMD Chemical Inc. 60, F254). Flash column chromatography was performed on CombiFlash Rf 200i system (Teledyne Isco, Lincoln, Nebr.). ¹H and ¹³C nuclear magnetic resonance spectra (NMR) were obtained on a Varian (Palo Alto, Calif.) Inova 400 MHz spectrometer recorded in ppm (δ) downfield of TMS (δ=0) in CDCl₃ unless noted otherwise. Signal splitting patterns were described as singlet (s), doublet (d), triplet (t), quartet (q), quintet (quint), or multiplet (m), with coupling constants (J) in hertz. High resolution mass spectra (HRMS) were performed on Waters Xevo G2-XS QToF LC-MS system, eluting with a water/MeCN (+0.1% formic acid) gradient at 0.6 mL/min.

Synthesis of 6:

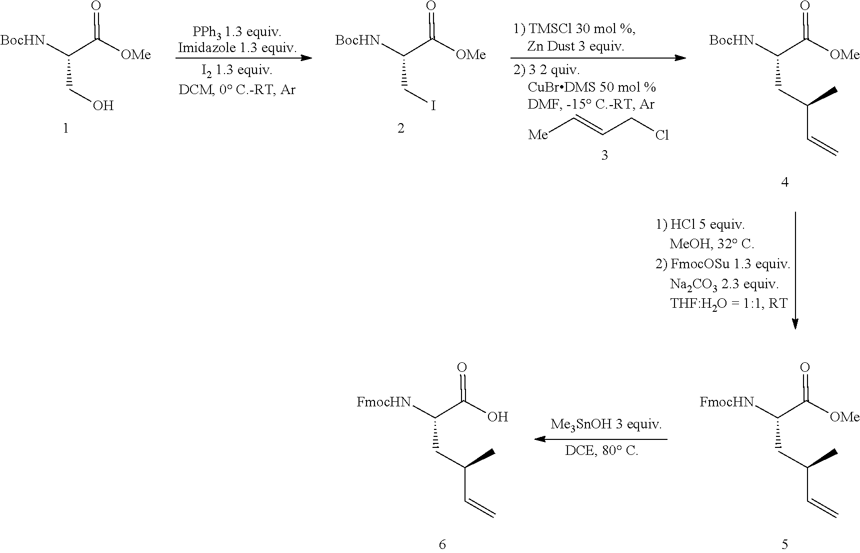

To an oven-dried flask was added PPh₃ (13 mmol, 3.4 g), imidazole (13 mmol, 885 mg), and anhydrous DCM (60 mL) under Ar. After cooling to 0° C., I₂ (13 mmol, 3.3 g) was added in three portions and the reaction was warmed up to RT and stirred for 10 min. The reaction was then re-cooled to 0° C., Boc-Ser-OMe 1 (10 mmol, 2.2 g) was added. After stirring at 0° C. for 1 h, the reaction was allowed to warm up to RT. After completion (˜2-3 h), the reaction was filtered through a pad of celite and concentrated in vacuo. To the above filtrate, Et₂O was added, and the resulting slurry was filtered through a pad of celite and concentrated in vacuo. CombiFlash system (0-10% EA in Hexane) was used to purify the crude mixture to afford 2 as the product (yellow solid, 2.33 g, 71% yield).

To an oven-dried flask was added Zn dust (26.1 mmol, 1.71 g), anhydrous DMF (10 mL), and TMSCl (2.61 mmol, 0.33 mL) at RT under Ar. After stirring for 30 min, the slurry was cooled to 0° C. and 2 (8.7 mmol, 2.86 g) was added. The mixture was allowed to warm up to RT and stirred for 1 h to form the corresponding Zn reagent. To a separated oven-dried flask was added CuBrDMS (4.35 mmol, 894 mg), 3 (17.4 mmol, 1.7 mL), and anhydrous DMF (10 mL) at RT under Ar. After the mixture was cooled to −15° C., the Zn reagent was added. The reaction was then warmed up to RT and stirred overnight. After completion, the reaction was quenched with NH₄Cl (aq), extracted by EA, dried over Na₂SO₄, and concentrated in vacuo. CombiFlash system (0-5% EA in Hexane) was used to purify the crude mixture to afford 4 as the product (colorless liquid, 868 mg, 40% yield). Spectral data of 4 are in accordance with literature.

To an oven-dried flask was added 4 (1.2 mmol, 303 mg) and 2 M HCl in MeOH (6 mmol, 3 mL). The reaction was stirred at 32° C. for 2 h. After completion, the mixture was concentrated in vacuo to afford a crude amine mixture, which was used in the next step without further purification. To an oven-dried flask was added the crude amine mixture, FmocOSu (1.56 mmol, 526 mg), Na₂CO₃ (2.76 mmol, 295 mg), THE (6 mL), and H₂O (6 mL). The reaction was stirred overnight at RT. After completion, the reaction was quenched with NH₄Cl (aq), extracted by EA, dried over Na₂SO₄, and concentrated in vacuo. CombiFlash system (0-10% EA in Hexane) was used to purify the crude mixture to afford 5 as the product (colorless liquid, 311 mg, 69% yield over 2 steps).

To an oven-dried flask was added 5 (0.2 mmol, 76 mg), Me₃SnOH (0.6 mmol, 108.5 mg), and DCE (2 mL). The reaction was stirred at 80° C. for 4h. After completion, the reaction was diluted with EA, washed with 1 M HCl and brine, dried over Na₂SO₄, and concentrated in vacuo. The crude mixture of 6 was used in the solid phase synthesis without further purification.

Synthesis of 12:

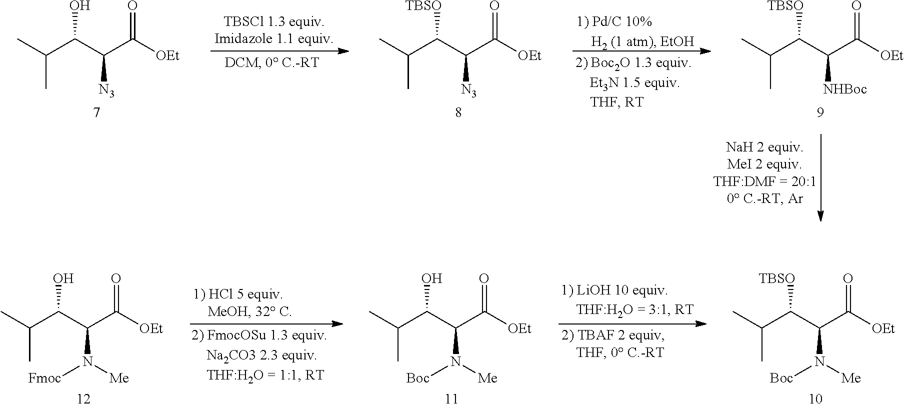

Compound 7 was synthesized according to the previously reported protocol.

To an oven-dried flask was added 7 (10 mmol, 2 g), imidazole (13 mmol, 885 mg), and anhydrous DCM (50 mL). After cooling to 0° C., TBSCl (13 mmol, 1.96 g) was added and the reaction was warmed up to RT and stirred overnight. After completion, the reaction was quenched with NH₄Cl (aq), extracted by DCM, dried over Na₂SO₄, and concentrated in vacuo. CombiFlash system (0-5% EA in Hexane) was used to purify the crude mixture to afford 8 as the product (colorless liquid, 3.16 g, 99% yield).

To an oven-dried flask was added 8 (10 mmol, 3.16 g), Pd/C (10%, 320 mg), and EtOH (50 mL) under H₂. The reaction stirred overnight at RT. After completion, the reaction was filtered through a pad of celite and concentrated in vacuo to afford a crude mixture. To an oven-dried flask was added the above crude mixture, Boc₂O (13 mmol, 3 mL), Et₃N (15 mmol, 2.1 mL) and THE (50 mL). The reaction stirred overnight at RT. After completion, the reaction was quenched with NH₄Cl (aq), extracted by DCM, dried over Na₂SO₄, and concentrated in vacuo. CombiFlash system (0-10% EA in Hexane) was used to purify the crude mixture to afford 9 as the product (yellow liquid, 3.2 g, 83% yield after 2 steps).

To an oven-dried flask was added 9 (5 mmol, 1.95 g), anhydrous THF (40 mL), and anhydrous DMF (2 mL). After the reaction was cooled to 0° C., NaH (60%, 10 mmol, 400 mg) was added and the reaction was stirred for 30 min at 0° C. Mel (10 mmol, 0.62 mL) was then added at 0° C. and the reaction was allowed to warm up to RT and stirred overnight. After completion, the reaction was quenched with NH₄Cl (aq), extracted by EA, dried over Na₂SO₄, and concentrated in vacuo. CombiFlash system (0-10% EA in Hexane) was used to purify the crude mixture to afford 10 as the product (yellow liquid, 1.24 g, 62% yield).

To an oven-dried flask was added 10 (3 mmol, 1.21 g), LiOH (30 mmol, 720 mg), THE (15 mL), and H₂O (15 mL). The reaction was stirred at RT for 3h. After completion, the reaction was quenched with 1 M HCl until pH=4 and concentrated in vacuo to afford a crude mixture, which was used in the next step without further purification. To an oven-dried flask was added the above crude mixture and THF (30 mL). After the reaction was cooled to 0° C., TBAF in THE solution (1M, 6 mmol, 0.6 mL) was added and the reaction was warmed to RT and stirred overnight. After completion, the reaction was quenched with NH₄Cl (aq), extracted by EA, dried over Na₂SO₄, and concentrated in vacuo. CombiFlash system (0-50% EA with 1% AcOH in Hexane) was used to purify the crude mixture to afford 11 as the product (yellow liquid, 510 mg, 65% yield after 2 steps). HRMS (ESI) for C₁₂H₂₃NO₅ (M−H), 260.1503 (Calc.), found 260.1537.

To an oven-dried flask was added 11 (1 mmol, 261 mg) and 2 M HCl in MeOH (5 mmol, 2.5 mL). The reaction was stirred at 32° C. for 2h. After completion, the mixture was concentrated in vacuo to afford a crude mixture, which was used in the next step without further purification. To an oven-dried flask was added the crude mixture, FmocOSu (1.3 mmol, 439 mg), Na₂CO₃ (2.3 mmol, 244 mg), THF (5 mL), and H₂O (5 mL). The reaction was stirred at RT overnight. After completion, the reaction was quenched with NH₄Cl (aq), extracted by EA, dried over Na₂SO₄, and concentrated in vacuo. CombiFlash system (0-10% MeOH in DCM) was used to purify the crude mixture to afford 12 as the product (white solid, 238 mg, 62% yield over 2 steps). HRMS (ESI) for C₂₂H₂₅NO₅ (M+H), 384.1805 (Calc.), found 384.1939.

Synthesis of 20:

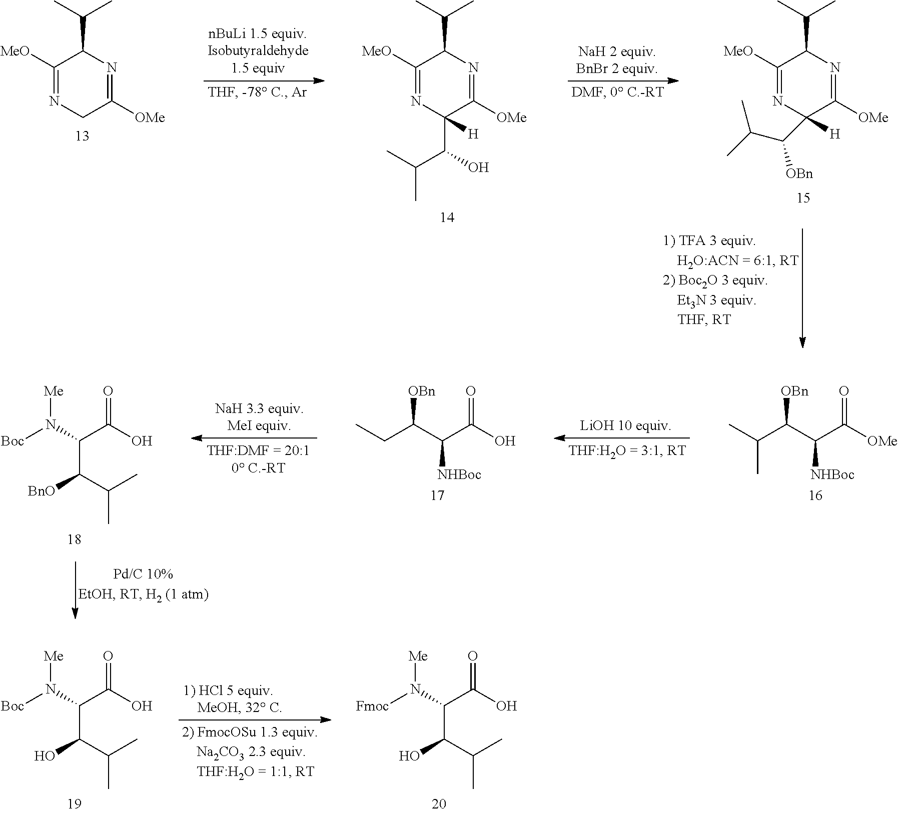

To an oven-dried flask was added 13 (10 mmol, 1.9 g) and anhydrous THF (50 mL) under Ar. After cooling to −78° C., nBuLi (2.5 M in Hexane, 15 mmol, 6 mL) was added. After stirring at −78° C. for 30 min, isobutyraldehyde (15 mmol, 1.4 mL) was added and the reaction was allowed to warm up to RT and stirred overnight. After completion, the reaction was quenched with NH₄Cl (aq), extracted by DCM, dried over Na₂SO₄, and concentrated in vacuo. CombiFlash system (0-20% EA in Hexane) was used to purify the crude mixture to afford 14 as the product (yellow liquid, 1.57 g, 61% yield).

To an oven-dried flask was added 14 (6 mmol, 1.52 g) and anhydrous DMF (50 mL). After cooling to 0° C., NaH (60%, 12 mmol, 480 mg) was added and the reaction was stirred at 0° C. for 30 min. BnBr (12 mmol, 1.42 mL) was then added and the reaction was allowed to warm up to RT and stirred overnight. After completion, the reaction was quenched with NH₄Cl (aq), extracted by EA, dried over Na₂SO₄, and concentrated in vacuo. CombiFlash system (0-10% EA in Hexane) was used to purify the crude mixture to afford 15 as the product (yellow liquid, 1.98 g, 95% yield).

To an oven-dried flask was added 15 (6 mmol, 1.98 g), MeCN (8 mL), and water (48 mL). TFA (18 mmol, 1.4 mL) was added dropwise and the reaction was stirred overnight. After completion, the reaction was concentrated in vacuo. The crude mixture was used in the next step without further purification. To an oven-dried flask was added above crude mixture, Boc₂O (18 mmol, 4.2 mL), Et₃N (18 mmol, 2.5 mL), and THE (50 mL). The reaction was stirred overnight at RT. After completion, the reaction was quenched with NH₄Cl (aq), extracted by EA, dried over Na₂SO₄, and concentrated in vacuo. CombiFlash system (0-10% EA in Hexane) was used to purify the crude mixture to afford 16 as the product (yellow liquid, 1.33 g, 63% yield after 2 steps). HRMS (ESI) for C₁₉H₂₉NO₅ (M+Na), 374.1938 (Calc.), found 374.1579.

To an oven-dried flask was added 16 (4 mmol, 1.41 g), LiOH (40 mmol, 1.68 g), THE (30 mL) and water (10 mL). The reaction was stirred at RT for 5h. After completion, the reaction was concentrated in vacuo. The crude mixture was used in the next step without further purification. To an oven-dried flask was added above crude mixture, anhydrous THE (40 mL) and anhydrous DMF (2 mL). After cooling to 0° C., NaH (60%, 13.2 mmol, 528 mg) was added. After stirring at 0° C. for 30 min, Mel (12 mmol, 0.75 mL) was added. The reaction was then allowed to warm up to RT and stirred overnight. After completion, the reaction was quenched with NH₄Cl (aq), extracted by EA, dried over Na₂SO₄, and concentrated in vacuo. CombiFlash system (0-30% EA (with 1% AcOH) in Hexane) was used to purify the crude mixture to afford 18 as the product (yellow liquid, 745 mg, 53% yield). HRMS (ESI) for C₁₉H₂₉NO₅ (M−H), 350.1973 (Calc.), found 350.2054. To an oven-dried flask was added 18 (2 mmol, 703 mg), Pd/C (10 wt %, ˜100 mg), and EtOH (20 mL). The reaction was vigorously stirred at RT under H₂ overnight. After completion, the reaction was filtered through a pad of celite and concentrated in vacuo. CombiFlash system (0-50% EA (with 1% AcOH) in Hexane) was used to purify the crude mixture to afford 19 as the product (yellow liquid, 345 mg, 66% yield).

To an oven-dried flask was added 19 (1 mmol, 261 mg) and 2 M HCl in MeOH (5 mmol, 2.5 mL). The reaction was stirred at 32° C. for 2h. After completion, the mixture was concentrated in vacuo. The crude mixture was used in the next step without further purification. To an oven-dried flask was added the crude mixture, FmocOSu (1.3 mmol, 439 mg), Na₂CO₃ (2.3 mmol, 244 mg), THF (5 mL), and H₂O (5 mL) at RT. The reaction was stirred overnight and monitored by TLC. After completion, the reaction was quenched with NH₄Cl (aq), extracted by EA, dried over Na₂SO₄, and concentrated in vacuo. CombiFlash system (0-10% MeOH in DCM) was used to purify the crude mixture to afford 20 as the product (white solid, 333 mg, 87% yield over 2 steps). HRMS (ESI) for C₂₂H2₅NO₅ (M+H), 384.1805 (Calc.), found 384.1939.

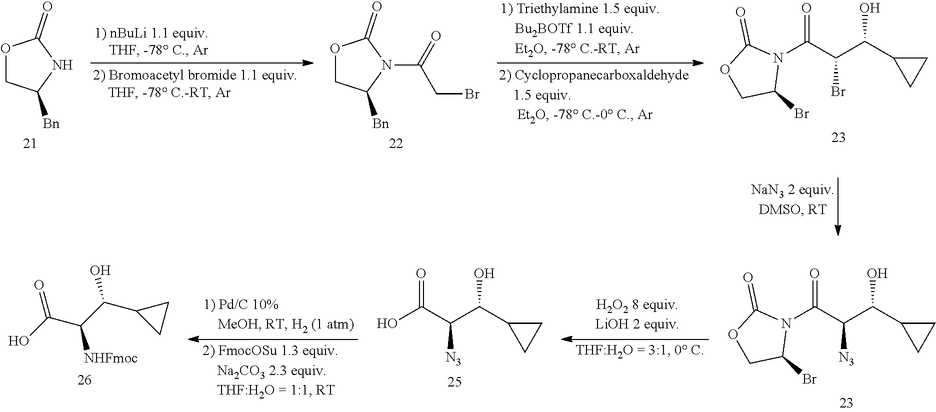

To an oven-dried flask was added 21 (10 mmol, 1.8 g) and anhydrous THF (50 mL) under Ar. After cooling to −78° C., nBuLi (2.5 M in Hexane, 11 mmol, 5.5 mL) was added. After stirring at −78° C. for 5 min, bromoacetyl bromide (11 mmol, 0.96 mL) was added and the reaction was kept at −78° C. for 10 min and then allowed to warm up to RT. After stirring at RT for 30 min, the reaction was quenched with NH₄Cl (aq), extracted by DCM, dried over Na₂SO₄, and concentrated in vacuo. CombiFlash system (0-30% EA in Hexane) was used to purify the crude mixture to afford 22 as the product (yellow solid, 2.73 g, 92% yield).

To an oven-dried flask was added 22 (9.2 mmol, 2.73 g), triethylamine (13.8 mmol, 1.93 mL), and anhydrous Et₂O (20 mL) under Ar. After cooling to −78° C., Bu₂BOTf (1 M in DCM, 10.2 mmol, 10.2 mL) was added and the reaction was allowed to warm up to RT. After stirring at RT for 1.5 h, the reaction was cooled to −78° C. and cyclopropanecarboxaldehyde (13.8 mmol, 1.03 mL) was added and the reaction was kept at 78° C. for 30 min and then allowed to warm up to 0° C. After stirring at 0° C. for 2 h, the reaction was diluted with Et₂O, washed with NaHSO₃ (aq) and water, and concentrated in vacuo. The crude mixture was redissolved in Et₂O and a 1:1 mixture (50 mL) of MeOH:H₂O₂ (30% in water) solution was added and the mixture was stirred at 0° C. for 1 h. The crude mixture solution was then washed with NaHCO₃ (aq), dried over Na₂SO₄, and concentrated in vacuo. CombiFlash system (0-40% EA in Hexane) was used to purify the crude mixture to afford 23 as the product (yellow solid, 1.56 g, 46% yield). HRMS (ESI) for C₁₆H₁₈BrNNaO₄ (M+Na), 390.0311 (Calc.), found 390.0308.

To an oven-dried flask was added 23 (4.2 mmol, 1.56 g), NaN₃ (8.4 mmol, 546 mg), and DMSO (15 mL). The reaction was stirred at 32° C. for 2 h. After completion, the reaction was diluted with a 2:1 mixture (150 mL) of Hexane:DCM solution, washed with water, dried over Na₂SO₄, and concentrated in vacuo. CombiFlash system (0-40% EA in Hexane) was used to purify the crude mixture to afford 24 as the product (yellow liquid, 1.14 g, 82% yield). HRMS (ESI) for C₁₆H₁₈N₄NaO₄ (M+Na), 353.1220 (Calc.), found 353.1229.

To an oven-dried flask was added 24 (3.5 mmol, 1.14 g) and a 3:1 mixture (60 mL) of THF:water solution. After cooling to 0° C., H₂O₂ (30% in water, 3 mL) and LiOH (6.9 mmol, 290 mg) were added and the reaction was kept stirring at 0° C. After completion, the reaction was quenched with 1M HCl (aq), extracted by EA, dried over Na₂SO₄, and concentrated in vacuo. The crude mixture containing 25 was used in the next step without further purification. HRMS (ESI) for C₆H₈N₃O₃(M−H), 170.0571 (Calc.), found 170.0552.

To an oven-dried flask was added crude mixture containing 25, Pd/C (10 wt %, ˜500 mg), and MeOH (50 mL). The reaction was vigorously stirred at RT under H₂ overnight. After completion, the reaction was filtered through a pad of celite and concentrated in vacuo. The crude mixture was used in the next step without further purification. To an oven-dried flask was added the crude mixture, FmocOSu (1.3 mmol, 1.51 g), Na₂CO₃ (7.9 mmol, 841 mg), THE (60 mL), and water (60 mL) at RT. The reaction was stirred overnight. After completion, the reaction was quenched with NH₄Cl (aq), extracted by EA, dried over Na₂SO₄, and concentrated in vacuo. CombiFlash system (0-50% EA (with 1% formic acid) in Hexane (with 1% formic acid)) was used to purify the crude mixture to afford 26 as the product (white solid, 800 mg, 63% yield over 3 steps). HRMS (ESI) for C₂₁H₂₀NO₅ (M−H), 366.1347 (Calc.), found 366.1365.

General Procedure for the Cyclic Peptide Synthesis:

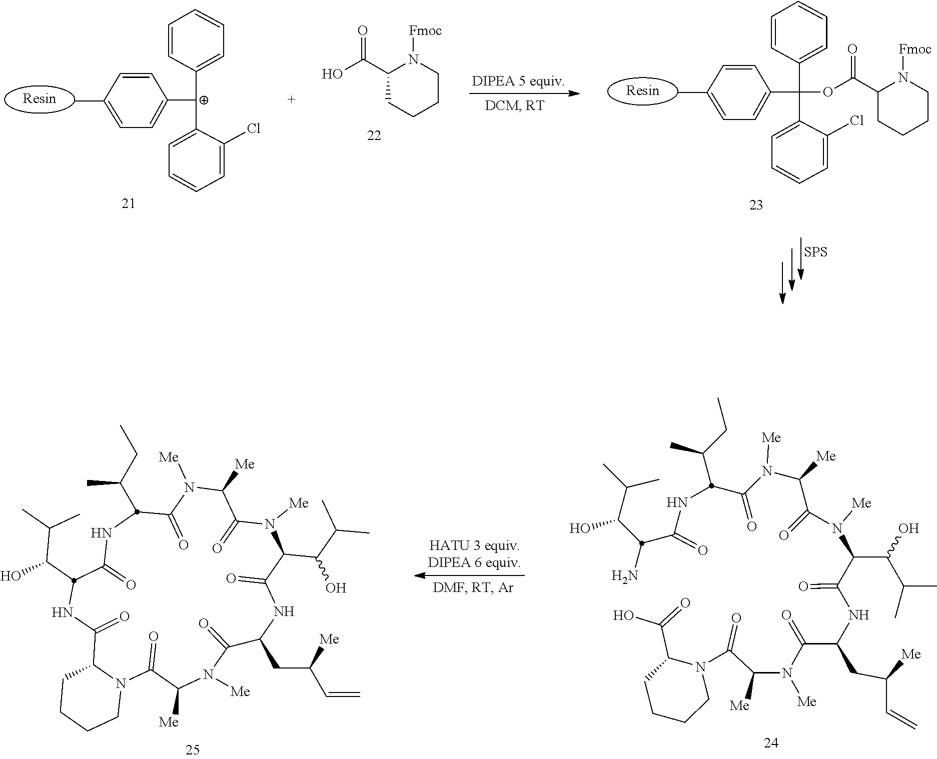

To a 50 mL syringe with a valve tip was added resin 21 (Loading=1.6 mmol/g, 1 mmol, 625 mg) and DCM (30 mL). The syringe was rocked for 1h at RT. After 1h, DCM was filtered out through the valve, and the swelled resin was washed with DCM (10 mL) for three times. 22 (2 mmol, 708.2 mg), DCM (25 mL), and DIPEA (5 mmol) were then added. After rocking the syringe overnight at RT, liquid was filtered out through the valve, and the resin was washed with DCM (10 mL) for three times. After washing, a mixture of DCM:MeOH:DIPEA=17:2:1 solution (20 mL) was added into the syringe and agitated for another 1h. After repeating the previous agitation step again, the resin was washed with DMF-IPA-DMF-IPA-DMF-DCM (×5) sequence with 10 mL of solvent each time. The resin was then dried under vacuum overnight. Loading calibration: To a 4 mL vial was added dried resin (1 mg) and 4-Me-Piperidine (20% in DMF, 3 mL). The vial was rocked for 30 min before 100 uL of the liquid was taken for loading UV measurement. Final resin 23 loading: L=1.33 mmol/g, 158 mg.

To a 12 mL syringe with a valve tip was added resin 23 (Loading=1.33 mmol/g, 0.05 mmol) and 4-Me-Piperidine (20% in DMF, 2.5 mL). The syringe was rocked for 5 min at RT twice before filtering out the liquid through the valve. After washing the resin with 5 mL DMF for three time, Fmoc-protected amino acid (0.1 mmol), HATU (0.1 mmol, 38 mg), DMF (2.5 mL), and DIPEA (0.2 mmol, 35 uL) were added to the syringe sequentially. The syringe was rocked for 2 hours at RT before filtering the liquid out through the valve. After acetaldehyde/chloranil test showed the reaction was finished, the resin was washed with DMF-IPA-DMF-IPA-DMF-DCM (×5) sequence with 5 mL of solvent each time. The above procedure was repeated to install each amino acid building blocks. After the installation of the last Fmoc-protected amino acid, the resin was mixed with 4-Me-Piperidine (20% in DMF, 2.5 mL). The syringe was rocked for 5 min twice before filtering the liquid out through the valve. 5 mL DMF was used to wash the resin for three time followed by 5 mL DCM wash for three times. After washing, 10 mL 20% HFIP in DCM was added into the syringe, and the syringe was rocked at RT for 15 min. The liquid that contained heptapeptide 24 was collected and concentrated. The crude product was used in the next macrocyclization step without further purification.

Macrocyclization was carried out using a pseudo-high dilution protocol with two syringe pumps carrying (1) the crude heptapeptide 24 in DMF (2.5 mL), and (2) HATU (0.15 mmol, 57 mg) in DMF (1.5 mL). These two reagents were added at 0.01 mL/min to a flask containing DIPEA (0.3 mmol, 52.3 uL) in DMF (3 mL). After addition, the reaction was allowed to stir at RT overnight. After full conversion of 24, the solvent was removed under reduced pressure. The resulting mixture was dissolved in EtOAc, washed with 1 M HCl, saturated aqueous NaHCO₃, and brine. The organic phase was collected and dried over Na₂SO₄, dried over Na₂SO₄, and concentrated in vacuo. CombiFlash system (0-30% Acetone in Hexane) was used to purify the crude mixture to afford 25 as the product.

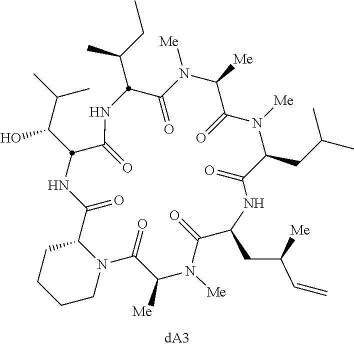 [SMILES: C=C[C@H](C)C[C@@H]1NC(=O)[C@H](CC(C)C)N(C)C(=O)[C@H](C)N(C)C(=O)[C@@H]([C@@H](C)CC)NC(=O)[C@@H]([C@H](O)C(C)C)NC(=O)[C@H]2CCCCN2C(=O)[C@H](C)N(C)C1=O]

dA3 was synthesized according to the general procedure to provide 0.05 mmol, 70% yield, 27 mg, white solid.

HRMS (ESI) for C₄₀H₆₉N₇O₈(M+H), 776.5280 (Calc.), found 776.5323.

Spectral data are in accordance with literature (Carelli et al., eLife, 2015).

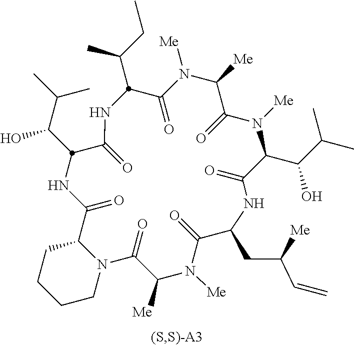 [SMILES: C=C[C@H](C)C[C@@H]1NC(=O)[C@H]([C@@H](O)C(C)C)N(C)C(=O)[C@H](C)N(C)C(=O)[C@@H]([C@@H](C)CC)NC(=O)[C@@H]([C@H](O)C(C)C)NC(=O)[C@H]2CCCCN2C(=O)[C@H](C)N(C)C1=O]

(S,S)-A3 (epi-SRA3) synthesized according to the general procedure, 0.03 mmol, 10% yield, 2.4 mg, white solid.

HRMS (ESI) for C₄₀H₆₉N₇O₉ (M+H), 792.5230 (Calc.), found 792.5279.

¹³C NMR (125 MHz, d⁶-Acetone): δ 173.7, 173.7, 172.5, 172.1, 169.5, 168.1, 167.6, 142.5, 115.1, 115.1, 75.4, 68.5, 60.5, 55.8, 54.7, 51.8, 50.7, 49.3, 48.2, 43.1, 43.1, 36.1, 36.1, 35.2, 33.7, 32.1, 31.3, 29.6, 29.4, 29.0, 25.6, 25.6, 25.2, 25.2, 25.2, 25.2, 22.1, 22.1, 22.1, 22.1, 22.1, 14.7, 14.7, 14.6, 14.0, 13.7, 11.7.

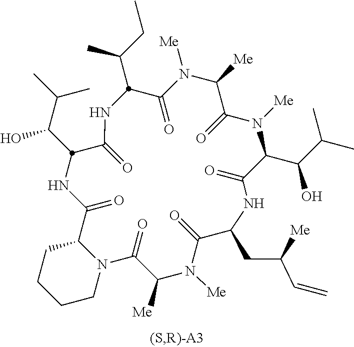 [SMILES: C=C[C@H](C)C[C@@H]1NC(=O)[C@H]([C@H](O)C(C)C)N(C)C(=O)[C@H](C)N(C)C(=O)[C@@H]([C@@H](C)CC)NC(=O)[C@@H]([C@H](O)C(C)C)NC(=O)[C@H]2CCCCN2C(=O)[C@H](C)N(C)C1=O]

(S,R)-A3 (SRA3), synthesized according to the general procedure, 0.05 mmol, 19% yield, 7.4 mg, white solid.

HRMS (ESI) for C₄₀H₆₉N₇O₉ (M+H), 792.5230 (Calc.), found 792.5279.

¹³C NMR (125 MHz, d⁶-Acetone): δ 173.6, 172.6, 172.6, 171.7, 170.6, 168.0, 167.9, 142.4, 115.0, 115.0, 75.0, 69.7, 62.5, 55.0, 54.3, 51.8, 50.4, 50.4, 49.5, 43.1, 43.1, 35.6, 35.6, 35.4, 33.1, 32.1, 31.0, 29.6, 29.4, 29.0, 25.7, 25.7, 25.2, 25.2, 25.2, 25.2, 21.0, 20.8, 20.8, 20.8, 20.7, 15.0, 14.9, 14.5, 13.7, 13.5, 11.6.

### BRIEF DESCRIPTION OF THE INVENTION

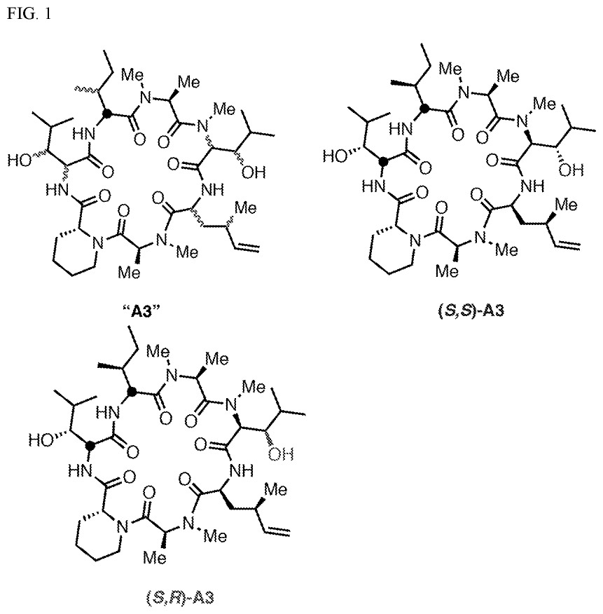

Cyclic peptide “A3” (FIG. 1), isolated from an Aspergillus fungal strain, was reported to have cytotoxic activity toward several cancer cell lines in vitro (WO2010062159 and US20110201642A1). However, the chemical structure of “A3” (and related congeners “A2”, “A4”, and “A5”) was only partially elucidated. In particular, only 4 out of 11 stereocenters in “A3” were assigned. Assuming the partial stereostructure of “A3” shown in FIG. 1 is correct, it would be necessary to synthesize and biologically characterize 128 distinct stereoisomers in order to unambiguously identify the structure of the isolated bioactive compound.

To develop and a market drug related to “A3” and related compounds, it would be ideal (if not essential) to know the complete structure, including the stereochemistry of every chiral center. Moreover, it is likely that different stereoisomers of “A3”, none of which have been previously synthesized or reported, would exhibit distinct bioactivities. This invention discloses the synthesis and biological characterization of 2 of the 128 possible stereoisomers of “A3”, termed (S,R)-A3 (SRA3) and (S,S)-A3 (SSA3, epi-SRA3). We demonstrate that (S,R)-A3 and (S,S)-A3 potently inhibit protein synthesis and cancer cell proliferation. Unexpectedly, we found that transient exposure of cancer cells to (S,R)-A3 [but not its epimer (S,S)-A3 or its deoxy analog dA3], followed by compound washout, induces profound and sustained loss of cell viability over 72 hours. In addition, (S,R)-A3 was efficacious in a mouse model of B-cell lymphoma. Based on these data, we propose that (S,R)-A3, (S,S)-A3, and related analogs may find utility as drugs for the treatment of various cancers.

Chemical Synthesis of (S,R)-A3 and (S,S)-A3

The linear heptapeptide precursors to (S,R)-A3 and (S,S)-A3 were synthesized on the solid phase using stereochemically defined amino acid building blocks, including Fmoc-protected (S,R)-Q-hydroxy N-methyl leucine and (S,S)-p-hydroxy N-methyl leucine, respectively. Cyclization of the linear peptide precursors was carried out in solution to provide the cyclic heptapeptides, (S,R)-A3 and (S,S)-A3.

### Example 3: Larger Scale Synthesis

In Vitro Biological Activity Assays

Chemical Synthesis

All reactions in non-aqueous media were conducted under a positive pressure of dry argon in glassware that had been dried in oven prior to use, unless noted otherwise. Anhydrous solutions of reaction mixtures were transferred via an oven-dried syringe or cannula. All solvents were dried prior to use unless noted otherwise. Thin layer chromatography was performed using precoated silica gel plates (EMD Chemical Inc. 60, F254). Flash column chromatography was performed on CombiFlash Rf 200i system (Teledyne Isco, Lincoln, Nebr.). ¹H and ¹³C nuclear magnetic resonance spectra (NMR) were obtained on a Varian (Palo Alto, Calif.) Inova 400 MHz spectrometer recorded in ppm (δ) downfield of TMS (δ=0) in CDCl₃ unless noted otherwise. Signal splitting patterns were described as singlet (s), doublet (d), triplet (t), quartet (q), quintet (quint), or multiplet (m), with coupling constants (J) in hertz. High resolution mass spectra (HRMS) were performed on Waters Xevo G2-XS QToF LC-MS system, eluting with a water/MeCN (+0.1% formic acid) gradient at 0.6 mL/min.

Synthesis of 5:

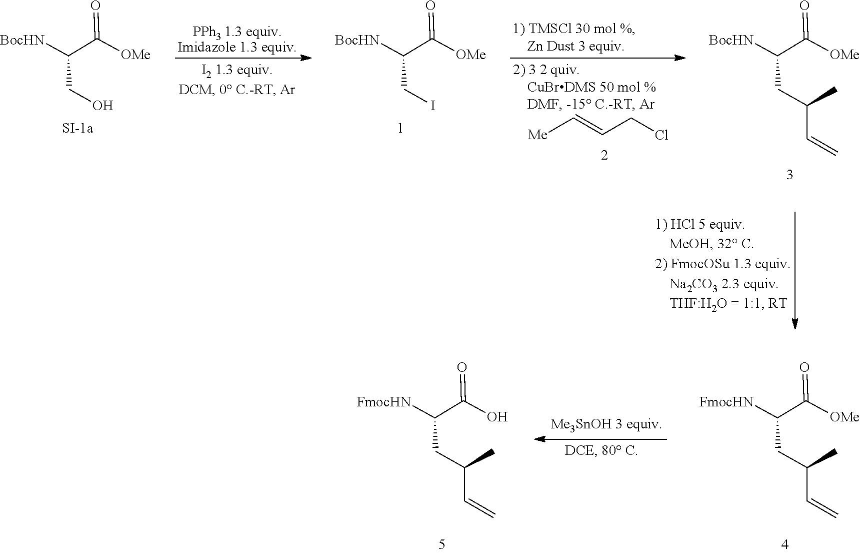

To an oven-dried flask was added PPh₃ (59.3 mmol, 15.6 g), imidazole (59.3 mmol, 4.1 g), and anhydrous DCM (250 mL) under Ar. After cooling the mixture to 0° C., I₂ (59.3 mmol, 15.1 g) was added in three portions and the reaction was warmed up to RT and stirred for 10 min. The reaction was then re-cooled to 0° C., Boc-Ser-OMe SI-1a (45.6 mmol, 10 g) was added. After stirring at 0° C. for 1 h, the reaction was allowed to warm up to RT. After completion (˜2-3 h), the reaction was filtered through a pad of Celite and concentrated in vacuo. To the above filtrate, Et₂O was added to crush out triphenyl phosphite, and the resulting slurry was filtered through a pad of Celite and concentrated in vacuo. CombiFlash chromatography (0-10% EA in Hexane) was used to afford 1 as the product (yellow solid, 12.1 g, 81% yield). Spectral data of 1 are in accordance with the literature.²

To an oven-dried flask was added Zn dust (44.4 mmol, 2.9 g), anhydrous DMF (20 mL), and TMSCl (4.44 mmol, 0.56 mL) at RT under Ar. After stirring for 30 min, the slurry was cooled to 0° C. and 1 (14.8 mmol, 4.87 g) was added. The mixture was allowed to warm up to RT and stirred for 1 h to form the corresponding Zn reagent. To a separated oven-dried flask was added CuBrDMS (7.4 mmol, 1.52 g), 2 (29.6 mmol, 2.9 mL), and anhydrous DMF (20 mL) at RT under Ar. After the mixture was cooled to −15° C., the Zn reagent was added. The reaction was then warmed up to RT and stirred overnight. After completion, the reaction was quenched with NH₄Cl (aq), extracted by EA, dried over Na₂SO₄, and concentrated in vacuo. CombiFlash chromatography (0-5% EA in Hexane) was used to afford 3 as the product (colorless liquid, 1.64 g, 43% yield). Spectral data of 3 are in accordance with our previous report.³

To an oven-dried flask was added 3 (8 mmol, 2.07 g) and 2 M HCl in MeOH (40 mmol, 20 mL). The reaction was stirred at 32° C. for 2 h. After completion, the mixture was concentrated in vacuo to afford a crude amine mixture, which was used in the next step without further purification. To an oven-dried flask was added the crude amine mixture, Fmoc-OSu (10.4 mmol, 3.5 g), Na₂CO₃ (18.4 mmol, 1.97 g), THE (20 mL), and H₂O (20 mL). The reaction was stirred overnight at RT. After completion, the reaction was quenched with NH₄Cl (aq), extracted by EA, dried over Na₂SO₄, and concentrated in vacuo. CombiFlash chromatography (0-10% EA in Hexane) was used to afford 4 as the product (colorless liquid, 1.92 g, 63% yield over 2 steps). HRMS (ESI) for C₂₃H₂₅NNaO₄ (M+Na), 402.1676 (Calc.), found 402.1716.

To an oven-dried flask was added 4 (0.2 mmol, 76 mg), Me₃SnOH (0.6 mmol, 108.5 mg), and DCE (2 mL). The reaction was stirred at 80° C. for 4 h. After completion, the reaction was diluted with EA, washed with 1 M HCl and brine, dried over Na₂SO₄, and concentrated in vacuo. The crude mixture of 5 was used in the solid phase synthesis without further purification.

Synthesis of SI-2g:

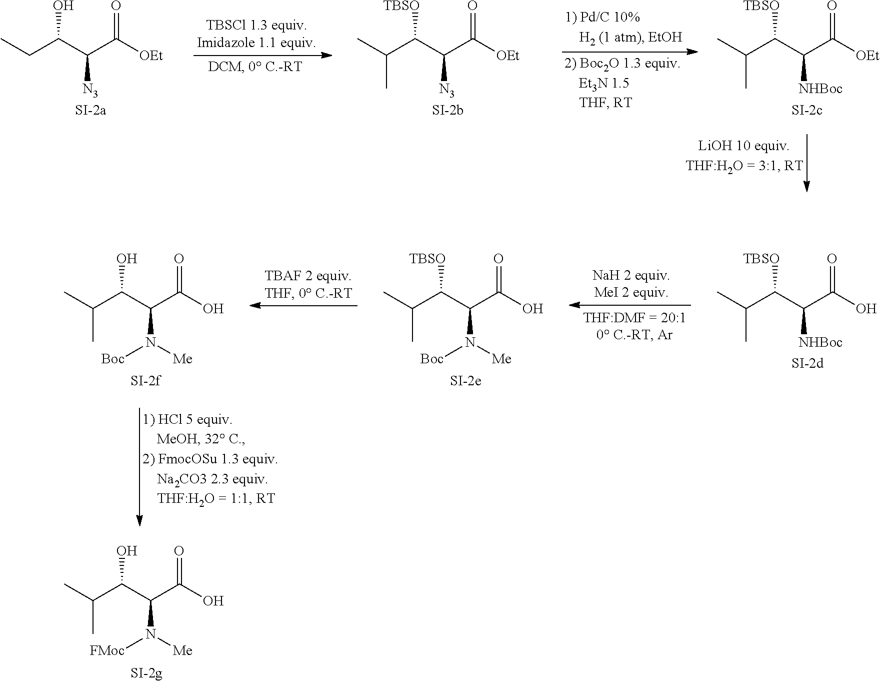

Compound SI-2a was synthesized according to the previously reported protocol.⁴

To an oven-dried flask was added SI-2a (10 mmol, 2 g), imidazole (13 mmol, 885 mg), and anhydrous DCM (50 mL). After cooling to 0° C., TBSCl (13 mmol, 1.96 g) was added and the reaction was warmed up to RT and stirred overnight. After completion, the reaction was quenched with NH₄Cl (aq), extracted by DCM, dried over Na₂SO₄, and concentrated in vacuo. The crude mixture was used in the next step without further purification. To an oven-dried flask was added SI-2b crude mixture, Pd/C (10%, 320 mg), and EtOH (50 mL) under H₂. The reaction was stirred overnight at RT. After completion, the reaction was filtered through a pad of Celite and concentrated in vacuo to afford a crude mixture. To an oven-dried flask was added the above crude mixture, Boc₂O (13 mmol, 3 mL), Et₃N (15 mmol, 2.1 mL) and THF (50 mL). The reaction stirred overnight at RT. After completion, the reaction was quenched with NH₄Cl (aq), extracted by DCM, dried over Na₂SO₄, and concentrated in vacuo. CombiFlash chromatography (0-10% EA in Hexane) was used to afford SI-2c as the product (yellow liquid, 3.2 g, 83% yield after 3 steps). HRMS (ESI) for C₁₉H₄₀NO₅Si (M+H), 390.2670 (Calc.), found 390.2664.

To an oven-dried flask was added SI-2c (5 mmol, 1.95 g), LiOH (50 mmol, 1.2 g), THE (30 mL), and H₂O (10 mL). The reaction was stirred at RT overnight. After completion, the reaction was quenched with 1 M HCl until pH=4 and concentrated in vacuo to afford a crude mixture, which was used in the next step without further purification. To an oven-dried flask was added SI-2d crude mixture, anhydrous THE (40 mL), and anhydrous DMF (2 mL). After the reaction was cooled to 0° C., NaH (60%, 10 mmol, 400 mg) was added and the reaction was stirred for 30 min at 0° C. Mel (10 mmol, 0.62 mL) was then added at 0° C. and the reaction was allowed to warm up to RT and stirred overnight. After completion, the reaction was quenched with NH₄Cl (aq), extracted by EA, dried over Na₂SO₄, and concentrated in vacuo. CombiFlash chromatography (0-10% EA in Hexane) was used to afford SI-2e as the product (yellow liquid, 1.24 g, 62% yield after 2 steps). HRMS (ESI) for C₁₈H₃₆NO₅Si (M−H), 374.2368 (Calc.), found 374.2409.

To an oven-dried flask was added SI-2e (3 mmol, 1.21 g) and THE (30 mL). After the reaction was cooled to 0° C., TBAF in THE solution (1M, 6 mmol, 6 mL) was added and the reaction was warmed to RT and stirred overnight. After completion, the reaction was quenched with NH₄Cl (aq), extracted by EA, dried over Na₂SO₄, and concentrated in vacuo. CombiFlash chromatography (0-50% EA with 1% AcOH in Hexane) was used to afford SI-2f as the product (yellow liquid, 510 mg, 65% yield). HRMS (ESI) for C₁₂H₂₂NO₅ (M−H), 260.1498 (Calc.), found 260.1504.

To an oven-dried flask was added SI-2f (1 mmol, 261 mg) and 2 M HCl in MeOH (5 mmol, 2.5 mL). The reaction was stirred at 32° C. for 2 h. After completion, the mixture was concentrated in vacuo to afford a crude mixture, which was used in the next step without further purification. To an oven-dried flask was added the crude mixture, Fmoc-OSu (1.3 mmol, 439 mg), Na₂CO₃ (2.3 mmol, 244 mg), THE (5 mL), and H₂O (5 mL). The reaction was stirred at RT overnight. After completion, the reaction was quenched with NH₄Cl (aq), extracted by EA, dried over Na₂SO₄, and concentrated in vacuo. CombiFlash chromatography (0-10% MeOH in DCM) was used to afford SI-2g as the product (white solid, 238 mg, 62% yield over 2 steps). HRMS (ESI) for C₂₂H₂₄NO₅ (M−H), 382.1660 (Calc.), found 382.1658.

Synthesis of SI-3h:

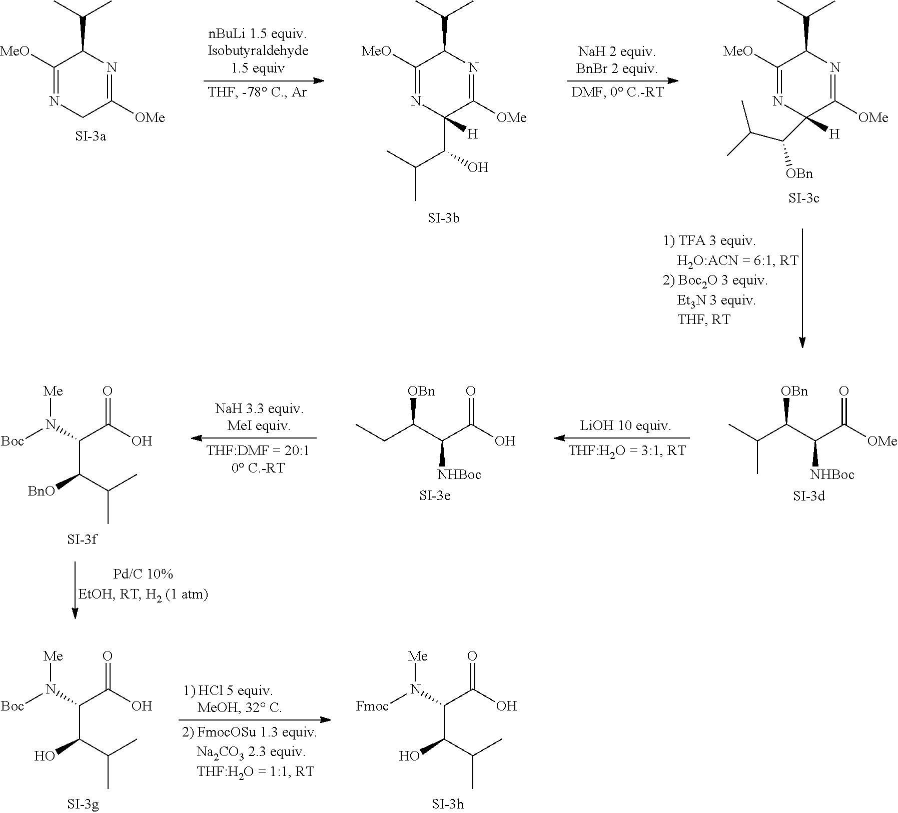

To an oven-dried flask was added SI-3a (10 mmol, 1.9 g) and anhydrous THF (50 mL) under Ar. After cooling to −78° C., nBuLi (2.5 M in Hexane, 15 mmol, 6 mL) was added. After stirring at −78° C. for 30 min, isobutyraldehyde (15 mmol, 1.4 mL) was added and the reaction was allowed to warm up to RT and stirred overnight. After completion, the reaction was quenched with NH₄Cl (aq), extracted by DCM, dried over Na₂SO₄, and concentrated in vacuo. CombiFlash chromatography (0-20% EA in Hexane) was used to afford SI-3b as the product (yellow liquid, 1.57 g, 61% yield). Spectral data of SI-3b are in accordance with the literature.⁵

To an oven-dried flask was added SI-3b (6 mmol, 1.52 g) and anhydrous DMF (50 mL). After cooling to 0° C., NaH (60%, 12 mmol, 480 mg) was added and the reaction was stirred at 0° C. for 30 min. BnBr (12 mmol, 1.42 mL) was then added and the reaction was allowed to warm up to RT and stirred overnight. After completion, the reaction was quenched with NH₄Cl (aq), extracted by EA, dried over Na₂SO₄, and concentrated in vacuo. CombiFlash chromatography (0-10% EA in Hexane) was used to afford SI-3c as the product (yellow liquid, 1.98 g, 95% yield). HRMS (ESI) for C₂₀H₃₁N₂O₃ (M+H), 347.2329 (Calc.), found 347.2341.

To an oven-dried flask was added SI-3c (6 mmol, 1.98 g), MeCN (8 mL), and water (48 mL). TFA (18 mmol, 1.4 mL) was added dropwise and the reaction was stirred overnight. After completion, the reaction was concentrated in vacuo. The crude mixture was used in the next step without further purification. To an oven-dried flask was added above crude mixture, Boc₂O (18 mmol, 4.2 mL), Et₃N (18 mmol, 2.5 mL), and THE (50 mL). The reaction was stirred overnight at RT. After completion, the reaction was quenched with NH₄Cl (aq), extracted by EA, dried over Na₂SO₄, and concentrated in vacuo. CombiFlash chromatography (0-10% EA in Hexane) was used to afford SI-3d as the product (yellow liquid, 1.33 g, 63% yield after 2 steps). HRMS (ESI) for C₁₉H₂₉NNaO₅ (M+Na), 374.1938 (Calc.), found 374.1935.

To an oven-dried flask was added SI-3d (4 mmol, 1.41 g), LiOH (40 mmol, 1.68 g), THE (30 mL) and water (10 mL). The reaction was stirred at RT for 5 h. After completion, the reaction was concentrated in vacuo. The crude mixture was used in the next step without further purification. To an oven-dried flask was added above SI-3e crude mixture, anhydrous THE (40 mL) and anhydrous DMF (2 mL). After cooling to 0° C., NaH (60%, 13.2 mmol, 528 mg) was added. After stirring at 0° C. for 30 min, Mel (12 mmol, 0.75 mL) was added. The reaction was then allowed to warm up to RT and stirred overnight. After completion, the reaction was quenched with NH₄Cl (aq), extracted by EA, dried over Na₂SO₄, and concentrated in vacuo. CombiFlash chromatography (0-30% EA (with 1% AcOH) in Hexane) was used to afford SI-3f as the product (yellow liquid, 745 mg, 53% yield). HRMS (ESI) for C₁₉H₂₈NO₅ (M−H), 350.1973 (Calc.), found 350.2015.

To an oven-dried flask was added SI-3f (2 mmol, 703 mg), Pd/C (10 wt %, ˜100 mg), and EtOH (20 mL). The reaction was vigorously stirred at RT under H₂ overnight. After completion, the reaction was filtered through a pad of Celite and concentrated in vacuo. CombiFlash chromatography (0-50% EA (with 1% AcOH) in Hexane) was used to afford SI-3g as the product (yellow liquid, 345 mg, 66% yield). HRMS (ESI) for C₁₂H₂₂NO₅ (M−H), 260.1498 (Calc.), found 260.1537.

To an oven-dried flask was added SI-3g (1 mmol, 261 mg) and 2 M HCl in MeOH (5 mmol, 2.5 mL). The reaction was stirred at 32° C. for 2 h. After completion, the mixture was concentrated in vacuo. The crude mixture was used in the next step without further purification. To an oven-dried flask was added the crude mixture, Fmoc-OSu (1.3 mmol, 439 mg), Na₂CO₃ (2.3 mmol, 244 mg), THE (5 mL), and H₂O (5 mL) at RT. The reaction was stirred overnight and monitored by TLC. After completion, the reaction was quenched with NH₄Cl (aq), extracted by EA, dried over Na₂SO₄, and concentrated in vacuo. CombiFlash chromatography (0-10% MeOH in DCM) was used to afford SI-3h as the product (white solid, 333 mg, 87% yield over 2 steps). HRMS (ESI) for C₂₂H2₄NO₅ (M−H), 382.1660 (Calc.), found 382.1658.

General Procedure for Cyclic Peptide Synthesis:

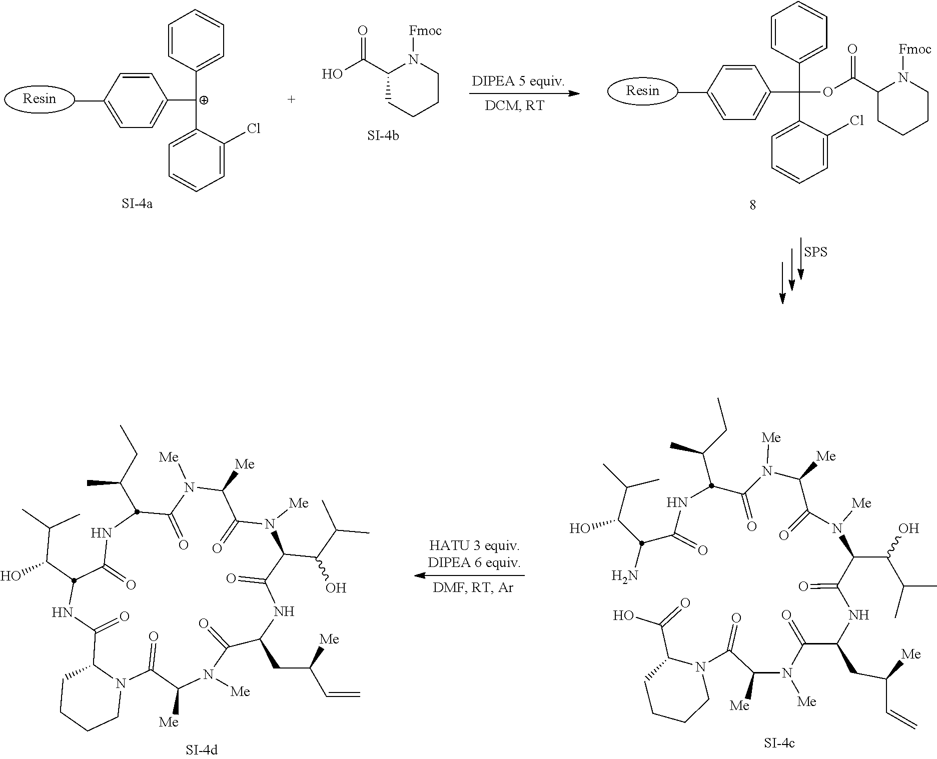

To a 50 mL syringe with a valve tip was added resin SI-4a (Loading=1.6 mmol/g, 1 mmol, 625 mg) and DCM (30 mL). The syringe was rocked for 1 hour at RT. After 1 h, DCM was filtered out through the valve, and the swelled resin was washed with DCM (10 mL) for three times. SI-4b (2 mmol, 708.2 mg), DCM (25 mL), and DIPEA (5 mmol) were then added. After rocking the syringe overnight at RT, liquid was filtered out through the valve, and the resin was washed with DCM (10 mL) for three times. After washing, a mixture of DCM:MeOH:DIPEA=17:2:1 solution (20 mL) was added into the syringe and agitated for another 1 h. After repeating the previous agitation step again, the resin was washed with DMF-IPA-DMF-IPA-DMF-DCM (×5) sequence with 10 mL of solvent each time. The resin was then dried under vacuum overnight. Loading calibration: To a 4 mL vial was added dried resin (1 mg) and 4-Me-piperidine (20% in DMF, 3 mL). The vial was rocked for 30 min before 100 μL of the liquid was taken for UV measurement. After fitting the UV value to a standard curve, the final loading of resin 8: L=1.33 mmol/g, 158 mg.

To a 12 mL syringe with a valve tip was added resin 8 (Loading=1.33 mmol/g, 0.05 mmol) and 4-Me-piperidine (20% in DMF, 2.5 mL). The syringe was rocked for 5 min at RT twice before filtering out the liquid through the valve. After washing the resin with 5 mL DMF for three time, Fmoc-protected amino acid (0.1 mmol), HATU (0.1 mmol, 38 mg), DMF (2.5 mL), and DIPEA (0.2 mmol, 35 μL) were added to the syringe sequentially. The syringe was rocked for 2 hours at RT before filtering the liquid out through the valve. After acetaldehyde/chloranil test showed the reaction was finished, the resin was washed with DMF-IPA-DMF-IPA-DMF-DCM (×5) sequence with 5 mL of solvent each time. The above procedure was repeated to install each amino acid building blocks. After the installation of the last Fmoc-protected amino acid, the resin was mixed with 4-Me-piperidine (20% in DMF, 2.5 mL). The syringe was rocked for 5 min twice before filtering the liquid out through the valve. 5 mL DMF was used to wash the resin for three time followed by 5 mL DCM wash for three times. After washing, 10 mL 20% HFIP in DCM was added into the syringe, and the syringe was rocked at RT for 15 min. The liquid that contained heptapeptide SI-4c was collected and concentrated. The crude product was used in the next macrocyclization step without further purification.

Macrocyclization reaction was carried out under a pseudo-high dilution protocol using two syringe pumps carrying (1) the crude heptapeptide SI-4c and DIPEA (0.15 mmol, 26.5 μL) in DMF (2.5 mL), and (2) HATU (0.15 mmol, 57 mg) in DMF (1.5 mL). These two reagents were added at 1.8 mL/h to a flask containing DIPEA (0.15 mmol, 26.5 μL) in DMF (3 mL). After addition, the reaction was allowed to stir at RT and monitored by LC-MS. After full conversion of SI-4c, the solvent was removed under reduced pressure. The resulting mixture was dissolved in EtOAc, washed with 1 M HCl, saturated aqueous NaHCO₃, and brine. The organic phase was collected and dried over Na₂SO₄, dried over Na₂SO₄, and concentrated in vacuo. CombiFlash chromatography (0-30% Acetone in Hexane) was used to afford SI-4d as the product.

### REFERENCES

1. Blunt, J.; Cole, T.; Munro, M.; Sun, L.; Weber, J.-F. R.; Ramasamy, K.; Bakar, H. A.; Majeed, A. B. B. A., Bioactive compounds. International Patent WO 2010/062159 A1.
2. Shimokawa, K.; Iwase, Y.; Miwa, R.; Yamada, K.; Uemura, D., Whole structure-activity relationships of the fat-accumulation inhibitor (−)-ternatin: recognition of the importance of each amino acid residue. J. Med. Chem. 2008, 51 (19), 5912-5914.
3. Carelli, J. D.; Sethofer, S. G.; Smith, G. A.; Miller, H. R.; Simard, J. L.; Merrick, W. C.; Jain, R. K.; Ross, N. T.; Taunton, J., Ternatin and improved synthetic variants kill cancer cells by targeting the elongation factor-1A ternary complex. Elife 2015, 4:e10222.
4. J. Hale, K.; Manaviazar, S.; M. Delisser, V., A practical new asymmetric synthesis of (2S,3S)- and (2R,3R)-3-hydroxyleucine. Tetrahedron 1994, 50 (30), 9181-9188.
5. Luo, S.; Krunic, A.; Kang, H. S.; Chen, W. L.; Woodard, J. L.; Fuchs, J. R.; Swanson, S. M.; Orjala, J., Trichormamides A and B with Antiproliferative Activity from the Cultured Freshwater Cyanobacterium Trichormus sp. UIC 10339. J. Nat. Prod. 2014, 77 (8), 1871-1880.

### Example 4: Characterization of Compounds

Compound Characterization

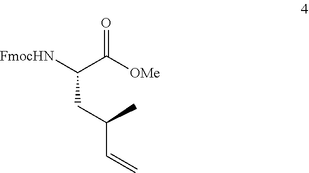

4: (2S,4R)-2-({[(9H-fluoren-9-yl)methoxy]carbonyl}amino)-4-methylhex-5-enoate.

HRMS (ESI) for C₂₃H2₅NNaO₄ (M+Na), 402.1676 (Calc.), found 402.1716.

¹H NMR (400 MHz, CDCl₃) δ 7.74 (d, J=8 Hz, 2H), 7.58 (t, J=8.0 Hz, 2H), 7.38 (t, J=8 Hz, 2H), 7.29 (t, J=8 Hz, 2H), 5.70-5.61 (m, 1H), 5.32 (d, J=8 Hz, 1H), 5.05-5.00 (m, 2H), 4.41-4.35 (m, 3H), 4.21 (t, J=8 Hz, 1H), 3.71 (s, 3H), 2.31-2.20 (m, 1H), 1.82-1.75 (m, 1H), 1.65-1.55 (m, 1H), 1.02 (d, J=4 Hz, 3H).

¹³C NMR (100 MHz, CDCl₃) δ 173.5, 156.0, 144.1, 143.9, 142.6, 141.4, 141.4, 127.8, 127.1, 125.2, 125.2, 120.17, 114.9, 67.0, 52.6, 52.4, 47.3, 39.4, 34.9, 20.9.

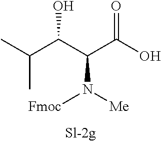

SI-2g: (2S,3S)-2-{[(9H-fluoren-9-ylmethoxy)carbonyl](methyl)amino}-3-hydroxy-4-methylpentanoic acid

HRMS (ESI) for C₂₂H2₄NO₅ (M−H), 382.1660 (Calc.), found 382.1658.

¹H NMR (400 MHz, MeOD) δ 7.78-7.75 (m, 2H), 7.64-7.57 (m, 2H), 7.39-7.35 (m, 2H), 7.33-7.26 (m, 2H), 4.57-4.29 (m, 3H), 4.23-4.17 (m, 1H), 3.81-3.78 (m, 1H), 2.89 (s, 1H), 2.78 (s, 2H), 1.71-1.60 (m, 1H), 0.97 (d, J=4 Hz, 2H), 0.88-0.84 (m, 3H), 0.77 (d, J=4 Hz, 1H). This compound has multiple rotamers; only major peaks are listed and integrated.

¹³C NMR (100 MHz, MeOD) δ 174.1, 173.8, 158.3, 158.0, 145.4, 145.4, 145.2, 145.2, 142.8, 142.8, 142.7, 142.7, 128.9, 128.9, 128.3, 128.3, 126.3, 126.1, 126.1, 126.1, 121.1, 121.1, 121.1, 75.3, 75.1, 69.1, 68.8, 62.2, 48.7, 37.9, 35.3, 32.5, 32.4, 31.0, 30.8, 20.5, 20.4, 16.2, 15.7. This compound has multiple rotamers; only major peaks are listed.

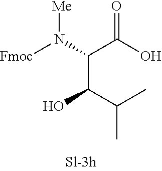

SI-3h: (2S,3R)-2-{[(9H-fluoren-9-ylmethoxy)carbonyl](methyl)amino}-3-hydroxy-4-methylpentanoic acid

HRMS (ESI) for C₂₂H₂₄NO₅ (M−H), 382.1660 (Calc.), found 382.1658.

¹H NMR (400 MHz, MeOD) δ 7.80-7.76 (m, 2H), 7.64-7.58 (m, 2H), 7.40-7.35 (m, 2H), 7.33-7.26 (m, 2H), 4.84 (d, J=4 Hz, 1H), 4.56-4.37 (m, 2H), 4.28-4.19 (m, 2H), 3.86-3.82 (m, 1H), 3.50-3.45 (m, 1H), 3.00 (s, 3H), 1.66-1.55 (m, 1H), 1.17 (t, J=8 Hz, 1H), 1.01 (d, J=4 Hz, 2H), 0.93 (d, J=4 Hz, 1H), 0.87 (d, J=4 Hz, 2H), 0.70 (d, J=8 Hz, 1H). This compound has multiple rotamers; only major peaks are listed and integrated.

¹³C NMR (100 MHz, MeOD) δ 173.9, 173.6, 159.3, 158.6, 145.4, 145.3, 145.3, 142.8, 142.8, 142.7, 142.7, 128.9, 128.9, 128.3, 128.3, 128.2, 126.1, 126.0, 126.0, 121.0, 77.5, 76.3, 69.0, 69.0, 67.0, 62.6, 62.1, 48.5, 33.8, 33.0, 32.6, 32.3, 26.4, 20.0, 19.9, 19.2, 18.4, 15.5. This compound has multiple rotamers; only major peaks are listed.

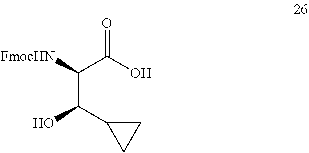

Compound 26: HRMS (ESI) for C₂₁H2₀NO₅ (M−H), 366.1347 (Calc.), found 366.1365.

¹H NMR (400 MHz, MeOD): δ 7.80 (d, J=4 Hz, 2H), 7.70-7.62 (m, 2H), 7.39 (t, J=8 Hz, 2H), 7.31 (t, J=8 Hz, 2H), 7.25-7.22 (m, 1H), 4.41-4.36 (m, 3H), 4.24 (t, J=8 Hz, 1H), 4.17-4.09 (m, 1H), 3.22-3.18 (m, 1H), 2.91-2.81 (m, 1H), 1.15-1.08 (m, 1H), 0.53-0.50 (m, 2H), 0.33 (d, J=8 Hz, 2H).

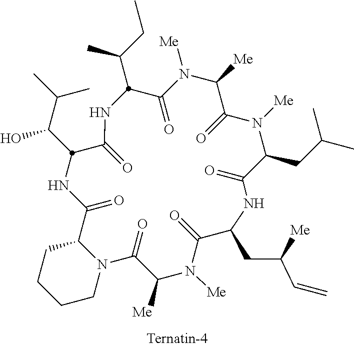 [SMILES: C=C[C@H](C)C[C@@H]1NC(=O)[C@H](CC(C)C)N(C)C(=O)[C@H](C)N(C)C(=O)[C@@H]([C@@H](C)CC)NC(=O)[C@@H]([C@H](O)C(C)C)NC(=O)[C@H]2CCCCN2C(=O)[C@H](C)N(C)C1=O]

Ternatin-4, 0.05 mmol scale, 70% overall yield, 27 mg, white solid.

Spectral data of ternatin-4 are in accordance with our previous report.³

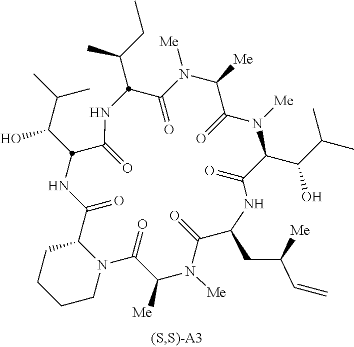 [SMILES: C=C[C@H](C)C[C@@H]1NC(=O)[C@H]([C@@H](O)C(C)C)N(C)C(=O)[C@H](C)N(C)C(=O)[C@@H]([C@@H](C)CC)NC(=O)[C@@H]([C@H](O)C(C)C)NC(=O)[C@H]2CCCCN2C(=O)[C@H](C)N(C)C1=O]

(S,S)-A3, 0.03 mmol scale, 21% overall yield, 5 mg, white solid.

HRMS (ESI) for C₄₀H₆₉N₇NaO₉ (M+Na), 814.5049 (Calc.), found 814.5059.

¹H NMR (400 MHz, Acetone-d⁶) δ 7.83-7.67 (m, 2H), 7.45 (m, 1H), 5.75-5.52 (m, 1H), 5.39 (m, 1H), 5.18-4.77 (m, 3H), 4.74-4.51 (m, 1H), 4.44-4.24 (m, 1H), 4.19-3.85 (m, 1H), 3.60-3.35 (m, 2H), 3.07 (d, J=5.6 Hz, 1H), 3.02-2.88 (m, 9H), 2.43-2.30 (m, 1H), 2.17 (m, 2H), 1.84-1.69 (m, 6H), 1.53-1.39 (m, 7H), 1.32-1.22 (m, 5H), 0.99-0.91 (m, 10H), 0.88-0.72 (m, 16H). This compound has multiple rotamers; only major peaks are listed and integrated.

¹³C NMR (100 MHz, Acetone-d⁶): δ 174.5, 174.4, 174.3, 174.0, 172.9, 170.2, 169.2, 143.5, 115.6, 80.8, 76.2, 72.9, 64.6, 56.3, 56.1, 53.0, 52.1, 50.4, 50.2, 44.2, 40.71, 36.7, 34.8, 34.18, 31.8, 27.2, 26.5, 26.3, 26.10, 25.96, 25.67, 23.33, 21.81, 21.48, 21.25, 21.06, 21.03, 20.73, 18.28, 15.70, 15.66, 15.60, 15.52, 21.9, 21.5, 15.5, 14.8, 14.4, 14.1, 12.2, 12.1. This compound has multiple rotamers; only major peaks are listed and integrated.

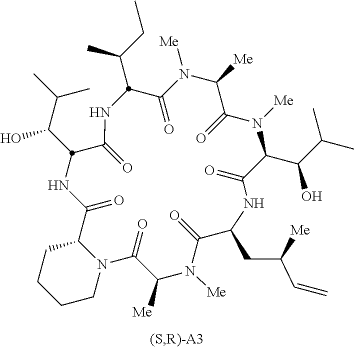 [SMILES: C=C[C@H](C)C[C@@H]1NC(=O)[C@H]([C@H](O)C(C)C)N(C)C(=O)[C@H](C)N(C)C(=O)[C@@H]([C@@H](C)CC)NC(=O)[C@@H]([C@H](O)C(C)C)NC(=O)[C@H]2CCCCN2C(=O)[C@H](C)N(C)C1=O]

(S,R)-A3, 0.1 mmol scale, 35% overall yield, 27.2 mg, white solid.

HRMS (ESI) for C₄₀H₆₉N₇NaO₉ (M+Na), 814.5049 (Calc.), found 814.5059.

¹H NMR (400 MHz, Acetone-d⁶) δ 7.78-7.75 (m, 2H), 7.52 (d, J=8 Hz, 1H), 5.85 (q, J=8 Hz, 1H), 5.76-5.67 (m, 1H), 5.18 (br, 1H), 5.08-4.95 (m, 3H), 4.90-4.85 (m, 1H), 4.80-4.72 (m, 2H), 4.09-4.02 (m, 2H), 3.92-3.89 (m, 1H), 3.63-3.59 (m, 1H), 3.10 (s, 3H), 3.05 (s, 3H), 2.98 (s, 3H), 2.52-2.49 (m, 1H), 2.22-2.14 (m, 1H), 2.00-1.72 (m, 6H), 1.65-1.56 (m, 4H), 1.53-1.44 (m, 2H), 1.40-1.35 (m, 3H), 1.33-1.26 (m, 3H), 1.13 (d, J=8 Hz, 3H), 1.05-0.94 (m, 18H), 0.90 (d, J=8 Hz, 3H). This compound has multiple rotamers; only major peaks are listed and integrated.

¹³C NMR (100 MHz, Acetone-d⁶) δ 174.6, 174.5, 174.4, 173.3, 171.7, 169.2, 168.7, 143.6, 115.5, 76.1, 71.8, 63.8, 56.3, 56.1, 53.1, 51.8, 50.9, 49.7, 44.3, 36.7, 34.6, 31.8, 27.4, 26.6, 26.2, 21.9, 21.5, 21.5, 21.2, 15.9, 15.5, 14.7, 14.2, 14.1, 12.1. This compound has multiple rotamers; only major peaks are listed.

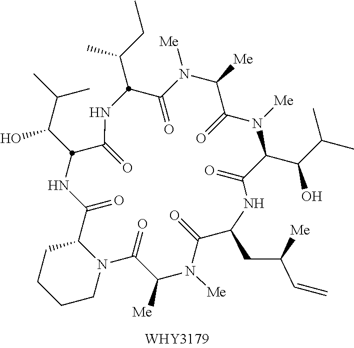 [SMILES: C=C[C@H](C)C[C@@H]1NC(=O)[C@H]([C@H](O)C(C)C)N(C)C(=O)[C@H](C)N(C)C(=O)[C@@H]([C@H](C)CC)NC(=O)[C@@H]([C@H](O)C(C)C)NC(=O)[C@H]2CCCCN2C(=O)[C@H](C)N(C)C1=O]

WHY3179, synthesized according to the general procedure, 0.03 mmol scale, 16% overall yield, 3.8 mg, white solid.

HRMS (ESI) for C₄₀H₆₈N₇O₉ (M−H), 790.5084 (Calc.), found 790.4703.

¹H NMR (400 MHz, d⁶-Acetone): δ 7.82-7.78 (m, 2H), 7.58 (d, J=8 Hz, 1H), 5.84 (q, J=8 Hz, 1H), 5.76-5.67 (m, 1H), 5.16-5.14 (m, 1H), 5.07-4.96 (m, 3H), 4.90-4.84 (m, 1H), 4.77-4.72 (m, 1H), 4.64-4.61 (m, 1H), 4.09-4.02 (m, 2H), 3.93-3.90 (m, 1H), 3.64-3.62 (m, 1H), 3.10 (s, 3H), 3.05 (s, 3H), 2.98 (s, 3H), 2.51-2.49 (m, 1H), 2.22-2.16 (m, 1H), 1.98-1.71 (m, 6H), 1.65-1.57 (m, 4H), 1.53-1.44 (m, 2H), 1.39 (d, J=8 Hz, 3H), 1.33-1.29 (m, 3H), 1.13 (d, J=8 Hz, 3H), 1.03-0.82 (m, 21H). This compound has multiple rotamers; only major peaks are listed and integrated.

¹³C NMR (100 MHz, d⁶-Acetone): δ 174.6, 174.4, 174.4, 173.3, 171.7, 169.2, 168.7, 143.6, 115.5, 75.9, 71.5, 63.7, 58.3, 56.0, 53.0, 51.8, 50.9, 49.7, 44.2, 36.8, 35.7, 31.7, 26.5, 26.1, 24.3, 21.8, 21.4, 21.4, 21.1, 16.3, 15.8, 15.4, 14.6, 14.1, 12.2. This compound has multiple rotamers; only major peaks are listed.

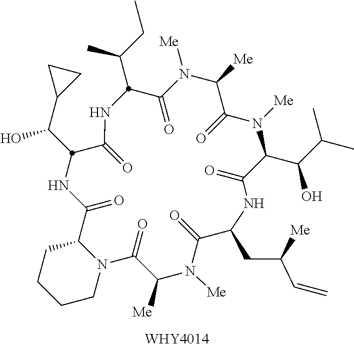 [SMILES: C=C[C@H](C)C[C@@H]1NC(=O)[C@H]([C@H](O)C(C)C)N(C)C(=O)[C@H](C)N(C)C(=O)[C@@H]([C@@H](C)CC)NC(=O)[C@@H]([C@H](O)C2CC2)NC(=O)[C@H]2CCCCN2C(=O)[C@H](C)N(C)C1=O]

WHY4014, synthesized according to the general procedure, 0.03 mmol scale, 11% overall yield, 2.6 mg, white solid.

HRMS (ESI) for C₄₀H₆₆N₇O₉ (M−H), 788.4928 (Calc.), found 788.5024.

¹H NMR (400 MHz, d⁶-Acetone): δ 7.77-7.72 (m, 2H), 7.61 (d, J=8 Hz, 1H), 5.83-5.67 (m, 2H), 5.34 (d, J=4 Hz, 1H), 5.19 (br, 1H), 5.09-4.69 (m, 3H), 4.89-4.82 (m, 1H), 4.77-4.72 (m, 1H), 4.20 (d, J=8 Hz, 1H), 4.06-3.97 (m, 2H), 3.93-3.88 (m, 1H), 3.48-3.43 (m, 1H), 3.30-3.21 (m, 1H), 3.10 (s, 3H), 3.04 (s, 3H), 2.97 (s, 3H), 2.51-2.48 (m, 1H), 2.24-2.17 (m, 1H), 1.98-1.80 (m, 3H), 1.75-1.72 (m, 1H), 1.68-1.57 (m, 4H), 1.53-1.44 (m, 2H), 1.42-1.29 (m, 6H), 1.20 (s, 1H), 1.12 (d, J=8 Hz, 3H), 1.02-0.90 (m, 13H), 0.48-0.41 (m, 3H), 0.31-0.26 (m, 2H). This compound has multiple rotamers; only major peaks are listed and integrated.

¹³C NMR (100 MHz, d⁶-Acetone): δ 174.5, 174.3, 173.9, 173.1, 171.7, 169.4, 168.7, 143.5, 115.5, 73.8, 71.7, 63.7, 58.8, 56.1, 53.1, 51.9, 50.8, 49.7, 44.2, 36.7, 36.6, 34.6, 31.8, 30.6, 27.3, 26.5, 26.1, 21.8, 21.5, 21.4, 15.7, 15.4, 14.6, 14.1, 14.0, 12.1, 4.1, 1.1. This compound has multiple rotamers; only major peaks are listed.

In Vitro Biological Activity Assays

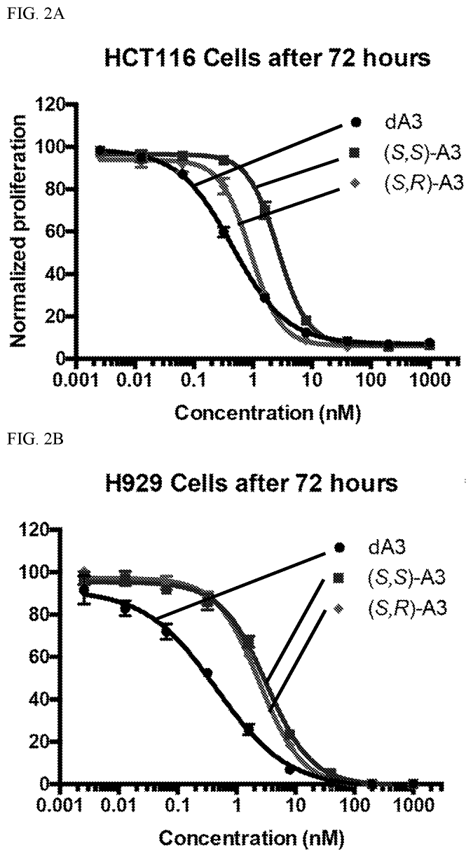

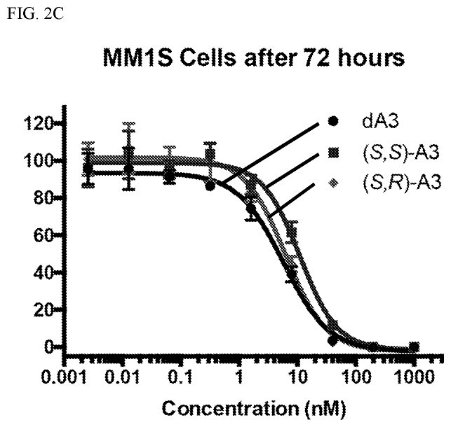

To assess in vitro anticancer activity, HCT116, H929, and MM1S cells were treated continuously for 72 hours with increasing concentrations of (S,S)-A3, (S,R)-A3, or dA3 (cyclic peptide 4 (Carelli, Taunton, et al., eLife, 2015), here termed “dA3” (deoxy-A3)). Cell viability was then assessed using the Alamar Blue assay. Under these continuous treatment conditions, all 3 compounds reduced cell viability at similar, low nanomolar concentrations, with dA3 being slightly more potent than (S,S)-A3 or (S,R)-A3 (FIG. 2A-2C).

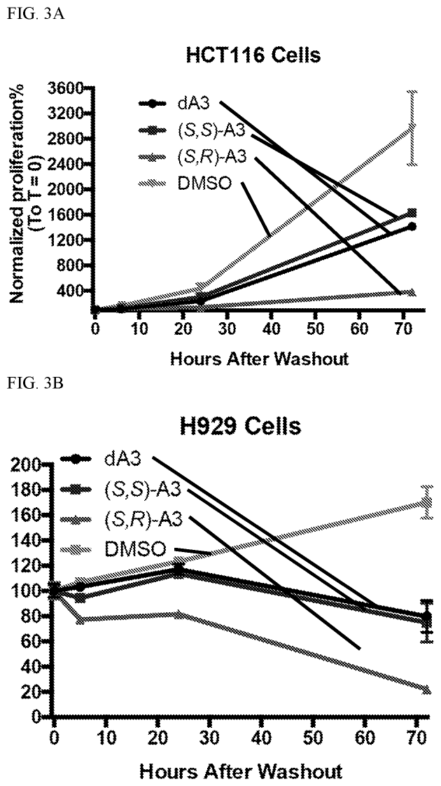

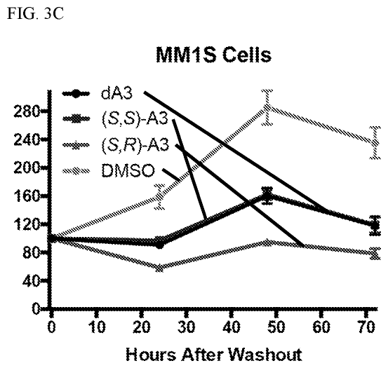

A second experiment produced unexpected results. HCT116, H929, and MM1S cells were transiently exposed to (S,S)-A3, (S,R)-A3, or dA3 (1 h or 4 h), followed by stringent washout and incubation in compound-free media for 72 hours (FIG. 3A-3C). In all 3 cell lines, transient exposure to (S,R)-A3 resulted in strong antiproliferative activity, whereas dA3 and (S,S)-A3 were less efficacious under these conditions. Hence, despite the structural similarity of the 3 cyclic peptides, (S,R)-A3 exhibited an unexpected resistance to stringent washout, suggesting that it may have a slower dissociation rate from its intracellular target and hence, a longer duration of action compared to the deoxy analog dA3 and the epimer (S,S)-A3. These data highlight the importance of the N-methyl β-hydoxy leucine group and its stereochemistry [(S,R)-A3 compared to dA3 and (S,S)-A3) with respect to the unexpected and pharmaceutically desirable property of sustained, durable efficacy.

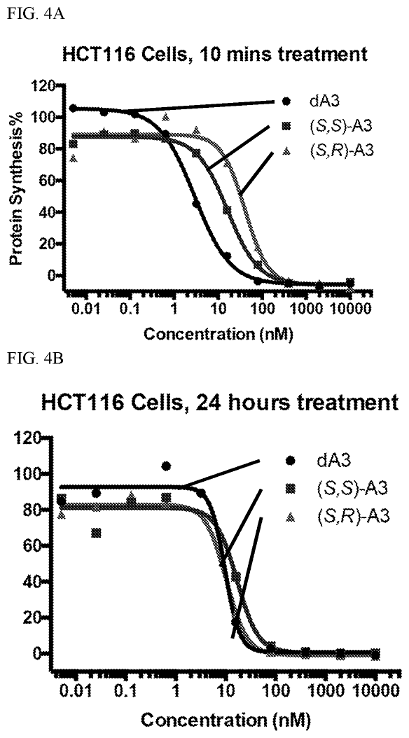

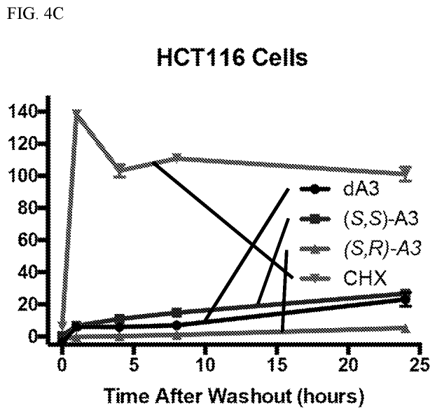

A standard O-propargyl puromycin (OPP) incorporation assay was employed to measure the effect of dA3, (S,S)-A3, and (S,R)-A3 on protein synthesis rates in HCT116 cells after continuous exposure (10 min or 24 h) or transient exposure (100 nM for 4 h) followed by stringent washout for 24 hours. The protein synthesis inhibitor cycloheximide (50 μg/mL) was employed as a positive control. After 10 minutes of continuous treatment, dA3 inhibited protein synthesis with slightly greater potency than (S,S)-A3 and (S,R)-A3, whereas after 24 hours of continuous treatment, all three compounds exhibited similar potencies (FIGS. 4A and 4B). By contrast, after transient exposure followed by washout into compound-free media, protein synthesis rates partially recovered over the next 24 hours in cells exposed to dA3 and (S,S)-A3, but not (S,R)-A3 (FIG. 4C). These results are consistent with the cell proliferation results (FIG. 3A-3C) and demonstrate that (S,R)-A3 inhibits cellular protein synthesis in a sustained, washout-resistant manner compared to dA3 and (S,S)-A3.

In Vivo Anticancer Activity of (S,R)-A3

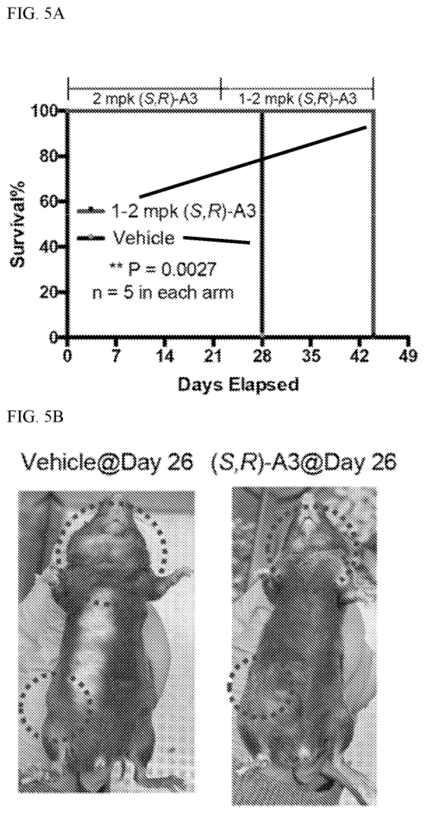

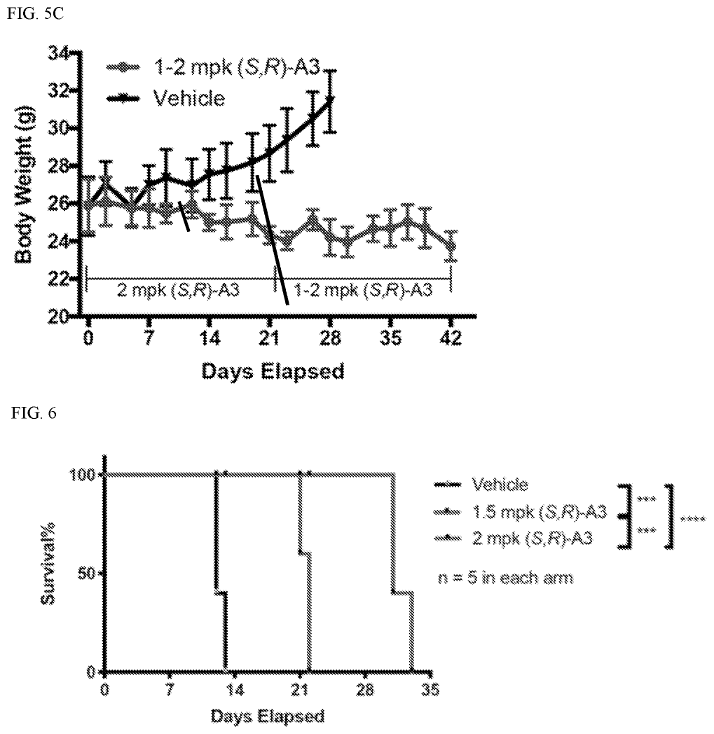

We next tested the effect of (S,R)-A3 in a preclinical mouse model of B-cell lymphoma using an intermittent dosing regimen (QOD×3, every other day, 3 doses per week via intraperitoneal injection). Eμ-Myc/+ mice are a well-characterized mouse lymphoma model that overexpress MYC under the transcriptional control of the IgH enhancer element. These mice recapitulate the genetic lesion underlying Burkitt's Lymphoma. Eμ-Myc/+ lymphoma cells were injected via the tail vein into eight-week-old male C57BL/6J mice. Seven days after lymphoma cell injection, mice were randomized to receive vehicle or (S,R)-A3 by intraperitoneal injection (QOD×3, n=5 mice/treatment arm). As shown in FIG. 5A-5C, treatment with (S,R)-A3 reduced tumor growth and significantly improved survival (P=0.0027), without causing excessive body weight loss (maximum BW loss<8%).

### Example 5: Characterization of Compounds

Efficient Synthesis of Dehydromethyl Leucine (dhML)

We previously found that replacing (S)-leucine in ternatin with (S,R)-dehydromethyl leucine (hereafter “dhML”) leads to increased potency. Because our original 6-step synthesis of dhML methyl ester was low yielding and required a costly chiral auxiliary, we developed a more efficient, second-generation synthesis suitable for preparing gram quantities of Fmoc-dhML.

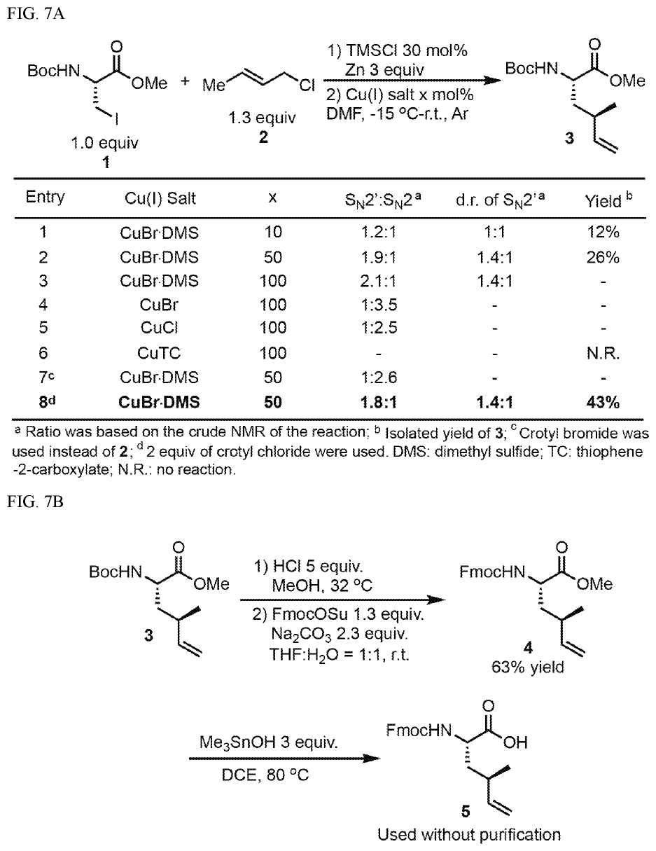

The copper(I)-promoted S_{N}2′ reaction between a serine-derived organozinc reagent and allylic electrophiles has been previously exploited to synthesize amino acids that contain a γ-stereogenic center.¹²⁻¹³ This method was appealing because it would provide dhML (as the Boc methyl ester) in only two steps from the inexpensive chiral building block, Boc-(S)-serine-OMe. Using previously reported conditions in which the organozinc reagent was generated in situ from Boc-iodoalanine-OMe 1,¹² the S_{N}2′ reaction with crotyl chloride 2 was slightly favored over the S_{N}2 pathway, providing the desired Boc-dhML-OMe 3 in 12% isolated yield (FIG. 7A-7B, entry 1). After extensive optimization, aimed at improving S_{N}2′ vs. S_{N}2 selectivity and conversion, we obtained Boc-dhML-OMe 3 in 43% isolated yield (1.6 g) through the use of 50 mol % CuBrDMS and 2 equivalents of crotyl chloride (FIG. 7A-7B, entry 8). When crotyl bromide was used, the S_{N}2 pathway became dominant (FIG. 7A-7B, entry 7), indicating a key role of the leaving group in controlling pathway selectivity. The use of the CuBrDMS complex was also critical, as other copper(I) salts, including CuBr, favored the S_{N}2 pathway or resulted in no reaction (FIG. 7A-7B, entries 4-6). S_{N}2′ diastereoselectivity is modest under the optimized reaction conditions, but the desired product is easily purified by silica gel chromatography. Boc to Fmoc exchange, followed by ester hydrolysis, provided Fmoc-dhML 5 (FIG. 7B), which was incorporated into the linear heptapeptide as described below.

Synthesis of Ternatin-4, SS-A3, and SR-A3 Via an Improved Macrocyclization Strategy

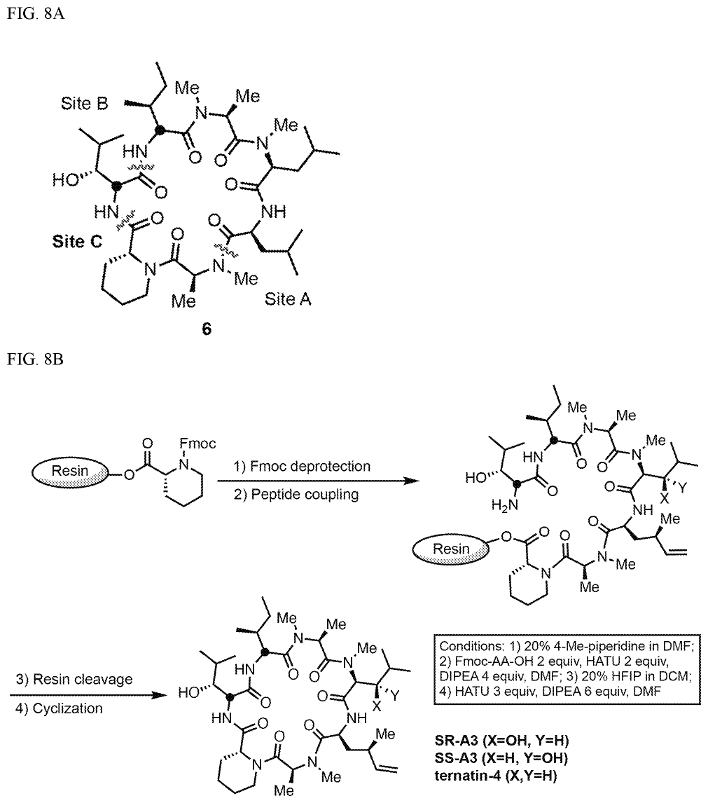

A solid-phase route was previously employed to synthesize a linear heptapeptide precursor of ternatin, followed by solution-phase cyclization.⁹ However, this strategy involved macrocyclization between the secondary amine of N-Me-Ala7 and the carboxylic acid of Leu1 (FIG. 8A, site A), which we found to be low-yielding in the context of peptides containing dhML at the carboxy terminus.¹⁰ Thus, we sought to identify an alternative cyclization site using the ternatin-related cyclic peptide 6 as a model system (FIG. 8A). Linear heptapeptide precursors were synthesized on the solid phase, deprotected and cleaved from the resin, and cyclized in solution. We failed to evaluate site B due to the poor resin loading of Fmoc-β-OH-Leu. Gratifyingly, cyclization at site C provided 6 in 63% overall yield (including the solid-phase linear heptapeptide synthesis), whereas cyclization at site A was less efficient (46% overall yield). By synthesizing the linear heptapeptide precursor on the solid phase and cyclizing in solution at site C, we were able to prepare ternatin-4 in 3 days and 70% overall yield (27 mg), a significant improvement over our previous route (FIG. 8B). Most importantly, by incorporating Fmoc-protected (S,R)— and (S,S)—N-Me-β-OH-Leu, we completed the first total syntheses of SR-A3 (21 mg, 35% overall yield) and SS-A3 (5 mg, 21% overall yield).

N-Me-β—OH-Leu Stereospecifically Confers Increased Cellular Residence Time

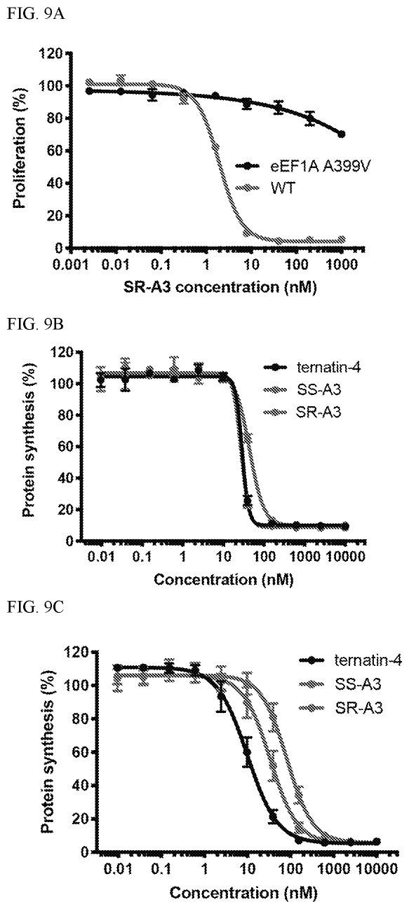

We previously demonstrated that the antiproliferative effects of ternatin-4 were abrogated in cells expressing a point mutant of eEF1A (A399V).¹⁰ eEF1A-mutant cells were similarly resistant to SR-A3 (IC₅₀>>1 μM), providing strong genetic evidence that eEF1A is a physiologically relevant target (FIG. 9A). Consistent with this interpretation, treatment of cells with SR-A3 for 24 h reduced the rate of protein synthesis with an IC₅₀ of ˜20 nM (FIG. 9B), as measured by a clickable puromycin incorporation assay (O-propargyl puromycin, OPP).¹⁴ Under these conditions—24 h of continuous treatment prior to a 1-h pulse with OPP—ternatin-4 behaved identically to SR-A3, whereas SS-A3 was slightly less potent. However, when the treatment time was shortened to 10 min before pulse-labeling with OPP for 1 h (in the continuous presence of the cyclic peptides), the dose-response curves shifted significantly, such that ternatin-4 was ˜10-fold more potent than SR-A3 and SS-A3 had intermediate potency (FIG. 9C). These data demonstrate: (1) replacing N-Me-Leu (ternatin-4) with N-Me-β-OH-Leu (SR-A3, SS-A3) results in decreased potency (or no significant difference) under continuous exposure conditions, and (2) the relative potency of SR-A3, SS-A3, and ternatin-4 is time-dependent. The latter effect is likely due to intrinsic differences in cell permeability and/or eEF1A binding kinetics.

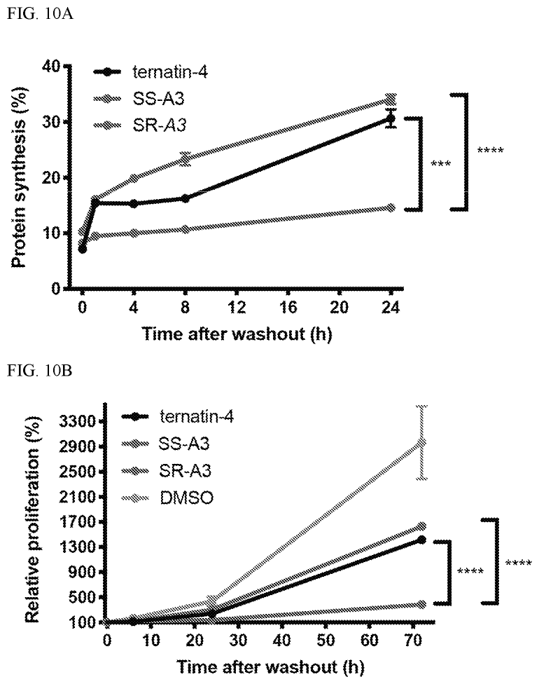

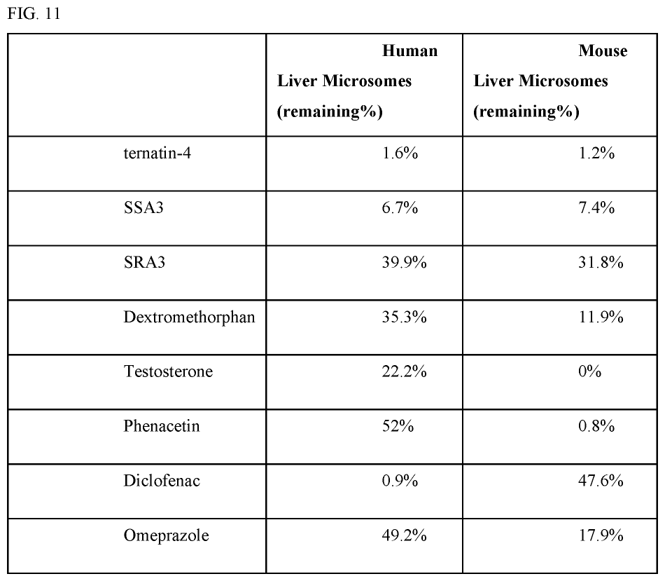

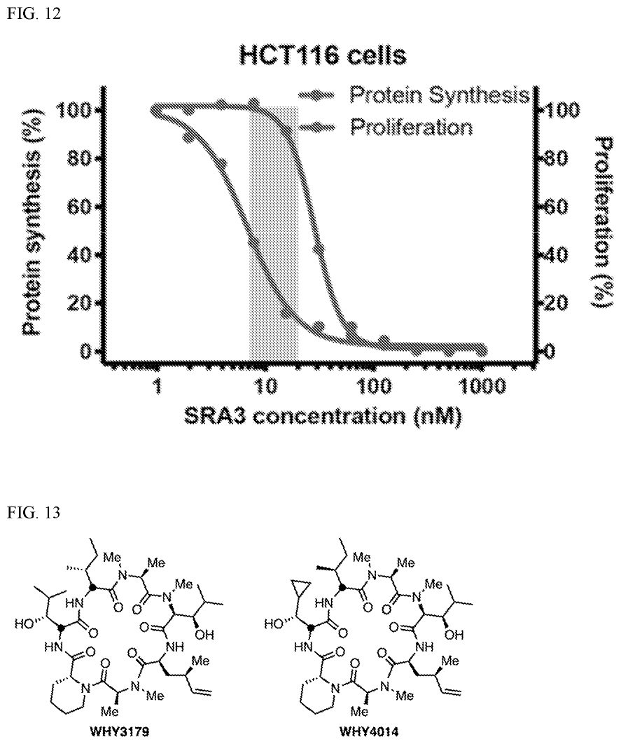

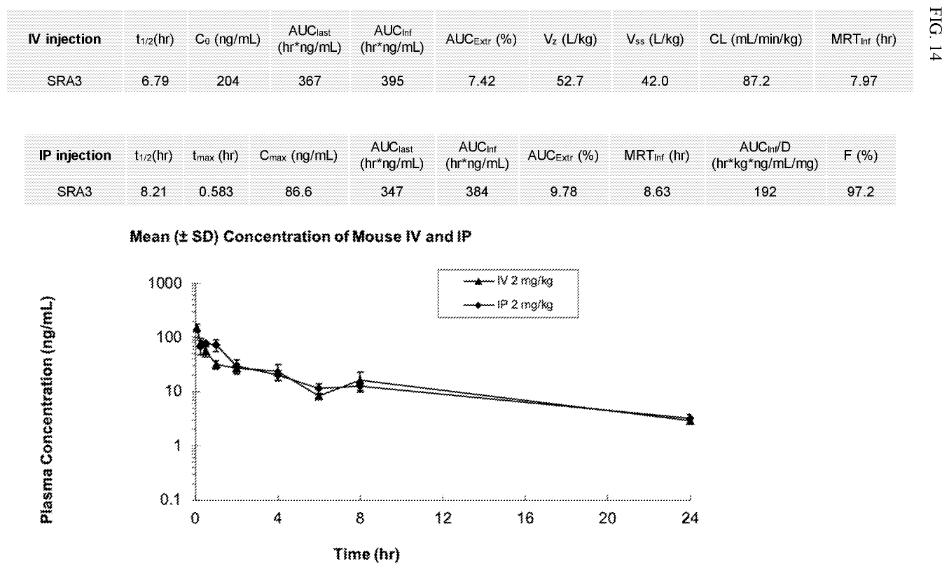

Drug-target residence time, which reflects not only the intrinsic biochemical off-rate, but also the rebinding rate and local target density in vivo, has emerged as a critical kinetic parameter in drug discovery.¹⁵⁻¹⁶ To test for potential differences in cellular residence time, we treated HCT116 cells with 100 nM SR-A3, SS-A3, or ternatin-4 for 4 h, followed by washout into compound-free media. At various times post-washout, cells were pulse-labeled with OPP for 1 h. Whereas protein synthesis rates partially recovered in cells treated with ternatin-4 or SS-A3 (˜30% of DMSO control levels, 24 h post-washout), transient exposure of cells to SR-A3 resulted in sustained inhibition (FIG. 10A). To confirm the extended duration of action observed with SR-A3, we assessed cell proliferation during a 72-h washout period. Strikingly, cell proliferation was nearly abolished after 4-h treatment with 100 nM SR-A3, followed by rigorous washout. By contrast, cell proliferation rates recovered to ˜50% of DMSO control levels after transient exposure to 100 nM ternatin-4 or SS-A3. These results demonstrate that the (R)-β-hydroxy group attached to N-Me-Leu endows SR-A3 with a kinetic advantage over SS-A3 and ternatin-4, as reflected by washout resistance and increased cellular residence time.

5,000 HCT116 cells were seeded in 100 μL complete growth media per well in 96-well plates. After allowing cells to grow/adhere overnight, cells were treated with increasing concentrations of the indicated compounds for 24 h (triplicate wells for each condition). Cell viability was assessed relative to DMSO controls using the Alamar Blue assay and IC50 values were determined and shown in Table of Compound Activity below.

Table of Compound Activity

| | IC50 in HCT116 cells (nM) |
|---|---|
| ternatin-4 | 1.4 |
| SSA3 | 3 |
| SRA3 | 2.8 |
| WHY3179 | 6.3 |
| WHY4014 | 14.8 |

1. Schuller, A. P.; Green, R., Roadblocks and resolutions in eukaryotic translation. Nat Rev Mol Cell Biol 2018, 19 (8), 526-541.
2. Abbas, W.; Kumar, A.; Herbein, G., The eEF1A Proteins: At the Crossroads of Oncogenesis, Apoptosis, and Viral Infections. Front Oncol 2015, 5, 75.
3. Crews, C. M.; Collins, J. L.; Lane, W. S.; Snapper, M. L.; Schreiber, S. L., GTP-dependent binding of the antiproliferative agent didemnin to elongation factor 1 alpha. J. Biol. Chem. 1994, 269 (22), 15411-15414.
4. Shao, S.; Murray, J.; Brown, A.; Taunton, J.; Ramakrishnan, V.; Hegde, R. S., Decoding Mammalian Ribosome-mRNA States by Translational GTPase Complexes. Cell 2016, 167 (5), 1229-1240 e1215.
5. Lindqvist, L.; Robert, F.; Merrick, W.; Kakeya, H.; Fraser, C.; Osada, H.; Pelletier, J., Inhibition of translation by cytotrienin A—a member of the ansamycin family. RNA 2010, 16 (12), 2404-2413.
6. Krastel, P.; Roggo, S.; Schirle, M.; Ross, N. T.; Perruccio, F.; Aspesi, P., Jr.; Aust, T.; Buntin, K.; Estoppey, D.; Liechty, B., et al., Nannocystin A: an Elongation Factor 1 Inhibitor from Myxobacteria with Differential Anti-Cancer Properties. Angew. Chem. Int. Ed. 2015, 54 (35), 10149-10154.
7. Spicka, I.; Ocio, E. M.; Oakervee, H. E.; Greil, R.; Banh, R. H.; Huang, S. Y.; D'Rozario, J. M.; Dimopoulos, M. A.; Martinez, S.; Extremera, S., et al., Randomized phase III study (ADMYRE) of plitidepsin in combination with dexamethasone vs. dexamethasone alone in patients with relapsed/refractory multiple myeloma. Ann Hematol 2019, 98 (9), 2139-2150.
8. Blunt, J.; Cole, T.; Munro, M.; Sun, L.; Weber, J.-F. R.; Ramasamy, K.; Bakar, H. A.; Majeed, A. B. B. A., Bioactive compounds. International Patent WO 2010/062159 A1.
9. Shimokawa, K.; Mashima, I.; Asai, A.; Yamada, K.; Kita, M.; Uemura, D., (−)-Ternatin, a highly N-methylated cyclic heptapeptide that inhibits fat accumulation: structure and synthesis. Tetrahedron Lett. 2006, 47 (26), 4445-4448.
10. Carelli, J. D.; Sethofer, S. G.; Smith, G. A.; Miller, H. R.; Simard, J. L.; Merrick, W. C.; Jain, R. K.; Ross, N. T.; Taunton, J., Ternatin and improved synthetic variants kill cancer cells by targeting the elongation factor-1A ternary complex. Elife 2015, 4:e10222.
11. Gordon, D. E.; Jang, G. M.; Bouhaddou, M.; Xu, J.; Obernier, K.; White, K. M.; O'Meara, M. J.; Rezelj, V. V.; Guo, J. Z.; Swaney, D. L., et al., A SARS-CoV-2 protein interaction map reveals targets for drug repurposing. Nature 2020, 583 (7816), 459-468.
12. Deboves, H. J. C.; Grabowska, U.; Rizzo, A.; Jackson, R. F. W., A new route to hydrophobic amino acids using copper-promoted reactions of serine-derived organozinc reagents. Journal of the Chemical Society-Perkin Transactions 1 2000, (24), 4284-4292.
13. Dunn, M. J.; Jackson, R. F. W.; Pietruszka, J.; Turner, D., Synthesis of Enantiomerically Pure Unsaturated .alpha.-Amino Acids Using Serine-Derived Zinc/Copper Reagents. The Journal of Organic Chemistry 1995, 60 (7), 2210-2215.
14. Liu, J.; Xu, Y.; Stoleru, D.; Salic, A., Imaging protein synthesis in cells and tissues with an alkyne analog of puromycin. Proc. Natl. Acad. Sci. U.S.A. 2012, 109 (2), 413-418.
15. Vauquelin, G., Rebinding: or why drugs may act longer in vivo than expected from their in vitro target residence time. Expert Opin Drug Discov 2010, 5 (10), 927-941.
16. Copeland, R. A., The drug-target residence time model: a 10-year retrospective. Nat Rev Drug Discov 2016, 15 (2), 87-95.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound having the formula: 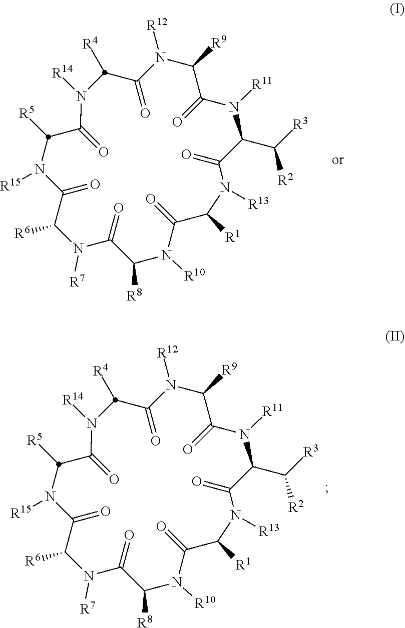
R¹ is substituted or unsubstituted alkyl or substituted or unsubstituted heteroalkyl;
R² is —OCX²₃, —OCH₂X², —OCHX²₂, —SR^{2B}, —NR^{2A}R^{2B}, or —OR^{2B};
R^{2A} and R^{2B} are independently
hydrogen, —CCl₃, —CF₃, —CHF₂, —CH₂F, —CN, —OH, —NH₂, —COOH, —CONH₂, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} and R^{2B} substituents bonded to the same nitrogen atom may optionally be joined to form a substituted or unsubstituted heterocycloalkyl or substituted or unsubstituted heteroaryl;
R³ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ and R⁷ are independently hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ and R⁷ substituents may optionally be joined to form, in combination with the —CHN— connecting the two substituents, a substituted or unsubstituted heterocycloalkyl;
R⁸ and R⁹ are independently hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰, R¹¹, R¹², R¹³, R¹⁴, and R¹⁵ are independently
hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X² is independently —F, —Cl, —Br, or —I;
or a pharmaceutically acceptable salt thereof.

2.-5. (canceled)

6. The compound of claim 1, having the formula: 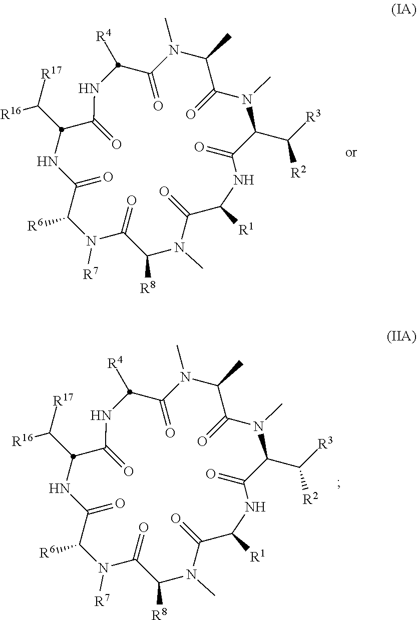
wherein
R¹ is substituted or unsubstituted alkyl or substituted or unsubstituted heteroalkyl;
R³ is substituted or unsubstituted alkyl or substituted or unsubstituted cycloalkyl;
R⁴ is substituted or unsubstituted alkyl or substituted or unsubstituted cycloalkyl;
R⁶ and R⁷ are independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, or substituted or unsubstituted heteroalkyl;
R⁶ and R⁷ substituents may optionally be joined to form, in combination with the —CHN— connecting the two substituents, a substituted or unsubstituted heterocycloalkyl;
R⁸ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or substituted or unsubstituted alkyl;
R¹⁶ is —OCX¹⁶₃, —OCH₂X¹⁶, —OCHX¹⁶₂, —SR^{16B}, —NR^{16A}R^{16B}, or —OR^{16B};
R^{16A} and R^{16B} are independently
hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{16A} and R^{16B} substituents bonded to the same nitrogen atom may optionally be joined to form a substituted or unsubstituted heterocycloalkyl or substituted or unsubstituted heteroaryl;
R¹⁷ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹⁶ is independently —F, —Cl, —Br, or —I;
or a pharmaceutically acceptable salt thereof.

7-21. (canceled)

22. The compound of claim 1, having the formula:

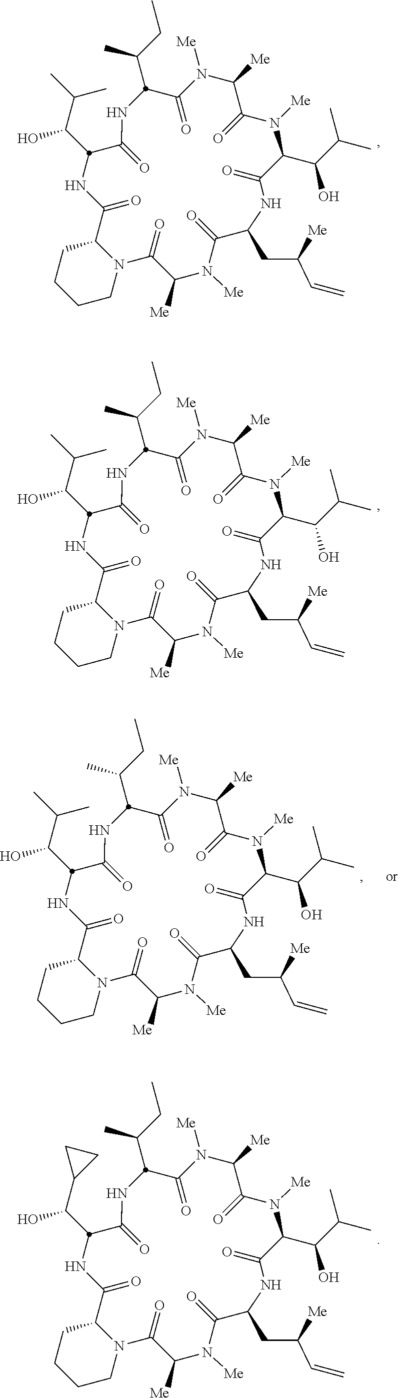

23.-25. (canceled)

26. A pharmaceutical composition comprising the compound of claim 1, or a pharmaceutically acceptable salt thereof, and a pharmaceutically acceptable excipient.

27. A method of decreasing the level of Elongation Factor 1-alpha protein activity in a subject, said method comprising administering a compound of claim 1 to said subject.

28. A method of inhibiting cancer growth in a subject in need thereof, said method comprising administering to the subject in need thereof an effective amount of a compound of claim 1, or a pharmaceutically acceptable salt thereof.

29. A method of inhibiting cancer cell growth, said method comprising contacting the cancer cell with an effective amount of a compound of claim 1.

30. A method of treating a cancer in a subject in need thereof, said method comprising administering to the subject in need thereof an effective amount of a compound of claim 1.

31. The method of claim 30, wherein the cancer is a hematological cancer, wherein the hematological cancer is acute lymphoblastic leukemia, acute myeloid leukemia, chronic myelogenous leukemia, or multiple myeloma.

32. (canceled)

33. The method of claim 30, wherein the cancer is resistant to treatment with a tyrosine kinase inhibitor, wherein the tyrosine kinase inhibitor is bosutinib, crizotinib, dasatinib, erlotinib, gefitinib, imatinib, afatinib, neratinib, lapatinib, nilotinib, ponatinib, midostaurin, gilteritinib, osimertinib, ibrutinib, or acalabrutinib.

34. (canceled)

35. The method of claim 30, further comprising co-administering an anti-cancer agent to said subject in need.

36. A compound having the formula: 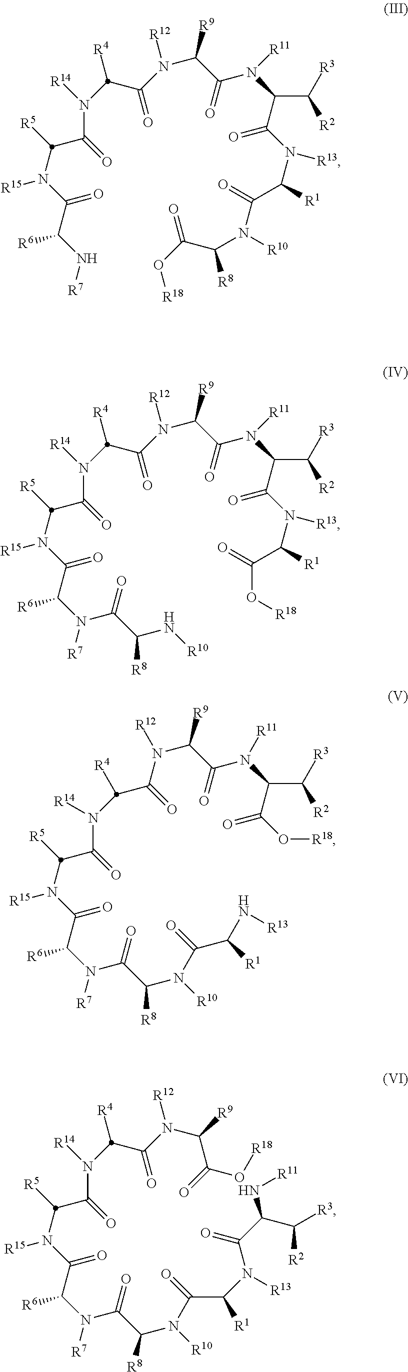 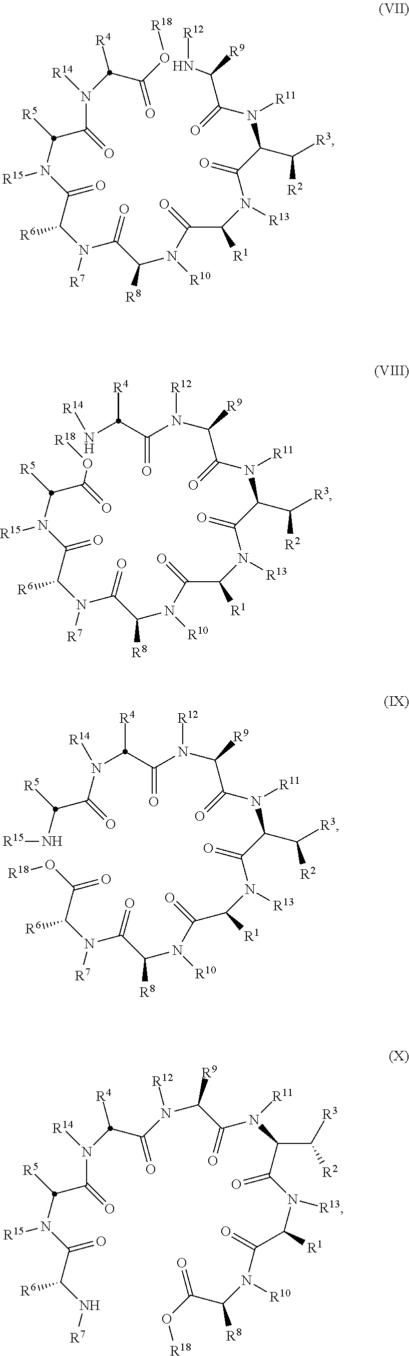 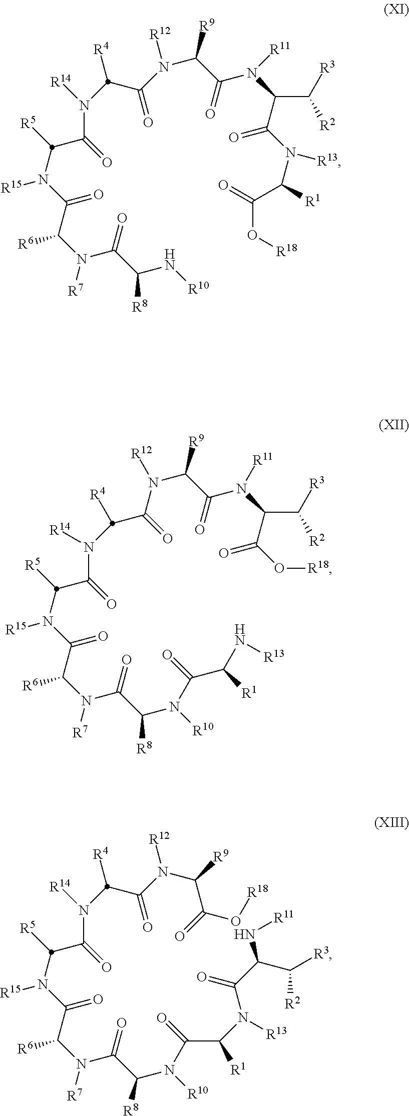 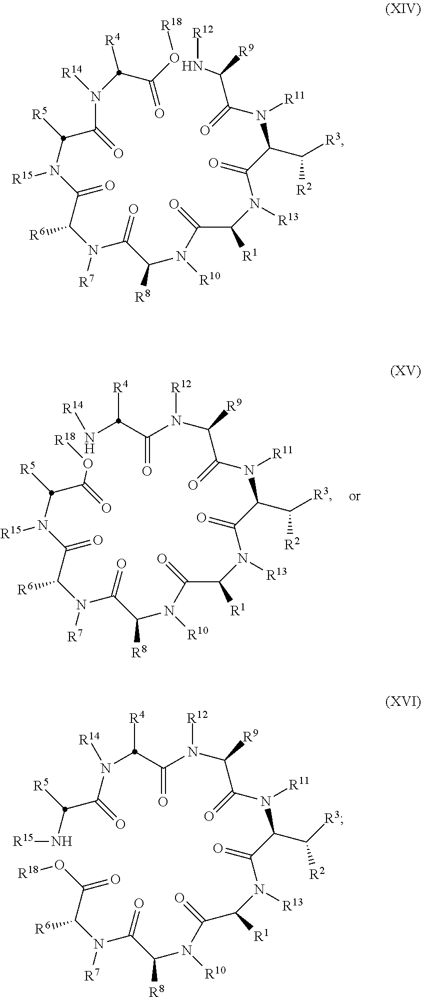
R¹ is substituted or unsubstituted alkyl or substituted or unsubstituted heteroalkyl;
R² is —OCX²₃, —OCH₂X², —OCHX²₂, —SR^{2B}, —NR^{2A}R^{2B}, or —OR^{2B};
R^{2A} and R^{2B} are independently
hydrogen, —CCl₃, —CF₃, —CHF₂, —CH₂F, —CN, —OH, —NH₂, —COOH, —CONH₂, —OCCl₃, —OCF₃, —OCHF₂, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} and R^{2B} Substituents bonded to the same nitrogen atom may optionally be joined to form a substituted or unsubstituted heterocycloalkyl or substituted or unsubstituted heteroaryl;
R³ is hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)N₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is —CCl₃, —CBr₃, —CF₃, —CI₃, —CHCl₂, —CHBr₂, —CHF₂, —CHI₂, —CH₂Cl, —CH₂Br, —CH₂F, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ and R⁷ are independently hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ and R⁷ substituents may optionally be joined to form, in combination with the —CHN— connecting the two substituents, a substituted or unsubstituted heterocycloalkyl;
R⁸ and R⁹ are independently hydrogen,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰, R¹¹, R¹², R¹³, R¹⁴, and R¹⁵ are independently
hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X² is independently —F, —Cl, —Br, or —I;
R¹⁸ is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a resin moiety;
or a pharmaceutically acceptable salt thereof.

37.-56. (canceled)

57. The compound of claim 36, having the formula:

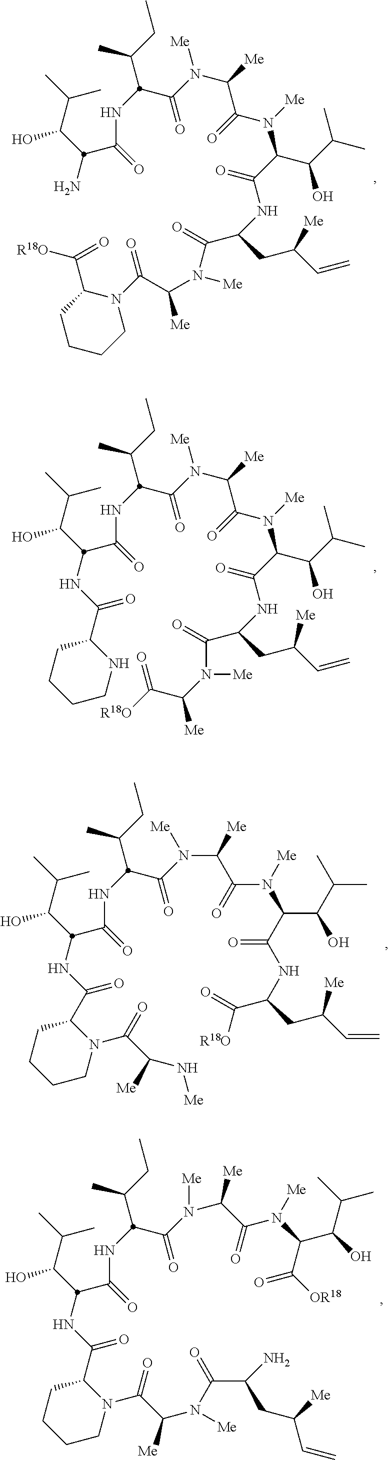

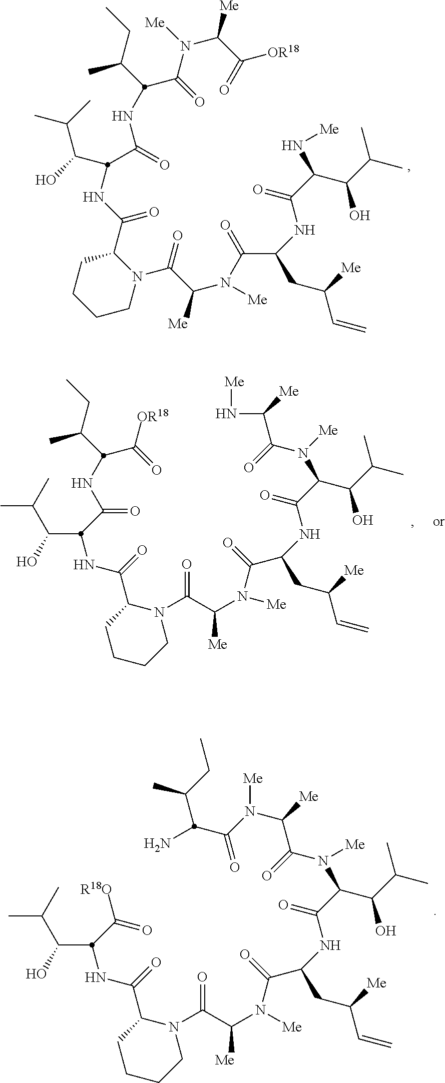

58. The compound of claim 36, having the formula:

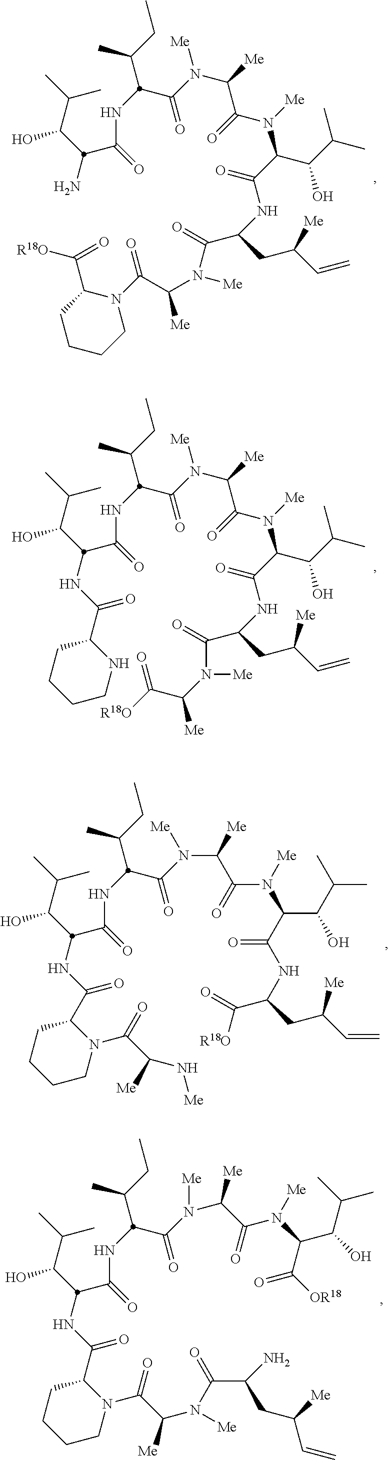

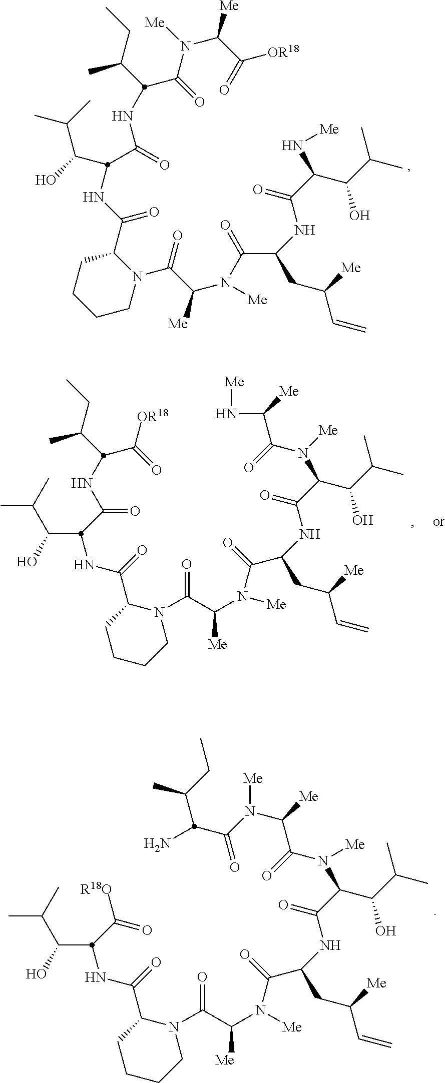

59.-63. (canceled)

64. A method of treating a viral infection in a subject in need thereof, the method comprising administering to the subject in need thereof an effective amount of a compound of claim 1, or a pharmaceutically acceptable salt thereof.

65.-70. (canceled)

71. A method of treating acute respiratory distress syndrome (ARDS) in a subject in need thereof, the method comprising administering to the subject in need thereof an effective amount of a compound of claim 1, or a pharmaceutically acceptable salt thereof.

72. A method of treating a coronavirus disease in a subject in need thereof, the method comprising administering to the subject in need thereof an effective amount of a compound of claim 1, or a pharmaceutically acceptable salt thereof.

73. A method of treating a SARS-CoV-2 infection in a subject in need thereof, the method comprising administering to the subject in need thereof an effective amount of a compound of claim 1, or a pharmaceutically acceptable salt thereof.

74. A method of treating a SARS-CoV-2 associated disease in a subject in need thereof, the method comprising administering to the subject in need thereof an effective amount of a compound of claim 1, or a pharmaceutically acceptable salt thereof.

75. A method of treating arrhythmia in a subject in need thereof, the method comprising administering to the subject in need thereof an effective amount of a compound of claim 1, or a pharmaceutically acceptable salt thereof.
